(19) United States
(12) Patent Application Publication
(10) Pub. No.: US 2024/0101693 A1
(43) Pub. Date: Mar. 28, 2024

(54) ANTAGONISTIC ANTI-TUMOR NECROSIS FACTOR RECEPTOR SUPERFAMILY ANTIBODIES

(71) Applicant: The General Hospital Corporation, Boston, MA (US)

(72) Inventor: Denise L. FAUSTMAN, Boston, MA (US)

(21) Appl. No.: 18/273,395

(22) PCT Filed: Jan. 21, 2022
(86) PCT No.: PCT/US2022/013273

§ 371 Date: Jul. 20, 2023

Related U.S. Application Data
(60) Provisional application No. 63/140,081, filed on Jan. 21, 2021.

(51) Int. Cl. C07K 16/28

(52) U.S. Cl. CPC C07K 16/2878; C07K 2317/34; C07K 2317/53; C07K 2317/622; C07K 2317/76

(57) ABSTRACT

The disclosure features antagonistic TNFR superfamily polypeptides, such as antibodies and antigen-binding fragments thereof, and the use of these polypeptides to inhibit the downstream signaling of TNFR superfamily members. The antibodies and antigen-binding fragments thereof can be used to treat a wide variety of cancers, infectious diseases, autoimmune disorders, obesity, type 2 diabetes, neurological disorders, and osteoporosis.

### SEQUENCE LISTING

The instant application contains a Sequence Listing which has been submitted electronically in ASCII format and is hereby incorporated by reference in its entirety. Said ASCII copy, created on Dec. 2, 2021, is named ‘00786-586WO2_Sequence_Listing_12_2_21_ST25’ and is 136,184 bytes in size.

### BACKGROUND OF THE DISCLOSURE

The use of naturally-occurring and genetically engineered T lymphocytes is a prominent paradigm for ameliorating various human pathologies. For instance, while traditional therapeutic platforms for the treatment of cancer include surgical removal of tumor mass, radiation therapy, and administration of chemotherapeutics (Shewach et al., Chem. Rev. 109:2859-2861, 2009), the last decade has witnessed a resurgence in the application of adoptive immunotherapy to cancer treatment regimens. With the advent of chimeric antigen receptor (CAR-T) therapy, new methods have emerged for the infusion of autologous and allogeneic tumor-reactive T cells to subjects (June, J. Clin. Invest. 117:1466-1476, 2007). CAR-T therapies harness the resources of the adaptive immune response in order to promote cancer cell cytotoxicity and eradicate tumor material. A common motif in adoptive immunotherapy is the use of T cells that exhibit the ability to selectively potentiate cytotoxicity in cells that display distinct tumor antigens. Examples of this technique include the administration of tumor-infiltrating lymphocytes (Dudley et al., J. Immunother. 26:332-342, 2003), as well as autologous or allogeneic T cells that have been genetically re-engineered so as to exhibit reactivity with a tumor-specific antigen (Yee et al., PNAS 99:16168-16173, 2002).

Despite the promise of T lymphocyte-based cancer immunotherapy, the development of this therapeutic platform has been hindered by the natural propensity of the immune system to suppress immune attacks mounted on self cells. Cancer cells express class I major histocompatibility complex (MHC) proteins that distinguish these cells from foreign cells. In order to prevent cell fratricide, regulatory T cells (T-reg cells) have evolved that suppress the activity of T cells that exhibit reactivity against “self” MHC antigens. T-reg cells represent a heterogeneous class of T cells that can be distinguished based on their unique surface protein presentation. The most well-understood populations of T-reg cells include CD4+, CD25+, FoxP3+ T-reg cells and CD17+ T-reg cells. The precise mechanisms by which these cells suppress autoreactive T cells is the subject of ongoing investigations, though it has been shown that certain classes of T-reg cells inhibit production of the proliferation-inducing cytokine IL-2 in target T cells and may additionally sequester IL-2 from autoreactive cells by virtue of the affinity of CD25 (a subdomain of the IL-2 receptor) for IL-2 (Josefowicz et al., Ann. Rev. Immun. 30:531-564, 2012).

Although T-reg cells play an important role in maintaining peripheral tolerance, the same biochemical features that underlie the ability of these cells to modulate autoreactive T cell activity also serve to undermine adoptive immunotherapy and the natural immune response by suppressing the activity of tumor-reactive T lymphocytes. The development of chemical modulators of T-reg cell activity has been the subject of many pharmacological investigations, as access to an agent capable of inhibiting T-reg-mediated T cell suppression could vastly improve the scope and efficacy of adoptive cancer immunotherapy, as well as improve the ability of the immune system to eradicate pathogenic organisms that give rise to infectious diseases.

Maintaining control of the cell-mediated and humoral immune responses is an important facet of healthy immune system activity. The aberrant regulation of lymphocyte driven immune responses (e.g., T cell and B cell driven immune reactions) has been associated with a wide array of human diseases, as the inappropriate mounting of an immune response against various self and foreign antigens plays a causal role in such pathologies as autoimmune disorders, asthma, allergic reactions, graft-versus-host disease, transplantation graft rejection, and a variety of other immunological disorders. These diseases are mediated by, e.g., T and B lymphocytes, that exhibit reactivity against self antigens and those derived from non-threatening sources, such as allergens or transplantation allografts.

There is a need for improved therapies for treating cell proliferation disorders, such as cancer, and a wide array of infectious diseases. There also exists a need for improved therapies that can augment T-reg cell survival and proliferation for use in treatments targeting such diseases as autoimmune disorders, graft-versus-host disease, allograft rejection, allergic reactions, asthma, and inflammation, among others.

### SUMMARY OF THE DISCLOSURE

Antagonistic polypeptides of the disclosure (e.g., single-chain polypeptides, antibodies, and antigen-binding fragments) that are specific for a human tumor necrosis factor receptor superfamily (TNFRSF) member, such as CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, inhibit the activation of a human TNFRSF member protein by binding this receptor (e.g., on the exterior surface of a T-reg cell, a cancer cell that expresses the TNFRSF member, a myeloid-derived suppressor cell (MDSC), a T cell, a B cell, a monocyte, a neutrophil, a platelet, a granulocyte, a bone marrow derived lymphoid cell, or a parenchymal cell), thereby preventing the protein from recruiting its cognate or natural ligand. The cognate or natural ligands of the TNFRSF member proteins described herein potentiate TNFRSF member protein signaling by nucleating a trimer of TNFRSF member proteins. It is this trimerization event that brings individual TNFRSF member proteins into close proximity and initiates signaling. TNFRSF member antagonist polypeptides (e.g., single-chain polypeptides, antibodies, and antibody fragments, such as anti-CD40 polypeptides that inhibit CD40 activation) can antagonize this interaction by binding the receptor and stabilizing an anti-parallel dimer conformation of the TNFRSF member protein. The stabilized anti-parallel dimer conformation of the TNFRSF member protein prevents the cognate or natural ligand from forming the active trimer conformation. Additionally, the disclosure features pharmaceutical compositions of the antagonistic polypeptides, as well as methods of treatment for a subject diagnosed with cancer, autoimmune disease, neurological disease, osteoporosis, and/or metabolic disorders (e.g., obesity, hyperlipidemia, and type 2 diabetes).

In a first aspect, the disclosure features polypeptides, such as a single-chain polypeptides, antibodies, antigen-binding fragments thereof, and constructs thereof, that specifically bind a human tumor necrosis factor receptor superfamily (TNFRSF) member protein selected from CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, and XEDAR.

In some embodiments, the antibody or antigen binding fragment thereof specifically binds: (a) CD40, wherein the antibody or antigen-binding fragment thereof specifically binds an epitope within amino acids 104-172 of SEQ ID NO: 4 (e.g., an epitope comprising five or more of amino acids 104-152 of SEQ ID NO: 4 and/or five or more of amino acids 123-172 of SEQ ID NO: 4, five or more of amino acids 114-142 of SEQ ID NO: 4 and/or five or more of amino acids 133-162 of SEQ ID NO: 4, five or more of amino acids 124-132 of SEQ ID NO: 4 and/or five or more of amino acids 143-152 of SEQ ID NO: 4) or an epitope with at least 80% or greater sequence identity thereto. In some embodiments, the antibody or antigen-binding fragment thereof specifically binds CD40, wherein the antibody or antigen-binding fragment thereof specifically binds an epitope comprising five or more of amino acids 124-132 of SEQ ID NO: 4. In some embodiments, the antibody or antigen-binding fragment thereof specifically binds CD40, wherein the antibody or antigen-binding fragment thereof specifically binds an epitope comprising five or more of amino acids 143-152 of SEQ ID NO: 4.

In some embodiments, the antibody or antigen binding fragment thereof specifically binds: (b) 4-1 BB, wherein the antibody or antigen-binding fragment thereof specifically binds an epitope within amino acids 81-149 of SEQ ID NO: 1 (e.g., an epitope comprising five or more of amino acids 91-139 of SEQ ID NO: 1, five or more of amino acids 101-129 of SEQ ID NO: 1) or an epitope with at least 80% or greater sequence identity thereto. In some embodiments, the antibody or antigen-binding fragment thereof specifically binds 4-1 BB, wherein the antibody or antigen-binding fragment thereof specifically binds an epitope comprising five or more of amino acids 101-129 of SEQ ID NO: 1.

In some embodiments, the antibody or antigen binding fragment thereof specifically binds: (c) CD27, wherein the antibody or antigen-binding fragment thereof specifically binds an epitope within amino acids 21-92 of SEQ ID NO: 2 (e.g., an epitope comprising five or more of amino acids 21-67 of SEQ ID NO: 2 and/or five or more of amino acids 41-92 of SEQ ID NO: 2, five or more of amino acids 31-57 of SEQ ID NO: 2 and/or five or more of amino acids 51-82 of SEQ ID NO: 2, five or more of amino acids 41-47 of SEQ ID NO: 2 and/or five or more of amino acids 61-72 of SEQ ID NO: 2) or an epitope with at least 80% or greater sequence identity thereto. In some embodiments, the antibody or antigen-binding fragment thereof specifically binds CD27, wherein the antibody or antigen-binding fragment thereof specifically binds an epitope comprising five or more of amino acids 41-47 of SEQ ID NO: 2. In some embodiments, the antibody or antigen-binding fragment thereof specifically binds CD27, wherein the antibody or antigen-binding fragment thereof specifically binds an epitope comprising five or more of amino acids 61-72 of SEQ ID NO: 2.

In some embodiments, the antibody or antigen binding fragment thereof specifically binds: (d) CD30, wherein the antibody or antigen-binding fragment thereof specifically binds an epitope within amino acids 109-177 of SEQ ID NO: 3 (e.g., an epitope comprising five or more of amino acids 109-160 of SEQ ID NO: 3 and/or five or more of amino acids 129-177 of SEQ ID NO: 3, five or more of amino acids 119-150 of SEQ ID NO: 3 and/or five or more of amino acids 139-167 of SEQ ID NO: 3, five or more of amino acids 129-140 of SEQ ID NO: 3 and/or five or more of amino acids 149-157 of SEQ ID NO: 3) or an epitope with at least 80% or greater sequence identity thereto. In some embodiments, the antibody or antigen-binding fragment thereof specifically binds CD30, wherein the antibody or antigen-binding fragment thereof specifically binds an epitope comprising five or more of amino acids 129-140 of SEQ ID NO: 3. In some embodiments, the antibody or antigen-binding fragment thereof specifically binds CD30, wherein the antibody or antigen-binding fragment thereof specifically binds an epitope comprising five or more of amino acids 149-157 of SEQ ID NO: 3.

In some embodiments, the antibody or antigen binding fragment thereof specifically binds: (e) DR6, wherein the antibody or antigen-binding fragment thereof specifically binds an epitope within amino acids 121-197 of SEQ ID NO: 5 (e.g., an epitope comprising five or more of amino acids 121-176 of SEQ ID NO: 5 and/or five or more of amino acids 149-197 of SEQ ID NO: 5, five or more of amino acids 131-166 of SEQ ID NO: 5 and/or five or more of amino acids 159-187 of SEQ ID NO: 5, five or more of amino acids 141-156 of SEQ ID NO: 5 and/or five or more of amino acids 169-177 of SEQ ID NO: 5) or an epitope with at least 80% or greater sequence identity thereto. In some embodiments, the antibody or antigen-binding fragment thereof specifically binds DR6, wherein the antibody or antigen-binding fragment thereof specifically binds an epitope comprising five or more of amino acids 141-156 of SEQ ID NO: 5. In some embodiments, the antibody or antigen-binding fragment thereof specifically binds DR6, wherein the antibody or antigen-binding fragment thereof specifically binds an epitope comprising five or more of amino acids 169-177 of SEQ ID NO: 5.

In some embodiments, the antibody or antigen binding fragment thereof specifically binds: (f) EDAR, wherein the antibody or antigen-binding fragment thereof specifically binds an epitope within amino acids 71-142 of SEQ ID NO: 6 (e.g., an epitope comprising five or more of amino acids 71-122 of SEQ ID NO: 6 and/or five or more of amino acids 91-142 of SEQ ID NO: 6, five or more of amino acids 81-112 of SEQ ID NO: 6 and/or five or more of amino acids 101-132 of SEQ ID NO: 6, five or more of amino acids 91-102 of SEQ ID NO: 6 and/or five or more of amino acids 111-122 of SEQ ID NO: 6) or an epitope with at least 80% or greater sequence identity thereto. In some embodiments, the antibody or antigen-binding fragment thereof specifically binds EDAR, wherein the antibody or antigen-binding fragment thereof specifically binds an epitope comprising five or more of amino acids 91-102 of SEQ ID NO: 6. In some embodiments, the antibody or antigen-binding fragment thereof specifically binds EDAR, wherein the antibody or antigen-binding fragment thereof specifically binds an epitope comprising five or more of amino acids 111-122 of SEQ ID NO: 6.

In some embodiments, the antibody or antigen binding fragment thereof specifically binds: (g) Fas, wherein the antibody or antigen-binding fragment thereof specifically binds an epitope an epitope within amino acids 128-193 of SEQ ID NO: 7 (e.g., an epitope comprising five or more of amino acids 128-176 of SEQ ID NO: 7 and/or five or more of amino acids 145-193 of SEQ ID NO: 7, five or more of amino acids 138-166 of SEQ ID NO: 7 and/or five or more of amino acids 155-183 of SEQ ID NO: 7, five or more of amino acids 148-156 of SEQ ID NO: 7 and/or five or more of amino acids 165-173 of SEQ ID NO: 7) or an epitope with at least 80% or greater sequence identity thereto. In some embodiments, the antibody or antigen-binding fragment thereof specifically binds Fas, wherein the antibody or antigen-binding fragment thereof specifically binds an epitope comprising five or more of amino acids 148-156 of SEQ ID NO: 7. In some embodiments, the antibody or antigen-binding fragment thereof specifically binds Fas, wherein the antibody or antigen-binding fragment thereof specifically binds an epitope comprising five or more of amino acids 165-173 of SEQ ID NO: 7.

In some embodiments, the antibody or antigen binding fragment thereof specifically binds: (h) GITR, wherein the antibody or antigen-binding fragment thereof specifically binds an epitope within amino acids 72-141 of SEQ ID NO: 8 (e.g., an epitope comprising five or more of amino acids 72-121 of SEQ ID NO: 8 and/or five or more of amino acids 92-141 of SEQ ID NO: 8, five or more of amino acids 82-111 of SEQ ID NO: 8 and/or five or more of amino acids 102-131 of SEQ ID NO: 8, five or more of amino acids 92-101 of SEQ ID NO: 8 and/or five or more of amino acids 112-121 of SEQ ID NO: 8) or an epitope with at least 80% or greater sequence identity thereto. In some embodiments, the antibody or antigen-binding fragment thereof specifically binds GITR, wherein the antibody or antigen-binding fragment thereof specifically binds an epitope comprising five or more of amino acids 92-101 of SEQ ID NO: 8. In some embodiments, the antibody or antigen-binding fragment thereof specifically binds GITR, wherein the antibody or antigen-binding fragment thereof specifically binds an epitope comprising five or more of amino acids 112-121 of SEQ ID NO: 8.

In some embodiments, the antibody or antigen binding fragment thereof specifically binds: (i) HVEM, wherein the antibody or antigen-binding fragment thereof specifically binds an epitope within amino acids 122-190 of SEQ ID NO: 9 (e.g., an epitope comprising five or more of amino acids 122-172 of SEQ ID NO: 9 and/or five or more of amino acids 142-190 of SEQ ID NO: 9, five or more of amino acids 132-162 of SEQ ID NO: 9 and/or five or more of amino acids 152-180 of SEQ ID NO: 9, five or more of amino acids 142-152 of SEQ ID NO: 9 and/or five or more of amino acids 162-170 of SEQ ID NO: 9) or an epitope with at least 80% or greater sequence identity thereto. In some embodiments, the antibody or antigen-binding fragment thereof specifically binds HVEM, wherein the antibody or antigen-binding fragment thereof specifically binds an epitope comprising five or more of amino acids 142-152 of SEQ ID NO: 9. In some embodiments, the antibody or antigen-binding fragment thereof specifically binds HVEM, wherein the antibody or antigen-binding fragment thereof specifically binds an epitope comprising five or more of amino acids 162-170 of SEQ ID NO: 9.

In some embodiments, the antibody or antigen binding fragment thereof specifically binds: (j) LT beta receptor, wherein the antibody or antigen-binding fragment thereof specifically binds an epitope within amino acids 127-196 of SEQ ID NO: 10 (e.g., an epitope comprising five or more of amino acids 127-175 of SEQ ID NO: 10 and/or five or more of amino acids 147-196 of SEQ ID NO: 10, five or more of amino acids 137-165 of SEQ ID NO: 10 and/or five or more of amino acids 157-186 of SEQ ID NO: 10, five or more of amino acids 147-155 of SEQ ID NO: 10 and/or five or more of amino acids 167-176 of SEQ ID NO: 10) or an epitope with at least 80% or greater sequence identity thereto. In some embodiments, the antibody or antigen-binding fragment thereof specifically binds LT beta receptor, wherein the antibody or antigen-binding fragment thereof specifically binds an epitope comprising five or more of amino acids 147-155 of SEQ ID NO: 10. In some embodiments, the antibody or antigen-binding fragment thereof specifically binds LT beta receptor, wherein the antibody or antigen-binding fragment thereof specifically binds an epitope comprising five or more of amino acids 167-176 of SEQ ID NO: 10.

In some embodiments, the antibody or antigen binding fragment thereof specifically binds: (k) NGFR, wherein the antibody or antigen-binding fragment thereof specifically binds an epitope within amino acids 108-176 of SEQ ID NO: 11 (e.g., an epitope comprising five or more of amino acids 108-155 of SEQ ID NO: 11 and/or five or more of amino acids 126-176 of SEQ ID NO: 11, five or more of amino acids 118-145 of SEQ ID NO: 11 and/or five or more of amino acids 136-166 of SEQ ID NO: 11, five or more of amino acids 128-135 of SEQ ID NO: 11 and/or five or more of amino acids 146-156 of SEQ ID NO: 11) or an epitope with at least 80% or greater sequence identity thereto. In some embodiments, the antibody or antigen-binding fragment thereof specifically binds NGFR, wherein the antibody or antigen-binding fragment thereof specifically binds an epitope comprising five or more of amino acids 128-135 of SEQ ID NO: 11. In some embodiments, the antibody or antigen-binding fragment thereof specifically binds NGFR, wherein the antibody or antigen-binding fragment thereof specifically binds an epitope comprising five or more of amino acids 146-156 of SEQ ID NO: 11.

In some embodiments, the antibody or antigen binding fragment thereof specifically binds: (I) OPG, wherein the antibody or antigen-binding fragment thereof specifically binds an epitope within amino acids 103-171 of SEQ ID NO: 12 (e.g., an epitope comprising five or more of amino acids 103-151 of SEQ ID NO: 12 and/or five or more of amino acids 122-171 of SEQ ID NO: 12, five or more of amino acids 113-141 of SEQ ID NO: 12 and/or five or more of amino acids 132-161 of SEQ ID NO: 12, five or more of amino acids 123-131 of SEQ ID NO: 12 and/or five or more of amino acids 142-151 of SEQ ID NO: 12) or an epitope with at least 80% or greater sequence identity thereto. In some embodiments, the antibody or antigen-binding fragment thereof specifically binds OPG, wherein the antibody or antigen-binding fragment thereof specifically binds an epitope comprising five or more of amino acids 123-131 of SEQ ID NO: 12. In some embodiments, the antibody or antigen-binding fragment thereof specifically binds OPG, wherein the antibody or antigen-binding fragment thereof specifically binds an epitope comprising five or more of amino acids 142-151 of SEQ ID NO: 12.

In some embodiments, the antibody or antigen binding fragment thereof specifically binds: (m) OX40, wherein the antibody or antigen-binding fragment thereof specifically binds an epitope within amino acids 98-144 of SEQ ID NO: 13 (e.g., an epitope comprising five or more of amino acids 98-154 of SEQ ID NO: 13, five or more of amino acids 108-144 of SEQ ID NO: 13, five or more of amino acids 118-134 of SEQ ID NO: 13), or an epitope with at least 80% or greater sequence identity thereto. In some embodiments, the antibody or antigen-binding fragment thereof specifically binds OX40, wherein the antibody or antigen-binding fragment thereof specifically binds an epitope comprising five or more of amino acids 118-134 of SEQ ID NO: 13.

In some embodiments, the antibody or antigen binding fragment thereof specifically binds: (n) RANK, wherein the antibody or antigen-binding fragment thereof specifically binds an epitope within amino acids 112-180 of SEQ ID NO: 14 (e.g., an epitope comprising five or more of amino acids 112-159 of SEQ ID NO: 14 and/or five or more of amino acids 131-180 of SEQ ID NO: 14, five or more of amino acids 122-149 of SEQ ID NO: 14 and/or five or more of amino acids 141-170 of SEQ ID NO: 14, five or more of amino acids 132-139 of SEQ ID NO: 14 and/or five or more of amino acids 151-160 of SEQ ID NO: 14) or an epitope with at least 80% or greater sequence identity thereto. In some embodiments, the antibody or antigen-binding fragment thereof specifically binds RANK, wherein the antibody or antigen-binding fragment thereof specifically binds an epitope comprising five or more of amino acids 132-139 of SEQ ID NO: 14. In some embodiments, the antibody or antigen-binding fragment thereof specifically binds OPG, wherein the antibody or antigen-binding fragment thereof specifically binds an epitope comprising five or more of amino acids 151-160 of SEQ ID NO: 14.

In some embodiments, the antibody or antigen binding fragment thereof specifically binds: (o) RELT (19L), wherein the antibody or antigen-binding fragment thereof specifically binds an epitope within amino acids 50-118 of SEQ ID NO: 15 (e.g., an epitope comprising five or more of amino acids 50-97 of SEQ ID NO: 15 and/or five or more of amino acids 70-118 of SEQ ID NO: 15, five or more of amino acids 60-87 of SEQ ID NO: 15 and/or five or more of amino acids 80-108 of SEQ ID NO: 15, five or more of amino acids 70-77 of SEQ ID NO: 15 and/or five or more of amino acids 90-98 of SEQ ID NO: 15) or an epitope with at least 80% or greater sequence identity thereto. In some embodiments, the antibody or antigen-binding fragment thereof specifically binds RELT (19L), wherein the antibody or antigen-binding fragment thereof specifically binds an epitope comprising five or more of amino acids 70-77 of SEQ ID NO 15. In some embodiments, the antibody or antigen-binding fragment thereof specifically binds RELT (19L), wherein the antibody or antigen-binding fragment thereof specifically binds an epitope comprising five or more of amino acids 90-98 of SEQ ID NO: 15.

In some embodiments, the antibody or antigen binding fragment thereof specifically binds: (p) TNFR1, wherein the antibody or antigen-binding fragment thereof specifically binds an epitope within amino acids 84-153 of SEQ ID NO: 16 (e.g., an epitope comprising five or more of amino acids 84-132 of SEQ ID NO: 16 and/or five or more of amino acids 104-153 of SEQ ID NO: 16, five or more of amino acids 94-122 of SEQ ID NO: 16 and/or five or more of amino acids 114-143 of SEQ ID NO: 16, five or more of amino acids 104-112 of SEQ ID NO: 16 and/or five or more of amino acids 124-133 of SEQ ID NO: 16) or an epitope with at least 80% or greater sequence identity thereto. In some embodiments, the antibody or antigen-binding fragment thereof specifically binds TNFR1, wherein the antibody or antigen-binding fragment thereof specifically binds an epitope comprising five or more of amino acids 104-112 of SEQ ID NO: 16. In some embodiments, the antibody or antigen-binding fragment thereof specifically binds TNFR1, wherein the antibody or antigen-binding fragment thereof specifically binds an epitope comprising five or more of amino acids 124-133 of SEQ ID NO: 16.

In some embodiments, the antibody or antigen binding fragment thereof specifically binds: (q) TRAIL-R2 (TNFRSF10B), wherein the antibody or antigen-binding fragment thereof specifically binds an epitope within amino acids 135-185-206 of SEQ ID NO: 17 (e.g., an epitope comprising five or more of amino acids 135-185 of SEQ ID NO: 17 and/or five or more of amino acids 158-206 of SEQ ID NO: 17, five or more of amino acids 145-175 of SEQ ID NO: 17 and/or five or more of amino acids 168-196 of SEQ ID NO: 17, five or more of amino acids 155-165 of SEQ ID NO: 17 and/or five or more of amino acids 178-186 of SEQ ID NO: 17) or an epitope with at least 80% or greater sequence identity thereto. In some embodiments, the antibody or antigen-binding fragment thereof specifically binds TRAIL-R2 (TNFRSF10B), wherein the antibody or antigen-binding fragment thereof specifically binds an epitope comprising five or more of amino acids 155-165 of SEQ ID NO: 17. In some embodiments, the antibody or antigen-binding fragment thereof specifically binds TRAIL-R2 (TNFRSF10B), wherein the antibody or antigen-binding fragment thereof specifically binds an epitope comprising five or more of amino acids 178-186 of SEQ ID NO: 17.

In some embodiments, the antibody or antigen binding fragment thereof specifically binds: (r) TRAIL-R1 (TNFRSF10A), wherein the antibody or antigen-binding fragment thereof specifically binds an within amino acids 149-216 of SEQ ID NO: 18 (e.g., an epitope comprising five or more of amino acids 149-197 of SEQ ID NO: 18 and/or five or more of amino acids 168-216 of SEQ ID NO: 18, five or more of amino acids 159-187 of SEQ ID NO: 18 and/or five or more of amino acids 178-206 of SEQ ID NO: 18, five or more of amino acids 169-177 of SEQ ID NO: 18 and/or five or more of amino acids 188-196 of SEQ ID NO: 18) or an epitope with at least 80% or greater sequence identity thereto. In some embodiments, the antibody or antigen-binding fragment thereof specifically binds TRAIL-R1 (TNFRSF10A), wherein the antibody or antigen-binding fragment thereof specifically binds an epitope comprising five or more of amino acids 169-177 of SEQ ID NO: 18. In some embodiments, the antibody or antigen-binding fragment thereof specifically binds TRAIL-R1 (TNFRSF10A), wherein the antibody or antigen-binding fragment thereof specifically binds an epitope comprising five or more of amino acids 188-196 of SEQ ID NO: 18.

In some embodiments, the antibody or antigen binding fragment thereof specifically binds: (s) TRAIL-R4, wherein the antibody or antigen-binding fragment thereof specifically binds an epitope within amino acids 141-210 of SEQ ID NO: 19 (e.g., an epitope comprising five or more of amino acids 141-189 of SEQ ID NO: 19 and/or five or more of amino acids 161-210 of SEQ ID NO: 19, five or more of amino acids 151-179 of SEQ ID NO: 19 and/or five or more of amino acids 171-200 of SEQ ID NO: 19, five or more of amino acids 161-169 of SEQ ID NO: 19 and/or five or more of amino acids 181-190 of SEQ ID NO: 19) or an epitope with at least 80% or greater sequence identity thereto. In some embodiments, the antibody or antigen-binding fragment thereof specifically binds TRAIL-R4, wherein the antibody or antigen-binding fragment thereof specifically binds an epitope comprising five or more of amino acids 161-169 of SEQ ID NO: 19. In some embodiments, the antibody or antigen-binding fragment thereof specifically binds TRAIL-R4, wherein the antibody or antigen-binding fragment thereof specifically binds an epitope comprising five or more of amino acids 181-190 of SEQ ID NO: 19.

In some embodiments, the antibody or antigen binding fragment thereof specifically binds: (t) TRAMP, wherein the antibody or antigen-binding fragment thereof specifically binds an epitope within amino acids 122-191 of SEQ ID NO: 20 (e.g., an epitope comprising five or more of amino acids 122-170 of SEQ ID NO: 20 and/or five or more of amino acids 142-191 of SEQ ID NO: 20, five or more of amino acids 132-160 of SEQ ID NO: 20 and/or five or more of amino acids 152-181 of SEQ ID NO: 20, five or more of amino acids 142-150 of SEQ ID NO: 20 and/or five or more of amino acids 162-171 of SEQ ID NO: 20) or an epitope with at least 80% or greater sequence identity thereto. In some embodiments, the antibody or antigen-binding fragment thereof specifically binds TRAMP, wherein the antibody or antigen-binding fragment thereof specifically binds an epitope comprising five or more of amino acids 142-150 of SEQ ID NO: 20. In some embodiments, the antibody or antigen-binding fragment thereof specifically binds TRAMP, wherein the antibody or antigen-binding fragment thereof specifically binds an epitope comprising five or more of amino acids 162-171 of SEQ ID NO: 20.

In some embodiments, the antibody or antigen binding fragment thereof specifically binds: (u) TROY, wherein the antibody or antigen-binding fragment thereof specifically binds an epitope within amino acids 31-102 of SEQ ID NO: 21 (e.g., an epitope comprising five or more of amino acids 31-79 of SEQ ID NO: 21 and/or five or more of amino acids 52-102 of SEQ ID NO: 21, five or more of amino acids 41-69 of SEQ ID NO: 21 and/or five or more of amino acids 62-92 of SEQ ID NO: 21, five or more of amino acids 51-59 of SEQ ID NO: 21 and/or five or more of amino acids 72-82 of SEQ ID NO: 21) or an epitope with at least 80% or greater sequence identity thereto. In some embodiments, the antibody or antigen-binding fragment thereof specifically binds TROY, wherein the antibody or antigen-binding fragment thereof specifically binds an epitope comprising five or more of amino acids 51-59 of SEQ ID NO: 21. In some embodiments, the antibody or antigen-binding fragment thereof specifically binds TROY, wherein the antibody or antigen-binding fragment thereof specifically binds an epitope comprising five or more of amino acids 72-82 of SEQ ID NO: 21.

In some embodiments, the antibody or antigen binding fragment thereof specifically binds: (v) XEDAR, wherein the antibody or antigen-binding fragment thereof specifically binds an epitope within amino acids 1-69 of SEQ ID NO: 22 (e.g., an epitope comprising five or more of amino acids 1-47 of SEQ ID NO: 22 and/or five or more of amino acids 22-69 of SEQ ID NO: 22, five or more of amino acids 10-37 of SEQ ID NO: 22 and/or five or more of amino acids 32-59 of SEQ ID NO: 22, five or more of amino acids 20-27 of SEQ ID NO: 22 and/or five or more of amino acids 42-49 of SEQ ID NO: 22) or an epitope with at least 80% or greater sequence identity thereto. In some embodiments, the antibody or antigen-binding fragment thereof specifically binds XEDAR, wherein the antibody or antigen-binding fragment thereof specifically binds an epitope comprising five or more of amino acids 20-27 of SEQ ID NO: 22. In some embodiments, the antibody or antigen-binding fragment thereof specifically binds XEDAR, wherein the antibody or antigen-binding fragment thereof specifically binds an epitope comprising five or more of amino acids 42-49 of SEQ ID NO: 22.

In some embodiments, the antibody or antigen-binding fragment thereof comprises a non-native constant region.

In some embodiments, the antibody or antigen-binding fragment thereof inhibits signaling associated with the TNFRSF member protein.

In some embodiments, the antibody or antigen-binding fragment thereof binds the TNFRSF member protein with a K_{d} of no greater than about 10 nM (e.g., no greater than about 1 nM). In some embodiments, the antibody or antigen-binding fragment thereof binds the TNFRSF member protein to form an antibody-antigen complex with a kₒₙ of at least about 10⁴ M⁻¹s⁻¹.

In some embodiments, the antibody or antigen-binding fragment thereof binds the TNFRSF member protein to form an antibody-antigen complex, and wherein the complex dissociates with a kₒₙ of no greater than about 10⁻³ s⁻¹.

In some embodiments, the antibody or antigen-binding fragment thereof has an isotype selected from the group consisting of IgG, IgA, IgM, IgD, and IgE. In some embodiments, the antibody or antigen-binding fragment thereof is an IgG isotype (e.g., IgG2 isotype).

In some embodiments, the polypeptides, such as single-chain polypeptides, antibodies, antigen-binding fragments thereof, and constructs thereof, contain a human IgG2 hinge region that lacks a cysteine residue at positions 232 and/or 233 of the amino acid sequence of the IgG2 hinge region. For example, the polypeptide (e.g., a single-chain polypeptide, antibody, antigen-binding fragment thereof, or construct thereof) may contain a human IgG2 hinge region having an amino acid other than cysteine, such as a serine residue, at positions 232 and/or 233 of the amino acid sequence of the IgG2 hinge region.

The polypeptide (e.g., a single-chain polypeptide, antibody, antigen-binding fragment thereof, or construct thereof) may contain, for example, a human IgG2 hinge region having an amino acid substitution or deletion at one or both of cysteine residues 232 and 233. The amino acid substitution may be a conservative amino acid substitution, such as a C232S and/or C233S amino acid substitution.

In some embodiments, the antibody or antigen-binding fragment thereof comprises antigen-binding sites separated from one another by a distance of at least about 133 Å (e.g., a distance of at least about 134 Å, about 139 Å, or about 150 Å).

In some embodiments, the antigen-binding sites are separated from one another by a distance of from about 133 Å to about 150 Å (e.g., a distance of from about 133 Å to about 145 Å, about 133 Å to about 139 Å, or about 134 Å to about 139 Å).

Also featured is a method of producing a TNFR2 antibody or antigen-binding fragment thereof by immunizing a non-human mammal with a peptide:
(a) comprising the amino acid sequence of any one of SEQ ID NOs: 67-153;
(b) containing between about 10 and about 30 continuous or discontinuous amino acids between positions 80 and 130 of SEQ ID NO: 154;
(c) comprising five or more continuous or discontinuous amino acid residues of amino acids 104-172 of SEQ ID NO: 4;
(d) comprising five or more continuous or discontinuous amino acid residues of amino acids 81-149 of SEQ ID NO: 1;
(e) comprising five or more continuous or discontinuous amino acid residues of amino acids 21-92 of SEQ ID NO: 2;
(f) comprising five or more continuous or discontinuous amino acid residues of amino acids 109-177 of SEQ ID NO: 3;
(g) comprising five or more continuous or discontinuous amino acid residues of amino acids 121-197 of SEQ ID NO: 5;
(h) comprising five or more continuous or discontinuous amino acid residues of amino acids 71-142 of SEQ ID NO: 6;
(i) comprising five or more continuous or discontinuous amino acid residues of amino acids 128-193 of SEQ ID NO: 7;
(j) comprising five or more continuous or discontinuous amino acid residues of amino acids 72-141 of SEQ ID NO: 8;
(k) comprising five or more continuous or discontinuous amino acid residues of amino acids 122-190 of SEQ ID NO: 9;
(I) comprising five or more continuous or discontinuous amino acid residues of amino acids 127-196 of SEQ ID NO: 10;
(m) comprising five or more continuous or discontinuous amino acid residues of amino acids 108-176 of SEQ ID NO: 11;
(m) comprising five or more continuous or discontinuous amino acid residues of amino acids 103-171 of SEQ ID NO: 12;
(n) comprising five or more continuous or discontinuous amino acid residues of amino acids 98-154 of SEQ ID NO: 13;
(o) comprising five or more continuous or discontinuous amino acid residues of amino acids 112-180 of SEQ ID NO: 14;
(p) comprising five or more continuous or discontinuous amino acid residues of amino acids 50-118 of SEQ ID NO: 52;
(q) comprising five or more continuous or discontinuous amino acid residues of amino acids 84-153 of SEQ ID NO: 16;
(r) comprising five or more continuous or discontinuous amino acid residues of amino acids 135-206 of SEQ ID NO: 17;
(s) comprising five or more continuous or discontinuous amino acid residues of amino acids 149-216 of SEQ ID NO: 18;
(t) comprising five or more continuous or discontinuous amino acid residues of amino acids 141-210 of SEQ ID NO: 19;
(u) comprising five or more continuous or discontinuous amino acid residues of amino acids 122-191 of SEQ ID NO: 20;
(v) comprising five or more continuous or discontinuous amino acid residues of amino acids 31-102 of SEQ ID NO: 21; or
(w) comprising five or more continuous or discontinuous amino acid residues of amino acids 1-69 of SEQ ID NO: 22,
and collecting serum containing the TNFR2 antagonist antibody or antigen-binding fragment thereof. Exemplary non-human mammals that can be immunized include a rabbit, mouse, rat, goat, guinea pig, hamster, horse, and sheep. In certain cases, the peptide used for immunization may contain the amino acid sequence KCRPG (SEQ ID NO: 68). In some cases, the peptide used for immunization may contain the amino acid sequence CAPLRKCR (SEQ ID NO: 67). Optionally, the peptide may contain the amino acid sequence KCRPGFGV (SEQ ID NO: 69).

In some embodiments, the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)2 molecule, and a tandem scFv (taFv). In some embodiments, the antibody or antigen-binding fragment thereof is a human, humanized, or chimeric antibody or antigen-binding fragment thereof.

In some embodiments, the antibody is conjugated to a therapeutic agent (e.g., a cytotoxic agent).

In some embodiments, the antibody or antigen-binding fragment thereof comprises a framework region from a human antibody or chimeric antibody.

In some embodiments, the antibody or antigen binding fragment thereof stabilizes an anti-parallel dimer conformation of the TNFRSF member.

In some embodiments, the antibody or antigen-binding fragment thereof destabilizes a trimeric conformation of the TNFRSF member.

In some embodiments, the antibody or antigen-binding fragment thereof reduces secretion of a soluble version of the TNFRSF member protein.

In some embodiments, the antibody or antigen-binding fragment thereof inhibits expression of one of more genes selected from the group consisting of CHUK, NFKBIE, NFKBIA, MAP3K11, TRAF2, TRAF3, relB, and cIAP2/BIRC3.

In some embodiments, the antibody or antigen-binding fragment thereof inhibits NFκB activation.

In some embodiments, the antibody or antigen-binding fragment thereof is capable of reducing or inhibiting proliferation of a population of T-reg cells and/or is capable of inducing proliferation of a population of CD8+ effector T cells.

In some embodiments, the antibody or antigen-binding fragment thereof is capable of reducing or inhibiting proliferation of a population of cancer cells, optionally wherein the cancer cells express the TNFRSF member protein.

In some embodiments, the cancer cells are selected from the group consisting of Hodgkin lymphoma cells, cutaneous non-Hodgkin lymphoma cells, T cell lymphoma cells, ovarian cancer cells, colon cancer cells, multiple myeloma cells, and renal cell carcinoma cells.

In some embodiments, the antibody or antigen-binding fragment thereof inhibits TNFRSF signaling in proliferating cells.

In some embodiments, the antibody or antigen-binding fragment thereof does not inhibit TNFRSF signaling in resting cells.

In some embodiments, the antibody or antigen-binding fragment thereof is capable of reducing or inhibiting proliferation of a population of myeloid-derived suppressor cells.

In some embodiments, the antibody or antigen-binding fragment thereof is capable of selectively reducing or inhibiting the proliferation of a population of T-reg cells expressing CD25^{Hi}.

In some embodiments, the antibody or antigen-binding fragment thereof is capable of reducing or inhibiting proliferation of a population of T-reg cells in the presence of a ligand for the TNFRSF member protein.

In some embodiments, the antibody or antigen-binding fragment thereof promotes proliferation of T-reg cells. In some embodiments, the antibody or antigen-binding fragment thereof directly kills, or promotes the death of, CD8+ cytotoxic T cells. In some embodiments, the antibody or antigen-binding fragment thereof is capable of reducing or inhibiting proliferation of a population of CD8+ cytotoxic T cells in the presence of a ligand for the TNFRSF member protein.

Also featured is a method of producing an antibody or antigen-binding fragment described herein by expressing a polynucleotide encoding the antibody or antigen-binding fragment in a host cell and recovering the antibody or antigen-binding fragment from host cell medium.

A second aspect features a construct comprising a first polypeptide domain and a second polypeptide domain, in which the first polypeptide domain and the second polypeptide domain are each, independently, an antigen-binding fragment described herein.

In some embodiments, the first polypeptide domain and the second polypeptide domain are bound by a covalent linker (e.g., an amide bond or a disulfide bond).

A third aspect features a polynucleotide encoding an antibody or antigen-binding fragment thereof described herein.

A fourth aspect features a polynucleotide encoding a construct described herein.

A fifth aspect features a vector comprising a polynucleotide described herein.

In some embodiments, the vector is an expression vector (e.g., a eukaryotic expression vector).

In some embodiments, the vector is a viral vector (e.g., adenovirus (Ad) (e.g., a serotype 1-60 adenovirus (e.g., a serotype 5, 26, 35, or 48 adenovirus)), retrovirus (e.g., a γ-retrovirus or a lentivirus), poxvirus, adeno-associated virus, baculovirus, herpes simplex virus, or a vaccinia virus (e.g., a modified vaccinia Ankara (MVA))).

A sixth aspect features an isolated host cell comprising a vector described herein.

In some embodiments, the host cell is a prokaryotic cell.

In some embodiments, the host cell is a eukaryotic cell (e.g., a mammalian cell (e.g., a CHO cell)).

A seventh aspect features a pharmaceutical composition comprising an antibody or antigen-binding fragment thereof described herein, a construct described herein, a polynucleotide described herein, a vector described herein, or a host cell described herein, and a pharmaceutically acceptable carrier or excipient.

In some embodiments, the pharmaceutical composition comprises an antibody or antigen biding fragment thereof described herein. In some embodiments, at least 50% (e.g., at least 75% (e.g., from about 75% to about 99.9%), 80% (e.g., from about 80% to about 99.9%), 85% (e.g., from about 85% to about 99.9%), 90% (e.g., from about 90% to about 99.9%), or at least 95% (e.g., from about 95% to about 99.9%)) of the antibody or antigen-binding fragment thereof in the pharmaceutical composition is present in a single disulfide-bonded isoform.

In some embodiments, the antibody or antigen-binding fragment thereof yields a single detectable band upon gel electrophoresis analysis performed under non-reducing conditions.

In some embodiments, the single disulfide-bonded isoform is IgG2-A.

In some embodiments, the antibody or antigen-binding fragment thereof is present in the pharmaceutical composition in an amount of from about 0.001 mg/ml to about 100 mg/ml.

In some embodiments, the pharmaceutical composition further comprises an additional therapeutic agent (e.g., an immunotherapy agent (e.g., an anti-CTLA-4 agent, an anti-PD-1 agent, an anti-PD-L1 agent, an anti-PD-L2 agent, a TNF-α cross-linking agent, a TRAIL cross-linking agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-LIGHT agent, an anti-DR4 agent, an anti-CR5 agent an anti-CD95 agent, an anti-TRAIL agent, an anti-BCMA agent, an anti-TACI agent, an anti-RANKL agent, or an anti-BAFFR agent, optionally wherein the immunotherapy agent is an anti-PD-1 antibody or an anti-PD-L1 antibody). In some embodiments, the immunotherapy agent is selected from the group consisting of an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-TWEAK antibody or antigen-binding fragment thereof, an anti-TWEAKR antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-LIGHT antibody or antigen-binding fragment thereof, an anti-DR4 antibody or antigen-binding fragment thereof, an anti-CR5 antibody or antigen-binding fragment thereof, an anti-CD95 antibody or antigen-binding fragment thereof, an anti-TRAIL antibody or antigen-binding fragment thereof, an anti-BCMA antibody or antigen-binding fragment thereof, an anti-TACI antibody or antigen-binding fragment thereof, an anti-RANKL antibody or antigen-binding fragment thereof, and an anti-BAFFR antibody or antigen-binding fragment thereof. In some embodiments, the immunotherapy agent is an anti-CTLA-4 agent or an anti-PD-1 agent. In some embodiments, the immunotherapy agent is an anti-CTLA-4 antibody (e.g., ipilimumab or tremelimumab) or antigen-binding fragment thereof or an anti-PD-1 antibody (e.g., nivolumab, pembrolizumab, avelumab, durvalumab, or atezolizumab) or antigen-binding fragment thereof.

In some embodiments, the additional therapeutic agent is a chimeric antigen receptor (CAR-T) agent, a chemotherapeutic agent, a small molecule anti-cancer agent, or a cancer vaccine.

An eighth aspect features a method of reducing or inhibiting an immune response mediated by a T-reg cell in a human by administering to the human an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of TRAMP, DR6, TRAIL-R3, TRAIL-R4, RANK, LT Beta, HVEM, CD30, TROY, or RELT (19L).

A ninth aspect features a method of treating a cell proliferation disorder in a human, the method comprising administering to the human an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of TRAMP, DR6, TRAIL-R3, TRAIL-R4, RANK, LT Beta, HVEM, CD30, TROY, or RELT (19L).

In some embodiments, the cell proliferation disorder is a cancer selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

In some embodiments, the cell proliferation disorder is a cancer selected from the group consisting of Hodgkin lymphoma, cutaneous non-Hodgkin lymphoma, T cell lymphoma, ovarian cancer, colon cancer, multiple myeloma, renal cell carcinoma, skin cancer, lung cancer, liver cancer, endometrial cancer, a cancer of the hematopoietic or lymphatic system, a cancer of the central nervous system, breast cancer, pancreatic cancer, stomach cancer, esophageal cancer, and a cancer of the upper gastrointestinal tract.

In some embodiments, the cell proliferation disorder is a cancer selected from the group consisting of T cell lymphoma, ovarian cancer, and colon cancer.

In some embodiments, the cell proliferation disorder is a cancer selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, Hodgkin lymphoma, anaplastic large cell lymphoma, hypopharyngeal cancer, islet cell tumors, pancreatic neuroendocrine tumors, wilms tumor and other childhood kidney tumors, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, renal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia.

In some embodiments, a sample obtained from the human has a ratio of T-reg cells to CD8+ T effector cells that is greater than a ratio of Treg cells to CD8+ T effector cells in a biological sample obtained from a human that does not have the cell proliferation disorder, optionally, wherein the ratio in the sample from the human is greater than the ratio in the sample from the human that does not have the cell proliferation disorder by 5%, 10%, 15%, 20%, 25%, 30%, 35%, 40%, 45%, 50%, 55%, 60%, 65%, 70%, 75%, 80%, 85%, 90%, 95%, 100%, 200%, or more. In some embodiments, the sample is a blood sample. In some embodiments, the sample is obtained from a tumor microenvironment. In some embodiments, the sample is a tumor biopsy.

A tenth aspect features a method of treating an infectious disease in a human, the method comprising administering to the human an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of TRAMP, DR6, TRAIL-R3, TRAIL-R4, HVEM, or RELT (19L). In some embodiments, the infectious disease is caused by one or more agents selected from the group consisting of a virus, a bacterium, a fungus, or a parasite.

In some embodiments, the infectious disease is caused by a virus selected from the group consisting of hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi's sarcoma associated-herpesvirus (KSHV), influenza virus, severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, mumps virus, poliovirus, human enterovirus (A, B, C, and D), hepatitis A virus, coxsackievirus, hepatitis B virus, human papilloma virus, adeno-associated virus, astrovirus, JC virus, BK virus, SV40 virus, Norwalk virus, rotavirus, human immunodeficiency virus (HIV), human T-lymphotropic virus Types I and II.

In some embodiments, the infectious disease is caused by a bacterium belonging to a genus selected from the group consisting of Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus.

In some embodiments, the infectious disease is caused by a fungus selected from the group consisting of Aspergillus, Candida, Malassezia, Trichosporon, Fusarium, Acremonium, Rhizopus, Mucor, Pneumocystis, and Absidia.

In some embodiments, the infectious disease is caused by a parasite selected from the group consisting of Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus.

In some embodiments, the human is further administered an additional therapeutic agent (e.g., an immunotherapy agent). In some embodiments, the immunotherapy agent is selected from the group consisting of an anti-CTLA-4 agent, an anti-PD-1 agent, an anti-PD-L1 agent, an anti-PD-L2 agent, a TNF-α cross-linking agent, a TRAIL cross-linking agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-LIGHT agent, an anti-DR4 agent, an anti-CR5 agent an anti-CD95 agent, an anti-TRAIL agent, an anti-BCMA agent, an anti-TACI agent, an anti-RANKL agent, and an anti-BAFFR agent, optionally wherein the immunotherapy agent is an anti-PD-1 antibody or an anti-PD-L1 antibody.

In some embodiments, the immunotherapy agent is selected from the group consisting of an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-TWEAK antibody or antigen-binding fragment thereof, an anti-TWEAKR antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-LIGHT antibody or antigen-binding fragment thereof, an anti-DR4 antibody or antigen-binding fragment thereof, an anti-CR5 antibody or antigen-binding fragment thereof, an anti-CD95 antibody or antigen-binding fragment thereof, an anti-TRAIL antibody or antigen-binding fragment thereof, an anti-BCMA antibody or antigen-binding fragment thereof, an anti-TACI antibody or antigen-binding fragment thereof, an anti-RANKL antibody or antigen-binding fragment thereof, and an anti-BAFFR antibody or antigen-binding fragment thereof.

In some embodiments, the immunotherapy agent is an anti-CTLA-4 agent or an anti-PD-1 agent. In some embodiments, the immunotherapy agent is an anti-CTLA-4 antibody (e.g., ipilimumab or tremelimumab) or antigen-binding fragment thereof or an anti-PD-1 antibody (e.g., nivolumab, pembrolizumab, avelumab, durvalumab, or atezolizumab) or antigen-binding fragment thereof.

In some embodiments, the additional therapeutic agent is a chimeric antigen receptor (CAR-T) agent, a chemotherapeutic agent, a small molecule anti-cancer agent, or a cancer vaccine. An eleventh aspect features a method of inhibiting an immune response mediated by a B cell or CD8+ T cell in a human subject comprising administering to the subject an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of CD40, TRAIL-R1 (TNFRSF10A), TRAIL-R2 (TNFRSF10B), DR6, NGFR, TNFR1, Fas, EDAR, RANK, CD27, 4-1BB, OX40, GITR, or XEDAR.

A twelfth aspect features a method of treating an immunological disease in a human subject by administering to the subject an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of CD40, TRAIL-R1 (TNFRSF10A), TRAIL-R2 (TNFRSF10B), DR6, NGFR, TNFR1, Fas, EDAR, RANK, CD27, 4-1 BB, OX40, GITR, or XEDAR.

In some embodiments, the subject is in need of a tissue or organ regeneration. In some embodiments, the tissue or organ is selected from the group consisting of a pancreas, salivary gland, pituitary gland, kidney, heart, lung, hematopoietic system, cranial nerves, heart, aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

In some embodiments, the immunological disease is selected from the group consisting of an autoimmune disease, a neurological condition, an allergy, asthma, macular degeneration, muscular atrophy, a disease related to miscarriage, atherosclerosis, bone loss, a musculoskeletal disease, obesity, a graft-versus-host disease, and an allograft rejection.

A thirteenth aspect features a method of treating obesity, hyperlipidemia, and/or type 2 diabetes in a human subject by administering to the subject an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of Fas.

A fourteenth aspect features a method of treating a neurological disorder in a human subject, the method comprising administering to the subject an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of DR6.

In some embodiments, the neurological disorder is selected from the group consisting of a brain tumor, a brain metastasis, a spinal cord injury, schizophrenia, epilepsy, Parkinson's disease, autism, Huntington's disease, stroke, Alzheimer's disease, and amyotrophic lateral sclerosis (ALS).

A fifteenth aspect features a method of treating osteoporosis or decreased bone loss in a human subject, the method comprising administering to the subject an antibody or antigen-binding fragment thereof described herein, a construct described herein, a polynucleotide described herein, a vector described herein, a host cell described herein, or a pharmaceutical composition described herein, wherein the antibody or antigen-binding fragment thereof is an antagonist of RANK.

In some embodiments, the method further comprises measuring a level of a secreted soluble TNFRSF member protein in a subject. In some embodiments, the method further comprises measuring a level of a secreted version of the TNFRSF member protein in a subject after the subject is administered the antibody or antigen-binding fragment thereof, optionally wherein the level of the secreted TNFRSF member protein is measured one day, two days, three days, four days, five days, six days, one week, or more after the antibody or antigen-binding fragment thereof is administered. In some embodiments, the method further comprises administering another dose of the antibody or antigen-binding fragment thereof to the subject if the level of the secreted version of the TNFRSF member protein is the same as, or greater than, a level of the secreted version of the TNFRSF member protein measured in the subject prior to administration of the antibody or antigen-binding fragment thereof.

A sixteenth aspect features a kit comprising an agent selected from the group consisting of an antibody or antigen-binding fragment thereof described herein, a construct described herein, a polynucleotide described herein, a vector described herein, a host cell described herein, or a pharmaceutical composition described herein.

In some embodiments, the kit comprises an antibody or antigen-binding fragment thereof described herein. In some embodiments, the kit comprises a construct described herein. In some embodiments, the kit comprises a polynucleotide described herein. In some embodiments, the kit comprises a vector described herein. In some embodiments, the kit further comprises instructions for transfecting the vector into a host cell. In some embodiments, the kit further comprises instructions for expressing the antibody, antigen-binding fragment thereof, or construct in the host cell. In some embodiments, the kit comprises a host cell described herein. In some embodiments, the kit further comprises a reagent that can be used to express the antibody, antigen-binding fragment thereof, or construct in the host cell. In some embodiments, the kit comprises a pharmaceutical composition described herein. In some embodiments, the kit further comprises instructions for administering the agent to a human subject. In some embodiments, the kit further comprises instructions for making or using the agent. In some embodiments, the kit further comprises instructions for measuring a level of secreted soluble TNFRSF member protein in a subject.

A seventeenth aspect features polypeptides, such as a single-chain polypeptides, antibodies, antigen-binding fragments thereof, and constructs thereof, that specifically bind CD40, wherein the antibody or antigen-binding fragment thereof specifically binds an epitope within amino acids 104-172 of SEQ ID NO: 4 (e.g., an epitope comprising five or more of amino acids 104-152 of SEQ ID NO: 4 and/or five or more of amino acids 123-172 of SEQ ID NO: 4, five or more of amino acids 114-142 of SEQ ID NO: 4 and/or five or more of amino acids 133-162 of SEQ ID NO: 4, five or more of amino acids 124-132 of SEQ ID NO: 4 and/or five or more of amino acids 143-152 of SEQ ID NO: 4).

In some embodiments, the antibody or antigen-binding fragment thereof comprises a non-native constant region.

In some embodiments, the antibody or antigen-binding fragment thereof inhibits signaling associated with CD40.

In some embodiments, the antibody or antigen-binding fragment thereof binds CD40 with a K_{d} of no greater than about 10 nM (e.g., no greater than about 1 nM).

In some embodiments, the antibody or antigen-binding fragment thereof binds CD40 to form an antibody-antigen complex with a kₒₙ of at least about 10⁴ M⁻¹s⁻¹.

In some embodiments, the antibody or antigen-binding fragment thereof binds CD40 to form an antibody-antigen complex, and wherein the complex dissociates with a k_{off} of no greater than about 10⁻³ s⁻¹.

In some embodiments, the antibody or antigen-binding fragment thereof has an isotype selected from the group consisting of IgG, IgA, IgM, IgD, and IgE.

In some embodiments, the antibody or antigen-binding fragment thereof is an IgG isotype (e.g., IgG2 isotype).

In some embodiments, the polypeptides, such as single-chain polypeptides, antibodies, antigen-binding fragments thereof, and constructs thereof, contain a human IgG2 hinge region that lacks a cysteine residue at positions 232 and/or 233 of the amino acid sequence of the IgG2 hinge region. For example, the polypeptide (e.g., a single-chain polypeptide, antibody, antigen-binding fragment thereof, or construct thereof) may contain a human IgG2 hinge region having an amino acid other than cysteine, such as a serine residue, at positions 232 and/or 233 of the amino acid sequence of the IgG2 hinge region.

The polypeptide (e.g., a single-chain polypeptide, antibody, antigen-binding fragment thereof, or construct thereof) may contain, for example, a human IgG2 hinge region having an amino acid substitution or deletion at one or both of cysteine residues 232 and 233. The amino acid substitution may be a conservative amino acid substitution, such as a C232S and/or C233S amino acid substitution.

In some embodiments, the antibody or antigen-binding fragment thereof comprises antigen-binding sites separated from one another by a distance of at least about 133 Å (e.g., a distance of at least about 134 Å, about 139 Å, or about 150 Å).

In some embodiments, the antigen-binding sites are separated from one another by a distance of from about 133 Å to about 150 Å (e.g., a distance of from about 133 Å to about 145 Å, about 133 Å to about 139 Å, or about 134 Å to about 139 Å).

In some embodiments, the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)2 molecule, and a tandem scFv (taFv). In some embodiments, the antibody or antigen-binding fragment thereof is a human, humanized, or chimeric antibody or antigen-binding fragment thereof.

In some embodiments, the antibody is conjugated to a therapeutic agent (e.g., a cytotoxic agent).

In some embodiments, the antibody or antigen-binding fragment thereof comprises a framework region from a human antibody or chimeric antibody.

In some embodiments, the antibody or antigen binding fragment thereof stabilizes an anti-parallel dimer conformation of CD40.

In some embodiments, the antibody or antigen-binding fragment thereof destabilizes a trimeric conformation of CD40.

In some embodiments, the antibody or antigen-binding fragment thereof reduces secretion of a soluble version of CD40.

In some embodiments, the antibody or antigen-binding fragment thereof inhibits expression of one of more genes selected from the group consisting of CHUK, NFκBIE, NFκBIA, MAP3K11, TRAF2, TRAF3, relB, and cIAP2/BIRC3.

In some embodiments, the antibody or antigen-binding fragment thereof promotes proliferation of T-reg cells.

In some embodiments, the antibody or antigen-binding fragment thereof directly kills, or promotes the death of, CD8+ cytotoxic T cells.

In some embodiments, the antibody or antigen-binding fragment thereof is capable of reducing or inhibiting proliferation of a population of CD8+ cytotoxic T cells in the presence of CD40L.

An eighteenth aspect features a construct comprising a first polypeptide domain and a second polypeptide domain, wherein the first polypeptide domain and the second polypeptide domain are each, independently, an antigen-binding fragment described herein.

In some embodiments, the first polypeptide domain and the second polypeptide domain are bound by a covalent linker (e.g., an amide bond or a disulfide bond).

A nineteenth aspect features a polynucleotide encoding an anti-CD40 antibody or antigen-binding fragment thereof described herein.

A twentieth aspect features a vector comprising a polynucleotide encoding an anti-CD40 antibody or antigen-binding fragment thereof described herein.

In some embodiments, the vector is an expression vector (e.g., a eukaryotic expression vector, a viral vector (e.g., adenovirus (Ad) (e.g., a serotype 1-60 adenovirus (e.g., a serotype 5, 26, 35, or 48 adenovirus)), retrovirus (e.g., a γ-retrovirus or a lentivirus), poxvirus, adeno-associated virus, baculovirus, herpes simplex virus, or a vaccinia virus (e.g., a modified vaccinia Ankara (MVA)))).

A twenty-first aspect features an isolated host cell comprising the vector of the twentieth aspect of the disclosure or any embodiments thereof.

In some embodiments, the host cell is a prokaryotic cell.

In some embodiments, the host cell is a eukaryotic cell (e.g., a mammalian cell (e.g., a CHO cell)).

A twenty-second aspect features a pharmaceutical composition comprising a CD40 antibody or antigen-binding fragment thereof described herein, a construct of the eighteenth aspect of the disclosure or any embodiments thereof, a polynucleotide of the nineteenth aspect of the disclosure or any embodiments thereof, a vector of the twentieth aspect of the disclosure or any embodiments thereof, or a host cell of the twenty first aspect of the disclosure or any embodiments thereof, and a pharmaceutically acceptable carrier or excipient.

In some embodiments, the pharmaceutical composition a CD40 antibody or antigen-binding fragment thereof described herein.

In some embodiments, at least 50% (e.g., at least 75% (e.g., from about 75% to about 99.9%), 80% (e.g., from about 80% to about 99.9%), 85% (e.g., from about 85% to about 99.9%), 90% (e.g., from about 90% to about 99.9%), or 95% (e.g., from about 95% to about 99.9%)) of the antibody or antigen-binding fragment thereof in the pharmaceutical composition is present in a single disulfide-bonded isoform.

In some embodiments, the antibody or antigen-binding fragment thereof yields a single detectable band upon gel electrophoresis analysis performed under non-reducing conditions.

In some embodiments, the single disulfide-bonded isoform is IgG2-A.

In some embodiments, the antibody or antigen-binding fragment thereof is present in the pharmaceutical composition in an amount of from about 0.001 mg/ml to about 100 mg/ml.

In some embodiments, the pharmaceutical composition further comprises an additional therapeutic agent.

A twenty-third aspect features a method of inhibiting an immune response mediated by a B cell or CD8+ T cell in a human subject, the method comprising administering to the subject a CD40 antibody or antigen-binding fragment thereof described herein, a construct of the eighteenth aspect of the disclosure or any embodiments thereof, a polynucleotide of the nineteenth aspect of the disclosure or any embodiments thereof, a vector of the twentieth aspect of the disclosure or any embodiments thereof, a host cell of the twenty first aspect of the disclosure or any embodiments thereof, or a pharmaceutical composition of the twenty second aspect of the disclosure or any embodiments thereof.

A twenty-fourth aspect features a method of treating an immunological disease in a human subject, the method comprising administering to the subject a CD40 antibody or antigen-binding fragment thereof described herein, a construct of the eighteenth aspect or any embodiments thereof, a polynucleotide of the nineteenth aspect or any embodiments thereof, a vector of the twentieth aspect or any embodiments thereof, a host cell of the twenty-first aspect or any embodiments thereof, or a pharmaceutical composition of the twenty-second aspect or any embodiments thereof.

In some embodiments, the subject is in need of a tissue or organ regeneration. In some embodiments, the tissue or organ is selected from the group consisting of a pancreas, salivary gland, pituitary gland, kidney, heart, lung, hematopoietic system, cranial nerves, heart, aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

In some embodiments, the immunological disease is selected from the group consisting of an autoimmune disease, a neurological condition, an allergy, asthma, macular degeneration, muscular atrophy, a disease related to miscarriage, atherosclerosis, bone loss, a musculoskeletal disease, obesity, a graft-versus-host disease, and an allograft rejection.

In some embodiments, the method further comprises measuring a level of secreted soluble CD40 in a subject. In some embodiments, the method further comprises measuring a level of a secreted version of CD40 in a subject after the subject is administered the antibody or antigen-binding fragment thereof, optionally wherein the level of the secreted CD40 is measured one day, two days, three days, four days, five days, six days, one week, or more after the antibody or antigen-binding fragment thereof is administered. In some embodiments, the method further comprises administering another dose of the antibody or antigen-binding fragment thereof to the subject if the level of the secreted version of CD40 is the same as, or greater than, a level of the secreted version of CD40 measured in the subject prior to administration of the antibody or antigen-binding fragment thereof.

A twenty-fifth aspect features a kit comprising an agent selected from the group consisting of a CD40 antibody or antigen-binding fragment thereof described herein, a construct of the eighteenth aspect or any embodiments thereof, a polynucleotide of the nineteenth aspect or any embodiments thereof, a vector of the twentieth aspect or any embodiments thereof, a host cell of the twenty-first aspect or any embodiments thereof, or a pharmaceutical composition of the twenty-second aspect or any embodiments thereof.

In some embodiments, the kit comprises a CD40 antibody or antigen-binding fragment thereof described herein. In some embodiments, the kit comprises the construct of any of claims 197-200. In some embodiments, the kit comprises a polynucleotide of the eighteenth aspect. In some embodiments, the kit comprises a vector of the twentieth aspect. In some embodiments, the kit further comprises instructions for transfecting the vector into a host cell. In some embodiments, the kit further comprises instructions for expressing the CD40 antibody, antigen-binding fragment thereof, or construct in the host cell. In some embodiments, the kit comprises a host cell of the twenty first aspect. In some embodiments, the kit further comprises a reagent that can be used to express the CD40 antibody, antigen-binding fragment thereof, or construct in the host cell. In some embodiments, the kit comprises a pharmaceutical composition of the twenty second aspect. In some embodiments, the kit further comprises instructions for administering the agent to a human subject. In some embodiments, the kit further comprises instructions for making or using the agent. In some embodiments, the kit further comprises instructions for measuring a level of secreted soluble CD40 in a subject.

### Definitions

As used herein, the term “about” refers to a value that is no more than 10% above or below the value being described. For example, the term “about 5 nM” indicates a range of from 4.5 nM to 5.5 nM.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, primatized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, IgG, and scFv fragments. Moreover, unless otherwise indicated, the term “monoclonal antibody” (mAb) is meant to include both intact molecules, as well as, antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. An “antigen-binding fragment” may also refer to a linear or non-linear peptide, which may be cyclic, bicylic, and/or conformationally biased. It will be appreciated by one of skill in the art that a conformationally biased antigen-binding fragment will entail a structure-based design such that the peptide includes an antigen-binding site mimic based on the 3D structure of the peptide-antigen complex. Such structural information enables the design and generation of mimics of continuous, as well as of sequentially discontinuous antigen-binding sites, which are composed of two or more protein segments that are distant in protein sequence but brought into spatial proximity through protein folding. Mimicking such discontinuous antigen-binding sites by synthetic peptides often involves splicing and/or molecular scaffolds (e.g., disulfide bonds) to enable conformation bias. See e.g., Groβ et al., Front. Bioeng. Biotechnol. 4(39). 2016. Antigen-binding fragments can be produced by recombinant DNA techniques, enzymatic or chemical cleavage of intact immunoglobulins, or, in some embodiments, by chemical peptide synthesis procedures known in the art.

As used herein, the terms “anti-tumor necrosis factor receptor superfamily member antibody,” “anti-TNFRSF member antibody,” “anti-TNFRSF member antibody portion,” and/or “anti-TNFRSF member antibody fragment,” and the like include any protein or peptide-containing molecule that includes at least a portion of an immunoglobulin molecule, such as, but not limited, to at least one complementarity determining region (CDR) of a heavy or light chain or a ligand-binding portion thereof, a heavy chain or light chain variable region, a heavy chain or light chain constant region, or any portion thereof, that is capable of specifically binding to a TNFRSF member protein (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40). For instance, two or more portions of an immunoglobulin molecule may be covalently bound to one another, e.g., via an amide bond, a thioether bond, a carbon-carbon bond, a disulfide bridge, or by a linker, such as a linker described herein or known in the art. TNFRSF member antibodies also include antibody-like protein scaffolds, such as the tenth fibronectin type III domain (¹⁰Fn3), which contains BC, DE, and FG structural loops similar in structure and solvent accessibility to antibody CDRs. The tertiary structure of the ¹⁰Fn3 domain resembles that of the variable region of the IgG heavy chain, and one of skill in the art can graft, e.g., the CDRs of a TNFRSF member monoclonal antibody onto the fibronectin scaffold by replacing residues of the BC, DE, and FG loops of ¹⁰Fn3 with residues from the CDR-H1, CDR-H2, or CDR-H3 regions of a TNFRSF member monoclonal antibody.

As used herein, the terms “antagonist TNFRSF member antibody” and “antagonistic TNFRSF member antibody” refer to TNFRSF member antibodies that are capable of inhibiting or reducing activation of a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40), attenuating one or more signal transduction pathways mediated by the TNFRSF member, and/or reducing or inhibiting at least one activity mediated by activation of the TNFRSF member. For example, antagonistic TNFRSF member antibodies may inhibit or reduce the growth and proliferation of regulatory T cells. Antagonistic TNFRSF member antibodies may inhibit or reduce TNFRSF member activation by blocking the TNFRSF member from binding one or more ligands in the TNF superfamily, e.g., CD40L for CD40, TNFα for TNFR1 and TNFR2,4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others. In this way, antagonistic TNFRSF member antibodies may block the trimerization of the TNFRSF member that would otherwise be induced by interacting with one or more ligands in the TNF superfamily (e.g., CD40L, TNFα, 4-1 BBL, CD70, CD153, or RANKL, among others), thus resulting in suppression of the TNFRSF member activity.

As used herein, the term “bispecific antibodies” refers to antibodies (e.g., monoclonal, often human or humanized antibodies) that have binding specificities for at least two different antigens. For example, one of the binding specificities can be directed towards a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40), the other can be for any other antigen, e.g., for a cell-surface protein, receptor, receptor subunit, tissue-specific antigen, virally derived protein, virally encoded envelope protein, bacterially derived protein, or bacterial surface protein, etc.

As used herein, the phrase “chemotherapeutic agent” refers to any chemical agent with therapeutic usefulness in the treatment of cancer, such as a cancer described herein. Chemotherapeutic agents encompass both chemical and biological agents. These agents can function to inhibit a cellular activity upon which a cancer cell depends for continued survival. Categories of chemotherapeutic agents include alkylating/alkaloid agents, antimetabolites, hormones, hormone analogs, and antineoplastic drugs. Exemplary chemotherapeutic agents suitable for use in conjunction with the compositions and methods described herein include, without limitation, those set forth in Slapak and Kufe, Principles of Cancer Therapy, Chapter 86 in Harrison's Principles of Internal medicine, 14^{th} edition; Perry et al., Chemotherapeutic, Chapter 17 in Abeloff, Clinical Oncology 2^{nd} ed., 2000; Baltzer L. and Berkery R. (eds): Oncology Pocket Guide to Chemotherapeutic, 2^{nd} ed. St. Louis, mosby-Year Book, 1995; Fischer D. S., Knobf M. F., Durivage H. J. (eds): The Cancer Chemotherapeutic Handbook, 4^{th} ed. St. Louis, Mosby-Year Handbook, the disclosures of each of which are incorporated herein by reference as they pertain to chemotherapeutic agents.

As used herein, the term “chimeric” antibody refers to an antibody having variable domain sequences (e.g., CDR sequences) derived from an immunoglobulin of one source organism, such as rat or mouse, and constant regions derived from an immunoglobulin of a different organism (e.g., a human, a non-human primate, pig, goat, rabbit, hamster, cat, dog, guinea pig, a member of the bovidae family (such as cattle, bison, buffalo, elk, and yaks, among others), cow, sheep, horse, or bison, among others). Methods for producing chimeric antibodies are known in the art. See, e.g., Morrison, Science 229:1202-7, 1985; Oi et al., Bio Techniques 4:214-221, 1986; Gillies et al., J. Immunol. Methods 125:191-202, 1985; U.S. Pat. Nos. 5,807,715; 4,816,567; and 4,816,397; incorporated herein by reference.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may comprising modifications in these hybrid hypervariable positions. The variable domains of native heavy and light chains each comprise four framework regions that primarily adopt a 6-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, 5 Bethesda, Md. 1987; incorporated herein by reference). As used herein, numbering of immunoglobulin amino acid residues is done according to the immunoglobulin amino acid residue numbering system of Kabat et al, unless otherwise indicated.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

Amino acid substitutions may be represented herein using the convention: (AA1)(N)(AA2), where “AA1” represents the amino acid normally present at particular site within an amino acid sequence, “N” represents the residue number within the amino acid sequence at which the substitution occurs, and “AA2” represents the amino acid present in the amino acid sequence after the substitution is effectuated. For example, the notation “0232S” in the context of an antibody hinge region, such as an IgG2 antibody hinge region, refers to a substitution of the naturally-occurring cysteine residue for a serine residue at amino acid residue 232 of the indicated hinge amino acid sequence. Likewise, the notation “0233S” in the context of an antibody hinge region, such as an IgG2 antibody hinge region, refers to a substitution of the naturally-occurring cysteine residue for a serine residue at amino acid residue 233 of the indicated hinge amino acid sequence.

As used herein, the term “conjugate” refers to a compound formed by the chemical bonding of a reactive functional group of one molecule with an appropriately reactive functional group of another molecule.

As used herein in the context of a TNFRSF member antagonist, the term “construct” refers to a fusion protein containing a first polypeptide domain bound to a second polypeptide domain. The polypeptide domains may each independently be antagonistic TNFRSF member single chain polypeptides, for instance, as described herein. The first polypeptide domain may be covalently bound to the second polypeptide domain, for instance, by way of a linker, such as a peptide linker or a disulfide bridge, among others. Exemplary linkers that may be used to join the polypeptide domains of an antagonistic TNFRSF member construct include, without limitation, those that are described in Leriche et al., Bioorg. Med. Chem. 20:571-582, 2012, the disclosure of which is incorporated herein by reference in its entirety.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes V_{H} and V_{L} domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain. In order to fold into their native structure, peptides configured in this way typically trimerize so as to position the VH and VL domains of neighboring peptide chains spatially proximal to one another to permit proper folding (see Holliger et al., Proc. Natl. Acad. Sci. USA 90:6444-48, 1993; incorporated herein by reference).

As used herein, a “disulfide-bonded isoform” of an antibody or antigen-binding fragment thereof is a form of the antibody or antigen-binding fragment thereof having a particular internal disulfide bonding pattern. Disulfide-bonded isoforms are structural isomers of a given antibody or antigen-binding fragment thereof that do not differ from one another in amino acid sequence but exhibit different disulfide bond connectivities. For example, in the context of a human IgG2 antibody or variant thereof, the antibody may exist in one of four possible disulfide-bonded isoforms, represented herein as isoforms IgG2-A, IgG2-B, IgG2-A/B₁, and IgG2-A/B₂. The disulfide bonding connectivities within each of these isoforms are shown graphically in FIGS. 2A-2D.

As used herein, a “dominant antagonist” of a TNFRSF member is an antagonist (e.g., an antagonistic polypeptide, such as a single-chain polypeptide, antibody, or antigen-binding fragment thereof) that is capable of inhibiting TNFRSF member activation even in the presence of a TNFRSF member ligand, such as CD40L, or a natural ligand of any one of 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, and XEDAR. For example, a TNFRSF member antagonist is a dominant antagonist if the IC₅₀ of the antagonist increases by less than 200% (e.g., less than 200%, 100%, 50%, 45%, 40%, 35%, 30%, 25%, 20%, 15%, 10%, 5%, 1%, or less) in the presence of a TNFRSF member ligand (e.g., CD40L for CD40, TNFα for TNFR1 and TNFR2,4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others) relative to the IC₅₀ of the antagonist as measured in the same assay in the absence of a TNFRSF member ligand, such as CD40L, TNFα, 4-1 BBL, CD70, CD153, or RANKL, among others. Inhibition of TNFRSF member activation can be assessed, for instance, by measuring the inhibition of proliferation of TNFRSF member expressing cells, such as T-reg cells, cancer cells that express the TNFRSF member, or myeloid-derived suppressor cells, as well as by measuring the inhibition of NFκB signaling (e.g., by monitoring the reduction in expression of one or more genes selected from the group consisting of CHUK, NFκBIE, NFκBIA, MAP3K11, TRAF2, TRAF3, relB, and clAP2/BIRC3 in a conventional gene expression assay).

As used herein, a “dual variable domain immunoglobulin” (“DVD-Ig”) refers to an antibody that combines the target-binding variable domains of two monoclonal antibodies via linkers to create a tetravalent, dual-targeting single agent. (Gu et al., Meth. Enzymol. 502:25-41, 2012; incorporated by reference herein).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “epitope” refers to a portion of an antigen that is recognized and bound by a polypeptide, such as an antibody, antigen-binding fragment thereof, single-chain polypeptide, or construct as described herein. In the context of a protein antigen (such as a TNFRSF member, e.g., human CD40 or CD40 of a non-human mammal, such as a non-human mammal described herein), an epitope may be a continuous epitope, which is a single, uninterrupted segment of one or more amino acids covalently linked to one another by peptide bonds in which all of the component amino acids bind the polypeptide (e.g., antibody, antigen-binding fragment thereof, single-chain polypeptide, or construct thereof). Exemplary assays for determining the binding of an antagonistic TNFRSF member polypeptide to specific amino acids within an antigen are described in Example 1, below. Continuous epitopes may be composed, for instance, of 1, 5, 10, 15, 20, or more amino acids within an antigen, such as a TNFRSF member protein such as CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, and XEDAR. For example, a continuous epitope may be composed of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more amino acids within an antigen). Examples of continuous epitopes on human TNFRSF members that are bound by antagonistic polypeptides (e.g., single-chain polypeptides, antibodies, antigen-binding fragments thereof, and constructs thereof) described herein include two or more continuous residues of, or all residues of, the MCEQDCKQGQELTKKGCKDCCFGTFNDQK motif (SEQ ID NO: 27) on 4-1 BB, two or more continuous residues of, or all residues of, the QMCEPGT motif (SEQ ID NO: 28) on CD27, two or more continuous residues of, or all residues of, the QCDPCIPGVSFS motif (SEQ ID NO: 29) on CD27, two or more continuous residues of, or all residues of, the SVCPAGMIVKFP motif (SEQ ID NO: 30) on CD30, two or more continuous residues of, or all residues of, the CEPASPGVS motif (SEQ ID NO:31) on CD30, two or more continuous residues of, or all residues of, the SCSPGFGVK motif (SEQ ID NO: 25) on CD40, two or more continuous residues of, or all residues of, the CEPCPVGFFS motif (SEQ ID NO:26) on CD40, two or more continuous residues of, or all residues of, the NGTCAPHTVCPVGWGV motif (SEQ ID NO: 32) on DR6, two or more continuous residues of, or all residues of, the KQCARGTFS motif (SEQ ID NO: 33) on DR 6, two or more continuous residues of, or all residues of, the KDCEGFFRATVL motif (SEQ ID NO: 34) on EDAR, two or more continuous residues of, or all residues of, the AECGPCLPGYYM motif (SEQ ID NO: 35) on EDAR, two or more continuous residues of, or all residues of, the KCEHGIIKE motif (SEQ ID NO: 36) on Fas, two or more continuous residues of, or all residues of, the CKEEGSRSN motif (SEQ ID NO: 37) on Fas, two or more continuous residues of, or all residues of, the HPCPPGQGVQ motif (SEQ ID NO: 38) on GITR, two or more continuous residues of, or all residues of, the CIDCASGTFS motif (SEQ ID NO: 39) on GITR, two or more continuous residues of, or all residues of, the TTCPPGQRVEK motif (SEQ ID NO: 40) on HVEM, two or more continuous residues of, or all residues of, the CADCLTGTF motif (SEQ ID NO: 41) on HVEM, two or more continuous residues of, or all residues of, the DCPPGTEAE motif (SEQ ID NO: 42) on LT beta receptor, two or more continuous residues of, or all residues of, the CVPCKAGHFQ motif (SEQ ID NO: 43) on LT beta receptor, two or more continuous residues of, or all residues of, the CEAGSGLV motif (SEQ ID NO: 44) on NGFR, two or more continuous residues of, or all residues of, the CEECPDGTYSD motif (SEQ ID NO: 45) on NGFR, two or more continuous residues of, or all residues of, the SCPPGFGVV motif (SEQ ID NO: 46) on OPG, two or more continuous residues of, or all residues of, the CKRCPDGFFS motif (SEQ ID NO: 47) on OPG, two or more continuous residues of, or all residues of, the SYKPGVDCAPCPPGHFS motif (SEQ ID NO: 48) on OX40, two or more continuous residues of, or all residues of, the ECAPGLGA motif (SEQ ID NO: 49) on RANK, two or more continuous residues of, or all residues of, the CKPCLAGYFS motif (SEQ ID NO: 50) on RANK, two or more continuous residues of, or all residues of, the RCSLWRRL motif (SEQ ID NO: 51) on RELT (19L), two or more continuous residues of, or all residues of, the CGDCWPGWF motif (SEQ ID NO: 52) on RELT (19L), two or more continuous residues of, or all residues of, the KCRKEMGQV motif (SEQ ID NO: 53) on TNFR1, two or more continuous residues of, or all residues of, the VCGCRKNQYR motif (SEQ ID NO: 54) on TNFR1, two or more continuous residues of, or all residues of, the KCRTGCPRGMV motif (SEQ ID NO: 55) on TRAIL-R2 (TNFRSF10B), two or more continuous residues of, or all residues of, the CVHKESGTK motif (SEQ ID NO: 56) on TRAIL-R2 (TNFRSF10B), two or more continuous residues of, or all residues of, the ACKSDEEER motif (SEQ ID NO: 57) on TRAIL-R1 (TNFRSF10A), two or more continuous residues of, or all residues of, the CQCKPGTFR motif (SEQ ID NO: 58) on TRAIL-R1 (TNFRSF10A), two or more continuous residues of, or all residues of, the GCPRGMVKV motif (SEQ ID NO: 59) on TRAIL-R4, two or more continuous residues of, or all residues of, the KNESAASSTG motif (SEQ ID NO: 60 on TRAIL-R4, two or more continuous residues of, or all residues of, the LDCGALHRH motif (SEQ ID NO: 61) on TRAMP, two or more continuous residues of, or all residues of, the CGTCLPGFYE motif (SEQ ID NO: 62) on TRAMP, two or more continuous residues of, or all residues of, the QCGPGMELS motif (SEQ ID NO: 63) on TROY, two or more continuous residues of, or all residues of, the CVTCRLHRFKE motif (SEQ ID NO: 64) on TROY, two or more continuous residues of, or all residues of, the RCGPGQEL motif (SEQ ID NO: 65) on XEDAR, and two or more continuous residues of, or all residues of, the TACPPRRY motif (SEQ ID NO: 66) on XEDAR, as well as corresponding regions on TNFRSF members of non-human mammals (e.g., bison, cattle, and others described herein). In some embodiments, an epitope may be a discontinuous epitope, which contains two or more amino acids each separated from one another in an antigen's amino acid sequence by one or more intervening amino acid residues. Discontinuous epitopes may be composed, for instance, of 1, 2, 3, 4, 5, 6, 7, 8, 9, 10, or more such segments of amino acid residues, such as one or more (e.g., 1, 2, 3, 4, 5, 6, 7, 8, 9, 10, or more) segments containing amino acids from within one or more of the MCEQDCKQGQELTKKGCKDCCFGTFNDQK motif (SEQ ID NO: 27) within human 4-1 BB; the QMCEPGT motif (SEQ ID NO:28) and/or the QCDPCIPGVSFS motif (SEQ ID NO: 29) within human CD27; the SVCPAGMIVKFP motif (SEQ ID NO: 30) and/or the CEPASPGVS motif (SEQ ID NO: 31) within human CD30; the SCSPGFGVK motif (SEQ ID NO: 25) and/or the CEPCPVGFFS motif (SEQ ID NO:26) within human CD40; the NGTCAPHTVCPVGWGV motif (SEQ ID NO: 32) and/or the KQCARGTFS motif (SEQ ID NO: 33) within human DR6; the KDCEGFFRATVL motif (SEQ ID NO: 34) and/or the AECGPCLPGYYM motif (SEQ ID NO: 35) within human EDAR; the KCEHGIIKE motif (SEQ ID NO: 36) and/or the CKEEGSRSN motif (SEQ ID NO: 37) within human Fas; the HPCPPGQGVQ motif (SEQ ID NO: 38) and/or the CIDCASGTFS motif (SEQ ID NO: 39) within human GITR; the TTCPPGQRVEK motif (SEQ ID NO: 40) and/or the CADCLTGTF motif (SEQ ID NO: 41) within human HVEM; the DCPPGTEAE motif (SEQ ID NO: 42) and/or the CVPCKAGHFQ motif (SEQ ID NO: 43) within human LT beta receptor; the CEAGSGLV motif (SEQ ID NO: 44) and/or the CEECPDGTYSD motif (SEQ ID NO: 45) within human NGFR; the SCPPGFGVV motif (SEQ ID NO: 46) and/or the CKRCPDGFFS motif (SEQ ID NO: 47) within human OPG; the SYKPGVDCAPCPPGHFS motif (SEQ ID NO: 48) within human OX40; the ECAPGLGA motif (SEQ ID NO: 49) and/or the CKPCLAGYFS motif (SEQ ID NO: 50) within human RANK; the RCSLWRRL motif (SEQ ID NO: 51) and/or the CGDCWPGWF motif (SEQ ID NO: 52) within human RELT (19L); the KCRKEMGQV motif (SEQ ID NO: 53) and/or the VCGCRKNQYR motif (SEQ ID NO: 54) within human TNFR1; the KCRTGCPRGMV motif (SEQ ID NO: 55) and/or the CVHKESGTK motif (SEQ ID NO: 56) within human TRAIL-R2 (TNFRSF10B); the ACKSDEEER motif (SEQ ID NO: 57) and/or the CQCKPGTFR motif (SEQ ID NO: 58) within human TRAIL-R1 (TNFRSF10A); the GCPRGMVKV motif (SEQ ID NO: 59) and/or the KNESAASSTG motif (SEQ ID NO: 60) within human TRAIL-R4; the LDCGALHRH motif (SEQ ID NO: 61) and/or the CGTCLPGFYE motif (SEQ ID NO: 62) within human TRAMP; the QCGPGMELS motif (SEQ ID NO: 63) and/or the CVTCRLHRFKE motif (SEQ ID NO: 64) within human TROY; the RCGPGQEL motif (SEQ ID NO: 65) and/or the TACPPRRY motif (SEQ ID NO: 66) within human XEDAR; as well as corresponding regions on TNFRSF members of non-human mammals (e.g., bison, cattle, and others described herein). Despite this separation by intervening amino acids, the segments that compose a discontinuous epitope may be, for instance, spatially proximal to one another in the three-dimensional conformation of the antigen.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem. 20:571-582, 2012).

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655, 1991, Suresh et al., Methods in Enzymology 121:210, 1986; incorporated herein by reference. Heterospecific antibodies can include Fc mutations that enforce correct chain association in multi-specific antibodies, as described by Klein et al., mAbs 4:653-663, 2012; incorporated herein by reference.

As used herein, the term “hinge region” refers to the domain of an antibody or antigen-binding fragment thereof (e.g., an IgG2 antibody or antigen-binding fragment thereof) located between the antigen-binding portion(s) of the antibody or antigen-binding fragment thereof, such as the Fab region of the antibody or antigen-binding fragment thereof, and the portion of the antibody or antigen-binding fragment thereof that dictates the isotype of the antibody or antigen-binding fragment thereof, such as the Fc region of the antibody or antigen-binding fragment thereof. For example, in the context of a monoclonal antibody, the hinge region is the polypeptide situated approximately in the center of each heavy chain, connecting the CH1 domain to the CH2 and CH3 domains. The hinge region of an antibody or antigen-binding fragment thereof may provide a chemical linkage between chains of the antibody or antigen-binding fragment thereof. For instance, in a monoclonal antibody, the cysteine residues within the hinge region form inter-chain disulfide bonds, thereby providing explicit covalent bonds between heavy chains. The amino acid sequence of wild-type human IgG2 is ERKCCVECPPCP (SEQ ID NO: 24). As used herein, antibody hinge regions are numbered according to the numbering system of Kabat et al, Sequences of Proteins of Immunological Interest (National Institute of Health, Bethesda, Md. 1987), the disclosure of which is incorporated herein by reference. For example, using the numbering scheme of Kabat et al, the wild-type human IgG2 hinge region set forth in SEQ ID NO: 24 is numbered from residues 226 to 243, such that the N-terminal glutamate residue of SEQ ID NO: 24 is residue 226 and the C-terminal proline residue of SEQ ID NO: 24 is residue 243. Throughout the present disclosure, variant IgG2 hinge regions, such as the variant set forth in SEQ ID NO: 23 (ERKCCVESPPCP), are numbered according to the convention of Kabat et al unless explicitly stated to the contrary.

As used herein, the term “human antibody” refers to an antibody in which substantially every part of the protein (e.g., CDR, framework, C_{L}, CH domains (e.g., CH1, CH2, CH3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; and EP519596; incorporated herein by reference.

As used herein, the term “hydrophobic side-chain” refers to an amino acid side-chain that exhibits low solubility in water relative due to, e.g., the steric or electronic properties of the chemical moieties present within the side-chain. Examples of amino acids containing hydrophobic side-chains include those containing unsaturated aliphatic hydrocarbons, such as alanine, valine, leucine, isoleucine, proline, and methionine, as well as amino acids containing aromatic ring systems that are electrostatically neutral at physiological pH, such as tryptophan, phenylalanine, and tyrosine.

As used herein, the term “immunotherapy agent” refers to a compound, such as an antibody, antigen-binding fragment thereof, single-chain polypeptide, or construct as described herein, that specifically binds an immune checkpoint protein (e.g., immune checkpoint receptor or ligand) and exerts an antagonistic effect on the receptor or ligand, thereby reducing or inhibiting the signal transduction of the receptor or ligand that would otherwise lead to a downregulation of the immune response. Immunotherapy agents include compounds, such as antibodies, antigen-binding fragments, single-chain polypeptides, and constructs, capable of specifically binding receptors expressed on the surfaces of hematopoietic cells, such as lymphocytes (e.g., T cells), and suppressing the signaling induced by the receptor or ligand that would otherwise lead to tolerance towards an endogenous (“self”) antigen, such as a tumor-associated antigen. Immunotherapy agents may reduce the signaling induced by the receptor or ligand by, for example, 1%, 2%, 3%, 4%, 5%, 10%, 20%, 30%, 40%, 50%, 60%, 70%, 80%, 90%, 95%, 96%, 97%, 98%, 99%, 99.9%, or 100% relative to the signaling induced by the receptor or ligand exhibited in the absence of the immunotherapy agent. Exemplary assays that can be used to measure the extent of receptor or ligand signaling include, for example, enzyme-linked immunosorbent assay (ELISA) techniques to measure protein expression alterations that are associated with a particular signal transduction pathway, as well as polymerase chain reaction (PCR)-based techniques, such as quantitative PCR, reverse-transcription PCR, and real-time PCR experiments useful for determining changes in gene expression associated with a particular signal transduction pathway, among others. Exemplary methods that can be used to determine whether an agent is an “immunotherapy agent” include the assays described in Mahoney et al., Cancer Immunotherapy 14:561-584, 2015, the disclosure of which is incorporated herein by reference in its entirety. Examples of immunotherapy agents include, e.g., antibodies or antigen-binding fragments thereof that specifically bind one or more of TL1A, CD40L, LIGHT, BTLA, LAG3, TIM3, Singlecs, ICOS, B7-H3, B7-H4, VISTA, TMIGD2, BTNL2, CD48, KIR, LIR, LIR antibody, ILT, NKG2D, NKG2A, MICA, MICB, CD244, CSF1R, IDO, TGFβ, CD39, CD73, CXCR4, CXCL12, SIRPA, CD47, VEGF, and neuropilin. Additional examples of immunotherapy agents include Targretin, Interferon-alpha, clobetasol, Peg Interferon (e.g., PEGASYS®), prednisone, Romidepsin, Bexarotene, methotrexate, Triamcinolone cream, anti-chemokines, Vorinostat, gabapentin, antibodies to lymphoid cell surface receptors and/or lymphokines, antibodies to surface cancer proteins, and/or small molecular therapies such as Vorinostat. Particular examples of immunotherapy agents that may be used in or in conjunction with the compositions and methods described herein include anti-PD-1 antibodies and antigen-binding fragments thereof, such as nivolumab, pembrolizumab, avelumab, durvalumab, and atezolizumab, as well as anti-PD-L1 antibodies and antigen-binding fragments thereof, such as atezolizumab and avelumab, and anti-CTLA-4 antibodies and antigen-binding fragments thereof, such as ipilimumab or tremelimumab.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen. Multi-specific antibodies can have structures similar to full immunoglobulin molecules and include Fc regions, for example IgG Fc regions. Such structures can include, but not limited to, IgG-Fv, IgG-(scFv)₂, DVD-Ig, (scFv)₂-(scFv)₂-Fc and (scFv)₂-Fc-(scFv)₂. In case of IgG-(scFv)₂, the scFv can be attached to either the N-terminal or the C-terminal end of either the heavy chain or the light chain. Exemplary multi-specific molecules that include Fc regions and into which anti-TNFRSF member antibodies or antigen-binding fragments thereof can be incorporated have been reviewed by Kontermann, mAbs 4:182-197, 2012, Yazaki et al., Protein Engineering, Design & Selection 26:187-193, 2013, and Grote et al., 2012, in Proetzel & Ebersbach (eds.), Antibody Methods and Protocols, Methods in Molecular Biology vol. 901, chapter 16:247-263; incorporated herein by reference. In some embodiments, antibody fragments can be components of multi-specific molecules without Fc regions, based on fragments of IgG or DVD or scFv. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, Nature Medicine 9:129-134, 2003; incorporated herein by reference.

As used herein, the term “myeloid-derived suppressor cell” or “MDSC” refers to a cell of the immune system that modulates the activity of a variety of effector cells and antigen-presenting cells, such as T cells, NK cells, dendritic cells, and macrophages, among others. Myeloid derived suppressor cells are distinguished by their gene expression profile, and express all or a subset of proteins and small molecules selected from the group consisting of B7-1 (CD80), B7-H1 (PD-L1), CCR2, CD1d, CD1d1, CD2, CD31 (PECAM-1), CD43, CD44, complement component C5a R1, F4/80 (EMR1), Fcγ RIII (CD16), Fcγ RII (CD32), Fcγ RIIA (CD32a), Fcγ RIIB (CD32b), Fcγ RIIB/C (CD32b/c), Fcγ RIIC (CD32c), Fcγ RIIIA (CD16A), Fcγ RIIIB (CD16b), galectin-3, GP130, Gr-1 (Ly-6G), ICAM-1 (CD54), IL-1 RI, IL-4Ra, IL-6Rα, integrin α4 (CD49d), integrin αL (CD11a), integrin αM (CD11 b), M-CSFR, MGL1 (CD301a), MGL1/2 (CD301a/b), MGL2 (CD301b), nitric oxide, PSGL-1 (CD162), L-selectin (CD62L), siglec-3 (CD33), transferrin receptor (TfR), VEGFR1 (Flt-1), and VEGFR2 (KDR or Flk-1). Particularly, MDSCs do not express proteins selected from the group consisting of B7-2 (CD86), B7-H4, CD11c, CD14, CD21, CD23 (FcεRII), CD34, CD35, CD40 (TNFRSF5), CD117 (c-kit), HLA-DR, and Sca-1 (Ly6).

As used herein, the terms “neutral TNFRSF member polypeptide” and “phenotype-neutral TNFRSF member polypeptide” refer to a polypeptide (such as a single-chain polypeptide, an antibody, or an antibody fragment) that binds a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) and does not exert an antagonistic or an agonistic effect on the TNFRSF member activation. For instance, a TNFRSF member polypeptide is a neutral TNFRSF member polypeptide if the polypeptide binds its specific TNFRSF member and neither potentiates nor suppresses the TNFRSF member activation, for instance, as assessed by measuring the proliferation of TNFRSF member expressing cells (e.g., T-reg cells, TNFRSF member expressing cancer cells, and/or MDSCs) and/or by measuring the expression of one or more NFκB target genes, such as CHUK, NFκBIE, NFκBIA, MAP3K11, TRAF2, TRAF3, relB, and/or cIAP2/BIRC3.

As used herein, the term “non-native constant region” refers to an antibody constant region that is derived from a source that is different from the antibody variable region or that is a human-generated synthetic polypeptide having an amino sequence that is different from the native antibody constant region sequence. For instance, an antibody containing a non-native constant region may have a variable region derived from a non-human source (e.g., a mouse, rat, or rabbit) and a constant region derived from a human source (e.g., a human antibody constant region), or a constant region derived from another primate, pig, goat, rabbit, hamster, cat, dog, guinea pig, member of the bovidae family (such as cattle, bison, buffalo, elk, and yaks, among others), cow, sheep, horse, or bison, among others).

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “primatized antibody” refers to an antibody comprising framework regions from primate-derived antibodies and other regions, such as CDRs and/or constant regions, from antibodies of a non-primate source. Methods for producing primatized antibodies are known in the art. See e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “proliferation” in the context of a population of cells, such as a population of TNFRSF member expressing cells (e.g., T-reg cells, MDSCs, or TNFRSF member expressing cancer cells) refers to mitotic and cytokinetic division of a cell so as to produce a plurality of cells. Cell proliferation may be evidenced, for example, by a finding that the quantity of cells (e.g., TNFRSF member expressing cells) in a subject or sample of cells has increased over a given time period, such as over the course of one or more hours, days, or weeks. One of skill in the art may monitor cell proliferation using a variety of known techniques, such as by way of visual microscopy, hemocytometry, flow cytometry, fluorescence activated cell sorting, and other assays known in the art. In the present disclosure, cell proliferation is considered to be “inhibited” when the rate of proliferation of a population of cells, such as a population of TNFRSF member expressing cells contacted with an antagonistic TNFRSF member polypeptide described herein, is decreased relative to the rate of proliferation of a population of control cells, such as a population of TNFRSF member expressing cells not contacted with the antagonistic TNFRSF member polypeptide. A decrease in the rate of proliferation may manifest, for example, as a reduction in the quantity of cells of interest in a subject or sample over a given time period, such as a reduction in the quantity of cells of interest in a subject or sample of 1%, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more, over a given time period. Additionally or alternatively, inhibition of cell proliferation may be evidenced by a finding that the rate at which cells of interest (e.g., TNFRSF member expressing cells contacted with an antagonistic TNFRSF member polypeptide described herein) are dividing is reduced, e.g., by %, 2%, 3%, 4%, 5%, 6%, 7%, 8%, 9%, 10%, 11%, 12%, 13%, 14%, 15%, 20%, 25%, 30%, 35%, 40%, 45%, 50%, 60%, 70%, 80%, 90%, or more, relative to the rate at which control cells (e.g., TNFRSF member expressing cells not contacted with the antagonistic TNFRSF member polypeptide) are dividing.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the term “pharmacokinetic profile” refers to the absorption, distribution, metabolism, and clearance of a drug over time following administration of the drug to a subject.

As used herein, a “recessive antagonist” of a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) is an antagonist (e.g., an antagonistic polypeptide, such as a single-chain polypeptide, antibody, or antigen-binding fragment thereof) that inhibits the TNFRSF member activation to a significantly lesser extent in the presence of a TNFRSF member ligand, such as CD40L for CD40, TNFα for TNFR1 and TNFR2,4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others, relative to the extent of inhibition of the same antagonist as measured in the absence of a TNFRSF member ligand, such as CD40L, TNFα, or 4-1 BBL. For example, a TNFRSF member antagonist is a recessive antagonist if the IC₅₀ of the antagonist increases by, e.g., 10-fold, 20-fold, 30-fold, 40-fold, 50-fold, 60-fold, 70-fold, 80-fold, 90-fold, 100-fold, or more in the presence of a TNFRSF member ligand (e.g., CD40L, TNFα, 4-1 BBL, CD70, CD153, or RANKL, among others) relative to the IC₅₀ of the antagonist as measured in the same assay the absence of a TNFRSF member ligand, such as CD40L, or TNFα, among others.

Inhibition of TNFRSF member activation can be assessed, for instance, by measuring the inhibition of proliferation of TNFRSF member expressing cells, such as T-reg cells, cancer cells that express the TNFRSF member, or myeloid-derived suppressor cells, as well as by measuring the inhibition of NFκB signaling (e.g., by monitoring the reduction in expression of one or more genes selected from the group consisting of CHUK, NFκBIE, NFκBIA, MAP3K11, TRAF2, TRAF3, relB, and cIAP2/BIRC3 in a conventional gene expression assay).

As used herein, the terms “TNFRSF member cognate ligand,” “TNFRSF member natural ligand,” “TNFRSF cognate or natural ligand,” “TNFRSF member ligand,” “endogenous TNFRSF member ligand,” and “TNFRSF member endogenous ligand” refer to an endogenous ligand of the TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) that may form a TNFRSF member ligand-TNFRSF member protein complex and induce the formation of the active, homotrimeric conformation of the TNFRSF member protein (FIG. 3). Exemplary TNFRSF member ligand-TNFRSF member protein complexes may include, but are not limited to, the CD40L-CD40 complex, the 4-1 BBL-4-1 BB complex, the CD70-CD27 complex, the CD153-CD30 complex, the N-APP-DR6 complex, the EDA-A1—EDAR complex, the FasL-Fas complex, the GITRL-GITR complex, the LTα-HVEM complex, the LT beta (TNF-C)—LT beta receptor complex, the NGF-NGFR complex, the TRAIL-OPG complex, the OX40L-OX40 complex, the RANKL-RANK complex, the TNFα-TNFR1 complex, the TRAIL-TRAIL-R2 (TNFRSF10B) complex, the TRAIL-TRAIL-R1 (TNFRSF10A) complex, the TRAIL-TRAIL-R4 complex, the TL1A-TRAMP complex, and the EDA-A2—XEDAR complex.

As used herein, the term “regulatory sequence” includes promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, C A, 1990); incorporated herein by reference.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., Gene 77:51, 1989; Bird et al., Science 242:423, 1988; Pantoliano et al., Biochemistry 30:10117, 1991; Milenic et al., Cancer Research 51:6363, 1991; and Takkinen et al., Protein Engineering 4:837, 1991. The VL and VH domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{d} of less than 100 nM. For example, an antibody or antigen-binding fragment thereof that specifically binds to an antigen will bind to the antigen with a K_{d} of up to 100 nM (e.g., between 1 pM and 100 nM). An antibody or antigen-binding fragment thereof that does not exhibit specific binding to a particular antigen or epitope thereof will exhibit a K_{d} of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “subject” and “patient” refer to an organism that receives treatment for a particular disease or condition as described herein (such as cancer or an infectious disease). Examples of subjects and patients include mammals, such as humans, primates, pigs, goats, rabbits, hamsters, cats, dogs, guinea pigs, members of the bovidae family (such as cattle, bison, buffalo, elk, and yaks, among others), cows, sheep, horses, and bison, among others, receiving treatment for diseases or conditions, for example, cell proliferation disorders, such as cancer or infectious diseases.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection and the like.

As used herein, the terms “treat” or “treatment” refer to therapeutic treatment, in which the object is to prevent or slow down (lessen) an undesired physiological change or disorder, such as the progression of a cell proliferation disorder, such as cancer, or an infectious disease. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in which the condition or disorder is to be prevented.

As used herein, the term “tumor microenvironment” refers to cancer cells that form a tumor and the population of non-cancer cells, molecules, and/or blood vessels within the tumor or that border or surround the cancer cells.

As used herein, the terms “tumor necrosis factor receptor superfamily,” “TNFR superfamily,” “TNFRS,” “TNFRSF,” or “TNFRSF members” refer to a group of type I transmembrane proteins with a carboxy-terminal intracellular domain and an amino-terminal extracellular domain characterized by a common cysteine-rich domain (CRD). The TNFR superfamily includes receptors that mediate cellular signaling as a consequence of binding to one or more ligands in the TNF superfamily. The TNFR superfamily can be divided into two subgroups: receptors containing the intracellular death domain and those lacking this domain. The death domain is an 80 amino acid motif that propagates apoptotic signal transduction cascades following receptor activation. Exemplary TNFR superfamily members that contain the intracellular death domain include TNFR1, while TNFR2 represents a TNFR superfamily protein that does not contain this domain. Members of the TNFR superfamily include CD40, TNFR1, TNFR2, RANK, CD30, Lymphotoxin beta receptor (LT beta receptor or LT-8R), OX40, Fas receptor, Decoy receptor 3 (DCR3), CD27, 4-1 BB, Death receptor 4 (DR4), Death receptor 5 (DR5), Decoy receptor 1 (DCR1), Decoy receptor 2 (DCR2), Osteoprotegrin, TWEAK receptor, TACT, BAFF receptor, Herpesvirus entry mediator, Nerve growth factor receptor, B cell maturation antigen, Glucocorticoid-induced TNFR-related, TROY, Death receptor 6 (DR6), Death receptor 3 (DR3), and Ectodysplasin A2 receptor.

As used herein, the terms “tumor necrosis factor receptor superfamily member signaling,” “TNFRSF signaling,” “TNFRSF member signaling,” “TNFRSF signal transduction,” “TNFRSF member signal transduction,” and the like, are used interchangeably and refer to the cellular events that normally occur upon activation of a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) on the surface of a TNFRSF member expressing cell, such as T-reg cell, MDSC, or TNFRSF member expressing cancer cell, by an endogenous TNFRSF member ligand, such as CD40L for CD40, TNFα for TNFR1 and TNFR2,4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others. TNFRSF member signaling may be evidenced by a finding that expression is increased for one or more genes selected from the group consisting of CHUK, NFκBIE, NFκBIA, MAP3K11, TRAF2, TRAF3, relB, and cIAP2/BIRC3. TNFRSF member signaling is considered to be “inhibited” as used herein when the expression (and/or post-translational modification in the event that such a modification is required for activity of the encoded protein) of one or more, or all, of the foregoing genes is decreased in a TNFRSF member expressing cell upon contacting the cell with an agent, such as a TNFRSF member antagonist polypeptide described herein, relative to a TNFRSF member expressing cell that is not contacted with the agent (e.g., TNFRSF member antagonist polypeptide). TNFRSF member signaling is considered to be “inhibited,” for example, when the expression or post-translational modification (e.g., phosphorylation) of one or more of CHUK, NFκBIE, NFκBIA, MAP3K11, TRAF2, TRAF3, relB, or cIAP2/BIRC3, in a TNFRSF member expressing cell contacted with an antagonistic TNFRSF member polypeptide is decreased by about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% relative to the expression or post-translational modification (e.g., phosphorylation) of one or more of these genes in a TNFRSF member expressing cell not contacted with the antagonistic TNFRSF member polypeptide. Exemplary assays that can be used to determine expression level and phosphorylation state are known in the art and include, e.g., Western blot assays to determine protein content and quantitative reverse transcription polymerase chain reaction (RT-PCR) experiments to determine mRNA content.

As used herein the term “variable region CDR” includes amino acids in a CDR or complementarity determining region as identified using sequence or structure-based methods. As used herein, the term “CDR” or “complementarity determining region” refers to the noncontiguous antigen-binding sites found within the variable regions of both heavy and light chain polypeptides. These particular regions have been described by Kabat et al. (J. Biol. Chem. 252:6609-6616, 1977), Kabat, et al. (Sequences of Proteins of Immunological Interest, Fifth Edition, U.S. Department of Health and Human Services, NIH Publication No. 91-3242, 1991), Chothia et al. (J. Mol. Biol. 196:901-917, 1987), MacCallum et al. (J. Mol. Biol. 262:732-745, 1996), where the definitions include overlapping or subsets of amino acid residues when compared against each other. The term “CDR” may be, for example, a CDR as defined by Kabat based on sequence comparisons.

As used herein, the term “vector” includes a nucleic acid vector, e.g., a DNA vector, such as a plasmid, an RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments described herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “V_{H}” refers to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (V_{H}) followed by a number of constant domains. Each light chain of a native antibody has a variable domain at the amino terminus (V_{L}) and a constant domain at the carboxy terminus.

### BRIEF DESCRIPTION OF THE FIGURES

FIGS. 1A through 1V show the amino acid sequence of human TNFRSF members CD40 (SEQ ID NO: 4), 4-1BB (SEQ ID NO: 1), CD27 (SEQ ID NO: 2), CD30 (SEQ ID NO: 3), DR6 (SEQ ID NO: 5), EDAR (SEQ ID NO: 6), Fas (SEQ ID NO: 7), GITR (SEQ ID NO: 8), HVEM (SEQ ID NO: 9), LT beta receptor (SEQ ID NO: 10), NGFR (SEQ ID NO: 11), OPG (SEQ ID NO: 12), OX40 (SEQ ID NO: 13), RANK (SEQ ID NO: 14), RELT (19L) (SEQ ID NO: 15), TNFR1 (SEQ ID NO:16), TRAIL-R2 (TNFRSF10B) (SEQ ID NO: 17), TRAIL-R1 (TNFRSF10A) (SEQ ID NO: 18), TRAIL-R4 (SEQ ID NO: 19), TRAMP (SEQ ID NO: 20), TROY (SEQ ID NO: 21), and XEDAR (SEQ ID NO: 22), respectively. All references to amino acid positions within TNFRSF members are made in the context of the TNFRSF member numbering scheme shown in FIGS. 1A through 1V.

FIG. 1A shows the amino acid sequence of human CD40 (SEQ ID NO: 4). Notably, CD40 is numbered herein starting with an N-terminal methionine at position 1 and concluding with a C-terminal glutamine at position 277 (SEQ ID NO: 4). Underlined amino acids SCSPGFGVK (SEQ ID NO: 25 (residues 124-132 of SEQ ID NO: 4)) and amino acids CEPCPVGFFS (SEQ ID NO: 26 (residues 143-152 of SEQ ID NO: 4)) define epitopes, one or more amino acids of one or both of which may be bound by a CD40 antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of CD40 and may be appropriately positioned for interaction with an antagonistic CD40 antibody of the disclosure. Some or all of the amino acids of the epitope(s), or one or more amino acids in a region next to the epitope(s), or spatially near or next to the epitopes based on the tertiary structure of CD40, may be bound by a CD40 antagonist to achieve antagonism of CD40.

FIG. 1B shows the amino acid sequence of human 4-1BB (SEQ ID NO: 1). Notably, 4-1BB is numbered herein starting with an N-terminal methionine at position 1 and concluding with a C-terminal leucine at position 255 (SEQ ID NO: 1). Underlined amino acids MCEQDCKQGQELTKKGCKDCCFGTFNDQK (SEQ ID NO: 27 (residues 101-129 of SEQ ID NO: 1)) define epitopes, one or more amino acids that may be bound by a 4-1 BB antagonist (e.g., an antagonistic polypeptide, such as a single-chain polypeptide, antibody, or antigen-binding fragment thereof). Some or all of the amino acids of the epitope(s), or one or more amino acids in a region next to the epitope(s), or spatially near or next to the epitopes based on the tertiary structure of 4-1 BB, may be bound by a 4-1 BB antagonist to achieve antagonism of 4-1 BB.

FIG. 1C shows the amino acid sequence of human CD27 (SEQ ID NO: 2). Notably, CD27 is numbered herein starting with an N-terminal methionine at position 1 and concluding with a C-terminal proline at position 260 (SEQ ID NO: 2). Underlined amino acids QMCEPGT (SEQ ID NO: 28 (residues 41-47 of SEQ ID NO: 2)) and amino acids QCDPCIPGVSFS (SEQ ID NO: 29 (residues 61-72 of SEQ ID NO: 2)) define epitopes, one or more amino acids of one or both of which may be bound by a CD27 antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of CD27 and may be appropriately positioned for interaction with an antagonistic CD27 antibody of the disclosure. Some or all of the amino acids of the epitope(s), or one or more amino acids in a region next to the epitope(s), or spatially near or next to the epitopes based on the tertiary structure of CD27, may be bound by a CD27 antagonist to achieve antagonism of CD27.

FIG. 1D shows the amino acid sequence of human CD30 (SEQ ID NO: 3). Notably, CD30 is numbered herein starting with an N-terminal methionine at position 1 and concluding with a C-terminal lysine at position 595 (SEQ ID NO: 3). Underlined amino acids SVCPAGMIVKFP (SEQ ID NO: 30 (129-140 of SEQ ID NO: 3)) and amino acids CEPASPGVS (SEQ ID NO: 31 (residues 149-157 of SEQ ID NO: 3)) define epitopes, one or more amino acids of one or both of which may be bound by a CD30 antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of CD30 and may be appropriately positioned for interaction with an antagonistic CD30 antibody of the disclosure. Some or all of the amino acids of the epitope(s), or one or more amino acids in a region next to the epitope(s), or spatially near or next to the epitopes based on the tertiary structure of CD30, may be bound by a CD30 antagonist to achieve antagonism of CD30.

FIG. 1E shows the amino acid sequence of human DR6 (SEQ ID NO: 5). Notably, DR6 is numbered herein starting with an N-terminal methionine at position 1 and concluding with a C-terminal leucine at position 655 (SEQ ID NO: 5). Underlined amino acids NGTCAPHTVCPVGWGV (SEQ ID NO: 32 (residues 141-156 of SEQ ID NO: 5)) and amino acids KQCARGTFS (SEQ ID NO: 33 (residues 169-177 of SEQ ID NO: 5)) define epitopes, one or more amino acids of one or both of which may be bound by a DR6 antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of DR6 and may be appropriately positioned for interaction with an antagonistic DR6 antibody of the disclosure. Some or all of the amino acids of the epitope(s), or one or more amino acids in a region next to the epitope(s), or spatially near or next to the epitopes based on the tertiary structure of DR6, may be bound by a DR6 antagonist to achieve antagonism of DR6.

FIG. 1F shows the amino acid sequence of human EDAR (SEQ ID NO: 6). Notably, EDAR is numbered herein starting with an N-terminal methionine at position 1 and concluding with a C-terminal serine at position 448 (SEQ ID NO: 6). Underlined amino acids KDCEGFFRATVL (SEQ ID NO: 34 (residues 91-102 of SEQ ID NO: 6)) and amino acids AECGPCLPGYYM (SEQ ID NO: 35 (residues 111-122 of SEQ ID NO: 6)) define epitopes, one or more amino acids of one or both of which may be bound by a EDAR antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of EDAR and may be appropriately positioned for interaction with an antagonistic EDAR antibody of the disclosure. Some or all of the amino acids of the epitope(s), or one or more amino acids in a region next to the epitope(s), or spatially near or next to the epitopes based on the tertiary structure of EDAR, may be bound by a EDAR antagonist to achieve antagonism of EDAR.

FIG. 1G shows the amino acid sequence of human Fas (SEQ ID NO: 7). Notably, Fas is numbered herein starting with an N-terminal methionine at position 1 and concluding with a C-terminal valine at position 335 (SEQ ID NO: 7). Underlined amino acids KCEHGIIKE (SEQ ID NO: 36 (residues 148-156 of SEQ ID NO: 7)) and amino acids CKEEGSRSN (SEQ ID NO: 37 (residues 165-173 of SEQ ID NO: 7)) define epitopes, one or more amino acids of one or both of which may be bound by a Fas antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of Fas and may be appropriately positioned for interaction with an antagonistic Fas antibody of the disclosure. Some or all of the amino acids of the epitope(s), or one or more amino acids in a region next to the epitope(s), or spatially near or next to the epitopes based on the tertiary structure of Fas, may be bound by a Fas antagonist to achieve antagonism of Fas.

FIG. 1H shows the amino acid sequence of human GITR (SEQ ID NO: 8). Notably, GITR is numbered herein starting with an N-terminal methionine at position 1 and concluding with a C-terminal valine at position 241 (SEQ ID NO: 8). Underlined amino acids HPCPPGQGVQ (SEQ ID NO: 38 (residues 92-101 of SEQ ID NO: 8)) and amino acids CIDCASGTFS (SEQ ID NO: 39 (residues 112-121 of SEQ ID NO: 8)) define epitopes, one or more amino acids of one or both of which may be bound by a GITR antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of GITR and may be appropriately positioned for interaction with an antagonistic GITR antibody of the disclosure. Some or all of the amino acids of the epitope(s), or one or more amino acids in a region next to the epitope(s), or spatially near or next to the epitopes based on the tertiary structure of GITR, may be bound by a GITR antagonist to achieve antagonism of GITR.

FIG. 11 shows the amino acid sequence of human HVEM (SEQ ID NO: 9). Notably, HVEM is numbered herein starting with an N-terminal methionine at position 1 and concluding with a C-terminal asparagine at position 275 (SEQ ID NO: 9). Underlined amino acids TTCPPGQRVEK (SEQ ID NO: 40 (residues 142-152 of SEQ ID NO: 9)) and amino acids CADCLTGTF (SEQ ID NO: 41 (residues 162-170 of SEQ ID NO: 9)) define epitopes, one or more amino acids of one or both of which may be bound by a HVEM antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of HVEM and may be appropriately positioned for interaction with an antagonistic HVEM antibody of the disclosure. Some or all of the amino acids of the epitope(s), or one or more amino acids in a region next to the epitope(s), or spatially near or next to the epitopes based on the tertiary structure of HVEM, may be bound by a HVEM antagonist to achieve antagonism of HVEM.

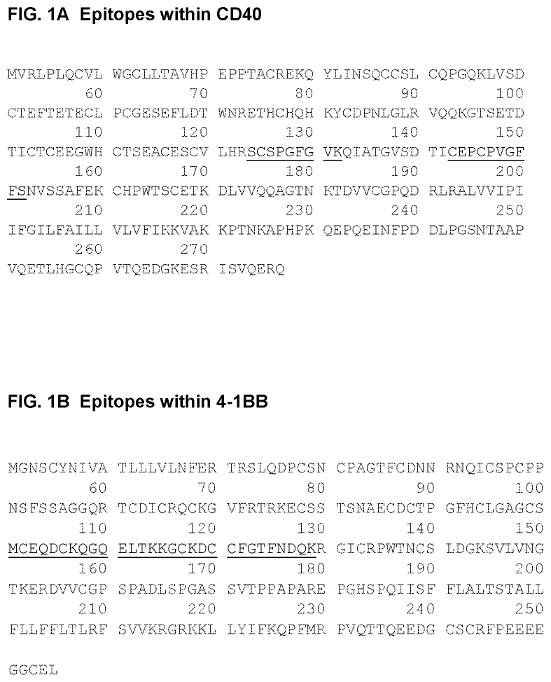

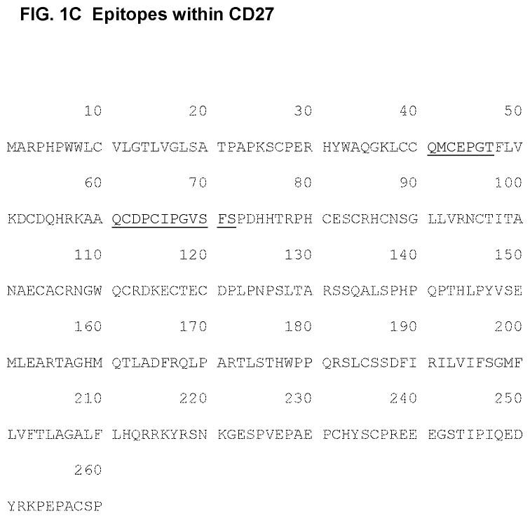

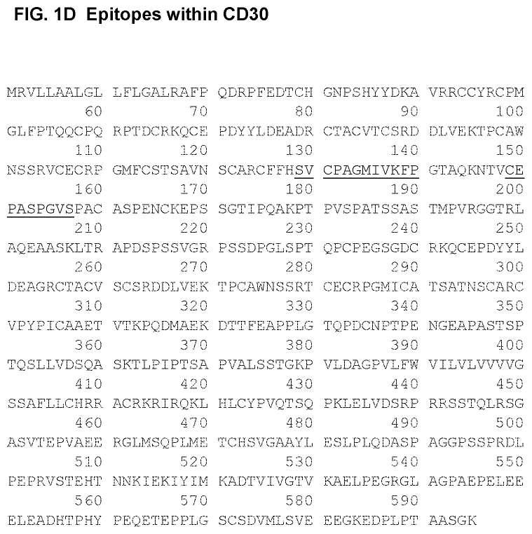

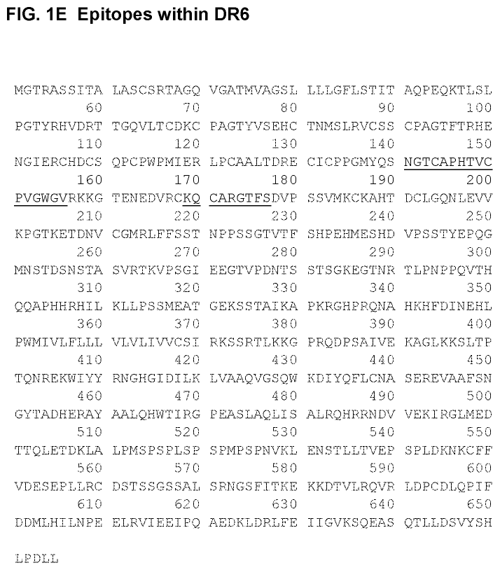

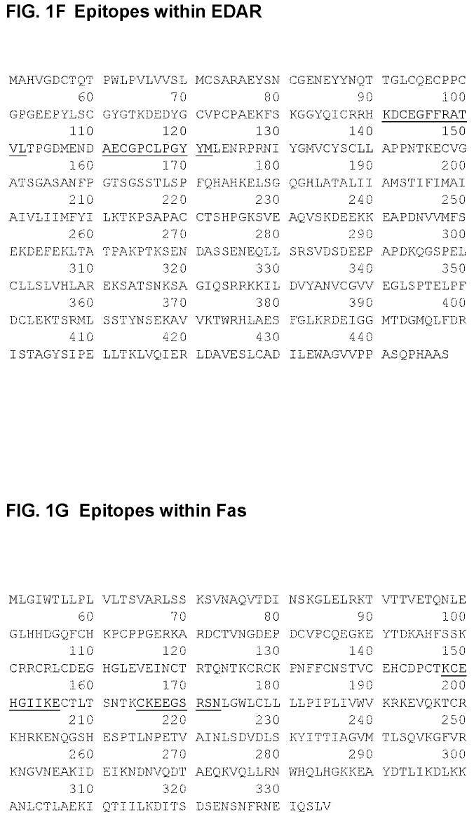

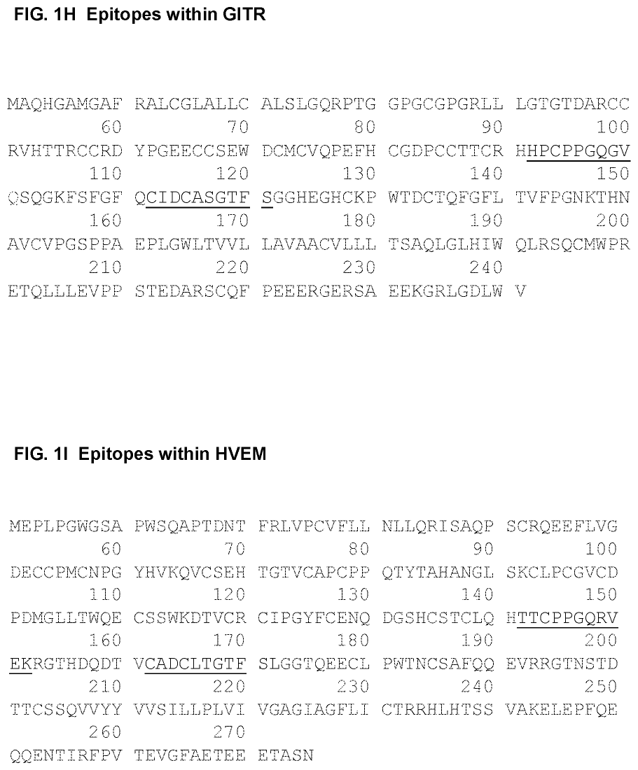

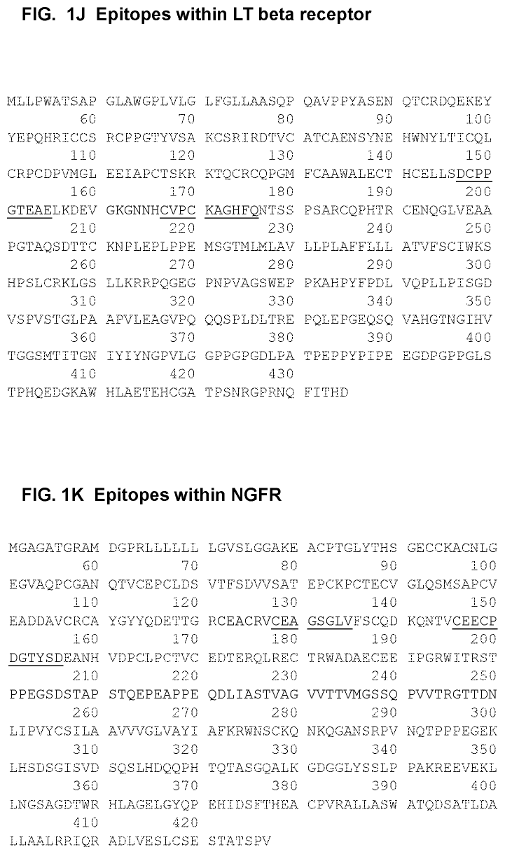

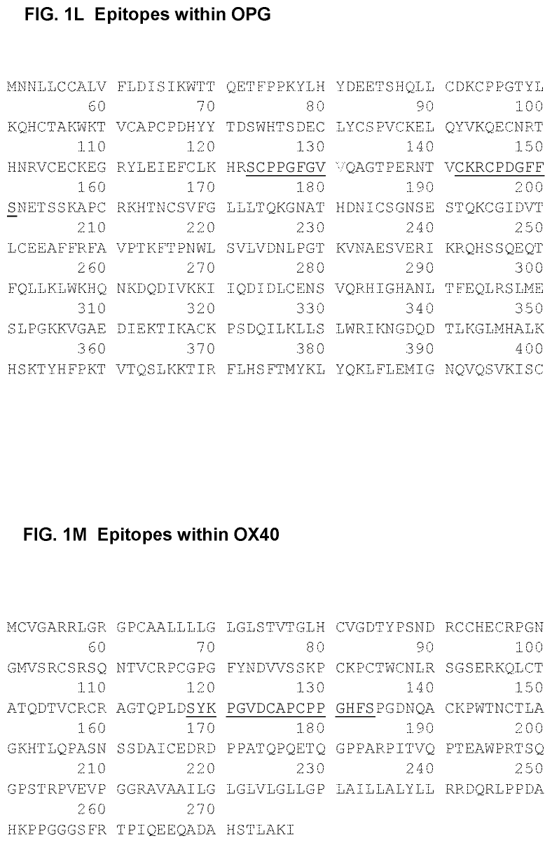

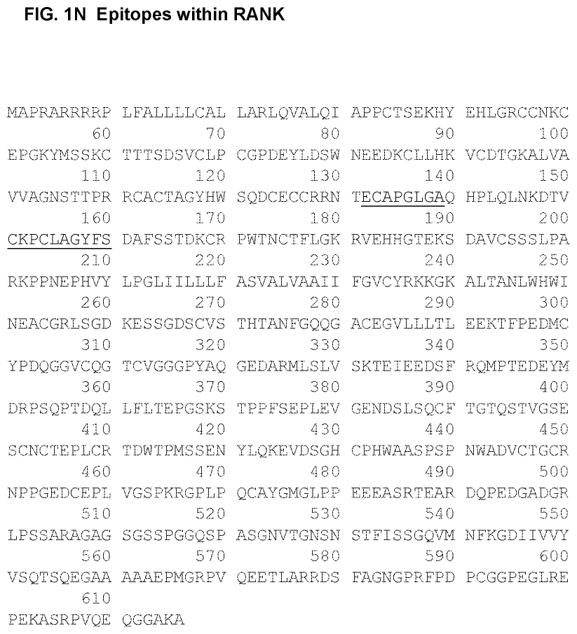

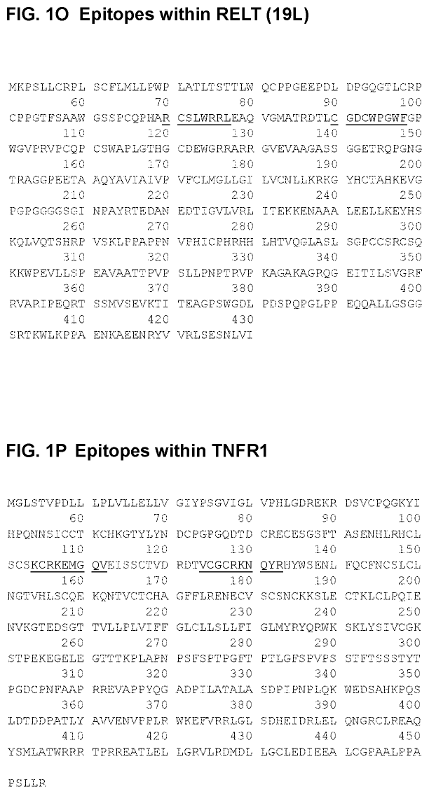

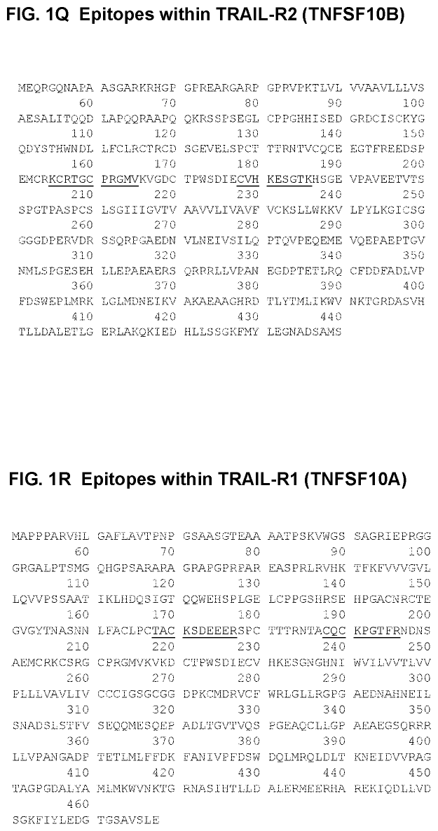

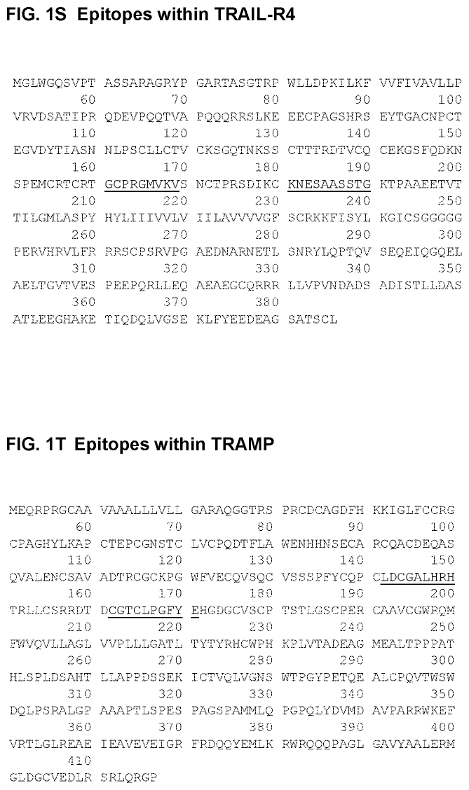

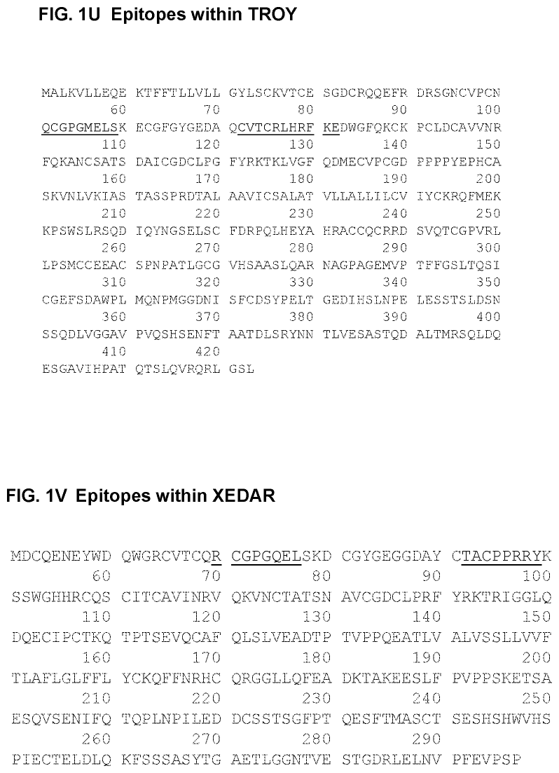

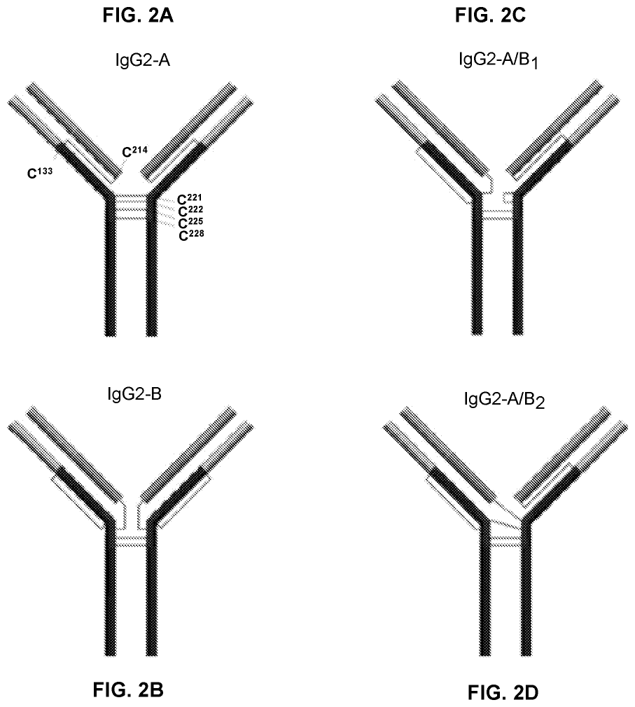

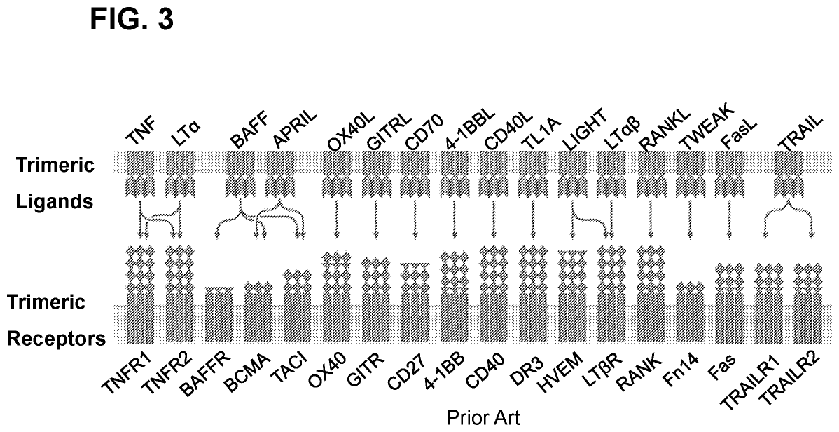

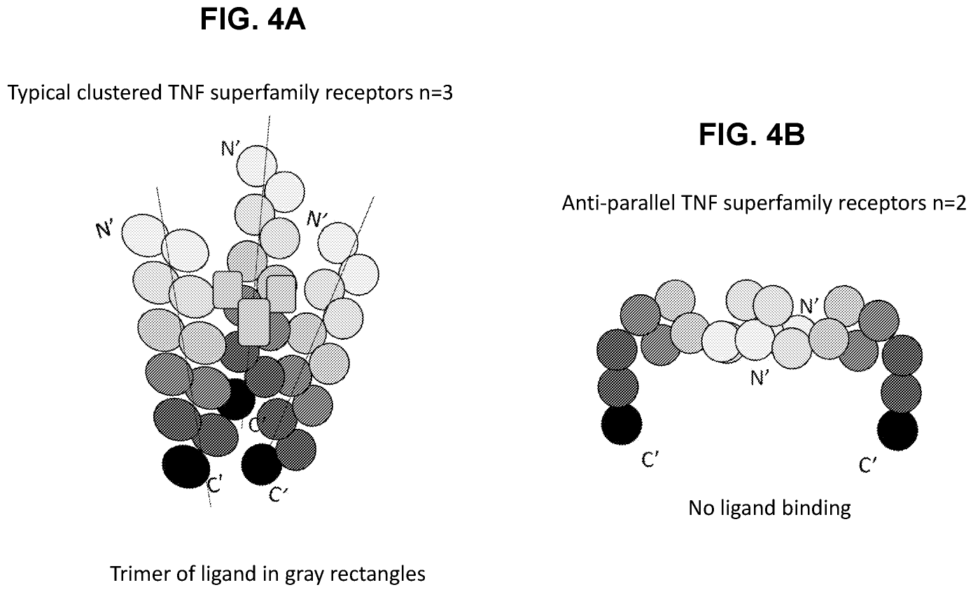

FIG. 1J shows the amino acid sequence of human LT beta receptor (SEQ ID NO: 10). Notably, LT beta receptor is numbered herein starting with an N-terminal methionine at position 1 and concluding with a C-terminal aspartic acid at position 435 (SEQ ID NO: 10). Underlined amino acids DCPPGTEAE (SEQ ID NO: 42 (residues 147-155 of SEQ ID NO: 10)) and amino acids CVPCKAGHFQ (SEQ ID NO: 43 (residues 167-176 of SEQ ID NO: 10)) define epitopes, one or more amino acids of one or both of which may be bound by a LT beta receptor antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of LT beta receptor and may be appropriately positioned for interaction with an antagonistic LT beta receptor antibody of the disclosure. Some or all of the amino acids of the epitope(s), or one or more amino acids in a region next to the epitope(s), or spatially near or next to the epitopes based on the tertiary structure of LT beta receptor, may be bound by a LT beta receptor antagonist to achieve antagonism of LT beta receptor.

FIG. 1K shows the amino acid sequence of human NGFR (SEQ ID NO: 11). Notably, NGFR is numbered herein starting with an N-terminal methionine at position 1 and concluding with a C-terminal valine at position 427 (SEQ ID NO: 11). Underlined amino acids CEAGSGLV (SEQ ID NO: 44 (residues 128-135 of SEQ ID NO: 11)) and amino acids CEECPDGTYSD (SEQ ID NO: 45 (residues 146-156 of SEQ ID NO: 11)) define epitopes, one or more amino acids of one or both of which may be bound by a NGFR antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of NGFR and may be appropriately positioned for interaction with an antagonistic NGFR antibody of the disclosure. Some or all of the amino acids of the epitope(s), or one or more amino acids in a region next to the epitope(s), or spatially near or next to the epitopes based on the tertiary structure of NGFR, may be bound by a NGFR antagonist to achieve antagonism of NGFR.

FIG. 1L shows the amino acid sequence of human OPG (SEQ ID NO: 12). Notably, OPG is numbered herein starting with an N-terminal methionine at position 1 and concluding with a C-terminal cysteine at position 400 (SEQ ID NO: 12). Underlined amino acids SCPPGFGVV (SEQ ID NO: 46 (residues 123-131 of SEQ ID NO: 12)) and amino acids CKRCPDGFFS (SEQ ID NO: 47 (residues 142-151 of SEQ ID NO: 12)) define epitopes, one or more amino acids of one or both of which may be bound by a OPG antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of OPG and may be appropriately positioned for interaction with an antagonistic OPG antibody of the disclosure. Some or all of the amino acids of the epitope(s), or one or more amino acids in a region next to the epitope(s), or spatially near or next to the epitopes based on the tertiary structure of OPG, may be bound by a OPG antagonist to achieve antagonism of OPG.

FIG. 1M shows the amino acid sequence of human OX40 (SEQ ID NO: 13). Notably, OX40 is numbered herein starting with an N-terminal methionine at position 1 and concluding with a C-terminal isoleucine at position 277 (SEQ ID NO: 13). Underlined amino acids SYKPGVDCAPCPPGHFS (SEQ ID NO: 48 (residues 118-134 of SEQ ID NO: 13)) define epitopes, one or more amino acids that may be bound by a OX40 antagonist (e.g., an antagonistic polypeptide, such as a single-chain polypeptide, antibody, or antigen-binding fragment thereof). Some or all of the amino acids of the epitope(s), or one or more amino acids in a region next to the epitope(s), or spatially near or next to the epitopes based on the tertiary structure of OX40, may be bound by a OX40 antagonist to achieve antagonism of OX40.

FIG. 1N shows the amino acid sequence of human RANK (SEQ ID NO: 14). Notably, RANK is numbered herein starting with an N-terminal methionine at position 1 and concluding with a C-terminal alanine at position 616 (SEQ ID NO: 14). Underlined amino acids ECAPGLGA (SEQ ID NO: 49 (residues 132-139 of SEQ ID NO: 14)) and amino acids CKPCLAGYFS (SEQ ID NO: 50 (residues 151-160 of SEQ ID NO: 14)) define epitopes, one or more amino acids of one or both of which may be bound by a RANK antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of RANK and may be appropriately positioned for interaction with an antagonistic RANK antibody of the disclosure. Some or all of the amino acids of the epitope(s), or one or more amino acids in a region next to the epitope(s), or spatially near or next to the epitopes based on the tertiary structure of RANK, may be bound by a RANK antagonist to achieve antagonism of RANK.

FIG. 1O shows the amino acid sequence of human RELT (19L) (SEQ ID NO: 15). Notably, RELT (19L) is numbered herein starting with an N-terminal methionine at position 1 and concluding with a C-terminal isoleucine at position 430 (SEQ ID NO: 15). Underlined amino acids RCSLWRRL (SEQ ID NO: 51 (residues 70-77 of SEQ ID NO: 15)) and amino acids CGDCWPGWF (SEQ ID NO: 52 (residues 90-98 of SEQ ID NO: 15)) define epitopes, one or more amino acids of one or both of which may be bound by a RELT (19L) antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of RELT (19L) and may be appropriately positioned for interaction with an antagonistic RELT (19L) antibody of the disclosure. Some or all of the amino acids of the epitope(s), or one or more amino acids in a region next to the epitope(s), or spatially near or next to the epitopes based on the tertiary structure of RELT (19L), may be bound by a RELT (19L) antagonist to achieve antagonism of RELT (19L).

FIG. 1P shows the amino acid sequence of human TNFR1 (SEQ ID NO: 16). Notably, TNFR1 is numbered herein starting with an N-terminal methionine at position 1 and concluding with a C-terminal arginine at position 455 (SEQ ID NO: 16). Underlined amino acids KCRKEMGQV (SEQ ID NO: 53 (residues 104-112 of SEQ ID NO: 16)) and amino acids VCGCRKNQYR (SEQ ID NO: 54 (residues 124-133 of SEQ ID NO: 16)) define epitopes, one or more amino acids of one or both of which may be bound by a TNFR1 antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of TNFR1 and may be appropriately positioned for interaction with an antagonistic TNFR1 antibody of the disclosure. Some or all of the amino acids of the epitope(s), or one or more amino acids in a region next to the epitope(s), or spatially near or next to the epitopes based on the tertiary structure of TNFR1, may be bound by a TNFR1 antagonist to achieve antagonism of TNFR1.

FIG. 1O shows the amino acid sequence of human TRAIL-R2 (TNFRSF10B) (SEQ ID NO: 17). Notably, TRAIL-R2 (TNFRSF10B) is numbered herein starting with an N-terminal methionine at position 1 and concluding with a C-terminal serine at position 440 (SEQ ID NO: 17). Underlined amino acids KCRTGCPRGMV (SEQ ID NO: 55 (residues 155-165 of SEQ ID NO: 17)) and amino acids CVHKESGTK (SEQ ID NO: 56 (residues 178-186 of SEQ ID NO: 17)) define epitopes, one or more amino acids of one or both of which may be bound by a TRAIL-R2 (TNFRSF10B) antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of TRAIL-R2 (TNFRSF10B) and may be appropriately positioned for interaction with an antagonistic TRAIL-R2 (TNFRSF10B) antibody of the disclosure. Some or all of the amino acids of the epitope(s), or one or more amino acids in a region next to the epitope(s), or spatially near or next to the epitopes based on the tertiary structure of TRAIL-R2 (TNFRSF10B), may be bound by a TRAIL-R2 (TNFRSF10B) antagonist to achieve antagonism of TRAIL-R2 (TNFRSF10B).

FIG. 1R shows the amino acid sequence of human TRAIL-R1 (TNFRSF10A) (SEQ ID NO: 18). Notably, TRAIL-R1 (TNFRSF10A) is numbered herein starting with an N-terminal methionine at position 1 and concluding with a C-terminal glutamic acid at position 468 (SEQ ID NO: 18). Underlined amino acids ACKSDEEER (SEQ ID NO: 57 (residues 169-177 of SEQ ID NO: 18)) and amino acids CQCKPGTFR (SEQ ID NO: 58 (residues 188-196 of SEQ ID NO: 18)) define epitopes, one or more amino acids of one or both of which may be bound by a TRAIL-R1 (TNFRSF10A) antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of TRAIL-R1 (TNFRSF10A) and may be appropriately positioned for interaction with an antagonistic TRAIL-R1 (TNFRSF10A) antibody of the disclosure. Some or all of the amino acids of the epitope(s), or one or more amino acids in a region next to the epitope(s), or spatially near or next to the epitopes based on the tertiary structure of TRAIL-R1 (TNFRSF10A), may be bound by a TRAIL-R1 (TNFRSF10A) antagonist to achieve antagonism of TRAIL-R1 (TNFRSF10A).

FIG. 1S shows the amino acid sequence of human TRAIL-R4 (SEQ ID NO: 19). Notably, TRAIL-R4 is numbered herein starting with an N-terminal methionine at position 1 and concluding with a C-terminal leucine at position 386 (SEQ ID NO: 19). Underlined amino acids GCPRGMVKV (SEQ ID NO: 59 (residues 161-169 of SEQ ID NO: 19)) and amino acids KNESAASSTG (SEQ ID NO: 60 (residues 181-190 of SEQ ID NO: 19)) define epitopes, one or more amino acids of one or both of which may be bound by a TRAIL-R4 antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of TRAIL-R4 and may be appropriately positioned for interaction with an antagonistic TRAIL-R4 antibody of the disclosure. Some or all of the amino acids of the epitope(s), or one or more amino acids in a region next to the epitope(s), or spatially near or next to the epitopes based on the tertiary structure of TRAIL-R4, may be bound by a TRAIL-R4 antagonist to achieve antagonism of TRAIL-R4.

FIG. 1T shows the amino acid sequence of human TRAMP (SEQ ID NO: 20). Notably, TRAMP is numbered herein starting with an N-terminal methionine at position 1 and concluding with a C-terminal proline at position 417 (SEQ ID NO: 20). Underlined amino acids LDCGALHRH (SEQ ID NO: 61 (residues 142-150 of SEQ ID NO: 20)) and amino acids CGTCLPGFYE (SEQ ID NO: 62 (residues 162-171 of SEQ ID NO: 20)) define epitopes, one or more amino acids of one or both of which may be bound by a TRAMP antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of TRAMP and may be appropriately positioned for interaction with an antagonistic TRAMP antibody of the disclosure. Some or all of the amino acids of the epitope(s), or one or more amino acids in a region next to the epitope(s), or spatially near or next to the epitopes based on the tertiary structure of TRAMP, may be bound by a TRAMP antagonist to achieve antagonism of TRAMP.

FIG. 1U shows the amino acid sequence of human TROY (SEQ ID NO: 21). Notably, TROY is numbered herein starting with an N-terminal methionine at position 1 and concluding with a C-terminal leucine at position 423 (SEQ ID NO: 21). Underlined amino acids QCGPGMELS (SEQ ID NO: 63 (residues 51-59 of SEQ ID NO: 21)) and amino acids CVTCRLHRFKE (SEQ ID NO: 64 (residues 72-82 of SEQ ID NO: 21)) define epitopes, one or more amino acids of one or both of which may be bound by a TROY antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of TROY and may be appropriately positioned for interaction with an antagonistic TROY antibody of the disclosure. Some or all of the amino acids of the epitope(s), or one or more amino acids in a region next to the epitope(s), or spatially near or next to the epitopes based on the tertiary structure of TROY, may be bound by a TROY antagonist to achieve antagonism of TROY.

FIG. 1V shows the amino acid sequence of human XEDAR (SEQ ID NO: 22). Notably, XEDAR is numbered herein starting with an N-terminal methionine at position 1 and concluding with a C-terminal proline at position 297 (SEQ ID NO: 22). Underlined amino acids RCGPGQEL (SEQ ID NO: 65 (residues 20-27 of SEQ ID NO: 22)) and amino acids TACPPRRY (SEQ ID NO: 66 (residues 42-49 of SEQ ID NO: 22)) define epitopes, one or more amino acids of one or both of which may be bound by a XEDAR antagonist (e.g., an antagonistic polypeptide, such as a single-chain polypeptide, antibody, or antigen-binding fragment thereof). Though these residues are not consecutive in primary sequence, they are likely spatially proximal in the three dimensional tertiary structure of XEDAR and may be appropriately positioned for interaction with an antagonistic XEDAR antibody of the disclosure. Some or all of the amino acids of the epitope(s), or one or more amino acids in a region next to the epitope(s), or spatially near or next to the epitopes based on the tertiary structure of XEDAR, may be bound by a XEDAR antagonist to achieve antagonism of XEDAR.

FIGS. 2A-2D are a series of schematics comparing the disulfide bonding arrangement present in each of the IgG2-A (FIG. 2A), IgG2-B (FIG. 2B), IgG2-A/Bi (FIG. 2C), and IgG2-A/B2 (FIG. 2D) isoforms of a human IgG2 isotype antibody. Thin lines represent disulfide bonds connecting various portions of each antibody heavy chain or light chain, which are represented by shaded rectangles. Heavy chains are represented by the longer, outermost rectangles of each antibody. Within each heavy chain, black shading denotes the constant region, and light shading denotes the variable region. Light chains are represented by the shorter, innermost rectangles of each antibody. Within each light chain, darker shading denotes the constant region, and lighter shading denotes the variable region.

FIG. 3 shows homotrimeric TNFRSF member proteins and their respective ligands. Antagonistic polypeptides of the disclosure can bind a specific TNFRSF member and stabilize an inactive, antiparallel homodimeric conformation, thus preventing the cognate or natural ligand of the TNFRSF member from binding and forming an active, homotrimeric conformation. FIG. 3 is adapted from Croft, et al. (Nat. Rev. Rheumatol. 13:217-233, 2017), which is incorporated herein by reference.

FIGS. 4A and 4B depict an active, homotrimeric conformation of a TNFRSF member protein (such as, e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR) (FIG. 4A) and an inactive, homodimeric conformation of a TNFRSF member protein (FIG. 4B). The active, homotrimeric conformation in FIG. 4A is stabilized by a bound, homotrimer of the natural ligand. The inactive, homodimeric conformation in FIG. 4B depicts the receptor in an antiparallel dimer conformation, which is a non-signaling state. An antagonist antibody (e.g., an antagonist TNFSF antibody described herein) can stabilize the antiparallel dimer conformation, thus preventing the natural ligand from binding the TNFRSF member.

### DETAILED DESCRIPTION

Antagonistic polypeptides of the disclosure (e.g., single-chain polypeptides, antibodies, and antigen-binding fragments) that are specific for a human TNFRSF member, such as CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, inhibit the activation of a human TNFRSF member protein by binding this receptor (e.g., on the exterior surface of a cell, such as T-reg cell, a cancer cell that expresses the TNFRSF member, a myeloid-derived suppressor cell (MDSC), a T cell, a B cell, a monocyte, a neutrophil, a platelet, a granulocyte, a bone marrow derived lymphoid cells, or a parenchymal cell), thereby preventing the protein from recruiting its cognate or natural ligand. The cognate or natural ligands of the TNFRSF member proteins described herein potentiate TNFRSF member protein signaling by nucleating a trimer of TNFRSF member proteins. It is this trimerization event that brings individual TNFRSF member proteins into close proximity, thereby initiating intracellular signaling. TNFRSF member antagonist polypeptides (e.g., single-chain polypeptides, antibodies, and antibody fragments, such as anti-CD40 polypeptides that inhibit CD40 activation) can antagonize this interaction by binding the receptor and preventing the cognate or natural ligand from triggering the structural change that initiates intracellular signaling.

A TNFRSF member protein can be categorized into one of three groups (e.g., death domain (DD)-containing receptors, decoy receptors, and TNF receptor-associated factor (TRAF)-binding receptors), depending on the specific intracellular signal induced by the TNFRSF member. DD-containing receptors, e.g., TRAIL-R1 (TNFRSF10A), TRAIL-R2 (TNFRSF10B, TRAMP, DR6, NGFR, TNFR1, Fas, and EDAR, mediate apoptotic signals in, e.g., T-reg cells, CD8+ cytotoxic T cells, B cells, monocytes, neutrophils, platelets, granulocytes, bone marrow derived lymphoid cells, and parenchymal cells. Decoy receptors, e.g., IL1 R2, DcR3, and VEGFR-1, are able to recognize and bind specific growth factors or cytokines, but are not able to signal or activate the intended receptor complex. TRAF-binding receptors, e.g., CD40, TRAIL-R3, TRAIL-R4, RANK, FN14, TNFR2, LT beta receptor, HVEM, CD30, CD27, 4-1BB, OX40, GITR, BCMA, TACT, BAFF-R, XEDAR, TROY, and RELT (19L), mediate proliferative signal pathways in, e.g., T-reg cells, CD8+ cytotoxic T cells, B cells, monocytes, neutrophils, platelets, granulocytes, bone marrow derived lymphoid cells, and parenchymal cells.

Tables 2 and 3 list DD-containing and TRAF-binding TNFRSF members, respectively. The tables include the natural ligand function of the receptor, as well as diseases that may be treated by a TNFRSF member antagonistic polypeptide (e.g., single-chain polypeptide, antibody, and antigen-binding fragments).

TABLE 2
DD-containing receptors and diseases that may be
treated by an antagonistic polypeptide thereof.

| | | | Diseases that may be |
|---|---|---|---|
| | Natural | Antagonist | treated by an |
| DD-containing | ligand | polypeptide | antagonistic |
| receptor | function | function | polypeptide |
| TRAIL-R1 | Death of cells | Suppresses apoptosis of, | Autoimmunity |
| (TNFRSF10A) | (e.g., tumor cells) | e.g., T-reg cells, MDSCs | |
| TRAIL-R2 | Death of cells | Suppresses apoptosis of, | Autoimmunity |
| (TNFRSF10B) | (e.g., tumor cells) | e.g., T-reg cells, MDSCs | |
| TRAMP | Stimulates T-regs | Suppresses apoptosis of, | Cancer, infectious diseases |
| | | e.g., CD8+ cytotoxic T | |
| | | cells | |
| DR6 | Causes cell death | Suppresses apoptosis of, | Neuron-specific diseases |
| | | e.g., CD8+ cytotoxic T | (e.g., Alzheimer's disease), |
| | | cells, B cells, neural cells | cancer, infectious diseases |
| NGFR | Maintenance of | Suppresses apoptosis of, | Cancer |
| | neurons | e.g., CD8+ cytotoxic T | |
| | | cells, B cells | |
| TNFR1 | Increase | Suppresses apoptosis of, | Autoimmunity, inflammation, |
| | inflammation | e.g., T-reg cells, MDSCs | transplant rejections, asthma |
| Fas | Kills cells (e.g., | Suppresses apoptosis of, | Obesity, diabetes (e.g., type 2 |
| | tumor cells) | e.g., T-reg cells, MDSCs | diabetes), hyperlipidemia |
| EDAR | Developmental | — | — |
| | biology, e.g., | | |
| | embryonic | | |
| | signaling pathway | | |
| | that allows | | |
| | development of | | |
| | ectoderm | | |
| | structures | | |
| | including skin, | | |
| | hair, nails, teeth, | | |
| | and sweat glands | | |

TABLE 3
TRAF-binding receptors and diseases that may be
treated by an antagonistic polypeptide thereof.

| | | | Diseases that may be |
|---|---|---|---|
| | Natural | Antagonist | treated by an |
| TRAF-binding | ligand | polypeptide | antagonistic |
| receptor | function | function | polypeptide |
| TRAIL-R3 | Decoy receptor | Inhibits proliferation of, | Cancer, infectious diseases |
| | | e.g., T-reg cells, tumor | |
| | | cells, MDSCs | |
| TRAIL-R4 | Incomplete death | Inhibits proliferation of, | Cancer, infectious diseases |
| | domain | e.g., T-reg cells, tumor | |
| | | cells, MDSCs | |
| RANK | Regulates | Inhibits proliferation of, | Osteoporosis, tumor |
| | osteoclast | e.g., osteoclast cells | metastatic disease, |
| | differentiation | | decreased bone loss in |
| | | | cancer |
| FN14 | Stimulate brain | Inhibits proliferation of, | Glioblastoma, brain tumors |
| | tumors | e.g., T-reg cells, tumor | |
| | | cells, MDSCs | |
| TNFR2 | Stimulation of T- | Inhibits proliferation of, | Cancer, infectious diseases |
| | regs; death of T | e.g., T-reg cells, tumor | |
| | effectors | cells, MDSCs | |
| LT beta | Lymphoid | Inhibits proliferation of, | Cancer (e.g., B cell |
| receptor | development, | e.g., T-reg cells, cancer | lymphomas, melanomas, liver |
| | tumor growth | cells, MDSCs | cancer, renal cancer, |
| | | | fibrosarcomas) |
| HVEM | Limits | Inhibits proliferation of, | Cancer, infectious diseases |
| | inflammation, TCR | e.g., T-reg cells, cancer | |
| | activation, B cells | cells, MDSCs | |
| CD40 | Proliferates T | Inhibits proliferation of, | Autoimmunity, allergies, |
| | effectors | e.g., CD8+ cytotoxic T | asthma |
| | | cells, B cells, platelets, | |
| | | macrophages, dendritic | |
| | | cells | |
| CD30 | Proliferation | Inhibits proliferation of, | Cancer (e.g., Hodgkin |
| | | e.g., T-reg cells, cancer | lymphoma, chronic Hodgkin |
| | | cells, MDSCs | lymphoma, refractory |
| | | | Hodgkin lymphoma, and |
| | | | anaplastic large cell |
| | | | lymphoma) |
| CD27 | NFKB activation | Inhibits proliferation of, | Autoimmunity, allergies, |
| | CD8 memory T | e.g., CD4+ and CD8+ | asthma |
| | cells | cytotoxic T cells | |
| 4-1BB | Activate CD8 T, | Inhibits proliferation of, | Autoimmunity, asthma, |
| | NK cells increased | e.g., CD4+ and CD8+ | transplant rejection |
| | cytokines, | cytotoxic T cells, NK cells | |
| | cytotoxicity | | |
| OX40 | Activate CD8+ T, | Inhibits proliferation of, | Autoimmunity, asthma, |
| | NK cells increased | e.g., CD4+ and CD8+ | transplant rejection |
| | cytokines, | cytotoxic T cells, B cells | |
| | cytotoxicity | | |
| GITR | Prevents cell | Inhibits proliferation of, | Transplant rejection, |
| | growth | e.g., CD8+ cytotoxic T | autoimmunity, asthma, |
| | | cells, NK cells | allergies |
| BCMA | NK and B cell | Inhibits proliferation of, | Cancer for B cell tumors, e.g., |
| (TNFRSF17) | activation | e.g., T-reg cells, B cells, | multiple myeloma (e.g., |
| | | MDSCs | refractory multiple myeloma) |
| TACI | NK and B cell | Inhibits proliferation of, | Multiple myeloma (e.g., |
| | activation | e.g., T-reg cells, B cells, | refractory multiple myeloma), |
| | | MDSCs | breast cancer |
| BAFF-R | B cell | Inhibits proliferation of, | Cancer for B cell tumors, e.g., |
| | development | e.g., T-reg cells, B cells, | B cell acute lymphoblastic |
| | | MDSCs | leukemia, multiple myeloma |
| XEDAR | B cell | Inhibits proliferation of, | Allergies, autoimmunity, |
| | maintenance | e.g., CD8+ cytotoxic T | transplant rejection |
| | | cells, B cells | |
| TROY | Cell maintenance | Inhibits proliferation of, | Cancer, e.g., colon cancer |
| | (e.g., in the colon) | e.g., T-reg cells, cancer | and glioblastoma |
| | | cells, MDSCs | |
| RELT (19L) | Cell death | Inhibits proliferation of, | Nerve/CNS tumors, infectious |
| | | e.g., T-reg cells, MDSCs, | diseases |
| | | cancer cells | |

### TRAF-Binding Receptors

TRAF-binding receptors are TNFRSF members that contain motifs with four to six amino acids called TRAF-interacting motifs (TIMs) which recruit TRAF proteins. TRAF proteins are adaptor molecules that activate multiple downstream signaling pathways such as, e.g., NFκB, Janus kinase (JAK), ERK, p38MAPK, and PI3K that help in cell survival, proliferation, and cytokine production (see Sonar et al. Front Immunol. 2015; 6:364., which is incorporated herein by reference in its entirety). Antagonistic polypeptides (e.g., single-chain polypeptides, antibodies, and antigen-binding fragments, such as anti-CD40 polypeptides that inhibit CD40 activation) of the disclosure may inhibit the activity of TNFRSF members that are responsible for initiating intracellular signaling that promotes cell survival (proliferation) and differentiation, as well as immune and inflammatory responses. TNFRSF members with a TRAF-binding domain include CD40, TRAIL-R3, TRAIL-R4, RANK, FN14, TNFR2, LT beta receptor, HVEM, CD30, CD27, 4-1BB, OX40, GITR, BCMA, TACI, BAFF-R, XEDAR, TROY, and RELT (19L). Inhibiting TNFRSF members, e.g., CD40, RANK, CD27, 4-1 BB, OX40, GITR, and XEDAR, can inhibit proliferation of cells that express these members, such as, e.g., CD8+ cytotoxic T cells and B cells. Antagonistic polypeptides that inhibit these TNFRSF members may be used to treat autoimmune diseases (e.g., allergies, asthma, graft versus host disease (GVHD), and the like). Alternatively, inhibiting TNFRSF members such as, e.g., TRAIL-R3, TRAIL-R4, FN14, TNFR2, LT beta receptor, HVEM, CD30, BCMA, TACI, BAFF-R, TROY, and RELT (19L) using an antagonistic polypeptide can be used to inhibit proliferation of cells that express these members, such as, e.g., T-reg cells, MDSCs, and TNFRSF member expressing cancer cells. Therefore, antagonistic polypeptides that inhibit these TNFRSF members can be used to treat diseases such as cancers and infectious diseases.

### Death Domain (DD)-Containing Receptors

Death domain (DD)-containing receptors (or death receptors) are characterized by a conserved C-terminally located protein-protein interaction domain called the death domain (see Lang et al., J. Biol. Chem. 291:5022-5037, 2016, which is incorporated herein by reference in its entirety); however, they also interact with other cytoplasmic DD-containing adaptor molecules. This receptor—adaptor complex acts as a scaffold for binding of immature pro-caspase, which then undergoes auto-cleavage, leading to the formation of the death-inducing signaling complex (DISC) and induction of apoptosis (see Sonar et al., Front. Immunol. 6:364, 2015, which is incorporated herein by reference in its entirety).

Antagonistic polypeptides (e.g., single-chain polypeptides, antibodies, and antigen-binding fragments) of the disclosure may inhibit the activity of TNFRSF members that are responsible for signaling pathways involving DD-containing receptors, such as, e.g., TRAIL-R1 (TNFRSF10A), TRAIL-R2 (TNFRSF10B), TRAMP, DR6, NGFR, TNFR1, Fas, and EDAR. Inhibiting TNFRSF members, e.g., TNFR1, RAIL-R1, and TRAIL-R2 (TNFRSF10B), can suppress apoptosis of cells that express these members, such as, e.g., T-reg cells and MDSCs. Thus, antagonist polypeptides that inhibit these TNFRSF members may be used to treat autoimmune diseases (e.g., allergies, asthma, GVHD, and the like). Inhibiting TNFRSF members, e.g., TRAMP, DR6, NGFR, and Fas, can suppress apoptosis of cells that express these members, such as, e.g., CD8+ cytotoxic T cells and B cells. Therefore, antagonistic polypeptides that inhibit these TNFRSF members can be used to treat diseases such as cancers and infectious diseases.

### Inactive Conformation: Anti-Parallel Dimer

Antagonistic polypeptides (e.g., single-chain polypeptides, antibodies, and antigen-binding fragments, such as anti-CD40 polypeptides that inhibit CD40 activation) of the disclosure may be used to inhibit the activity of TNFRSF members by, for example, binding a TNFRSF member in an anti-parallel dimer conformation. By binding a TNFRSF member in an anti-parallel dimer structure, antibodies or antigen-binding fragments thereof form a complex with the TNFRSF member in which receptor residues that bind a cognate or natural ligand (such as CD40 ligand in the case of CD40) are sequestered within the interior of the complex. Thus, antibodies or antigen-binding fragments thereof of the disclosure may prevent or substantially inhibit or reduce ligand-mediated trimerization, and hence activation, of a TNFRSF member by forming a complex with the TNFRSF member that sterically precludes the endogenous ligand from accessing its cognate binding sites within the receptor. Exemplary TNFRSF members that are known to naturally adopt an anti-parallel dimer conformation include CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, XEDAR, DCR3, DR3, TRAIL-R3, CD271, TWEAK-R, BCMA, TACT, and BAFFR, among others.

Structural studies of unliganded TNFRSF member receptors, such as TNFR1, have revealed that these proteins adopt an anti-parallel dimer conformation, as described, e.g., in Naismith et al., J. Biol. Chem. 290:13303, 1995, the disclosure of which is incorporated herein by reference in its entirety. This principle extends to other TNFRSF member proteins, as it has been shown, for example, that the human death receptor 3 (DR3) naturally adopts an anti-parallel dimer conformation (Tengchuan, Original Archival Copy of Thesis, Structural Characterization of TNF Receptors and Ligands, Chicago, I L, 2008, the disclosure of which is incorporated herein by reference in its entirety). Similarly, the TRAIL receptor is known to form an anti-parallel dimer (Shirley et al., Rec. Pat. Anticanc. Drug Disc. 6:311, 2011, the disclosure of which is incorporated herein by reference in its entirety). TRAF6 has additionally been shown to adopt this structural conformation (Marienfeld et al., Mol. Cell. Biol. 26:9209, 2006; Yin et al., Biochem. 48:10558, 2009; and Yin et al., Nat. Struct. Mol. Biol. 16:658, 2009, the disclosures of each of which are incorporated herein by reference in their entirety). NGFR has been shown to adopt an anti-parallel dimer conformation (Bibel et al., Genes Dev. 14:2919, 2000, the disclosure of which is incorporated herein by reference in its entirety). Additionally, CD40 is known to exhibit this structural motif (Smulski et al. J. Biol. Chem. 288:10914, 2013, the disclosure of which is incorporated herein by reference in its entirety). Additionally, CD137 (4-1BB) has been shown to exist in an anti-parallel dimer state (Vinay et al. (CD137 Pathway: Immunology and Diseases, New York, NY, 2006), the disclosure of which is incorporated herein by reference in its entirety). Fas, CD40, OX40, and CD27 have additionally been shown to adopt an anti-parallel dimer conformation naturally (Tartaglia et al., J. Biol. Chem. 267:4304, 1992, the disclosure of which is incorporated herein by reference in its entirety). BAFF-R has also been found to exhibit this structural motif unliganded (Kim et al., Nat. Struct. Biol. 10:342, 2003, the disclosure of which is incorporated herein by reference in its entirety).

Polypeptides (e.g., single-chain polypeptides, antibodies, and antigen-binding fragments, such as anti-CD40 polypeptides that bind CD40) of the disclosure may, therefore, be used to bind a TNFRSF member in an anti-parallel dimer conformation in order to inhibit the activity of the TNFRSF member in a target cell, such as, e.g., a T-reg cell, a CD8+ cytotoxic T cell, a TNFRSF member expressing cancer cell, a myeloid-derived suppressor cell, a B cell, a monocyte, a neutrophil, a platelet, a granulocyte, a bone marrow-derived lymphoid cell, or a parenchymal cell. Polypeptides described herein are capable of binding TNFRSF members and epitopes therein, such as epitopes containing two or more continuous or discontinuous residues within CRD3 and/or CRD4 of human TNFRSF members.

For instance, antagonistic CD40 antibodies or antigen-binding fragments thereof of the disclosure may bind an epitope within amino acids 104-172 of SEQ ID NO: 4. For example, antagonistic CD40 antibodies or antigen-binding fragments thereof of the disclosure may bind an epitope of, within, or including one or more of amino acids 104-152 (e.g., amino acids 124-132, SCSPGFGVK, SEQ ID NO: 25) of the CD40 amino acid sequence (SEQ ID NO:4) and/or amino acids 123-172 (e.g., amino acids 143-152, CEPCPVGFFS, SEQ ID NO: 26) of the CD40 amino acid sequence (SEQ ID NO: 4). In an embodiment, the epitope bound by the antibody or antigen-binding fragment thereof is or includes one or more of amino acids 124-132 of SEQ ID NO: 4. In an embodiment, the epitope bound by the antibody or antigen-binding fragment thereof is or includes one or more of amino acids 143-152 of SEQ ID NO: 4. Antagonistic CD40 antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-CD40 polypeptides (e.g., single-chain polypeptides, antibodies, and antigen-binding fragments) of the disclosure may also specifically bind an epitope within human CD40 that includes at least five continuous or discontinuous amino acid residues of amino acids 104-172 of SEQ ID NO: 4 (e.g., at least five continuous or discontinuous amino acid residues of amino acids 114-142 of SEQ ID NO: 4 and/or at least five continuous or discontinuous amino acid residues of amino acids 133-162 of SEQ ID NO: 4, or at least five continuous or discontinuous amino acid residues of amino acids 124-132 of SEQ ID NO: 4 and/or at least five continuous or discontinuous amino acid residues of amino acids 143-152 of SEQ ID NO: 4). Furthermore, anti-CD40 polypeptides (e.g., single-chain polypeptides, antibodies, and antigen-binding fragments) of the disclosure may also specifically bind an epitope within human CD40 that includes at least five continuous or discontinuous amino acid residues of amino acids 104-172 of SEQ ID NO: 4 (e.g., at least five continuous or discontinuous amino acid residues of amino acids 114-142 of SEQ ID NO: 4 and/or at least five continuous or discontinuous amino acid residues of amino acids 123-172 of SEQ ID NO: 4, or at least five continuous or discontinuous amino acid residues of amino acids 124-132 of SEQ ID NO: 4 and/or at least five continuous or discontinuous amino acid residues of amino acids 133-162 of SEQ ID NO: 4), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic CD40 antibodies or antigen-binding fragments thereof can target cells that express CD40, such as, e.g., T cells, B cells, platelets, macrophages, dendritic cells, epithelial cells, endothelial cells, and mesenchymal cells. Antagonistic 4-1 BB antibodies or antigen-binding fragments thereof of the disclosure may bind an epitope within amino acids 81-149 of SEQ ID NO: 1. For example, antagonistic CD40 antibodies or antigen-binding fragments thereof of the disclosure may bind an epitope of, within, or including one or more of amino acids 81-149 (e.g., amino acids 101-129, MCEQDCKQGQELTKKGCKDCCFGTFNDQK, SEQ ID NO: 27) of the 4-1 BB amino acid sequence (SEQ ID NO: 1). In an embodiment, the epitope bound by the antibody or antigen-binding fragment thereof is or includes one or more of amino acids 101-129 of SEQ ID NO: 1. Antagonistic 4-1BB antibodies or antigen-binding fragments thereof may also bind an epitope(s) that exhibits at least 80% sequence identity (e.g., 80%, 85%, 90%, 95%, 97%, 99%, or 100% sequence identity) to this sequence and an epitope(s) that contains conservative amino acid substitutions relative to this sequence. Anti-4-1 BB polypeptides (e.g., single-chain polypeptides, antibodies, and antigen-binding fragments) of the disclosure may also specifically bind an epitope within human 4-1 BB that includes at least five continuous or discontinuous amino acid residues of amino acids 81-149 of SEQ ID NO: 1 (e.g., at least five continuous or discontinuous amino acid residues of amino acids 91-139 of SEQ ID NO: 1, or at least five continuous or discontinuous amino acid residues of amino acids 101-129 of SEQ ID NO: 1). Furthermore, anti-4-1BB polypeptides (e.g., single-chain polypeptides, antibodies, and antigen-binding fragments) of the disclosure may also specifically bind an epitope within human 4-1 BB that includes at least five continuous or discontinuous amino acid residues of amino acids 81-149 of SEQ ID NO: 1 (e.g., at least five continuous or discontinuous amino acid residues of amino acids 91-139 of SEQ ID NO: 1, or at least five continuous or discontinuous amino acid residues of amino acids 101-129 of SEQ ID NO: 1), as well as an epitope(s) that exhibits at least 80% sequence identity (e.g., 80%, 85%, 90%, 95%, 97%, 99%, or 100% sequence identity) to this sequence and an epitope(s) that contains conservative amino acid substitutions relative to this sequence. Antagonistic 4-1 BB antibodies or antigen-binding fragments thereof can target cells that express 4-1 BB, such as, e.g., CD4+ and CD8+ cytotoxic T cells, and NK cells.

Antagonistic CD27 antibodies or antigen-binding fragments thereof of the disclosure may bind an epitope within amino acids 21-92 of SEQ ID NO: 2. For example, antagonistic CD40 antibodies or antigen-binding fragments thereof of the disclosure may bind an epitope of, within, or including one or more of amino acids 21-67 (e.g., amino acids 41-47, QMCEPGT, SEQ ID NO: 28) of the CD27 amino acid sequence (SEQ ID NO: 2) and/or amino acids 41-92 (e.g., amino acids 61-72, QCDPCIPGVSFS, SEQ ID NO: 29) of the CD27 amino acid sequence (SEQ ID NO: 2). In an embodiment, the epitope bound by the antibody or antigen-binding fragment thereof is or includes one or more of amino acids 41-47 of SEQ ID NO: 2. In an embodiment, the epitope bound by the antibody or antigen-binding fragment thereof is or includes one or more of amino acids 61-72 of SEQ ID NO: 2. Antagonistic CD27 antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-CD27 polypeptides (e.g., single-chain polypeptides, antibodies, and antigen-binding fragments) of the disclosure may also specifically bind an epitope within human CD27 that includes at least five continuous or discontinuous amino acid residues of amino acids 21-92 of SEQ ID NO: 2 (e.g., at least five continuous or discontinuous amino acid residues of amino acids 31-57 and/or positions 51-82 of SEQ ID NO: 2, or at least five continuous or discontinuous amino acid residues of amino acids 41-47 and/or positions 61-72 of SEQ ID NO: 2. Furthermore, anti-CD27 polypeptides (e.g., single-chain polypeptides, antibodies, and antigen-binding fragments) of the disclosure may also specifically bind an epitope within human CD27 that includes at least five continuous or discontinuous amino acid residues of amino acids 21-92 of SEQ ID NO: 2 (e.g., at least five continuous or discontinuous amino acid residues of amino acids 31-57 and/or positions 51-82 of SEQ ID NO: 2, or at least five continuous or discontinuous amino acid residues of amino acids 41-47 and/or positions 61-72 of SEQ ID NO: 2), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic CD27 antibodies or antigen-binding fragments thereof can target cells that express CD27, such as, e.g., CD4+ and CD8+ cytotoxic T cells.

Antagonistic CD30 antibodies or antigen-binding fragments thereof of the disclosure may bind an epitope within amino acids 109-177 of SEQ ID NO: 3. For example, antagonistic CD40 antibodies or antigen-binding fragments thereof of the disclosure may bind an epitope of, within, or including one or more of amino acids 109-160 (e.g., amino acids 129-140, SVCPAGMIVKFP, SEQ ID NO: 30) of the CD30 amino acid sequence (SEQ ID NO:3) and/or amino acids 129-177 (e.g., amino acids 149-157, CEPASPGVS, SEQ ID NO: 31) of the CD30 amino acid sequence (SEQ ID NO: 3). In an embodiment, the epitope bound by the antibody or antigen-binding fragment thereof is or includes one or more of amino acids 129-140 of SEQ ID NO: 3. In an embodiment, the epitope bound by the antibody or antigen-binding fragment thereof is or includes one or more of amino acids 149-157 of SEQ ID NO: 3. Antagonistic CD30 antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-CD30 polypeptides (e.g., single-chain polypeptides, antibodies, and antigen-binding fragments) of the disclosure may also specifically bind an epitope within human CD30 that includes at least five continuous or discontinuous amino acid residues of amino acids 109-177 of SEQ ID NO: 3 (e.g., at least five continuous or discontinuous amino acid residues of amino acids 119-150 and/or positions 139-167 of SEQ ID NO: 3, or at least five continuous or discontinuous amino acid residues of amino acids 129-140 and/or positions 149-157 of SEQ ID NO: 3). Furthermore, anti-CD30 polypeptides (e.g., single-chain polypeptides, antibodies, and antigen-binding fragments) of the disclosure may also specifically bind an epitope within human CD30 that includes at least five continuous or discontinuous amino acid residues of amino acids 109-177 of SEQ ID NO: 3 (e.g., at least five continuous or discontinuous amino acid residues of amino acids 119-150 and/or positions 139-167 of SEQ ID NO: 3, or at least five continuous or discontinuous amino acid residues of amino acids 129-140 and/or positions 149-157 of SEQ ID NO: 3), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic CD30 antibodies or antigen-binding fragments thereof can target cells that express CD30, such as, e.g., T and B lymphocytes, T cell lymphomas, and B cell lymphomas.

Antagonistic DR6 antibodies or antigen-binding fragments thereof of the disclosure may bind an epitope within amino acids 121-197 of SEQ ID NO: 5. For example, antagonistic CD40 antibodies or antigen-binding fragments thereof of the disclosure may bind an epitope of, within, or including one or more of amino acids 121-176 (e.g., amino acids 141-156, NGTCAPHTVCPVGWGV, SEQ ID NO: 32) of the DR6 amino acid sequence (SEQ ID NO: 5) and/or amino acids 149-197 (e.g., amino acids 169-177, KQCARGTFS, SEQ ID NO: 33) of the DR6 amino acid sequence (SEQ ID NO: 5). In an embodiment, the epitope bound by the antibody or antigen-binding fragment thereof is or includes one or more of amino acids 141-156 of SEQ ID NO: 5. In an embodiment, the epitope bound by the antibody or antigen-binding fragment thereof is or includes one or more of amino acids 169-177 of SEQ ID NO: 5. Antagonistic DR6 antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-DR6 polypeptides (e.g., single-chain polypeptides, antibodies, and antigen-binding fragments) of the disclosure may also specifically bind an epitope within human DR6 that includes at least five continuous or discontinuous amino acid residues of amino acids 121-197 of SEQ ID NO: 5 (e.g., at least five continuous or discontinuous amino acid residues of amino acids 131-166 and/or positions 159-187 of SEQ ID NO: 5, or at least five continuous or discontinuous amino acid residues of amino acids 141-156 and/or positions 169-177 of SEQ ID NO: 5). Furthermore, anti-DR6 polypeptides (e.g., single-chain polypeptides, antibodies, and antigen-binding fragments) of the disclosure may also specifically bind an epitope within human DR6 that includes at least five continuous or discontinuous amino acid residues of amino acids 121-197 of SEQ ID NO: 5 (e.g., at least five continuous or discontinuous amino acid residues of amino acids 131-166 and/or positions 159-187 of SEQ ID NO: 5, or at least five continuous or discontinuous amino acid residues of amino acids 141-156 and/or positions 169-177 of SEQ ID NO: 5), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic DR6 antibodies or antigen-binding fragments thereof can target cells that express DR6, such as, e.g., thymus cells, spleen cells, T cells, nerve cells, and gliomas.

Antagonistic EDAR antibodies or antigen-binding fragments thereof of the disclosure may bind an epitope within amino acids 71-142 of SEQ ID NO: 6. For example, antagonistic CD40 antibodies or antigen-binding fragments thereof of the disclosure may bind an epitope of, within, or including one or more of amino acids 71-122 (e.g., amino acids 91-102, KDCEGFFRATVL, SEQ ID NO: 34) of the EDAR amino acid sequence (SEQ ID NO: 6) and/or amino acids 91-142 (e.g., amino acids 111-122, AECGPCLPGYYM, SEQ ID NO: 35) of the EDAR amino acid sequence (SEQ ID NO: 6). In an embodiment, the epitope bound by the antibody or antigen-binding fragment thereof is or includes one or more of amino acids 91-102 of SEQ ID NO: 6. In an embodiment, the epitope bound by the antibody or antigen-binding fragment thereof is or includes one or more of amino acids 111-122 of SEQ ID NO: 6. Antagonistic EDAR antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-EDAR polypeptides (e.g., single-chain polypeptides, antibodies, and antigen-binding fragments) of the disclosure may also specifically bind an epitope within human EDAR that includes at least five continuous or discontinuous amino acid residues of amino acids 71-142 of SEQ ID NO: 6 (e.g., at least five continuous or discontinuous amino acid residues of amino acids 81-112 and/or positions 101-132 of SEQ ID NO: 6, or at least five continuous or discontinuous amino acid residues of amino acids 91-102 and/or positions 111-122 of SEQ ID NO: 6). Furthermore, anti-EDAR polypeptides (e.g., single-chain polypeptides, antibodies, and antigen-binding fragments) of the disclosure may also specifically bind an epitope within human EDAR that includes at least five continuous or discontinuous amino acid residues of amino acids 71-142 of SEQ ID NO: 6 (e.g., at least five continuous or discontinuous amino acid residues of amino acids 81-112 and/or positions 101-132 of SEQ ID NO: 6, or at least five continuous or discontinuous amino acid residues of amino acids 91-102 and/or positions 111-122 of SEQ ID NO: 6), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic EDAR antibodies or antigen-binding fragments thereof can target cells that express EDAR, such as, e.g., embryonic cells.

Antagonistic Fas antibodies or antigen-binding fragments thereof of the disclosure may bind an epitope within amino acids 128-193 of SEQ ID NO: 7. For example, antagonistic CD40 antibodies or antigen-binding fragments thereof of the disclosure may bind an epitope of, within, or including one or more of amino acids 128-176 (e.g., amino acids 148-156, KCEHGIIKE, SEQ ID NO: 36) of the Fas amino acid sequence (SEQ ID NO: 7) and/or amino acids 145-193 (e.g., amino acids 165-173, CKEEGSRSN, SEQ ID NO: 37) of the Fas amino acid sequence (SEQ ID NO: 7). In an embodiment, the epitope bound by the antibody or antigen-binding fragment thereof is or includes one or more of amino acids 148-156 of SEQ ID NO: 7. In an embodiment, the epitope bound by the antibody or antigen-binding fragment thereof is or includes one or more of amino acids 165-173 of SEQ ID NO: 7. Antagonistic Fas antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-Fas polypeptides (e.g., single-chain polypeptides, antibodies, and antigen-binding fragments) of the disclosure may also specifically bind an epitope within human Fas that includes at least five continuous or discontinuous amino acid residues of amino acids 128-193 of SEQ ID NO: 7 (e.g., at least five continuous or discontinuous amino acid residues of amino acids 138-166 and/or positions 155-183 of SEQ ID NO: 7, or at least five continuous or discontinuous amino acid residues of amino acids 148-156 and/or positions 165-173 of SEQ ID NO: 7). Furthermore, anti-Fas polypeptides (e.g., single-chain polypeptides, antibodies, and antigen-binding fragments) of the disclosure may also specifically bind an epitope within human Fas that includes at least five continuous or discontinuous amino acid residues of amino acids 128-193 of SEQ ID NO: 7 (e.g., at least five continuous or discontinuous amino acid residues of amino acids 138-166 and/or positions 155-183 of SEQ ID NO: 7, or at least five continuous or discontinuous amino acid residues of amino acids 148-156 and/or positions 165-173 of SEQ ID NO: 7), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic Fas antibodies or antigen-binding fragments thereof can target cells that express Fas, such as, e.g., thymus cells, liver cells, kidney cells, and B cells.

Antagonistic GITR antibodies or antigen-binding fragments thereof of the disclosure may bind an epitope within amino acids 72-141 of SEQ ID NO: 8. For example, antagonistic CD40 antibodies or antigen-binding fragments thereof of the disclosure may bind an epitope of, within, or including one or more of amino acids 72-121 (e.g., amino acids 92-101, HPCPPGQGVQ, SEQ ID NO: 38) of the GITR amino acid sequence (SEQ ID NO: 8) and/or amino acids 92-141 (e.g., amino acids 112-121, CIDCASGTFS, SEQ ID NO: 39) of the GITR amino acid sequence (SEQ ID NO: 8). In an embodiment, the epitope bound by the antibody or antigen-binding fragment thereof is or includes one or more of amino acids 92-101 of SEQ ID NO: 8. In an embodiment, the epitope bound by the antibody or antigen-binding fragment thereof is or includes one or more of amino acids 112-121 of SEQ ID NO: 8. Antagonistic GITR antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-GITR polypeptides (e.g., single-chain polypeptides, antibodies, and antigen-binding fragments) of the disclosure may also specifically bind an epitope within human GITR that includes at least five continuous or discontinuous amino acid residues of amino acids 72-141 of SEQ ID NO: 8 (e.g., at least five continuous or discontinuous amino acid residues of amino acids 82-111 and/or positions 102-131 of SEQ ID NO: 8, or at least five continuous or discontinuous amino acid residues of amino acids 92-101 and/or positions 112-121 of SEQ ID NO: 8). Furthermore, anti-GITR polypeptides (e.g., single-chain polypeptides, antibodies, and antigen-binding fragments) of the disclosure may also specifically bind an epitope within human GITR that includes at least five continuous or discontinuous amino acid residues of amino acids 72-141 of SEQ ID NO: 8 (e.g., at least five continuous or discontinuous amino acid residues of amino acids 82-111 and/or positions 102-131 of SEQ ID NO: 8, or at least five continuous or discontinuous amino acid residues of amino acids 92-101 and/or positions 112-121 of SEQ ID NO: 8), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic GITR antibodies or antigen-binding fragments thereof can target cells that express GITR, such as, e.g., T cells, and NK cells.

Antagonistic HVEM antibodies or antigen-binding fragments thereof of the disclosure may bind an epitope within amino acids 122-190 of SEQ ID NO: 9. For example, antagonistic CD40 antibodies or antigen-binding fragments thereof of the disclosure may bind an epitope of, within, or including one or more of amino acids 122-172 (e.g., amino acids 142-152, TTCPPGQRVEK, SEQ ID NO: 40) of the HVEM amino acid sequence (SEQ ID NO: 9) and/or amino acids 142-190 (e.g., amino acids 162-170, CADCLTGTF, SEQ ID NO: 41) of the HVEM amino acid sequence (SEQ ID NO: 9). In an embodiment, the epitope bound by the antibody or antigen-binding fragment thereof is or includes one or more of amino acids 142-152 of SEQ ID NO: 9. In an embodiment, the epitope bound by the antibody or antigen-binding fragment thereof is or includes one or more of amino acids 162-170 of SEQ ID NO: 9. Antagonistic HVEM antibodies or antigen-binding fragments thereof may also bind an epitope(s) that exhibits at least 80% sequence identity (e.g., 80%, 85%, 90%, 95%, 97%, 99%, 100% sequence identity) to one or both of these sequences and an epitope(s) that contains conservative amino acid substitutions relative to one or both of these sequences. Anti-HVEM polypeptides (e.g., single-chain polypeptides, antibodies, and antigen-binding fragments) of the disclosure may also specifically bind an epitope within human HVEM that includes at least five continuous or discontinuous amino acid residues of amino acids 122-190 of SEQ ID NO: 9 (e.g., at least five continuous or discontinuous amino acid residues of amino acids 132-162 and/or positions 152-180 of SEQ ID NO: 9, or at least five continuous or discontinuous amino acid residues of amino acids 142-152 and/or positions 162-170 of SEQ ID NO: 9). Furthermore, anti-HVEM polypeptides (e.g., single-chain polypeptides, antibodies, and antigen-binding fragments) of the disclosure may also specifically bind an epitope within human HVEM that includes at least five continuous or discontinuous amino acid residues of amino acids 122-190 of SEQ ID NO: 9 (e.g., at least five continuous or discontinuous amino acid residues of amino acids 132-162 and/or positions 152-180 of SEQ ID NO: 9, or at least five continuous or discontinuous amino acid residues of amino acids 142-152 and/or positions 162-170 of SEQ ID NO: 9), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic HVEM antibodies or antigen-binding fragments thereof can target cells that express HVEM, such as, e.g., colorectal cancers, esophageal carcinomas, gastric cancers, hepatocarcinomas, breast cancers, lymphomas, spleen cells, thymus cells, bone marrow cells, T-reg cells, T cells, B cells, lung cells, and intestinal cells.

Antagonistic LT beta receptor antibodies or antigen-binding fragments thereof of the disclosure may bind an epitope within amino acids 127-196 of SEQ ID NO: 10. For example, antagonistic CD40 antibodies or antigen-binding fragments thereof of the disclosure may bind an epitope of, within, or including one or more of amino acids 127-175 (e.g., amino acids 147-155, DCPPGTEAE, SEQ ID NO: 42) of the LT beta receptor amino acid sequence (SEQ ID NO: 10) and/or amino acids 147-196 (e.g., amino acids 167-176, CVPCKAGHFQ, SEQ ID NO: 43) of the LT beta receptor amino acid sequence (SEQ ID NO: 10). In an embodiment, the epitope bound by the antibody or antigen-binding fragment thereof is or includes one or more of amino acids 147-155 of SEQ ID NO: 10. In an embodiment, the epitope bound by the antibody or antigen-binding fragment thereof is or includes one or more of amino acids 167-176 of SEQ ID NO: 10. Antagonistic LT beta receptor antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-LT beta receptor polypeptides (e.g., single-chain polypeptides, antibodies, and antigen-binding fragments) of the disclosure may also specifically bind an epitope within human LT beta receptor that includes at least five continuous or discontinuous amino acid residues of amino acids 127-196 of SEQ ID NO: 10 (e.g., at least five continuous or discontinuous amino acid residues of amino acids 137-165 and/or positions 157-186 of SEQ ID NO: 10, or at least five continuous or discontinuous amino acid residues of amino acids 147-155 and/or positions 167-176 of SEQ ID NO: 10). Furthermore, anti-LT beta receptor polypeptides (e.g., single-chain polypeptides, antibodies, and antigen-binding fragments) of the disclosure may also specifically bind an epitope within human LT beta receptor that includes at least five continuous or discontinuous amino acid residues of amino acids 127-196 of SEQ ID NO: 10 (e.g., at least five continuous or discontinuous amino acid residues of amino acids 137-165 and/or positions 157-186 of SEQ ID NO: 10, or at least five continuous or discontinuous amino acid residues of amino acids 147-155 and/or positions 167-176 of SEQ ID NO: 10), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic LT beta receptor antibodies or antigen-binding fragments thereof can target cells that express LT beta receptor, such as, e.g., cancer cells, stromal cells in lymphoid tissue, myeloid lineage cells, monocytes, alveolar macrophages, mast cells, and dendritic cells.

Antagonistic NGFR antibodies or antigen-binding fragments thereof of the disclosure may bind an epitope within amino acids 108-176 of SEQ ID NO: 11. For example, antagonistic CD40 antibodies or antigen-binding fragments thereof of the disclosure may bind an epitope of, within, or including one or more of amino acids 108-155 (e.g., amino acids 128-135, CEAGSGLV, SEQ ID NO: 44) of the NGFR amino acid sequence (SEQ ID NO: 11) and/or amino acids 126-176 (e.g., amino acids 146-156, CEECPDGTYSD, SEQ ID NO: 45) of the NGFR amino acid sequence (SEQ ID NO: 11). In an embodiment, the epitope bound by the antibody or antigen-binding fragment thereof is or includes one or more of amino acids 128-135 of SEQ ID NO: 11. In an embodiment, the epitope bound by the antibody or antigen-binding fragment thereof is or includes one or more of amino acids 146-156 of SEQ ID NO: 11. Antagonistic NGFR antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-NGFR polypeptides (e.g., single-chain polypeptides, antibodies, and antigen-binding fragments) of the disclosure may also specifically bind an epitope within human NGFR that includes at least five continuous or discontinuous amino acid residues of amino acids 108-176 of SEQ ID NO: 11 (e.g., at least five continuous or discontinuous amino acid residues of amino acids 118-145 and/or positions 136-166 of SEQ ID NO: 11, or at least five continuous or discontinuous amino acid residues of amino acids 128-135 and/or positions 146-156 of SEQ ID NO: 11). Furthermore, anti-NGFR polypeptides (e.g., single-chain polypeptides, antibodies, and antigen-binding fragments) of the disclosure may also specifically bind an epitope within human NGFR that includes at least five continuous or discontinuous amino acid residues of amino acids 108-176 of SEQ ID NO: 11 (e.g., at least five continuous or discontinuous amino acid residues of amino acids 118-145 and/or positions 136-166 of SEQ ID NO: 11, or at least five continuous or discontinuous amino acid residues of amino acids 128-135 and/or positions 146-156 of SEQ ID NO: 11), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic NGFR antibodies or antigen-binding fragments thereof can target cells that express NGFR, such as, e.g., neural cells and tumor cells.

Antagonistic OPG antibodies or antigen-binding fragments thereof of the disclosure may bind an epitope within amino acids 103-171 of SEQ ID NO: 12. For example, antagonistic CD40 antibodies or antigen-binding fragments thereof of the disclosure may bind an epitope of, within, or including one or more of amino acids 103-151 (e.g., amino acids 123-131, SCPPGFGVV, SEQ ID NO: 46) of the OPG amino acid sequence (SEQ ID NO: 12) and/or amino acids 122-171 (e.g., amino acids 142-151, CKRCPDGFFS, SEQ ID NO: 47) of the OPG amino acid sequence (SEQ ID NO: 12). In an embodiment, the epitope bound by the antibody or antigen-binding fragment thereof is or includes one or more of amino acids 123-131 of SEQ ID NO: 12. In an embodiment, the epitope bound by the antibody or antigen-binding fragment thereof is or includes one or more of amino acids 142-151 of SEQ ID NO: 12. Antagonistic OPG antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-OPG polypeptides (e.g., single-chain polypeptides, antibodies, and antigen-binding fragments) of the disclosure may also specifically bind an epitope within human OPG that includes at least five continuous or discontinuous amino acid residues of amino acids 103-171 of SEQ ID NO: 12 (e.g., at least five continuous or discontinuous amino acid residues of amino acids 113-141 and/or positions 132-161 of SEQ ID NO: 12, or at least five continuous or discontinuous amino acid residues of amino acids 123-131 and/or positions 142-151 of SEQ ID NO: 12). Furthermore, anti-OPG polypeptides (e.g., single-chain polypeptides, antibodies, and antigen-binding fragments) of the disclosure may also specifically bind an epitope within human OPG that includes at least five continuous or discontinuous amino acid residues of amino acids 103-171 of SEQ ID NO: 12 (e.g., at least five continuous or discontinuous amino acid residues of amino acids 113-141 and/or positions 132-161 of SEQ ID NO: 12, or at least five continuous or discontinuous amino acid residues of amino acids 123-131 and/or positions 142-151 of SEQ ID NO: 12), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic OPG antibodies or antigen-binding fragments thereof can target cells that express OPG, such as, e.g., osteoblast lineage cells, epithelial cells (e.g., of the gastrointestinal tract, lung, breast, and skin), vascular endothelial cells, B cells, and dendritic cells.

Antagonistic OX40 antibodies or antigen-binding fragments thereof of the disclosure may bind an epitope within amino acids 98-154 of SEQ ID NO: 13. For example, antagonistic CD40 antibodies or antigen-binding fragments thereof of the disclosure may bind an epitope of, within, or including one or more of amino acids 98-154 (e.g., amino acids 118-134, SYKPGVDCAPCPPGHFS, SEQ ID NO: 48) of the OX40 amino acid sequence (SEQ ID NO: 13). In an embodiment, the epitope bound by the antibody or antigen-binding fragment thereof is or includes one or more of amino acids 118-134 of SEQ ID NO: 13. Antagonistic OX40 antibodies or antigen-binding fragments thereof may also bind an epitope(s) that identity) to this sequence and an epitope(s) that contains conservative amino acid substitutions relative to this sequence. Anti-OX40 polypeptides (e.g., single-chain polypeptides, antibodies, and antigen-binding fragments) of the disclosure may also specifically bind an epitope within human OX40 that includes at least five continuous or discontinuous amino acid residues of amino acids 98-154 of SEQ ID NO: 13 (e.g., at least five continuous or discontinuous amino acid residues of amino acids 108-144 of SEQ ID NO: 13, or at least five continuous or discontinuous amino acid residues of amino acids 118-134 of SEQ ID NO: 13). Furthermore, anti-OX40 polypeptides (e.g., single-chain polypeptides, antibodies, and antigen-binding fragments) of the disclosure may also specifically bind an epitope within human OX40 that includes at least five continuous or discontinuous amino acid residues of amino acids 98-154 of SEQ ID NO: 13 (e.g., at least five continuous or discontinuous amino acid residues of amino acids 108-144 of SEQ ID NO: 13, or at least five continuous or discontinuous amino acid residues of amino acids 118-134 of SEQ ID NO: 13), as well as an epitope(s) that exhibits at least 80% sequence identity (e.g., 80%, 85%, 90%, 95%, 97%, 99%, or 100% sequence identity) to this sequence and an epitope(s) that contains conservative amino acid substitutions relative to this sequence. Antagonistic OX40 antibodies or antigen-binding fragments thereof can target cells that express OX40, such as activated CD4+ and CD8+ cytotoxic T cells and a number of lymphoid and non-lymphoid cells.

Antagonistic RANK antibodies or antigen-binding fragments thereof of the disclosure may bind an epitope within amino acids 112-180 of SEQ ID NO: 14. For example, antagonistic CD40 antibodies or antigen-binding fragments thereof of the disclosure may bind an epitope of, within, or including one or more of amino acids 112-159 (e.g., amino acids 132-139, ECAPGLGA, SEQ ID NO: 49) of the RANK amino acid sequence (SEQ ID NO: 14) and/or amino acids 131-180 (e.g., amino acids 151-160, CKPCLAGYFS, SEQ ID NO: 50) of the RANK amino acid sequence (SEQ ID NO: 14). In an embodiment, the epitope bound by the antibody or antigen-binding fragment thereof is or includes one or more of amino acids 132-139 of SEQ ID NO: 14. In an embodiment, the epitope bound by the antibody or antigen-binding fragment thereof is or includes one or more of amino acids 151-160 of SEQ ID NO: 14.

Antagonistic RANK antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-RANK polypeptides (e.g., single-chain polypeptides, antibodies, and antigen-binding fragments) of the disclosure may also specifically bind an epitope within human RANK that includes at least five continuous or discontinuous amino acid residues of amino acids 112-180 of SEQ ID NO: 14 (e.g., at least five continuous or discontinuous amino acid residues of amino acids 122-149 and/or positions 141-170 of SEQ ID NO: 14, or at least five continuous or discontinuous amino acid residues of amino acids 132-139 and/or positions 151-160 of SEQ ID NO: 14). Furthermore, anti-RANK polypeptides (e.g., single-chain polypeptides, antibodies, and antigen-binding fragments) of the disclosure may also specifically bind an epitope within human RANK that includes at least five continuous or discontinuous amino acid residues of amino acids 112-180 of SEQ ID NO: 14 (e.g., at least five continuous or discontinuous amino acid residues of amino acids 122-149 and/or positions 141-170 of SEQ ID NO: 14, or at least five continuous or discontinuous amino acid residues of amino acids 132-139 and/or positions 151-160 of SEQ ID NO: 14), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic RANK antibodies or antigen-binding fragments thereof can target cells that express RANK, such as, e.g., skeletal muscle, thymus, liver, colon, small intestine, adrenal gland, osteoclast, mammary gland epithelial, prostate, vascular, and pancreatic cells.

Antagonistic RELT (19L) antibodies or antigen-binding fragments thereof of the disclosure may bind an epitope within amino acids 50-118 of SEQ ID NO: 52. For example, antagonistic CD40 antibodies or antigen-binding fragments thereof of the disclosure may bind an epitope of, within, or including one or more of amino acids 50-97 (e.g., amino acids 70-77, RCSLWRRL, SEQ ID NO: 51) of the RELT (19L) amino acid sequence (SEQ ID NO: 15) and/or amino acids 70-118 (e.g., amino acids 90-98, CGDCWPGWF, SEQ ID NO: 52) of the RELT (19L) amino acid sequence (SEQ ID NO: 15). In an embodiment, the epitope bound by the antibody or antigen-binding fragment thereof is or includes one or more of amino acids 70-77 of SEQ ID NO 15. In an embodiment, the epitope bound by the antibody or antigen-binding fragment thereof is or includes one or more of amino acids 90-98 of SEQ ID NO: 15. Antagonistic RELT (19L) antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-RELT (19L) polypeptides (e.g., single-chain polypeptides, antibodies, and antigen-binding fragments) of the disclosure may also specifically bind an epitope within human RELT (19L) that includes at least five continuous or discontinuous amino acid residues of amino acids 50-118 of SEQ ID NO: 15 (e.g., at least five continuous or discontinuous amino acid residues of amino acids 60-87 and/or positions 80-108 of SEQ ID NO: 15, or at least five continuous or discontinuous amino acid residues of amino acids 70-77 and/or positions 90-98 of SEQ ID NO: 15). Furthermore, anti-RELT (19L) polypeptides (e.g., single-chain polypeptides, antibodies, and antigen-binding fragments) of the disclosure may also specifically bind an epitope within human RELT (19L) that includes at least five continuous or discontinuous amino acid residues of amino acids 50-118 of SEQ ID NO: 15 (e.g., at least five continuous or discontinuous amino acid residues of amino acids 60-87 and/or positions 80-108 of SEQ ID NO: 15, or at least five continuous or discontinuous amino acid residues of amino acids 70-77 and/or positions 90-98 of SEQ ID NO: 15), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic RELT (19L) antibodies or antigen-binding fragments thereof can target cells that express RELT (19L), such as, e.g., hematologic tissue cells (e.g., tissues of the blood leukocytes, lymph, spleen, and bone marrow), T cells, B cells, and myeloid cells.

Antagonistic TNFR1 antibodies or antigen-binding fragments thereof of the disclosure may bind an epitope within amino acids 84-153 of SEQ ID NO: 16. For example, antagonistic CD40 antibodies or antigen-binding fragments thereof of the disclosure may bind an epitope of, within, or including one or more of amino acids 84-132 (e.g., amino acids 104-112, KCRKEMGQV, SEQ ID NO: 53) of the TNFR1 amino acid sequence (SEQ ID NO: 16) and/or amino acids 104-153 (e.g., amino acids 124-133, VCGCRKNQYR, SEQ ID NO: 54) of the TNFR1 amino acid sequence (SEQ ID NO: 16). In an embodiment, the epitope bound by the antibody or antigen-binding fragment thereof is or includes one or more of amino acids 104-112 of SEQ ID NO: 16. In an embodiment, the epitope bound by the antibody or antigen-binding fragment thereof is or includes one or more of amino acids 124-133 of SEQ ID NO: 16. Antagonistic TNFR1 antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-TNFR1 polypeptides (e.g., single-chain polypeptides, antibodies, and antigen-binding fragments) of the disclosure may also specifically bind an epitope within human TNFR1 that includes at least five continuous or discontinuous amino acid residues of amino acids 84-153 of SEQ ID NO: 16 (e.g., at least five continuous or discontinuous amino acid residues of amino acids 94-122 and/or positions 114-143 of SEQ ID NO: 16, or at least five continuous or discontinuous amino acid residues of amino acids 104-112 and/or positions 124-133 of SEQ ID NO: 16). Furthermore, anti-TNFR1 polypeptides (e.g., single-chain polypeptides, antibodies, and antigen-binding fragments) of the disclosure may also specifically bind an epitope within human TNFR1 that includes at least five continuous or discontinuous amino acid residues of amino acids 84-153 of SEQ ID NO: 16 (e.g., at least five continuous or discontinuous amino acid residues of amino acids 94-122 and/or positions 114-143 of SEQ ID NO: 16, or at least five continuous or discontinuous amino acid residues of amino acids 104-112 and/or positions 124-133 of SEQ ID NO: 16), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic TNFR1 antibodies or antigen-binding fragments thereof can target cells that express TNFR1, i.e., nearly all cells since TNFR1 is ubiquitously expressed on the surface of most cells.

Antagonistic TRAIL-R2 (TNFRSF10B) antibodies or antigen-binding fragments thereof of the disclosure may bind an epitope within amino acids 135-206 of SEQ ID NO: 17. For example, antagonistic CD40 antibodies or antigen-binding fragments thereof of the disclosure may bind an epitope of, within, or including one or more of amino acids 135-185 (e.g., amino acids 155-165, KCRTGCPRGMV, SEQ ID NO: 55) of the TRAIL-R2 (TNFRSF10B) amino acid sequence (SEQ ID NO: 17) and/or amino acids 158-206 (e.g., amino acids 178-186, CVHKESGTK, SEQ ID NO: 56) of the TRAIL-R2 (TNFRSF10B) amino acid sequence (SEQ ID NO: 17). In an embodiment, the epitope bound by the antibody or antigen-binding fragment thereof is or includes one or more of amino acids 155-165 of SEQ ID NO: 17. In an embodiment, the epitope bound by the antibody or antigen-binding fragment thereof is or includes one or more of amino acids 178-186 of SEQ ID NO: 17. Antagonistic TRAIL-R2 (TNFRSF10B) antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-TRAIL-R2 (TNFRSF10B) polypeptides (e.g., single-chain polypeptides, antibodies, and antigen-binding fragments) of the disclosure may also specifically bind an epitope within human TRAIL-R2 (TNFRSF10B) that includes at least five continuous or discontinuous amino acid residues of amino acids 135-206 of SEQ ID NO: 17 (e.g., at least five continuous or discontinuous amino acid residues of amino acids 145-175 and/or positions 168-196 of SEQ ID NO: 17, or at least five continuous or discontinuous amino acid residues of amino acids 155-165 and/or positions 178-186 of SEQ ID NO: 17). Furthermore, anti-TRAIL-R2 (TNFRSF10B) polypeptides (e.g., single-chain polypeptides, antibodies, and antigen-binding fragments) of the disclosure may also specifically bind an epitope within human TRAIL-R2 (TNFRSF10B) that includes at least five continuous or discontinuous amino acid residues of amino acids 135-206 of SEQ ID NO: 17 (e.g., at least five continuous or discontinuous amino acid residues of amino acids 145-175 and/or positions 168-196 of SEQ ID NO: 17, or at least five continuous or discontinuous amino acid residues of amino acids 155-165 and/or positions 178-186 of SEQ ID NO: 17), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic TRAIL-R2 (TNFRSF 10B) antibodies or antigen-binding fragments thereof can target cells that express TRAIL-R2 (TNFRSF 10B), such as, e.g., hepatocytes, brain cells, kidney cells, heart myocytes, colon cells, germ cells, Leydig cells, alveolar septum cells, bronchial epithelial cells, and brain vascular epithelial cells.

Antagonistic TRAIL-R1 (TNFRSF10A) antibodies or antigen-binding fragments thereof of the disclosure may bind an epitope within amino acids 149-216 of SEQ ID NO: 18. For example, antagonistic CD40 antibodies or antigen-binding fragments thereof of the disclosure may bind an epitope of, within, or including one or more of amino acids 149-197 (e.g., amino acids 169-177, ACKSDEEER, SEQ ID NO: 57) of the TRAIL-R1 (TNFRSF10A) amino acid sequence (SEQ ID NO: 18) and/or amino acids 168-216 (e.g., amino acids 188-196, CQCKPGTFR, SEQ ID NO: 58) of the TRAIL-R1 (TNFRSF10A) amino acid sequence (SEQ ID NO: 18). In an embodiment, the epitope bound by the antibody or antigen-binding fragment thereof is or includes one or more of amino acids 169-177 of SEQ ID NO: 18. In an embodiment, the epitope bound by the antibody or antigen-binding fragment thereof is or includes one or more of amino acids 188-196 of SEQ ID NO: 18. Antagonistic TRAIL-R1 (TNFRSF10A) antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-TRAIL-R1 (TNFRSF10A) polypeptides (e.g., single-chain polypeptides, antibodies, and antigen-binding fragments) of the disclosure may also specifically bind an epitope within human TRAIL-R1 (TNFRSF10A) that includes at least five continuous or discontinuous amino acid residues of amino acids 149-216 of SEQ ID NO: 18 (e.g., at least five continuous or discontinuous amino acid residues of amino acids 159-187 and/or positions 178-206 of SEQ ID NO: 18, or at least five continuous or discontinuous amino acid residues of amino acids 169-177 and/or positions 188-196 of SEQ ID NO: 18). Furthermore, anti-TRAIL-R1 (TNFRSF10A) polypeptides (e.g., single-chain polypeptides, antibodies, and antigen-binding fragments) of the disclosure may also specifically bind an epitope within human TRAIL-R1 (TNFRSF10A) that includes at least five continuous or discontinuous amino acid residues of amino acids 149-216 of SEQ ID NO: 18 (e.g., at least five continuous or discontinuous amino acid residues of amino acids 159-187 and/or positions 178-206 of SEQ ID NO: 18, or at least five continuous or discontinuous amino acid residues of amino acids 169-177 and/or positions 188-196 of SEQ ID NO: 18), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic TRAIL-R1 (TNFRSF10A) antibodies or antigen-binding fragments thereof can target cells that express TRAIL-R1 (TNFRSF10A), such as, e.g., hepatocytes, bile duct epithelial cells, brain cells, kidney cells, heart myocytes, colon cells, germ cells, and Leydig cells.

Antagonistic TRAIL-R4 antibodies or antigen-binding fragments thereof of the disclosure may bind an epitope within amino acids 141-210 of SEQ ID NO: 19. For example, antagonistic CD40 antibodies or antigen-binding fragments thereof of the disclosure may bind an epitope of, within, or including one or more of amino acids 141-189 (e.g., amino acids 161-169, GCPRGMVKV, SEQ ID NO: 59) of the TRAIL-R4 amino acid sequence (SEQ ID NO:19) and/or amino acids 161-210 (e.g., amino acids 181-190, KNESAASSTG, SEQ ID NO: 60) of the TRAIL-R4 amino acid sequence (SEQ ID NO:19). In an embodiment, the epitope bound by the antibody or antigen-binding fragment thereof is or includes one or more of amino acids 161-169 of SEQ ID NO: 19. In an embodiment, the epitope bound by the antibody or antigen-binding fragment thereof is or includes one or more of amino acids 181-190 of SEQ ID NO: 19. Antagonistic TRAIL-R4 antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-TRAIL-R4 polypeptides (e.g., single-chain polypeptides, antibodies, and antigen-binding fragments) of the disclosure may also specifically bind an epitope within human TRAIL-R4 that includes at least five continuous or discontinuous amino acid residues of amino acids 141-210 of SEQ ID NO: 19 (e.g., at least five continuous or discontinuous amino acid residues of amino acids 151-179 and/or positions 171-200 of SEQ ID NO: 19, or at least five continuous or discontinuous amino acid residues of amino acids 161-169 and/or positions 181-190 of SEQ ID NO: 19). Furthermore, anti-TRAIL-R4 polypeptides (e.g., single-chain polypeptides, antibodies, and antigen-binding fragments) of the disclosure may also specifically bind an epitope within human TRAIL-R4 that includes at least five continuous or discontinuous amino acid residues of amino acids 141-210 of SEQ ID NO: 19 (e.g., at least five continuous or discontinuous amino acid residues of amino acids 151-179 and/or positions 171-200 of SEQ ID NO: 19, or at least five continuous or discontinuous amino acid residues of amino acids 161-169 and/or positions 181-190 of SEQ ID NO: 19), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic TRAIL-R4 antibodies or antigen-binding fragments thereof can target cells that express TRAIL-R4, such as, e.g., natural killer cells, CD8+ cytotoxic T cells, and tumor cells.

Antagonistic TRAMP antibodies or antigen-binding fragments thereof of the disclosure may bind an epitope within amino acids 122-191 of SEQ ID NO: 20. For example, antagonistic CD40 antibodies or antigen-binding fragments thereof of the disclosure may bind an epitope of, within, or including one or more of amino acids 122-170 (e.g., amino acids 142-150, LDCGALHRH, SEQ ID NO: 61) of the TRAMP amino acid sequence (SEQ ID NO: 20) and/or amino acids 142-191 (e.g., amino acids 162-171, CGTCLPGFYE, SEQ ID NO: 62) of the TRAMP amino acid sequence (SEQ ID NO: 20). In an embodiment, the epitope bound by the antibody or antigen-binding fragment thereof is or includes one or more of amino acids 142-150 of SEQ ID NO: 20. In an embodiment, the epitope bound by the antibody or antigen-binding fragment thereof is or includes one or more of amino acids 162-171 of SEQ ID NO: 20. Antagonistic TRAMP antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-TRAMP polypeptides (e.g., single-chain polypeptides, antibodies, and antigen-binding fragments) of the disclosure may also specifically bind an epitope within human TRAMP that includes at least five continuous or discontinuous amino acid residues of amino acids 122-191 of SEQ ID NO: 20 (e.g., at least five continuous or discontinuous amino acid residues of amino acids 132-160 and/or positions 152-181 of SEQ ID NO: 20, or at least five continuous or discontinuous amino acid residues of amino acids 142-150 and/or positions 162-171 of SEQ ID NO: 20). Furthermore, anti-TRAMP polypeptides (e.g., single-chain polypeptides, antibodies, and antigen-binding fragments) of the disclosure may also specifically bind an epitope within human TRAMP that includes at least five continuous or discontinuous amino acid residues of amino acids 122-191 of SEQ ID NO: 20 (e.g., at least five continuous or discontinuous amino acid residues of amino acids 132-160 and/or positions 152-181 of SEQ ID NO: 20, or at least five continuous or discontinuous amino acid residues of amino acids 142-150 and/or positions 162-171 of SEQ ID NO: 20), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic TRAMP antibodies or antigen-binding fragments thereof can target cells that express TRAMP, such as, e.g., activated T cells and FoxP3+ T-reg cells.

Antagonistic TROY antibodies or antigen-binding fragments thereof of the disclosure may bind an epitope within amino acids 31-102 of SEQ ID NO: 21. For example, antagonistic CD40 antibodies or antigen-binding fragments thereof of the disclosure may bind an epitope of, within, or including one or more of amino acids 31-79 (e.g., amino acids 51-59, QCGPGMELS, SEQ ID NO: 63) of the TROY amino acid sequence (SEQ ID NO: 21) and/or amino acids 52-102 (e.g., amino acids 72-82, CVTCRLHRFKE, SEQ ID NO: 64) of the TROY amino acid sequence (SEQ ID NO: 21). In an embodiment, the epitope bound by the antibody or antigen-binding fragment thereof is or includes one or more of amino acids 51-59 of SEQ ID NO: 21. In an embodiment, the epitope bound by the antibody or antigen-binding fragment thereof is or includes one or more of amino acids 72-82 of SEQ ID NO: 21. Antagonistic TROY antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-TROY polypeptides (e.g., single-chain polypeptides, antibodies, and antigen-binding fragments) of the disclosure may also specifically bind an epitope within human TROY that includes at least five continuous or discontinuous amino acid residues of amino acids 31-102 of SEQ ID NO: 21 (e.g., at least five continuous or discontinuous amino acid residues of amino acids 41-69 and/or positions 62-92 of SEQ ID NO: 21, or at least five continuous or discontinuous amino acid residues of amino acids 51-59 and/or positions 72-82 of SEQ ID NO: 21). Furthermore, anti-TROY polypeptides (e.g., single-chain polypeptides, antibodies, and antigen-binding fragments) of the disclosure may also specifically bind an epitope within human TROY that includes at least five continuous or discontinuous amino acid residues of amino acids 31--102 of SEQ ID NO: 21 (e.g., at least five continuous or discontinuous amino acid residues of amino acids 41-69 and/or positions 62-92 of SEQ ID NO: 21, or at least five continuous or discontinuous amino acid residues of amino acids 51-59 and/or positions 72-82 of SEQ ID NO: 21), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic TROY antibodies or antigen-binding fragments thereof can target cells that express TROY, such as, e.g., embryonic cells, colon cells, hair follicle cells, and brain cells.

Antagonistic XEDAR antibodies or antigen-binding fragments thereof of the disclosure may bind an epitope within amino acids 1-69 of SEQ ID NO: 22. For example, antagonistic CD40 antibodies or antigen-binding fragments thereof of the disclosure may bind an epitope of, within, or including one or more of amino acids 1-47 (e.g., amino acids 20-27, RCGPGQEL, SEQ ID NO: 65) of the XEDAR amino acid sequence (SEQ ID NO:22) and/or amino acids 22-69 (e.g., amino acids 42-29, TACPPRRY, SEQ ID NO: 66) of the XEDAR amino acid sequence (SEQ ID NO:22). In an embodiment, the epitope bound by the antibody or antigen-binding fragment thereof is or includes one or more of amino acids 20-27 of SEQ ID NO: 22. In an embodiment, the epitope bound by the antibody or antigen-binding fragment thereof is or includes one or more of amino acids 42-49 of SEQ ID NO: 22.

Antagonistic XEDAR antibodies or antigen-binding fragments thereof may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Anti-XEDAR polypeptides (e.g., single-chain polypeptides, antibodies, and antigen-binding fragments) of the disclosure may also specifically bind an epitope within human XEDAR that includes at least five continuous or discontinuous amino acid residues of amino acids 1-69 of SEQ ID NO: 22 (e.g., at least five continuous or discontinuous amino acid residues of amino acids 10-37 and/or positions 32-59 of SEQ ID NO: 22, or at least five continuous or discontinuous amino acid residues of amino acids 20-27 and/or positions 42-49 of SEQ ID NO: 22). Furthermore, anti-XEDAR polypeptides (e.g., single-chain polypeptides, antibodies, and antigen-binding fragments) of the disclosure may also specifically bind an epitope within human XEDAR that includes at least five continuous or discontinuous amino acid residues of amino acids 1-69 of SEQ ID NO: 22 (e.g., at least five continuous or discontinuous amino acid residues of amino acids 10-37 and/or positions 32-59 of SEQ ID NO: 22, or at least five continuous or discontinuous amino acid residues of amino acids 20-27 and/or positions 42-49 of SEQ ID NO: 22), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. Antagonistic XEDAR antibodies or antigen-binding fragments thereof can target cells that express XEDAR, such as, e.g., epidermal cells.

Polypeptides (e.g., single-chain polypeptides, antibodies, and antigen-binding fragments) of the disclosure may be dominant antagonists (single-chain polypeptides, antibodies, or antigen-binding fragments thereof). Dominant antagonistic TNFRSF member polypeptides are those that are capable of binding a TNFRSF member (e.g., in an anti-parallel dimer conformation) and inhibiting TNFRSF member-mediated signal transduction even in the presence of a natural ligand, such as CD40L for CD40, TNFα for TNFR1 and TNFR2, 4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others. For example, in the presence of a TNFRSF member ligand, a dominant antagonistic TNFRSF member polypeptide may inhibit the proliferation of a population of cells, such as T-reg cells, cancer cells that express the TNFRSF member, or myeloid-derived suppressor cells by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to a population of such cells that is not treated with a dominant antagonistic TNFRSF member polypeptide. In contrast, recessive antagonistic TNFRSF member polypeptides are capable of binding its specific TNFRSF member and inhibiting TNFRSF member-mediated signaling, but the ability of these polypeptides to do so is attenuated in the presence of a TNFRSF member ligand. For instance, the IC₅₀ of a recessive antagonistic TNFRSF member polypeptide as measured in the presence of a TNFRSF member ligand (such as CD40L for CD40, TNFα for TNFR1 and TNFR2, 4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others) in a T-reg cell death assay, a TNFRSF member expressing cancer cell death assay, or a myeloid-derived suppressor cell death assay may be augmented, e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to that of a dominant antagonistic TNFRSF member polypeptide. Dominant antagonistic TNFRSF member polypeptides, such as single-chain polypeptides, antibodies, or antigen-binding fragments thereof of the disclosure may be used to suppress the proliferation of a TNFRSF member expressing cell (e.g., a T-reg cell, a TNFRSF member expressing cancer cell, such as an ovarian cancer cell, or a myeloid-derived suppressor cell) even in the presence of a growth-inducing signal (such as CD40L for CD40, TNFα for TNFR1 and TNFR2, 4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others) by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to a population of such cells that is not treated with a dominant antagonistic TNFRSF member polypeptide.

### Antagonistic TNFRSF Member Polypeptides

The anti-TNFRSF member polypeptides (e.g., single-chain polypeptides, antibodies, and antigen-binding fragments, such as anti-CD40 polypeptides that bind CD40) of the disclosure are capable of interacting with and inhibiting the activity of TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40). Thus, the anti-TNFRSF member antibodies of the disclosure can selectively antagonize the TNFRSF member ligand-TNFRSF member interaction rather than promote TNFRSF member signaling.

The TNFRSF member polypeptides of the disclosure may bind a specific TNFRSF member with high affinity and may sterically sequester the receptor from the TNFRSF member ligand (e.g., CD40L for CD40, TNFα for TNFR1 and TNFR2, 4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others) rather than to allow TNFRSF member ligand binding to the TNFRSF member to initiate TNFRSF member signaling, e.g., by binding the TNFRSF member in an anti-parallel dimer conformation in which the cognate TNFRSF member ligand binding sites are sterically inaccessible.

### Antagonists Targeting Cell Proliferation Disorders and Infectious Diseases

Antagonistic polypeptides (e.g., single-chain polypeptides, antibodies, or fragments thereof) of the disclosure that bind TRAF-binding receptors (e.g., TNFRSF members listed in Table 3, in which antagonism thereof may be efficacious for treating diseases such as, e.g., cancer and infectious diseases) can be used to suppress T-reg cell growth and proliferation and can be administered to a mammalian subject, such as a human subject with a cell proliferation disorder or an infectious disease, in order to enhance the effectiveness of an immune response (e.g., an immune response against cancerous cells or pathogenic organisms) in the subject. This is particularly important for therapeutic applications, e.g., cancer immunotherapy, as TRAF-binding TNFRSF member activation upon association with TNFRSF member ligand leads to propagation of the MAPK and TRAF2/3 signal cascade and activation of NFκB-mediated transcription of genes involved in T-reg cell growth and escape from apoptosis (Faustman, et al., Nat. Rev. Drug Disc. 9:482-493, 2010).

Antagonistic TNFRSF member polypeptides (e.g., single-chain polypeptides, antibodies, or fragments thereof) of the disclosure may demonstrate the ability to attenuate T-reg and/or cancer cell proliferation even in the presence of a TNFRSF member agonist (such as TL1A, TRAIL, LT beta, LTα, CD153, N-APP, or RANKL, among others) or an agonistic TNFRSF member antibody, or growth-promoting molecules, such as IL-2. Without being limited by mechanism, antagonistic TNFRSF member single-chain polypeptides, antibodies, or antigen-binding fragments thereof of the disclosure may exhibit this property due to the ability of these antibodies or antigen-binding fragments thereof to bind a specific TNFRSF member and stabilize the dimeric, anti-parallel dimer conformation of this receptor. This structural configuration is not capable of potentiating NFκB signaling. By maintaining the TNFRSF member in an inactive structural state, antagonistic TNFRSF member single-chain polypeptides, antibodies, or antigen-binding fragments thereof of the disclosure may prevent TNFRSF member agonists from restoring cell growth.

Another property that may be exhibited by antagonistic TRAF-binding TNFRSF member polypeptides is the ability to not only reduce proliferation of a T-reg cell, a TNFRSF member expressing cancer cell, an MDSC, a T cell, a B cell, a monocyte, a neutrophil, a platelet, a granulocyte, a bone marrow-derived lymphoid cell, and/or a parenchymal cell, but also the ability to reduce the total quantity of these cells within a subject or sample (e.g., within a subject, such as a human subject who was administered the antagonist). Antagonistic TRAF-binding TNFRSF member single-chain polypeptides, antibodies, or antigen-binding fragments thereof of the disclosure may be capable of reducing the total quantity of T-reg cells, cancer cells (such as cutaneous T cell lymphoma cells, ovarian cancer cells, colon cancer cells, renal cell carcinoma cells or multiple myeloma cells, among others), and/or MDSCs in a subject or in a sample treated with an antagonist TNFRSF member polypeptide (such as a sample isolated from a human subject undergoing treatment for cancer or an infectious disease as described herein) by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to a subject or sample not treated with an antagonist TNFRSF member antibody or antigen-binding fragment thereof.

The ability of antagonistic TRAF-binding TNFRSF member polypeptides of the disclosure to attenuate T-reg and/or cancer cell growth may be due to the ability of these polypeptides to diminish the quantity of soluble TNFRSF members within a subject or sample (e.g., a sample isolated from a human subject undergoing treatment for cancer, an infectious disease, or an autoimmune disease as described herein). Soluble TNFRSF members can be secreted by, e.g., T-reg cells and can interfere with the ability of TNFRSF member antagonists to localize to the respective TNFRSF member at the surface of a T-reg cell, TNFRSF member expressing cancer cell, or MDSC by binding and sequestering such antagonists in the extracellular environment. By reducing TNFRSF member secretion, antagonistic TNFRSF member single-chain polypeptides, antibodies, or antigen-binding fragments thereof of the disclosure may render T-reg cells, TNFRSF member expressing cancer cells, MDSCs, T cells, B cells, monocytes, neutrophils, platelets, granulocytes, bone marrow-derived lymphoid cells, and/or parenchymal cells increasingly susceptible to therapeutic molecules, such as an antagonistic TNFRSF member antibody or antigen-binding fragment thereof, and/or additional anti-cancer agents described herein or known in the art that may be used in conjunction with the compositions and methods of the disclosure.

Antagonistic TRAF-binding TNFRSF member polypeptides of the disclosure may be capable of inhibiting the proliferation or reducing the total quantity of a population of T-reg cells in a subject or sample (e.g., a sample isolated from a human subject undergoing treatment for cancer or an infectious disease as described herein) and may act selectively on T-reg cells in an actively-dividing state. Antagonistic TRAF-binding TNFRSF member single-chain polypeptides, antibodies, or antigen-binding fragments thereof of the disclosure may selectively target active T-reg cells that express CD25^{Hi} and CD45RA^{Low}, e.g., over resting T-reg cells that express CD25Med and CD45RAF 1 ‘. For instance, antagonistic TRAF-binding TNFRSF member polypeptides of the disclosure may be capable of reducing the proliferation of a population of T-reg cells expressing CD25″’ and CD45RA^{Low} by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more relative to a population of T-reg cells that does not express the CD25^{Hi} and CD45RA^{Low} proteins, such as a population of T-reg cells that expresses CD25^{Med} and CD45RA^{Hi} proteins.

Antagonistic TRAF-binding TNFRSF member antibodies of the disclosure may inhibit growth of cells that express a TNFRSF members, such as, e.g., a T-reg, a cancer cell, a MDSC, a T cell, a B cell, a monocyte, a neutrophil, a platelet, a granulocyte, a bone marrow-derived lymphoid cell, and/or a parenchymal cell with a similar potency as that exhibited by antigen-binding fragments of such antibodies. For instance, removal of the Fc region of an antagonistic TRAF-binding TNFRSF member antibody of the disclosure may not alter the ability of the molecule to attenuate the proliferation or reduce the total quantity of T-reg cells and/or cancer cells in a subject or sample (e.g., a sample isolated from a human subject undergoing treatment for cancer or an infectious disease as described herein). Antagonistic TNFRSF member antibodies and antigen-binding fragments thereof of the disclosure may function by a pathway distinct from antibody-dependent cellular cytotoxicity (ADCC), in which an Fc region is required to recruit effector proteins in order to induce cell death. Additionally, antagonistic TNFRSF member antibodies or antigen-binding fragments thereof may not be susceptible to a loss of inhibitory capacity in the presence of cross-linking agents.

Antagonistic TNFRSF member antibodies or antigen-binding fragments thereof of the disclosure may therefore exhibit therapeutic activity in a variety of isotypes, such as IgG, IgA, IgM, IgD, or IgE, or in a variety of forms, such as a monoclonal antibody or antigen-binding fragment thereof, a polyclonal antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv).

Antagonistic polypeptides (e.g., single-chain polypeptides, antibodies, or fragments thereof) of the disclosure that stabilize the anti-parallel dimer conformation of a TRAF-binding TNFRSF member can interrupt cell growth and thus be used to treat diseases such as cancer and infectious diseases. For example, anti-TRAIL-R3 or anti-TRAIL-R4 antibodies of the disclosure can be administered to a subject to treat cancer (such as, e.g., breast cancer, pancreatic cancer, and adenocarcinoma) or infectious diseases by inhibiting the proliferation of, e.g., T-reg cells, cancer cells expressing TRAIL-R3 or TRAIL-R4, MDSCs, T cells, B cells, monocytes, neutrophils, platelets, and/or granulocytes. Anti-FN14 antibodies of the disclosure can be administered to a subject to treat glioblastomas and other brain tumors by inhibiting the proliferation of, e.g., T-reg cells, cancer cells expressing FN14, and/or MDSCs. Anti-LT beta receptor antibodies of the disclosure can be administered to a subject to treat melanoma, B cell lymphoma, or fibrosarcoma by inhibiting the proliferation of, e.g., T-reg cells, cancer cells expressing LT beta receptor, and/or MDSCs. Anti-HVEM antibodies of the disclosure can be administered to a subject to treat cancer (e.g., colorectal cancer, esophageal carcinoma, gastric cancer, hepatocarcinoma, breast cancer, and lymphoma) or infectious diseases by inhibiting the proliferation of, e.g., T-reg cells, cancer cells expressing HVEM, and MDSCs. Anti-CD30 antibodies of the disclosure can be administered to a subject to treat cancer (e.g., Hodgkin lymphoma or anaplastic large cell lymphoma) by inhibiting the proliferation of, e.g., lymphoma cells expressing CD30, T-reg cells, and/or MDSCs. Anti-BCMA antibodies or anti-TACI antibodies can be administered to a subject to treat cancer for B cell tumors, such as multiple myeloma and refractory multiple myeloma, by inhibiting proliferation of, e.g., cancer cells expressing BCMA (e.g., dominant antagonist BCMA polypeptides of the disclosure can inhibit the proliferation of rapidly dividing cells expressing BCMA without affecting dormant or normal B cells) or TACI, malignant B cells, T-reg cells, and/or MDSCs. Anti-BAFF-R antibodies of the disclosure may be administered to a subject to treat cancer for B cell tumors such as multiple myeloma or B cell acute lymphoblastic leukemia (B-ALL), by inhibiting the proliferation of, e.g., B cells, T-reg cells, and MDSCs. Anti-TROY antibodies of the disclosure can be administered to a subject to treat cancer such as colon cancer and glioblastomas by inhibiting the proliferation of, e.g., cancer cells expressing TROY, T-reg cells, and/or MDSCs. Anti-RELT (19L) antibodies of the disclosure can be administered to a subject to treat nerve/CNS tumors or infectious disease by inhibiting the proliferation of, e.g., T-reg cells, cancer cells expressing RELT (19L), and/or MDSCs.

Antagonistic polypeptides (e.g., single-chain polypeptides, antibodies, or fragments thereof) of the disclosure that bind DD-containing receptors (e.g., TNFRSF members listed in Table 2, in which antagonism thereof may be efficacious for treating diseases such as, e.g., cancer and infectious diseases) may be used to suppress apoptosis of, e.g., CD8+ cytotoxic T cells and B cells, and can be administered to a mammalian subject, such as a human subject with a cell proliferation disorder or an infectious disease. Antagonistic polypeptides of the disclosure that stabilize the anti-parallel dimer conformation of a DD-containing TNFRSF member can suppress apoptosis of, e.g., CD8+ cytotoxic T cells and B cells, and thus be used to treat diseases such as cancer and infectious diseases. For example, anti-TRAMP antibodies of the disclosure may be administered to a subject to treat cancer or infectious disease by suppressing apoptosis of, e.g., CD8+ cytotoxic T cells and B cells. Anti-DR6 antibodies of the disclosure can be administered to a subject to treat Alzheimer's disease, cancer, or infectious disease by suppressing apoptosis of, e.g., CD8+ cytotoxic T cells and B cells. Anti-NGFR antibodies of the disclosure can be administered to a subject to enhance maintenance of neurons, and can thus be used to treat a variety of neurological disorders by suppressing apoptosis of, e.g., CD8+ cytotoxic T cells and B cells.

### Antagonists Targeting Autoimmune Diseases

Antagonistic polypeptides (e.g., single-chain polypeptides, antibodies, or fragments thereof) of the disclosure may be used to elevate T-reg cell and/or MDSC activity, and to diminish the activity of T effector cells, thereby suppressing autoimmunity, graft-versus-host disease (GVHD), inflammation, and graft rejection, among other pathologies described herein. The compositions and methods of the disclosure may also be used to directly kill B cells, monocytes, neutrophils, platelets, macrophages, dendritic cells, epithelial cells, endothelial cells, granulocytes, mesenchymal cells, and effector T cells, such as CD8+ T cells, including those that react with endogenous (“self”) antigens.

Antagonistic polypeptides (e.g., single-chain polypeptides, antibodies, or fragments thereof) of the disclosure that bind TRAF-binding TNFRSF members (e.g., TNFRSF members listed in Table 3, in which antagonism thereof can be efficacious for treating an autoimmune disease, such as, e.g., allergies, asthma, and GVHD) can be used to inhibit growth and proliferation of, e.g., CD8+ cytotoxic T cells and B cells, platelets, macrophages, dendritic cells, and mesenchymal cells. Antagonistic polypeptides of the disclosure that bind TRAF-binding TNFRSF members can be administered to a mammalian subject, e.g., a human subject with an autoimmune disease. For example, anti-CD40 antibodies of the disclosure can be administered to a subject to treat autoimmune disease, allergies, asthma, or the like by inhibiting the proliferation of, e.g., CD8+ cytotoxic T cells, B cells, platelets, macrophages, dendritic cells, and mesenchymal cells. Anti-RANK antibodies of the disclosure can be administered to a subject to treat osteoporosis, tumor metastatic disease, or decreased bone loss in cancer by suppressing osteoclast differentiation through inhibiting the proliferation of, e.g., osteoclast cells, T-reg cells, and MDSCs. Anti-CD27 antibodies of the disclosure can be administered to a subject to treat autoimmune diseases, allergies, asthma, or the like by inhibiting the proliferation of, e.g., CD4+ and CD8+ cytotoxic T cells. Anti-4-1 BB antibodies or anti-OX40 antibodies of the disclosure can be administered to a subject to treat autoimmune diseases, asthma, and transplant rejection by inhibiting the proliferation of, e.g., CD4+ and CD8+ cytotoxic T cells, and NK cells. Anti-GITR antibodies of the disclosure may be administered to a subject to treat transplant rejection, autoimmune disease, or asthma by inhibiting the proliferation of, e.g., CD8+ cytotoxic T cells and NK cells. Anti-XEDAR antibodies of the disclosure can be administered to a subject to treat allergies, autoimmune disease, or transplant rejections by inhibiting the proliferation of, e.g., B cells and CD8+ cytotoxic T cells.

Antagonistic polypeptides (e.g., single-chain polypeptides, antibodies, or fragments thereof) of the disclosure that stabilize the anti-parallel dimer conformation of a DD-containing TNFRSF member (e.g., a TNFRSF member listed in Table 2 in which antagonism thereof can be efficacious for treating autoimmune diseases such as, e.g., allergies, asthma, and GVHD) may also be used to treat autoimmune diseases. For example, anti-TRAIL-R1 (TNFRSF10A) or anti-TRAIL-R2 (TNFRSF10B) antibodies of the disclosure can be administered to a subject to treat autoimmune disease by suppressing apoptosis of, e.g., T-reg cells and MDSCs. Anti-TNFR1 antibodies of the disclosure may be administered to a subject to treat autoimmune disease, inflammation, transplant rejection, or asthma by suppressing apoptosis of, e.g. T-reg cells and MDSCs.

### Framework Regions of Antagonistic TNFRSF Member Polypeptides

Antagonistic TNFRSF member polypeptides of the disclosure (e.g., a single-chain polypeptide, antibody, antigen-binding fragment thereof, or construct thereof, such as anti-CD40 polypeptides that bind CD40) may contain a framework (FW) region between each of the CDR sequences of the polypeptides described herein. The amino acid sequences of the FW regions can be natural sequences, such as those present in an antibody that is produced by immunization of an animal (e.g., a non-human animal) with an antigen sequence, such as one or more of those described herein. Native FW region sequence(s) or human FW sequence(s) can be used in the polypeptides of the present disclosure. The FW sequence(s) can also be prepared by using human FW sequence(s) that are modified to include one or more amino acids of a native FW region sequence.

The FW region amino acid sequences recognize, and are bound by, MHC class II proteins, including human leukocyte antigens (HLA) DR and DQ, among others. Typically, antibodies that are found to contain amino acid sequences that bind MHC proteins are engineered to remove such motifs, since antibodies that bind MHC proteins are susceptible to being degraded upon administration to a subject (e.g., a mammalian subject, such as a human subject) and positioned on the exterior of an antigen-presenting cell of the immune system, thereby triggering an inappropriate immune response against the administered antibody. Methods of determining whether a particular amino acid sequence is prone to bind MHC molecules are known in the art, and are described, e.g., in Wang et al., BMC Bioinformatics 11:568, 2010; Nielsen et al., BMC Bioinformatics 8:238, 2007; Gonzalez-Galarza et al., Nucleic Acid Research 39:D913-D919, 2011; and Greenbaum et al., Immunogenetics 63:325, 2011, the disclosures of each of which are incorporated herein by reference in their entirety.

The FW regions described above are not immunogenic peptides, despite their propensity to bind MHC class II molecules. Antagonistic TNFRSF member polypeptides of the disclosure (e.g., a single-chain polypeptide, antibody, antigen-binding fragment thereof, or construct thereof, such as anti-CD40 polypeptides that bind CD40) that contain FW region sequences exhibit the unexpected and beneficial property of TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) binding affinity without inducing an immunogenic response against the polypeptide upon administration of the polypeptide to a subject (e.g., a mammalian subject, such as a human).

### Specific Binding Properties of Antagonistic TNFRSF Member Polypeptides

The specific binding of a single-chain polypeptide, antibody or antibody fragment of the disclosure to human TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) can be determined by any of a variety of established methods. The affinity can be represented quantitatively by various measurements, including the concentration of antibody needed to achieve half-maximal inhibition of the TNFRSF member ligand-TNFRSF member interaction in vitro (IC50 and the equilibrium constant (K_{d}) of the polypeptide-TNFRSF member complex dissociation. The equilibrium constant, K_{d}, that describes the interaction of the TNFRSF member with a polypeptide of the disclosure is the chemical equilibrium constant for the dissociation reaction of a TNFRSF member-antibody complex into solvent-separated TNFRSF member protein and antibody molecules that do not interact with one another.

Polypeptides (e.g., single-chain polypeptides, antibodies, and antigen-binding fragments, such as anti-CD40 polypeptides that bind CD40) of the disclosure include those that specifically bind to its cognate TNFRSF member protein with a K_{d} value of less than 100 nM (e.g., 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 4 nM, 3 nM, 2 nM, or 1 nM). In some examples, antibodies of the disclosure are those that specifically bind to a TNFRSF member with a K_{d} value of less than 1 nM (e.g., (e.g., 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, 10 pM, 5 pM, or 1 pM).

Polypeptides (e.g., single-chain polypeptides, antibodies, and antigen-binding fragments, such as anti-CD40 polypeptides that bind CD40) of the disclosure can also be characterized by a variety of in vitro binding assays. Examples of experiments that can be used to determine the K_{d} or IC₅₀ of a TNFRSF member single-chain polypeptide, antibody, or fragment thereof include, e.g., surface plasmon resonance, isothermal titration calorimetry, fluorescence anisotropy, and ELISA-based assays, among others. ELISA represents a particularly useful method for analyzing antibody activity, as such assays typically require minimal concentrations of antibodies. A common signal that is analyzed in a typical ELISA assay is luminescence, which is typically the result of the activity of a peroxidase conjugated to a secondary antibody that specifically binds a primary antibody (e.g., a TNFRSF member antibody of the disclosure). Polypeptides of the disclosure are capable of binding TNFRSF members and epitopes derived thereof. For instance, polypeptides of the disclosure may bind peptides containing the amino acid sequence of any one of SEQ ID NOs: 25-66, or a variant thereof with up to 80% or greater sequence identity thereto. In a direct ELISA experiment, this binding can be quantified, e.g., by analyzing the luminescence that occurs upon incubation of an HRP substrate (e.g., 2,2′-azino-di-3-ethylbenzthiazoline sulfonate) with an antigen-antibody complex bound to a HRP-conjugated secondary antibody. For instance, polypeptides of the disclosure may induce a luminescence response of about 400 absorbance units or more when incubated with surface-immobilized antigen and a HRP-conjugated secondary antibody in the presence of an HRP substrate (see, e.g., Example 1). In some examples, the luminescence observed can be from about 400 to about 900 absorbance units (e.g., 400-900 absorbance units, 500-800 absorbance units, or 600-700 absorbance units). In particular cases, the luminescence observed can be from about 600 to about 900 absorbance units (e.g., 600-900 absorbance units or 700-800 absorbance units).

### Kinetic Properties of Antagonistic TNFRSF Member Polypeptides

In addition to the thermodynamic parameters of a TNFRSF member protein-polypeptide interaction, it is also possible to quantitatively characterize the kinetic association and dissociation of a single-chain polypeptide, antibody, or antibody fragment of the disclosure with a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40). This can be done, e.g., by monitoring the rate of antibody-antigen complex formation according to established procedures. For example, one can use surface plasmon resonance (SPR) to determine the rate constants for the formation (kₒₙ) and dissociation (kₒₙ) of an antibody-TNFRSF member complex. These data also enable calculation of the equilibrium constant of (K_{d}) of antibody-TNFRSF member protein complex dissociation, since the equilibrium constant of this unimolecular dissociation can be expressed as the ratio of the k_{off} to kₒₙ values. SPR is a technique that is particularly advantageous for determining kinetic and thermodynamic parameters of receptor-antibody interactions since the experiment does not require that one component be modified by attachment of a chemical label. Rather, the receptor is typically immobilized on a solid metallic surface which is treated in pulses with solutions of increasing concentrations of antibody. Antibody-receptor binding induces distortion in the angle of reflection of incident light at the metallic surface, and this change in refractive index over time as antibody is introduced to the system can be fit to established regression models in order to calculate the association and dissociation rate constants of an antibody-receptor interaction.

Polypeptides (e.g., single-chain polypeptides, antibodies, and antigen-binding fragments, such as anti-CD40 polypeptides that bind CD40) of the disclosure exhibit high kₒₙ and low k_{off} values upon interaction with TNFRSF members, consistent with high-affinity receptor binding. For example, antibodies TNFRAB1 and TNFRAB2 are exemplary as they bind TNFRSF member TNFR2 in an anti-parallel dimer configuration, and have kₒₙ values of 4.98×10⁶ M⁻¹s⁻¹, and 3.61×10⁵ M⁻¹s⁻¹, respectively, as shown in WO 2016/187068, which is incorporated herein by reference. Analogously, polypeptides of the disclosure may exhibit kₒₙ values in the presence of TNFRSF members of greater than 104 M⁻¹s⁻¹ (e.g., 1.0×10⁴ M⁻¹s⁻¹, 1.5×10⁴ M⁻¹s⁻¹, 2.0×10⁴ M⁻¹s⁻¹, 2.5×10⁴ M⁻¹s⁻¹, 3.0×10⁴ M⁻¹s⁻¹, 3.5×10⁴ M⁻¹s⁻¹, 4.0×10⁴ M⁻¹s⁻¹, 4.5×10⁴ M⁻¹s⁻¹, 5.0×10⁴ M⁻¹s⁻¹, 5.5×10⁴ M⁻¹s⁻¹, 6.0×10⁴ M⁻¹s⁻¹, 6.5×10⁴ M⁻¹s⁻¹, 7.0×10⁴ M⁻¹s⁻¹, 7.5×10⁴ M⁻¹s⁻¹, 8.0×10⁴ M⁻¹s⁻¹, 8.5×10⁴ M⁻¹s⁻¹, 9.0×10⁴ M⁻¹s⁻¹, 9.5×10⁴ M⁻¹s⁻¹, 1.0×10⁵ M⁻¹s⁻¹, 1.5×10⁵ M⁻¹s⁻¹, 2.0×10⁵ M⁻¹s⁻¹, 2.5×10⁵ M⁻¹s⁻¹, 3.0×10⁵ M⁻¹s⁻¹, 3.5×10⁵ M⁻¹s⁻¹, 4.0×10⁵ M⁻¹s⁻¹, 4.5×10⁵ M⁻¹s⁻¹, 5.0×10⁵ M⁻¹s⁻¹, 5.5×10⁵ M⁻¹s⁻¹, 6.0×10⁵ M⁻¹s⁻¹, 6.5×10⁵ M⁻¹s⁻¹, 7.0×10⁵ M⁻¹s⁻¹, 7.5×10⁵ M⁻¹s⁻¹, 8.0×10⁵ M⁻¹s⁻¹, 8.5×10⁵ M⁻¹s⁻¹, 9.0×10⁵ M⁻¹s⁻¹, 9.5×10⁵ M⁻¹s⁻¹, or 1.0×10⁶ M⁻¹s⁻¹). Polypeptides of the disclosure exhibit low k_{off} values when bound to TNFRSF members, since antibodies are capable of interacting with distinct TNFRSF member epitopes with a high affinity. Residues within these epitopes form strong intermolecular contacts with TNFRSF members, which serves to slow the dissociation of the polypeptide-TNFRSF member complex. This high receptor affinity is manifested in low k_{off} values. For example, TNFRAB1 and TNFRAB2 bind TNFRSF member TNFR2, and have k_{off} values of 2.21×10⁴ s⁻¹ and 2.24×10⁻⁴ s⁻¹, respectively, as shown in WO 2016/187068. Similarly, antibodies of the disclosure may exhibit k_{off} values of less than 10⁻³ s⁻¹ when complexed to TNFRSF members (e.g., 1.0×10⁻³ s⁻¹, 9.5×10⁻⁴ s⁻¹, 9.0×10⁻⁴ s⁻¹, 8.5×10⁻⁴ s⁻¹, 8.0×10⁻⁴ s⁻¹, 7.5×10⁻⁴ s⁻¹, 7.0×10⁻⁴ s⁻¹, 6.5×10⁻⁴ s⁻¹, 6.0×10⁻⁴ s⁻¹, 5.5×10⁻⁴ s⁻¹, 5.0×10⁻⁴ s⁻¹, 4.5×10⁻⁴ s⁻¹, 4.0×10⁻⁴ s⁻¹, 3.5×10⁻⁴ s⁻¹, 3.0×10⁻⁴ s⁻¹, 2.5×10⁻⁴ s⁻¹, 2.0×10⁻⁴ s⁻¹, 1.5×10⁻⁴ s⁻¹, 1.0×10⁻⁴ s⁻¹, 9.5×10⁻⁵ s⁻¹, 9.0×10⁻⁵ s⁻¹, 8.5×10⁻⁵ s⁻¹, 8.0×10⁻⁵ s⁻¹, 7.5×10⁻⁵ s⁻¹, 7.0×10⁻⁵ s⁻¹, 6.5×10⁻⁵ s⁻¹, 6.0×10⁻⁵ s⁻¹, 5.5×10⁻⁵ s⁻¹, 5.0×10⁻⁵ s⁻¹, 4.5×10⁻⁵ s⁻¹, 4.0×10⁻⁵ s⁻¹, 3.5×10⁻⁵ s⁻¹, 3.0×10⁻⁵ s⁻¹, 2.5×10⁻⁵ s⁻¹, 2.0×10⁻⁵ s⁻¹, 1.5×10⁻⁵ s⁻¹, or 1.0×10⁻⁵ s⁻¹).

Epitopes within TNFRSF Members Bound by Antagonistic TNFRSF Member Polypeptides

The affinities (e.g., K_{d}<1 μM) of polypeptides (e.g., single-chain polypeptides, antibodies, and antigen-binding fragments, such as anti-CD40 polypeptides that bind CD40) of the disclosure for TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) coupled with the rapid onset of polypeptide-TNFRSF member complex formation and the slow dissociation of these complexes render these polypeptides well-suited for therapeutic applications as modulators (e.g., suppressors or enhancers) of, e.g., T-reg and CD8+ cytotoxic cell growth and proliferation via antagonism of a TNFRSF member. The high kₒₙ values, for instance, indicate that polypeptides (e.g., single-chain polypeptides, antibodies, and antigen-binding fragments, such as anti-CD40 polypeptides that bind CD40) of the disclosure are capable of localizing to the surface of a TNFRSF member expressing cell (e.g., a T-reg cell or a CD8+ cytotoxic cell) and rapidly associating with the TNFRSF member, thereby preventing receptor activation that may otherwise be induced by a cognate or natural TNFRSF member ligand (such as CD40L for CD40, TNFα for TNFR1 and TNFR2, 4-1 BBL for 4-1 BB, CD70 for CD27, CD153 for CD30, N-APP for DR6, EDA-A1 for EDAR, FasL for Fas, GITRL for GITR, LTα for HVEM, LT beta (TNF-C) for LT beta receptor complex, NGF for NGFR, TRAIL for OPG, OX40L for OX40, RANKL for RANK, TRAIL for TRAIL-R2 (TNFRSF10B), TRAIL-R1 (TNFRSF10A), and TRAIL-R4, TL1A for TRAMP, or EDA-A2 for XEDAR, among others), e.g., by inhibiting the trimerization of the TNFRSF member by its cognate or natural TNFRSF member ligand. Moreover, the slow dissociation of the polypeptide-TNFRSF member complex can be indicative of a long half-life of the complex in vivo, which results in stable, sustained modulation (e.g., down-regulation or up-regulation) of the growth of the TNFRSF member expressing cell (e.g., T-reg or CD8+ cytotoxic cell growth). These ideal thermodynamic and kinetic parameters of TNFRSF member binding are consistent with the strong intermolecular contacts that are established upon association of polypeptides of the disclosure with TNFRSF members.

Among the difficulties in developing anti-TNFRSF member polypeptides (e.g., single-chain polypeptides, antibodies, and antigen-binding fragments, such as anti-CD40 polypeptides that bind CD40) that are capable of antagonizing TNFRSF members has been the elucidation of epitopes within TNFRSF members that participate in antagonistic complex formation rather than epitopes that promote signal transduction. Various discrete peptide fragments found within the TNFRSF member primary structure bind antagonistic antibodies of the disclosure by virtue of the spatial orientation of these residues in the native conformation of the receptor. Significantly, these residues have been difficult to identify, as many isolated linear TNFRSF member-derived peptides do not appear to interact with antagonistic TNFRSF member polypeptides (e.g., single-chain polypeptides, antibodies, and antibody fragments, such as anti-CD40 polypeptides that bind CD40) due to the different conformations these peptides exhibit when structurally pre-organized within the full-length protein and when isolated in solution. Epitope mapping analysis using constrained cyclic and bicyclic peptides derived from various regions of TNFRSF members indicates that antagonistic TNFRSF member antibodies of the disclosure bind epitopes from distinct regions of the TNFRSF member amino acid sequence in a conformation-dependent manner.

The present disclosure also features anti-TNFRSF member polypeptides in the form of an IgG2 isotype, which demonstrate substantially improved TNFRSF member antagonist effects. As described in the examples below, it has presently been discovered that this class of TNFRSF member polypeptides exhibits a surprisingly superior ability to disrupt TNFRSF member signaling, modulate (e.g., down-regulate or up-regulate) T-reg cell and CD8+ cytotoxic cell growth, and/or augment the proliferation of effector T cells relative to TNFRSF member-binding polypeptides of other isotypes.

Another discovery underlying the present disclosure is the finding that antagonistic TNFRSF member polypeptides that contain antigen-binding sites spatially separated from one another by about 133 Å or more exhibit unexpectedly superior TNFRSF member antagonist effects relative to polypeptides that specifically bind TNFRSF member at one or more of the epitopes described above but that contain antigen-binding sites separated from one another by fewer than about 133 Å. Examples of such polypeptides include IgG1 antibodies and antigen-binding fragments thereof that contain antigen-binding sites separated from one another by about 117 Å and IgG3 antibodies and antigen-binding fragments thereof that contain antigen-binding sites separated from one another by 125 Å.

Antagonistic TNFRSF member polypeptides of the disclosure can be formulated into pharmaceutical compositions. The pharmaceutical composition can be formulated with polypeptides of the disclosure that adopt a single disulfide-bonded isoform. For example, pharmaceutical compositions of the disclosure include those containing an antagonist TNFRSF member polypeptide in which, e.g., 10%, 20%, 30%, 40%, 50%, 60%, 70%, 80%, 90%, 95%, 96%, 97%, 98%, 99%, 99.9%, 99.99%, or more, of the polypeptide in the pharmaceutical composition is present in a single disulfide-bonded isoform. Antagonistic TNFRSF member polypeptides of the disclosure may advantageously adopt an IgG2-A disulfide-bonded isoform, which has surprisingly been found to promote a substantially more robust level of TNFRSF member antagonism relative to other IgG2 disulfide-bonded isoforms, such as the IgG2-B, IgG2-A/B1, and IgG2-A/B2 isoforms. Polypeptides of the disclosure may be engineered to predominantly adopt an IgG2-A isoform, for example, by introducing mutations into the IgG2 hinge region that prohibit the formation of other disulfide-bonded isoforms. Exemplary mutations in the amino acid sequence of a human IgG2 hinge region that promote the formation of the IgG2-A isoform at the exclusion of the remaining isoforms described above include the deletions and/or substitutions of the cysteine residues at positions 232 and 233 of the wild-type human IgG2 hinge amino acid sequence.

### IgG2 Isotype Antibodies Promotes Optimal TNFRSF Member Antagonism

As described above and herein, optimal TNFRSF member antagonism among human, humanized, and chimeric TNFRSF member antagonist antibodies and antigen-binding fragment thereof is achieved when the antibody or antibody fragment has a human IgG2 isotype, particularly when the antibody or antibody fragment has an IgG2-A disulfide-bonded isoform.

To stabilize the IgG2-A disulfide-bonded isoform, mutations can be introduced into the IgG2 hinge region so as to prevent, or reduce the occurrence of, disulfide bonding between cysteine residues that are present as nonbonded thiols in the IgG2-A isoform. Examples of such mutations are amino acid substitutions or deletions at residues C232 and C233 of the human IgG2 hinge region. By removing one or both of these residues and optionally replacing these residues with amino acids that are incapable of forming disulfide bonds, one can bias the disulfide bonding pattern in a population of IgG2 isoforms towards the IgG2-A isoform. Examples of amino acid substitutions that can be used to obtain a population of IgG2-A isoform antibodies include conservative amino acid substitutions, such as the C232S and C233S amino acid substitutions. Due to the similar molecular volume and polarity of cysteine and serine, the C232S and C233S substitutions feature the beneficial effect of preserving the steric and electronegativity properties of the naturally-occurring cysteine residue while prohibiting the formation of a disulfide bond at position 232 and/or 233 of the IgG2 hinge region. By incorporating C232S and/or C233S substitutions into a TNFRSF member antibody or fragment thereof, a population of TNFRSF member antagonist antibodies or fragments having an IgG2-A isoform can be obtained. Methods of effectuating amino acid substitutions and deletions into an antibody or antigen-binding fragment thereof include mutagenesis techniques described herein and known in the art.

### Spacing Between Antigen-Binding Sites

Antagonistic TNFRSF member polypeptide (e.g., single-chain polypeptides, antibody, antigen-binding fragment thereof, or construct thereof) described herein may contain antigen-binding sites (i.e., antigen-binding arms) that are separated from one another by a distance of at least about 133 Å, which is the spacing observed between antigen-binding arms in human IgG2 isotype antibodies. As described in the examples below, it has been discovered that this spacing gives rise to antibodies having optimal TNFRSF member antagonistic properties. TNFRSF member antagonist polypeptides of the disclosure include those containing antigen-binding arms separated by, e.g., a distance of from about 133 Å to about 160 Å, such as a distance of about 133 Å, 134 Å, 135 Å, 136 Å, 137 Å, 138 Å, 139 Å, 140 Å, 141 Å, 142 Å, 143 Å, 144 Å, 145 Å, 146 Å, 147 Å, 148 Å, 149 Å, 150 Å, 151 Å, 152 Å, 153 Å, 154 Å, 155 Å, 156 Å, 157 Å, 158 Å, 159 Å, or 160 Å). For example, the polypeptide (e.g., a single-chain polypeptide, antibody, antigen-binding fragment thereof, or construct thereof) may contain antigen-binding sites that are separated from one another by a distance of from about 133 Å to about 150 Å, such as by a distance of about 133 Å, 134 Å, 135 Å, 136 Å, 137 Å, 138 Å, 139 Å, 140 Å, 141 Å, 142 Å, 143 Å, 144 Å, 145 Å, 146 Å, 147 Å, 148 Å, 149 Å, or 150 Å. In some examples, the antigen-binding sites are separated from one another by a distance of from about 133 Å to about 145 Å, such as by a distance of about 133 Å, 134 Å, 135 Å, 136 Å, 137 Å, 138 Å, 139 Å, 140 Å, 141 Å, 142 Å, 143 Å, 144 Å, or 145 Å. In some examples, the antigen-binding sites are separated from one another by a distance of from about 133 Å to about 139 Å, such as by a distance of about 133 Å, 134 Å, 135 Å, 136 Å, 137 Å, 138 Å, or 139 Å. In some examples, the antigen-binding sites are separated from one another by a distance of from about 134 Å to about 139 Å, such as by a distance of about 134 Å, 135 Å, 136 Å, 137 Å, 138 Å, or 139 Å.

The TNFRSF member antagonist polypeptides described herein may have, e.g., two, three, four, five, or more, antigen-binding arms separated by a distance specified above. Examples of antibody fragments that have two or more antigen-binding arms include, without limitation, diabodies, triabodies, F(ab′)₂ molecules, and tandem scFv (taFv) molecules, among others. Methods of generating these antibody fragments include peptide synthesis and recombinant protein expression techniques described herein and known in the art.

There exist a variety of methods for measuring the distance between antigen-binding arms of an antibody or antibody fragment. For example, distances between antigen-binding arms of an antibody can be made by analyzing the three-dimensional structure of an antibody or antibody fragment using computer software, such as through the use of PYMOL® and other molecular imaging software. Three-dimensional structures of polypeptides, such as antibodies and antibody fragments, can be calculated using the data obtained from X-ray crystallography experiments and nuclear magnetic resonance (NMR) techniques known in the art. Examples of X-ray crystallography and NMR methods that can be used to obtain three-dimensional polypeptide structures are described, e.g., in Eigenbrot et al., Journal of Molecular Biology 229:969-995, 1993; and Huang et al., Science 317:1930-1934, 2007, the disclosures of each of which are incorporated herein by reference in their entirety.

### Uniformity of Populations of TNFRSF Member Antagonist Polypeptides

Pharmaceutical compositions can be generated in which the TNFRSF member antagonist polypeptide (e.g., antibody, antigen-binding fragment thereof, single-chain polypeptide, or construct thereof) described herein is present as a single disulfide-bonded isoform. For example, at least 10%, or more, of the polypeptide in the pharmaceutical composition may be present as a single disulfide-bonded isoform (e.g., the IgG2-A isoform). This may be achieved, for example, by way of amino acid substitutions or deletions at one or both of cysteine residues 232 and 233 of the wild-type human IgG2 hinge region, thereby preventing or reducing the occurrence of disulfide bonding that could give rise to an IgG2 isoform other than IgG2-A. The pharmaceutical compositions of the disclosure include those in which, for example, about 10% to about 99.999% of the antagonist TNFRSF member polypeptide in the pharmaceutical composition is present in a single disulfide-bonded isoform, such as the IgG2-A isoform. For example, pharmaceutical compositions of the disclosure include those containing an antagonist TNFRSF member polypeptide in which, e.g., 10%, 20%, 30%, 40%, 50%, 60%, 70%, 80%, 90%, 95%, 96%, 97%, 98%, 99%, 99.9%, 99.99%, or more, of the polypeptide in the pharmaceutical composition is present in a single disulfide-bonded isoform.

Techniques for measuring the relative quantities of various disulfide-bonded isoforms present in a subject or sample of an antagonist TNFRSF member polypeptide include liquid chromatography techniques known in the art and described herein, such as those exemplified in Wypych et al., The Journal of Biological Chemistry 283:16194-16205, 2008, the disclosure of which is incorporated herein by reference in its entirety.

### Effects on T-Reg Cell Proliferation Using Antagonistic TNFRSF Member Polypeptides for Treating Cancer and Infectious Disease

Antagonistic TRAF-binding TNFRSF member (e.g., a TNFRSF member listed in Table 3, in which antagonism thereof may be efficacious for treating diseases such as, e.g., cancer and infectious disease) described herein, can be used to attenuate the activity of T-reg cells that typically accompanies T cell-mediated cytotoxicity against self cells, such as the attack of a tumor cell by a T lymphocyte. This can be achieved, for instance, due to the ability of antagonistic TNFRSF member polypeptides described herein to inhibit the proliferation of, and/or to directly kill, T-reg cells. Antagonistic TNFRSF member polypeptides (e.g., single-chain polypeptides, antibodies, and antigen-binding fragments thereof) can, thus, be administered (e.g., by any of a variety of routes of administration described herein) to a mammalian subject, such as a human, in order to prolong the duration of an adaptive immune response, such as a response against a cancer cell or a pathogenic organism. In this way, for example, antagonistic TNFRSF member polypeptides, such as single-chain polypeptides, antibodies, or antigen-binding fragments thereof described herein, may synergize with existing techniques to enhance T lymphocyte-based therapy for cancer and for infectious diseases. For instance, TRAF-binding TNFRSF member antagonists described herein may be administered to suppress T-reg cell activity, thereby enhancing the cytotoxic effect of tumor reactive T cells. TNFRSF member antagonists may also synergize with existing strategies to promote tumor-reactive T cell survival, such as lymphodepletion and growth factor therapy, and in turn prolong the duration of anti-tumor reactivity in vivo.

Antagonistic TRAF-binding TNFRSF member polypeptides, such as single-chain polypeptides, antibodies, and antigen-binding fragments thereof can also be used to treat a broad array of infectious diseases in a mammalian subject (e.g., a human), as inhibition of T-reg proliferation promotes the activity of CD8+ T lymphocytes capable of mounting an attack on pathogenic organisms. Additionally, antagonistic TNFRSF member antibodies and antigen-binding fragments thereof described herein can be used to treat a wide variety of infectious diseases, such as Mycobacterium tuberculosis, in a human or an agricultural farm animal (e.g., a bovine mammal, pig, cow, horse, sheep, goat, cat, dog, rabbit, hamster, guinea pig, or other non-human mammal).

Antagonistic DD-containing TNFRSF member polypeptides, such as single-chain polypeptides, antibodies, and antigen-binding fragments thereof may be used to treat infectious diseases in a mammalian subject (e.g., a human), as an upregulation of CD8+ cytotoxic T lymphocytes and B cells may facilitate an effective immune response against the invasive pathogenic organism.

### Direct Effects on TNFRSF Member Expressing Cancer Cells

Antagonistic TNFRSF member polypeptides, such as single-chain polypeptides, antibodies, or antigen-binding fragments thereof described herein may bind and inactivate TNFRSF members (e.g., TNFRSF members listed in Tables 2 and 3, in which antagonism thereof may be efficacious for treating diseases such as, e.g., cancer and infectious diseases) on the surface of a cancer cell, such as a TNFRSF member expressing tumor cell. For instance, antagonistic TNFRSF member antibodies and antigen-binding fragments thereof described herein may bind a TNFRSF member on the surface a T cell lymphoma cell (e.g., a Hodgkin or cutaneous non-Hodgkin lymphoma cell), ovarian cancer cell, colon cancer cell, multiple myeloma cell, or renal cell carcinoma cell, among others. The ability of antagonistic TNFRSF member antibodies and antigen-binding fragments thereof described herein to bind specific TNFRSF members directly on a cancer cell provides another pathway by which these molecules may attenuate cancer cell survival and proliferation. For instance, an antagonistic TNFRSF member polypeptide described herein, such as an antagonistic TNFRSF member single-chain polypeptide, antibody, antigen-binding fragment thereof, or construct, may bind a TNFRSF member directly on the surface of a cancer cell (e.g., a cutaneous T cell lymphoma cell, ovarian cancer cell, colon cancer cell, or multiple myeloma cell, such as an ovarian cancer cell) in order to suppress the ability of the cell to proliferate and/or to promote apoptosis of the cell.

### TNFRSF Member Antagonist Polypeptides are not Reliant on Additional TNFRSF Member-Binding Agents for Activity

Significantly, antagonistic TNFRSF member polypeptides, such as single-chain polypeptides, antibodies, or antigen-binding fragments thereof described herein, are capable of binding specific TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40), thereby suppressing TNFRSF member-mediated signaling without the need for an endogenous TNFRSF member-binding agent, such as CD40L, TNFα, 4-1 BBL, CD70, CD153, or RANKL, among others. Antagonistic TNFRSF member polypeptides, such as single-chain polypeptides, antibodies, and antigen-binding fragments thereof described herein do not require TNFRSF member ligand to suppress the natural ligand functions listed in Tables 2 and 3. Without being limited by mechanism, antagonistic TNFRSF member antibodies or antigen-binding fragments thereof described herein may exhibit this property due to the ability of these antibodies or antigen-binding fragments thereof to bind TNFRSF members at particular epitopes that, when bound, stabilize the anti-parallel dimer conformation of the receptor (e.g., as shown in FIG. 4). This structural configuration is not capable of potentiating a signal. By maintaining the TNFRSF member in an inactive structural state, antagonistic TNFRSF member polypeptides described herein may prevent TNFRSF member agonists from restoring activity (e.g., the natural ligand functions of each TNFRSF member listed in Tables 2 and 3) of a TNFRSF member expressing cell.

For instance, antagonistic TRAF-binding TNFRSF member polypeptides, such as single-chain polypeptides, antibodies, antigen-binding fragments thereof, and constructs thereof described herein, may bind TNFRSF members on the surface of a TRAF-binding TNFRSF member expressing cell, such as a T-reg cell, cancer cell, myeloid-derived suppressor cell (MDSC), T cells, B cells, monocytes, neutrophils, platelets, granulocytes, bone marrow-derived lymphoid cells, and parenchymal cells, and inhibit the proliferation of such cells in the presence or absence of a cognate or natural TNFRSF member ligand. For example, antagonistic TNFRSF member polypeptides, such as single-chain polypeptides, antibodies, and antigen-binding fragments thereof described herein, may inhibit the proliferation of such cells by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to such cells that are not treated with the TRAF-binding TNFRSF member antagonist polypeptide. The antagonistic TNFRSF member polypeptide (e.g., single-chain polypeptide, antibody, or antigen-biding fragment thereof) may exhibit an IC₅₀ value in such a cell proliferation assay that is largely unchanged by the presence or absence of the TNFRSF member cognate or natural ligand (e.g., an IC₅₀ value in the presence of TNFα that is changed by less than 50%, 45%, 40%, 35%, 25%, 20%, 15%, 10%, 9%, 8%, 7%, 6%, 5%, 4%, 3%, 2%, or less than 1% relative to the IC₅₀ value of the antagonistic TNFRSF member polypeptide (e.g., single-chain polypeptide, antibody, or antigen-binding fragment thereof) in the same cell proliferation assay in the absence of the TNFRSF member cognate or natural ligand). Similarly, antagonistic TRAF-binding TNFRSF member polypeptides, such as single-chain polypeptides, antibodies, antigen-binding fragments thereof, and constructs thereof described herein, may inhibit TNFRSF member signaling as assessed by measuring the expression of one or more genes selected from the group consisting of CHUK, NFKBIE, NFKBIA, MAP3K11, TRAF2, TRAF3, relB, and cIAP2/BIRC3 by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to such cells that are not treated with the TRAF-binding TNFRSF member antagonist polypeptide. The antagonistic TNFRSF member polypeptide (e.g., single-chain polypeptide, antibody, or antigen-biding fragment thereof) may exhibit an IC₅₀ value in such a gene expression assay that is largely unchanged by the presence or absence of TNFRSF member cognate or natural ligand (e.g., an IC₅₀ value in the presence of TNFRSF member cognate or natural ligand that is changed by less than 50%, 45%, 40%, 35%, 25%, 20%, 15%, 10%, 9%, 8%, 7%, 6%, 5%, 4%, 3%, 2%, or less than 1% relative to the IC₅₀ value of the antagonistic TNFRSF member polypeptide (e.g., single-chain polypeptide, antibody, or antigen-binding fragment thereof) in the same gene expression assay in the absence of TNFRSF member cognate or natural ligand such as CD153).

### Direct Killing of T-Reg Cells, MDSCs, and TNFRSF Member Expressing Cancer Cells

Antagonistic TRAF-binding TNFRSF member polypeptides disclosed herein (e.g., single-chain polypeptides, antibodies, or fragments thereof), and constructs thereof, may, for instance, not only reduce the proliferation of T-reg cells, TNFRSF member expressing cancer cells, and/or MDSCs, but may also induce the death of T-reg cells, TNFRSF member expressing cancer cells, and/or MDSCs within a sample (e.g., within a subject, such as a human subject administered the antagonist). Antagonistic TRAF-binding TNFRSF member polypeptides described herein may be capable, for instance, of reducing the total quantity of T-reg cells, cancer cells (such as cutaneous T cell lymphoma cells, ovarian cancer cells, colon cancer cells, renal cell carcinoma cells or multiple myeloma cells, among others), and/or MDSCs in a subject or in a sample treated with an antagonist TNFRSF member antibody or antigen-binding fragment thereof (such as a sample isolated from a human subject undergoing treatment for cancer or an infectious disease as described herein) by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to a subject or sample not treated with an antagonist TNFRSF member antibody or antigen-binding fragment thereof.

The ability of antagonistic TRAF-binding TNFRSF member polypeptides (e.g., single-chain polypeptides, antibodies, or fragments thereof) described herein to attenuate T-reg, MDSC, and/or cancer cell growth may be due, in part, to the ability of these polypeptides to diminish the quantity of soluble TRAF-binding TNFRSF member within a subject or sample (e.g., a sample isolated from a human subject undergoing treatment for cancer or an infectious disease as described herein). In the absence of this beneficial activity, soluble TRAF-binding TNFRSF member can be secreted by, e.g., T-reg cells, and could otherwise interfere with the ability of TNFRSF member antagonists to localize to the TNFRSF member at the surface of a T-reg cell, TNFRSF member expressing cancer cell, or MDSC by binding and sequestering such antagonists in the extracellular environment. By reducing TRAF-binding TNFRSF member secretion, antagonistic TNFRSF member antibodies or antigen-binding fragments thereof described herein may render T-reg cells, TRAF-binding TNFRSF member expressing cancer cells, and/or MDSCs increasingly susceptible to therapeutic molecules, such as an antagonistic TRAF-binding TNFRSF member antibody or antigen-binding fragment thereof, and/or additional anti-cancer agents, such as those described herein or known in the art, that may be used in conjunction with the compositions and methods described herein.

### Modulation of T-Reg Cells, MDSCs, and T Effector Cells in the Tumor Microenvironment

Antagonist TRAF-binding TNFRSF member polypeptides described herein (e.g., single-chain polypeptides, antibodies, or fragments thereof), may inhibit the proliferation of T-reg cells with a greater potency in a subject suffering from cancer relative to a subject that does not have cancer. The antagonist TRAF-binding TNFRSF member polypeptides described herein, such as single-chain polypeptides, antibodies, and antigen-binding fragments thereof, may inhibit the proliferation of T-reg cells with a greater potency in the microenvironment of a tumor relative to a site that is free of cancer cells, such as a site distal from a tumor in a subject suffering from cancer or in a subject without cancer. This effect may be determined using, for example, a cell death assay as described herein. For instance, the polypeptides described herein, such as single-chain polypeptides, antibodies, antigen-binding fragments thereof, and constructs thereof, may exhibit an IC₅₀ for reducing or inhibiting the proliferation of T-reg cells in the microenvironment of a tumor that is less than the IC₅₀ of the polypeptides for reducing or inhibiting the proliferation of T-reg cells in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. The polypeptides described herein, such as single-chain polypeptides, antibodies, antigen-binding fragments thereof, and constructs thereof, may inhibit the proliferation of T-reg cells or may promote the apoptosis of T-reg cells with a potency that is greater in the microenvironment of a tumor containing TRAF-binding TNFRSF member expressing cancer cells, such as Hodgkin lymphoma cells, cutaneous non-Hodgkin lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, than in a site that is free of such cancer cells, such as a site distal from a tumor in a subject suffering from one or more of the foregoing cancers or a in a subject without cancer.

Depending on the TNFRSF receptor that is inhibited, the polypeptides described herein (e.g., single-chain polypeptides, antibodies, or fragments thereof), and constructs thereof, may inhibit the proliferation of MDSCs with a greater potency in a subject suffering from cancer relative to a subject that does not have cancer. The polypeptides described herein, such as single-chain polypeptides, antibodies, antigen-binding fragments thereof, and constructs thereof, may inhibit the proliferation of MDSCs with a greater potency in the microenvironment of a tumor relative to a site that is free of cancer cells, such as a site distal from a tumor in a subject suffering from cancer or in a subject without cancer. This effect may be determined using, for example, a cell death assay described herein. For instance, the polypeptides described herein, such as single-chain polypeptides, antibodies, antigen-binding fragments thereof, and constructs thereof, may have an IC₅₀ for reducing or inhibiting the proliferation of MDSCs in the microenvironment of a tumor that is less than the IC₅₀ of the polypeptides for reducing or inhibiting the proliferation of MDSCs in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. The polypeptides described herein, such as single-chain polypeptides, antibodies, antigen-binding fragments thereof, and constructs thereof, may inhibit the proliferation of MDSCs or may promote the apoptosis of MDSCs with a potency that is greater in the microenvironment of a tumor containing TRAF-binding TNFRSF member expressing cancer cells, such as Hodgkin lymphoma cells, cutaneous non-Hodgkin lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, than in a site that is free of such cancer cells, such as a site distal from a tumor in a subject suffering from one or more of the foregoing cancers or a in a subject without cancer.

Antagonistic polypeptides described herein (e.g., single-chain polypeptides, antibodies, or fragments thereof), which antagonize a DD-containing TNFRSF member (e.g., a TNFRSF member listed in Table 2, in which antagonism thereof may be efficacious for treating diseases such as, e.g., cancer and infectious diseases), may expand T effector cells, such as CD8+ cytotoxic T cells, with a greater potency in a subject suffering from cancer relative to a subject that does not have cancer. In some cases, the polypeptides described herein, such as single-chain polypeptides, antibodies, and antigen-binding fragments thereof, expand T effector cells, such as CD8+ cytotoxic T cells, with a greater potency in the microenvironment of a tumor relative to a site that is free of cancer cells, such as a site distal from a tumor in a subject suffering from cancer or a in a subject without cancer. This effect may be determined using, for example, a cell proliferation assay described herein. For instance, the polypeptides described herein (e.g., single-chain polypeptides, antibodies, or fragments thereof), and constructs thereof, may have an EC₅₀ for the expansion of T effector cells in the microenvironment of a tumor that is less than the EC₅₀ of the polypeptides for expanding T effector cells in a site that is free of cancer cells by, for example, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 1,000-fold, 10,000-fold, or more. The polypeptides described herein, such as single-chain polypeptides, antibodies, antigen-binding fragments thereof, and constructs thereof, may directly expand T effector cells, such as CD8+ cytotoxic T cells, with a potency that is greater in the microenvironment of a tumor containing TNFRSF member expressing cancer cells, such as Hodgkin lymphoma cells, cutaneous non-Hodgkin lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, than in a site that is free of such cancer cells, such as a site distal from a tumor in a subject suffering from one or more of the foregoing cancers or a in a subject without cancer. The T effector cells (e.g., CD8+ cytotoxic T cells) may, for example, specifically react with an antigen present on one or more cancer cells, such as Hodgkin lymphoma cells, cutaneous non-Hodgkin lymphoma cells, T cell lymphoma cells, ovarian cancer cells, colon cancer cells, multiple myeloma cells, renal cell carcinoma cells, skin cancer cells, lung cancer cells, liver cancer cells, endometrial cancer cells, hematopoietic or lymphoid cancer cells, central nervous system cancer cells, breast cancer cells, pancreatic cancer cells, stomach cancer cells, esophageal cancer cells, and upper gastrointestinal cancer cells, among cells of other cancers described herein.

### Dual Proliferative and Cell Killing Effects of TNFRSF Member Antagonist Polypeptides

Antagonistic DD-containing TNFRSF member (e.g., a TNFRSF member listed in Table 2, in which antagonism thereof may be efficacious for treating autoimmune disorders such as, e.g., asthma, allergies, and GVHD) polypeptides disclosed herein, such as single-chain polypeptides, antibodies, antigen-binding fragments thereof, and constructs thereof, may not only promote the proliferation of T-reg cells, MDSCs, and/or TNFRSF member-positive parenchymal cells, but may also induce the death of B cells, monocytes, neutrophils, platelets, macrophages, dendritic cells, epithelial cells, endothelial cells, granulocytes, mesenchymal cells, and T effector cells (e.g., within a subject, such as a human subject). Antagonistic DD-containing TNFRSF member polypeptides described herein may be capable, for instance, of increasing the total quantity of T-reg cells, MDSCs, and/or TNFRSF member-positive parenchymal cells in a subject or in a sample treated with an antagonist DD-containing TNFRSF member antibody or antigen-binding fragment thereof (such as a sample isolated from a human subject undergoing treatment for autoimmunity, GVHD, transplant rejection, allergies, chronic inflammatory disease, asthma, or another disorder described herein) by, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or more, relative to a subject or sample not treated with an antagonistic TNFRSF member antibody or antigen-binding fragment thereof. The ability of antagonistic DD-containing TNFRSF member polypeptides (e.g., single-chain polypeptides, antibodies, and antigen-binding fragments) described herein to augment T-reg, MDSC, and/or parenchymal cell growth may be due, in part, to the ability of these polypeptides to decrease the quantity of soluble DD-containing TNFRSF member within a subject or sample (e.g., a sample isolated from a human subject undergoing treatment for or autoimmunity, GVHD, transplant rejection, allergies, chronic inflammatory disease, asthma, or another disorder described herein).

The stimulation of T-reg and MDSC proliferation engenders important therapeutic benefits, as these cells suppress the activity of T effector cells, such as autoreactive CD8+ T cells that mount an inappropriate immune response against self tissue. Similarly, the direct killing of B cells, monocytes, neutrophils, platelets, macrophages, dendritic cells, epithelial cells, endothelial cells, granulocytes, mesenchymal cells, and T effector cells imparts therapeutic activity, as TRAF-binding TNFRSF member (e.g., a TNFRSF member listed in Table 3 in which antagonism thereof can be efficacious for treating autoimmune diseases such as, e.g., asthma, allergies, and GVHD) antagonist polypeptides can be used to reduce the quantity of self-reactive T cells in a subject suffering from autoimmunity, GVHD, transplant rejection, allergies, chronic inflammatory disease, asthma, or another disorder described herein.

Through one or both of these mechanisms, DD-containing and TRAF-binding TNFRSF member antagonist polypeptides of the disclosure can be used to suppress immunological conditions in a subject, such as a mammalian subject (e.g., a human subject).

Additionally, the ability of TNFRSF member antagonist polypeptides of the disclosure to stimulate proliferation of TNFRSF member-positive parenchymal cells provides the beneficial effect of inducing tissue and organ regeneration. Exemplary TNFRSF member-positive parenchymal cells that can be induced to proliferate using the TNFRSF member antagonist polypeptides of the disclosure include, without limitation, cells of the pancreas, salivary gland, pituitary gland, kidney, heart, lung, hematopoietic system, cranial nerves, heart, aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes. Thus, TNFRSF member antagonist polypeptides described herein can be used to treat disorders in which the regeneration, protection, and/or healing of one or more of these cell types is desired. Exemplary diseases that can be treated using the TNFRSF member antagonist polypeptides of the disclosure are described herein.

Antagonistic TNFRSF Member Polypeptides that Bind TNFRSF Members from Non-Human Animals

In addition to binding epitopes within human TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40), antagonistic TNFRSF member polypeptides (e.g., single-chain polypeptides, antibodies, and antigen-binding fragments, such as anti-CD40 polypeptides that bind CD40) of the disclosure also include those that specifically bind epitopes containing the equivalent motif within the TNFRSF member derived from non-human animals, such as non-human mammals, e.g., in a cow, bison, mouse, or rat, among others.

### Antagonistic TNFRSF Member Single-Chain Polypeptides

TNFRSF member antagonists of the disclosure may be in the form of a single-chain polypeptide, such as a single-chain polypeptide that contains one, two, or three heavy chain CDRs of a monoclonal TNFRSF member antagonist antibody described herein, and/or one, two, or three light chain CDRs of a monoclonal TNFRSF member antagonist antibody described. Single-chain polypeptides may be in the form of an antibody fragment, e.g., an antibody fragment described herein or known in the art, such as a scFv fragment. Single chain polypeptides may alternatively contain one or more CDRs described herein covalently bound to one another using conventional bond-forming techniques known in the art, for instance, by an amide bond, a thioether bond, a carbon-carbon bond, or by a linker, such as a peptide linker or a multi-valent electrophile (e.g., a bis(bromomethyl) arene derivative, such as a bis(bromomethyl)benzene or bis(bromomethyl)pyridine) described herein or known in the art.

Single-chain polypeptides can be produced by a variety of recombinant and synthetic techniques, such as by recombinant gene expression or solid-phase peptide synthesis procedures described herein or known in the art. For instance, one of skill in the art can design polynucleotides encoding, e.g., two or more of the above CDRs operably linked to one another in frame so as to produce a continuous, single-chain peptide containing these CDRs. Optionally, the CDRs may be separated by a spacer, such as by a framework region (e.g., a framework sequence described herein or a framework region of a germline consensus sequence of a human antibody) or a flexible linker, such as a poly-glycine or glycine/serine linker described herein or known in the art. When produced by chemical synthesis methods, native chemical ligation can optionally be used as a strategy for the synthesis of long peptides (e.g., greater than 50 amino acids). Native chemical ligation protocols are known in the art and have been described, e.g., by Dawson et al. (Science, 266:776-779, 1994); incorporated herein by reference. A detailed description of techniques for the production of single-chain polypeptides, full-length antibodies, and antibody fragments is provided in the sections that follow.

### Nucleic Acids and Expression Systems

Antagonistic TNFRSF member polypeptides (e.g., single-chain polypeptides, antibodies, or antigen-binding fragments thereof, such as anti-CD40 polypeptides that bind CD40) of the disclosure can be prepared by any of a variety of established techniques. For instance, an antagonistic TNFRSF member polypeptide (e.g., single-chain polypeptides, antibodies, or antigen-binding fragments thereof) of the disclosure can be prepared by recombinant expression of one or more immunoglobulin light and heavy chain genes in a host cell. For instance, to express an antibody recombinantly, a host cell can be transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N. Y., 1989), Current Protocols in Molecular Biology (Ausubel et al., eds., Greene Publishing Associates, 1989), and in U.S. Pat. No. 4,816,397; incorporated herein by reference.

### Vectors for Expression of Antagonistic TNFRSF Member Polypeptides

Viral genomes provide a rich source of vectors that can be used for the efficient delivery of exogenous genes into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the disclosure include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030); incorporated herein by reference.

### Genome Editing Techniques

In addition to viral vectors, a variety of additional methods have been developed for the incorporation of genes, e.g., those encoding antibody light and heavy chains, single-chain polypeptides, single-chain variable fragments (scFvs), tandem scFvs, Fab domains, F(ab′)₂ domains, diabodies, and triabodies, among others, into the genomes of target cells for polypeptide expression. One such method that can be used for incorporating polynucleotides encoding anti-TNFRSF member polypeptides (e.g., single-chain polypeptides, antibodies, or antigen-binding fragments thereof, such as anti-CD40 polypeptides that bind CD40) into prokaryotic or eukaryotic cells includes transposons. Transposons are polynucleotides that encode transposase enzymes and contain a polynucleotide sequence or gene of interest flanked by excision sites at the 5′ and 3′ positions. Once a transposon has been delivered into a cell, expression of the transposase gene commences and results in active enzymes that cleave the gene of interest from the transposon. This activity is mediated by the site-specific recognition of transposon excision sites by the transposase. In some cases, these excision sites may be terminal repeats or inverted terminal repeats. Once excised from the transposon, the gene of interest can be integrated into the genome of a prokaryotic or eukaryotic cell by transposase-catalyzed cleavage of similar excision sites that exist within nuclear genome of the cell. This allows the gene encoding, e.g., an anti-TNFRSF member antibody or fragment or domain thereof to be inserted into the cleaved nuclear DNA at the excision sites, and subsequent ligation of the phosphodiester bonds that join the gene of interest to the DNA of the prokaryotic or eukaryotic cell genome completes the incorporation process. In some cases, the transposon may be a retrotransposon, such that the gene encoding the antibody is first transcribed to an RNA product and then reverse-transcribed to DNA before incorporation in the prokaryotic or eukaryotic cell genome. Exemplary transposon systems include the piggybac transposon (described in detail in WO 2010/085699) and the sleeping beauty transposon (described in detail in US20050112764); incorporated herein by reference.

Another useful method for the integration of nucleic acid molecules encoding anti-TNFRSF member polypeptides (e.g., single-chain polypeptides, antibodies, or antigen-binding fragments thereof, such as anti-CD40 polypeptides that bind CD40) into the genome of a prokaryotic or eukaryotic cell is the clustered regularly interspaced short palindromic repeats (CRISPR)/Cas system, which is a system that originally evolved as an adaptive defense mechanism in bacteria and archaea against infection by viruses. The CRISPR/Cas system consists of palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al., Nat. Biotech., 31:227-229, 2013) and can be used as an efficient means of site-specifically editing eukaryotic or prokaryotic genomes in order to cleave DNA prior to the incorporation of a polynucleotide encoding an anti-TNFRSF member polypeptide of the disclosure. The use of CRISPR/Cas to modulate gene expression has been described in U.S. Pat. No. 8,697,359, which is incorporated herein by reference.

Alternative methods for site-specifically cleaving genomic DNA prior to the incorporation of a polynucleotide encoding a TNFRSF member antibody or antibody fragment of the disclosure include the use of zinc finger nucleases and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. Zinc finger nucleases and TALENs for use in genome editing applications are described in Urnov et al. (Nat. Rev. Genet. 11:636-646, 2010); and in Joung et al., (Nat. Rev. Mol. Cell. Bio. 14:49-55, 2013); incorporated herein by reference. Additional genome editing techniques that can be used to incorporate polynucleotides encoding antibodies of the disclosure into the genome of a prokaryotic or eukaryotic cell include the use of ARCUS™ meganucleases that can be rationally designed so as to site-specifically cleave genomic DNA. The use of these enzymes for the incorporation of polynucleotides encoding antagonistic TNFRSF member polypeptides (e.g., single-chain polypeptides, antibodies, or antibody fragments, such as anti-CD40 polypeptides that bind CD40) of the disclosure into the genome of a prokaryotic or eukaryotic cell is particularly advantageous in view of the structure-activity relationships that have been established for such enzymes. Single-chain meganucleases can thus be modified at certain amino acid positions in order to create nucleases that selectively cleave DNA at desired locations. These single-chain nucleases have been described extensively, e.g., in U.S. Pat. Nos. 8,021,867 and 8,445,251; incorporated herein by reference.

### Polynucleotide Sequence Elements

To express antagonistic TNFRSF member polypeptides (e.g., single-chain polypeptides, antibodies, or antibody fragments thereof, such as anti-CD40 polypeptides that bind CD40) of the disclosure, polynucleotides encoding partial or full-length light and heavy chains, or CDRs thereof, e.g., obtained as described above, can be inserted into expression vectors such that the genes are operatively linked to transcriptional and translational control sequences. The expression vector and expression control sequences are chosen to be compatible with the expression host cell used. Polynucleotides encoding, e.g., the light chain gene and the heavy chain of a TNFRSF member antibody can be inserted into separate vectors, or, optionally, both polynucleotides can be incorporated into the same expression vector using established techniques described herein or known in the art.

In addition to polynucleotides encoding the heavy and light chains of an antibody (or a polynucleotide encoding a single-chain polypeptide or an antibody fragment, such as a scFv molecule), the recombinant expression vectors of the disclosure may carry regulatory sequences that control the expression of the antibody chain genes in a host cell. The design of the expression vector, including the selection of regulatory sequences, may depend on such factors as the choice of the host cell to be transformed or the level of expression of protein desired. For instance, suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. Nos. 5,168,062, 4,510,245, and 4,968,615.

In addition to the antibody chain or CDR genes and regulatory sequences, the recombinant expression vectors of the disclosure can carry additional sequences, such as sequences that regulate replication of the vector in host cells (e.g., origins of replication) and selectable marker genes. A selectable marker gene facilitates selection of host cells into which the vector has been introduced (see e.g., U.S. Pat. Nos. 4,399,216, 4,634,665 and 5,179,017). For example, typically the selectable marker gene confers resistance to cytotoxic drugs, such as G418, puromycin, blasticidin, hygromycin or methotrexate, to a host cell into which the vector has been introduced. Suitable selectable marker genes include the dihydrofolate reductase (DHFR) gene (for use in DHFR″ host cells with methotrexate selection/amplification) and the neo gene (for G418 selection). In order to express the light and heavy chains of a TNFRSF member antibody or a TNFRSF member antibody fragment, the expression vector(s) containing polynucleotides encoding the heavy and light chains can be transfected into a host cell by standard techniques.

### Polynucleotides Encoding Modified Antagonistic TNFRSF Member Polypeptides

Antagonistic TNFRSF member polypeptides (e.g., single-chain polypeptides, antibodies, or antibody fragments of the disclosure, such as anti-CD40 polypeptides that bind CD40) may feature differences in the sequence of one or more CDRs. In other cases, the polypeptides of the disclosure may feature differences in one or more framework regions. Exemplary framework regions include, for example, human framework regions described in U.S. Pat. No. 7,829,086, and primate framework regions as described in EP 1945668; incorporated herein by reference. Alternatively, polypeptides (e.g., single-chain polypeptides, antibodies, or antigen-binding fragments thereof, such as anti-CD40 polypeptides that bind CD40) of the disclosure may exhibit differences in the sequence of one or more CDRs and differences in one or more framework regions. To generate nucleic acids encoding such TNFRSF member antagonist polypeptides, DNA fragments encoding, e.g., one or more CDRs, or at least one, or both, of the light chain variable regions and the heavy chain variable regions can be produced by chemical synthesis (e.g., by solid-phase polynucleotide synthesis techniques), in vitro gene amplification (e.g., by polymerase chain reaction techniques), or by replication of the polynucleotide in a host organism. For instance, nucleic acids encoding anti-TNFRSF member polypeptides of the disclosure may be obtained by amplification and modification of germline DNA or cDNA encoding light and heavy chain variable sequences.

This can be achieved, for example, by performing site-directed mutagenesis of germline DNA or cDNA and amplifying the resulting polynucleotides using the polymerase chain reaction (PCR) according to established procedures. Germline DNA sequences for human heavy and light chain variable region genes are known in the art (see, e.g., the “VBASE” human germline sequence database; see also Kabat et al., Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al., Eur. J. Immunol. 24:827-836, 1994; incorporated herein by reference). Chimeric nucleic acid constructs encoding human heavy and light chain variable regions containing one or more of the CDRs of the disclosed antibody can be produced, e.g., using established cloning techniques known in the art. Additionally, a polynucleotide encoding the heavy or light chain variable region of the disclosed antibody can be synthesized and used as a template for mutagenesis to generate a variant as described herein using routine mutagenesis techniques. Alternatively, a DNA fragment encoding the variant can be directly synthesized (e.g., by established solid phase nucleic acid chemical synthesis procedures).

Once DNA fragments encoding related VH and VL segments are obtained, these DNA fragments can be further manipulated by standard recombinant DNA techniques, e.g., to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker.

The isolated DNA encoding the VH region of an anti-TNFRSF member antibody of the disclosure can be converted to a full-length heavy chain gene (as well as a Fab heavy chain gene), e.g., by operatively linking the VH-encoding DNA to another DNA molecule encoding heavy chain constant region domains (CH1, CH2, CH3, and, optionally, CH4). The sequences of human heavy chain constant region genes are known in the art (see e.g., Kabat et al., Sequences of Proteins of Immunological Interest, Fifth Edition, U.S. Department of Health and Human Services, NIH Publication No. 91-3242, 1991) and DNA fragments encompassing these regions can be obtained by standard PCR amplification. The heavy chain constant region can be an IgG1, IgG2, IgG3, IgG4, IgA, IgE, IgM or IgD constant region, and in certain examples is an IgG1 constant region. For a Fab fragment heavy chain gene, the V_{H}-encoding DNA can be operatively linked to another DNA molecule encoding only the heavy chain CH1 domain.

The isolated DNA encoding the V_{L} region of an anti-TNFRSF member polypeptide (e.g., single-chain polypeptide, antibody, or antigen-binding fragment thereof) of the disclosure can be converted to a full-length light chain gene (as well as a Fab light chain gene) by operatively linking the V_{L}-encoding DNA to another DNA molecule encoding the light chain constant region, CL. The sequences of human light chain constant region genes are known in the art (see e.g., Kabat et al., Sequences of Proteins of Immunological Interest, Fifth Edition (U.S. Department of Health and Human Services, NIH Publication No. 91-3242, 1991)) and DNA fragments encompassing these regions can be obtained, e.g., by amplification in a prokaryotic or eukaryotic cell of a polynucleotide encoding these regions, by PCR amplification, or by chemical polynucleotide synthesis. The light chain constant region can be a kappa (κ) or lambda (A) constant region, but in certain examples is a kappa constant region. To create a scFv gene, the V_{H} and V_{L}-encoding DNA fragments are operatively linked to another fragment encoding a flexible linker, e.g., a polynucleotide encoding a flexible, hydrophilic amino acid sequence, such as the amino acid sequence (Gly₄Ser)₃, such that the V_{H} and V_{L} sequences can be expressed as a contiguous single-chain protein, with the V_{L} and V_{H} regions joined by the linker (see e.g., Bird et al., Science 242:423-426, 1988; Huston et al., Proc. Natl. Acad. Sci. USA 85:5879-5883, 1988; McCafferty et al., Nature 348:552-554, 1990).

Recombinant DNA technology can also be used to remove some or all of the DNA encoding either or both of the light and heavy chains that is not necessary for binding to a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40). The molecules expressed from such truncated DNA molecules are also encompassed by the polypeptides of the disclosure. Dual specific antibodies, i.e., antibodies that bind TNFRSF members and a different antigen using the same binding site, can be produced by mutating amino acid residues in the light chain and/or heavy chain CDRs.

Dual specific antibodies, e.g., antibodies that bind a TNFRSF member and a different antigen using the same binding site, can be produced by mutating amino acid residues in the light chain and/or heavy chain CDRs. Dual functional antibodies can be made by expressing a polynucleotide engineered to encode a dual specific antibody.

Modified antagonistic TNFRSF member antibodies and antibody fragments of the disclosure can also be produced by chemical synthesis (e.g., by the methods described in Solid Phase Peptide Synthesis, 2^{nd} ed., 1984 The Pierce Chemical Co., Rockford, 111; incorporated herein by reference). Variant antibodies can also be generated using a cell-free synthetic platform (see, e.g., Chu et al., Biochemia No. 2, 2001 (Roche Molecular Biologicals); incorporated herein by reference).

### Host Cells for Expression of Antagonistic TNFRSF Member Polypeptides

It is possible to express the polypeptides (e.g., single-chain polypeptides, antibodies, and antigen-binding fragments thereof, such as anti-CD40 polypeptides that bind CD40) of the disclosure in either prokaryotic or eukaryotic host cells. In some examples, expression of polypeptides (e.g., single-chain polypeptides, antibodies, or antigen-binding fragments thereof, such as anti-CD40 polypeptides that bind CD40) is performed in eukaryotic cells, e.g., mammalian host cells, for optimal secretion of a properly folded and immunologically active antibody. Exemplary mammalian host cells for expressing the recombinant polypeptides (e.g., single-chain polypeptides, antibodies, or antigen-binding fragments thereof, such as anti-CD40 polypeptides that bind CD40) of the disclosure include Chinese Hamster Ovary (CHO cells) (including DHFR CHO cells, described in Urlaub and Chasin (Proc. Natl. Acad. Sci. USA 77:4216-4220, 1980), used with a DHFR selectable marker, e.g., as described in Kaufman and Sharp (Mol. Biol. 159:601-621, 1982), NSO myeloma cells, COS cells, 293 cells, and SP2/0 cells. Additional cell types that may be useful for the expression of single-chain polypeptides, antibodies, and fragments thereof include bacterial cells, such as BL-21(DE3) E. coli cells, which can be transformed with vectors containing foreign DNA according to established protocols. Additional eukaryotic cells that may be useful for expression of polypeptides include yeast cells, such as auxotrophic strains of S. cerevisiae, which can be transformed and selectively grown in incomplete media according to established procedures known in the art. When recombinant expression vectors encoding antibody genes (e.g., genes encoding one or more CDRs, an antibody heavy chain, or an antibody light chain) are introduced into mammalian host cells, the antibodies are produced by culturing the host cells for a period of time sufficient to allow for expression of the antibody in the host cells or secretion of the antibody into the culture medium in which the host cells are grown.

Polypeptides (e.g., single-chain polypeptides, antibodies, and antigen-binding fragments thereof, such as anti-CD40 polypeptides that bind CD40) can be recovered from the culture medium using standard protein purification methods. Host cells can also be used to produce portions of intact antibodies, such as Fab fragments or scFv molecules. The disclosure also includes methods in which the above procedure is varied according to established protocols known in the art. For example, it can be desirable to transfect a host cell with DNA encoding either the light chain or the heavy chain (but not both) of an anti-TNFRSF member antibody of this disclosure in order to produce an antigen-binding fragment of the antibody.

Once an anti-TNFRSF member polypeptide (e.g., single-chain polypeptide, antibody, or antigen-binding fragment) thereof of the disclosure has been produced by recombinant expression, it can be purified by any method known in the art, such as a method useful for purification of an immunoglobulin molecule, for example, by chromatography (e.g., ion exchange, affinity, particularly by affinity for a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) after Protein A or Protein G selection, and sizing column chromatography), centrifugation, differential solubility, or by any other standard technique for the purification of proteins. Further, the anti-TNFRSF member polypeptides (e.g., single-chain polypeptides, antibodies, and antigen-binding fragments thereof, such as anti-CD40 polypeptides that bind CD40) of the disclosure can be fused to heterologous polypeptide sequences described herein or otherwise known in the art to facilitate purification or to produce therapeutic conjugates (see “Antibody conjugates,” below).

Once isolated, an anti-TNFRSF member antibody or antigen-binding fragments thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a SUPERDEX™ 75 column (Pharmacia Biotech AB, Uppsala, Sweden).

### Platforms for Generating and Affinity-Maturing Antagonistic Anti-TNFRSF Member Polypeptides

Mapping Epitopes of TNFRSF Members that Promote Receptor Antagonism

Antagonistic TNFRSF member polypeptides (e.g., single-chain polypeptides, antibodies and antigen-binding fragments thereof, such as anti-CD40 polypeptides that bind CD40) of the disclosure can be produced by screening libraries of polypeptides (e.g., single-chain polypeptides, antibodies, and antigen-binding fragments thereof, such as anti-CD40 polypeptides that bind CD40) for functional molecules that are capable of binding epitopes within TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) that selectively promote receptor antagonism rather than receptor activation. Linear peptides isolated from the TNFRSF members may not adopt the same three dimensional conformations as those peptide sequences located within the protein. TNFRSF members provide a structurally rigidified framework that biases the conformations of individual peptide fragments and reinforces these spatial orientations by establishing intramolecular contacts (e.g., hydrogen bonds, dipole-dipole interactions, salt bridges) and by differentially positioning various regions for exposure to solvent depending on the relative hydrophilicity and lipophilicity of these areas (Mukai et al., Sci. Signal., 3:ra83-ra83, 2010). The conformational constraint of a peptide fragment within a TNFRSF member can be achieved by incorporating the amino acid residues of a TNFRSF member epitope (e.g., an epitope that promotes receptor antagonism) into a structurally pre-organized peptide scaffold, such as a cyclic, bicyclic, tricyclic, or tetracyclic peptide. Cyclic and polycyclic peptides such as these confine a peptide fragment to a distinct three-dimensional conformation. This can be achieved by synthesizing peptide epitopes isolated from TNFRSF members by established chemical synthetic methods (e.g., by solid-phase peptide synthesis as described herein) and incorporating cysteine residues into the sequence at the N- and C-terminal positions or at various internal positions within the peptide chain. It may be advantageous to incorporate cysteine residues that are chemically protected at the thiol moiety with a protecting group that can be removed under conditions different from those used to remove other protecting groups within the peptide being synthesized and different from those used to assemble the peptide chain. Exemplary orthogonal protecting groups for the cysteine thiol include the 4-methyltrityl group and 4-methoxtrityl group, each of which can be removed using dilute trifluoracetic acid (examples are described, e.g., in Isidro-Llobet et al., Chem Rev. 109:2455-2504, 2009).

After introducing a cysteine residue into a synthetic peptide fragment derived from an epitope within a TNFRSF member, the peptide can be cyclized by treating the peptide with a multivalent electrophile, such as a bis(bromomethyl) or tris(bromomethyl)arene derivative. Alternative multivalent thiol-reactive electrophiles can be used, e.g., 1,5-difluoro-2,4-dinitrobenzene, acyclic dibromoalkanes, and others (see, e.g., Jo et al., J. Am. Chem. Soc. 134:17704-17713, 2012; incorporated herein by reference). In some examples, it may be advantageous to prevent the participation of a cysteine residue in the synthetic peptide fragment in a cyclization reaction. For instance, it may be desirable to synthesize a polycyclic peptide containing multiple cysteine residues such that only select cysteine thiols participate in the intramolecular crosslinking process. To prevent unwanted participation of these additional Cys thiol groups in the coupling reaction, a simple approach is for instance to use Fmoc-Cys(Acm) (Fmoc-acetamidomethyl-L-cysteine) for introduction of a protected Cys residue during the course of peptide synthesis. Alternatively, Fmoc-Cys(StBu)-OH can be used, and/or the corresponding t-butyloxycarbonyl (Boc)-protected amino acids. The Acm or StBu group is not removed during the course of a normal TFA deprotection-cleavage reaction but requires oxidative (e.g., iodine, 12) treatment in case of Acm group, or reductive treatment (e.g., 8-mercaptoethanol (excess) or 1,4-dithiothreiotol (excess)) in case of the StBu group to give the reduced sulfhydryl form of the peptide, which can either be used directly or subsequently oxidized to the corresponding cystinyl peptide. In one embodiment, a peptide is used which contains at least one Cys derivative, such as Cys(Acm) or Cys(StBu), to allow selective deprotection of a Cys-thiol group. Selective deprotection of a Cys-thiol group renders the Cys-thiol group available for reacting at a desired moment, such as following completion of peptide chain assembly and prior to the deprotection of other residues within the peptide (see, e.g., WO 2008/013454; incorporated herein by reference).

As an example, libraries of cyclic and polycyclic peptides containing individual fragments isolated from TNFRSF members and combinations of fragments from distinct regions of TNFRSF members can be synthesized by techniques such as those described above in order to incorporate cysteine residues at various positions within the peptide scaffold and using different electrophilic crosslinking reagents. These peptides can be immobilized on a solid surface and screened for molecules that bind antagonistic TNFRSF member polypeptides (e.g., single-chain polypeptides, antibodies, or antigen-binding fragments thereof, such as anti-CD40 polypeptides that bind CD40) using an ELISA-based screening platform using established procedures. Using this assay, for instance, peptides that specifically bind TNFRSF member antibodies with high affinity therefore contain residues within epitopes of the TNFRSF member that preferentially bind the TNFRSF member antibody and may structurally pre-organize these residues such that they resemble the conformations of the corresponding peptide in the native protein. Cyclic and polycyclic peptides thus obtained (e.g., peptides having the sequence of any one of SEQ ID NOs: 25-66) can be used to screen libraries of antibodies and antigen-binding fragments thereof in order to identify anti-TNFRSF member polypeptides of the disclosure. Moreover, since these constrained peptides act as surrogates for epitopes within TNFRSF members that promote receptor antagonism, polypeptides (e.g., single-chain polypeptides, antibodies, and antigen-binding fragments thereof, such as anti-CD40 polypeptides that bind CD40) generated using this screening technique may bind the corresponding epitopes in TNFRSF members and are expected to be antagonistic of receptor activity. For instance, CD40 antibodies may bind peptides having the sequence of SEQ ID NOs: 25 and/or 26, or a variant thereof with up to 80% or greater sequence identity thereto. For instance, 4-1 BB antibodies may bind peptides having the sequence of SEQ ID NO: 27, or a variant thereof with up to 80% or greater sequence identity thereto. For instance, CD27 antibodies may bind peptides having the sequence of SEQ ID NOs: 28 and/or 29, or a variant thereof with up to 80% or greater sequence identity thereto. For instance, CD30 antibodies may bind peptides having the sequence of SEQ ID NOs: 30 and/or 31, or a variant thereof with up to 80% or greater sequence identity thereto. For instance, DR6 antibodies may bind peptides having the sequence of SEQ ID NOs: 32 and/or 33, or a variant thereof with up to 80% or greater sequence identity thereto. For instance, EDAR antibodies may bind peptides having the sequence of SEQ ID NOs: 34 and/or 35, or a variant thereof with up to 80% or greater sequence identity thereto. For instance, Fas antibodies may bind peptides having the sequence of SEQ ID NOs: 36 and/or 37, or a variant thereof with up to 80% or greater sequence identity thereto. For instance, GITR antibodies may bind peptides having the sequence of SEQ ID NOs: 38 and/or 39, or a variant thereof with up to 80% or greater sequence identity thereto. For instance, HVEM antibodies may bind peptides having the sequence of SEQ ID NOs: 40 and/or 41, or a variant thereof with up to 80% or greater sequence identity thereto. For instance, LT beta receptor antibodies may bind peptides having the sequence of SEQ ID NOs: 42 and/or 43, or a variant thereof with up to 80% or greater sequence identity thereto. For instance, NGFR antibodies may bind peptides having the sequence of SEQ ID NOs: 44 and/or 45, or a variant thereof with up to 80% or greater sequence identity thereto. For instance, OPG antibodies may bind peptides having the sequence of SEQ ID NOs: 46 and/or 47, or a variant thereof with up to 80% or greater sequence identity thereto. For instance, OX40 antibodies may bind peptides having the sequence of SEQ ID NO: 48 or a variant thereof with up to 80% or greater sequence identity thereto. For instance, RANK antibodies may bind peptides having the sequence of SEQ ID NOs: 49 and/or 50, or a variant thereof with up to 80% or greater sequence identity thereto. For instance, RELT (19L) antibodies may bind peptides having the sequence of SEQ ID NOs: 51 and/or 52, or a variant thereof with up to 80% or greater sequence identity thereto. For instance, TNFR1 antibodies may bind peptides having the sequence of SEQ ID NOs: 53 and/or 54, or a variant thereof with up to 80% or greater sequence identity thereto. For instance, TRAIL-R2 (TNFRSF10B) antibodies may bind peptides having the sequence of SEQ ID NOs: 55 and/or 56, or a variant thereof with up to 80% or greater sequence identity thereto. For instance, TRAIL-R1 (TNFRSF10A) antibodies may bind peptides having the sequence of SEQ ID NOs: 57 and/or 58, or a variant thereof with up to 80% or greater sequence identity thereto. For instance, TRAIL-R4 antibodies may bind peptides having the sequence of SEQ ID NOs: 59 and/or 60, or a variant thereof with up to 80% or greater sequence identity thereto. For instance, TRAMP antibodies may bind peptides having the sequence of SEQ ID NOs: 61 and/or 62, or a variant thereof with up to 80% or greater sequence identity thereto. For instance, TROY antibodies may bind peptides having the sequence of SEQ ID NOs: 63 and/or 64, or a variant thereof with up to 80% or greater sequence identity thereto. For instance, XEDAR antibodies may bind peptides having the sequence of SEQ ID NOs: 65 and/or 66, or a variant thereof with up to 80% or greater sequence identity thereto.

### Screening of Libraries for Antagonistic TNFRSF Member Polypeptides

Methods for high throughput screening of polypeptide (e.g., single-chain polypeptide, antibody, or antibody fragment) libraries for molecules capable of binding epitopes (e.g., epitopes presented by peptides having the sequence of any one of SEQ ID NOs: 25-66, or a variant thereof with up to 80% or greater sequence identity thereto) within TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) include, without limitation, display techniques including phage display, bacterial display, yeast display, mammalian display, ribosome display, mRNA display, and cDNA display. The use of phage display to isolate ligands that bind biologically relevant molecules has been reviewed, e.g., in Felici et al. (Biotechnol. Annual Rev. 1:149-183, 1995), Katz (Annual Rev. Biophys. Biomol. Struct. 26:27-45, 1997), and Hoogenboom et al. (Immunotechnology 4:1-20, 1998). Several randomized combinatorial peptide libraries have been constructed to select for polypeptides that bind different targets, e.g., cell surface receptors or DNA (reviewed by Kay (Perspect. Drug Discovery Des. 2, 251-268, 1995), Kay et al., (Mol. Divers. 1:139-140, 1996)). Proteins and multimeric proteins have been successfully phage-displayed as functional molecules (see EP 0349578A, EP 4527839A, EP 0589877A; Chiswell and McCafferty (Trends Biotechnol. 10, 80-84 1992)). In addition, functional antibody fragments (e.g. Fab, single-chain Fv [scFv]) have been expressed (McCafferty et al. (Nature 348: 552-554, 1990), Barbas et al. (Proc. Natl. Acad Sci. USA 88:7978-7982, 1991), Clackson et al. (Nature 352:624-628, 1991)). These references are hereby incorporated by reference in their entirety.

### (i) Phage Display Techniques

As an example, phage display techniques can be used in order to screen libraries of polypeptides, such as single-chain polypeptides, antibodies, and antigen-binding fragments thereof, for functional molecules capable of binding cyclic or polycyclic peptides containing epitopes within TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) that promote receptor antagonism (e.g., peptides having the sequence of any one of SEQ ID NOs: 25-66, or a variant thereof with up to 80% or greater sequence identity thereto). For instance, libraries of polynucleotides encoding single-chain antibody fragments, such as scFv fragments, that contain randomized hypervariable regions can be obtained using established procedures (e.g., solid-phase polynucleotide synthesis or error-prone PCR techniques, see McCullum et al. (Meth. Mol. Biol., 634:103-109, 2010); incorporated herein by reference). These randomized polynucleotides can subsequently be incorporated into a viral genome such that the randomized antibody chains encoded by these genes are expressed on the surface of filamentous phage, e.g., by a covalent bond between the antibody chain and a coat protein (e.g., pill coat protein on the surface of M13 phage). This provides a physical connection between the genotype and phenotype of the antibody chain. In this way, libraries of phage that display diverse antibody chains containing random mutations in hypervariable regions can be screened for the ability of the exterior antibody chains to bind TNFRSF member epitopes (e.g., peptides having the sequence of any one of SEQ ID NOs: 25-66, or a variant thereof with up to 80% or greater sequence identity thereto) that are immobilized to a surface using established procedures. For instance, cyclic peptides such as those represented by SEQ ID NOs: 25-66, or a variant thereof with up to 80% or greater sequence identity thereto, can be physically bound to the surface of a microtiter plate by forming a covalent bond between the peptide and an epitope tag (e.g., biotin) and incubating the peptide in wells of a microtiter plate that have been previously coated with a complementary tag (e.g., avidin) that binds the tag attached to the peptide with high affinity. Suitable epitope tags include, without limitation, maltose-binding protein, glutathione-S-transferase, a poly-histidine tag, a FLAG-tag, a myc-tag, human influenza hemagglutinin (HA) tag, biotin, streptavidin. Peptides containing the epitopes presented by these molecules are capable of being immobilized on surfaces containing such complementary molecules as maltose, glutathione, a nickel-containing complex, an anti-FLAG antibody, an anti-myc antibody, an anti-HA antibody, streptavidin, or biotin, respectively. In this way, phage can be incubated with a surface containing an immobilized TNFRSF member-derived peptide for a time suitable to allow binding of the antibody to the constrained peptide and in the presence of an appropriate buffer system (e.g., one that contains physiological salt concentration, ionic strength, and is maintained at physiological pH by a buffering agent). The surface can then be washed (e.g., with phosphate buffer containing 0.1% Tween-20) so as to remove phage that do not present antibody chains that interact with the TNFRSF member-derived peptides with an affinity greater than a particular threshold value.

The affinity of the polypeptides that remain after this initial panning (i.e., screening) step can be modulated by adjusting the conditions of the washing step (e.g., by including mildly acidic or basic components, or by including other TNFRSF member-derived peptides at a low concentration in order to compete with immobilized peptides for antigen-binding sites). In this way, the population of phage that remains bound to the surfaces of the microtiter plate following the washing step is enriched for phage that bind TNFRSF member-derived peptide epitopes that promote receptor antagonism. The remaining phage can then be amplified by eluting the phage from the surface containing these peptides (e.g., by altering the ambient pH, ionic strength, or temperature) so as to diminish protein-protein interaction strength. The isolated phage can then be amplified, e.g., by infecting bacterial cells, and the resulting phage can optionally be subjected to panning by additional iterations of screening so as to further enrich the population of phage for those harboring higher-affinity anti-TNFRSF member polypeptides. Following these stages, phage that display high-affinity antibodies or antigen-binding fragments thereof can subsequently be isolated and the genomes of these phage can be sequenced in order to identify the polynucleotide and polypeptide sequences of the encoded antibodies. Phage display techniques such as this can be used to generate, e.g., antibody chains, such as scFv fragments, tandem scFv fragments, and other antigen-binding fragments of the disclosure that can be used as antagonists of TNFRSF members. Exemplary phage display protocols for the identification of antibody chains and antigen-binding fragments thereof that bind a particular antigen with high affinity are well-established and are described, e.g., in U.S. Pat. No. 7,846,892, WO 1997/002342, U.S. Pat. No. 8,846,867, and WO 2007/132917; incorporated herein by reference. Similar phage display techniques can be used to generate antibody-like scaffolds (e.g., ¹⁰Fn3 domains) of the disclosure that bind epitopes within TNFRSF members that promote receptor antagonism (e.g., epitopes presented by peptides with the sequence of any one of SEQ ID NOs: 25-66, or a variant thereof with up to 80% or greater sequence identity thereto). Exemplary phage display protocols for the identification of antibody-like scaffold proteins are described, e.g., in WO 2009/086116; incorporated herein by reference).

### (ii) Cell-Based Display Techniques

Other in vitro display techniques that exploit the linkage between genotype and phenotype of a solvent-exposed polypeptide (e.g., single-chain polypeptide, antibody, or antigen-binding fragment thereof) include yeast and bacterial display. Yeast display techniques are established in the art and are often advantageous in that high quantities of antibodies (often up to 30,000) can be presented on the surface of an individual yeast cell (see, e.g., Boder et al., Nat Biotechnol. 15:553, 1997; incorporated herein by reference). The larger size of yeast cells over filamentous phage enables an additional screening strategy, as one can use flow cytometry to both analyze and sort libraries of yeast. For instance, established procedures can be used to generate libraries of bacterial cells or yeast cells that express polypeptides, such as single-chain polypeptides, antibodies, or antibody fragments, containing randomized hypervariable regions (see, e.g., see U.S. Pat. No. 7,749,501 and US 2013/0085072; the teachings of each which are incorporated herein by reference). For instance, large libraries of yeast cells that express polynucleotides encoding naïve scFv fragments can be made using established procedures (de Bruin et al., Nat. Biotechnol. 17:397, 1999; incorporated herein by reference). Yeast cells expressing these polynucleotides can then be incubated with two different fluorescent molecules during the panning steps: one dye that binds conserved residues within the antibody and thus reflects the amount of antibody displayed, and another dye that fluoresces at a different wavelength and binds the antigen and thus indicates the amount of antigen bound. In these cases, it is useful to use a cyclic or polycyclic peptide containing the sequence of any one of SEQ ID NOs: 25-66, or a variant thereof with up to 80% or greater sequence identity thereto that has been conjugated to an epitope tag (e.g., biotin), optionally at a residue that is not expected to interfere with antibody-antigen binding. This enables a fluorescent dye labeled with a complementary tag (e.g., avidin) to localize to the antibody-antigen complex. This results in great flexibility and immediate feedback on the progress of a selection. In contrast to phage display, by normalizing to antibody display levels, antibodies with higher affinities, rather than greater expression levels can easily be selected. In fact, it is possible to distinguish and sort antibodies whose affinities differ by only two-fold (VanAntwerp and Wittrup, Biotechnol. Prog. 16:31, 2000).

(iii) Nucleotide Display Techniques

Display techniques that utilize in vitro translation of randomized polynucleotide libraries also provide a powerful approach to generating anti-TNFRSF member antibodies of the disclosure. For instance, randomized DNA libraries encoding single-chain polypeptides, antibodies, or antigen-binding fragments thereof that contain mutations within designated hypervariable regions can be obtained, e.g., using established PCR-based mutagenesis techniques as described herein. The polynucleotides of these libraries may contain transcription regulating sequences, such as promoters and transcription terminating sequences, and may additionally encode sequences that increase the rate of translation of the resulting mRNA construct (e.g., IRES sequences, 5′ and 3′ UTRs, a poly-adenylation tract, etc.). These polynucleotide libraries can be incubated in an appropriately buffered solution containing RNA polymerase and RNA nucleoside triphosphates (NTPs) in order to enable transcription of the DNA sequences to competent mRNA molecules, which can subsequently be translated by large and small ribosomal subunits, aminoacyl tRNA molecules, and translation initiation and elongation factors present in solution (e.g., using the PURExpress® In Vitro Protein Synthesis Kit, New England Biolabs®). Designed mRNA modifications can enable the antibody product to remain covalently bound to the mRNA template by a chemical bond to puromycin (e.g., see Keefe (Curr. Protoc. Mol. Biol., Chapter 24, Unit 24.5, 2001); incorporated herein by reference). This genotype-phenotype linkage can thus be used to select for antibodies that bind a TNFRSF member-derived peptide (e.g., a peptide that has the sequence of any one of SEQ ID NOs: 25-66, or a variant thereof with up to 80% or greater sequence identity thereto) by incubating mRNA:antibody fusion constructs with a peptide immobilized to a surface and panning in a fashion similar to phage display techniques (see, e.g., WO 2006/072773; incorporated herein by reference).

Optionally, polypeptides (e.g., single-chain polypeptides, antibodies, or antigen-binding fragments thereof, such as anti-CD40 polypeptides that bind CD40) of the disclosure can be generated using a similar technique, except the polypeptide product may be bound non-covalently to the ribosome-mRNA complex rather than covalently via a puromycin linker. This platform, known as ribosome display, has been described, e.g., in U.S. Pat. No. 7,074,557; incorporated herein by reference. Alternatively, antibodies can be generated using cDNA display, a technique analogous to mRNA display with the exception that cDNA, rather than mRNA, is covalently bound to an antibody product via a puromycin linker. cDNA display techniques offer the advantage of being able to perform panning steps under increasingly stringent conditions, e.g., under conditions in which the salt concentration, ionic strength, pH, and/or temperature of the environment is adjusted in order to screen for antibodies with particularly high affinity for TNFRSF member-derived peptides. This is due to the higher natural stability of double-stranded cDNA over single-stranded mRNA. cDNA display screening techniques are described, e.g., in Ueno et al., Methods Mol. Biol. 805:113-135, 2012; incorporated herein by reference.

In addition to generating anti-TNFRSF member polypeptides of the disclosure, in vitro display techniques (e.g., those described herein and those known in the art) also provide methods for improving the affinity of an anti-TNFRSF member polypeptide of the disclosure. For instance, rather than screening libraries of single-chain polypeptides, antibodies, and fragments thereof containing completely randomized hypervariable regions, one can screen narrower libraries of single-chain polypeptides, antibodies, and antigen-binding fragments thereof that feature targeted mutations at specific sites within hypervariable regions. This can be accomplished, e.g., by assembling libraries of polynucleotides encoding antibodies or antigen-binding fragments thereof that encode random mutations only at particular sites within hypervariable regions. These polynucleotides can then be expressed in, e.g., filamentous phage, bacterial cells, yeast cells, mammalian cells, or in vitro using, e.g., ribosome display, mRNA display, or cDNA display techniques in order to screen for polypeptides, such as single-chain polypeptides, antibodies, or antigen-binding fragments thereof that specifically bind TNFRSF member epitopes (e.g., peptides containing the sequence of any one of SEQ ID NOs: 25-66, or a variant thereof with up to 80% or greater sequence identity thereto) with improved binding affinity. Yeast display is particularly well-suited for affinity maturation and has been used previously to improve the affinity of a single-chain antibody to a K_{d} of 48 fM (Boder et al., Proc Natl Aced Sci USA 97:10701, 2000).

Additional in vitro techniques that can be used for the generation and affinity maturation of antagonistic TNFRSF member antibodies of the disclosure include the screening of combinatorial libraries of polypeptides, such as single-chain polypeptides, antibodies, or antigen-binding fragments thereof for functional molecules capable of specifically binding TNFRSF member-derived peptides (e.g., a peptide having the amino acid sequence of any one of SEQ ID NOs: 25-66, or a variant thereof with up to 80% or greater sequence identity thereto). Combinatorial polypeptide libraries, such as antibody or antibody fragment libraries, can be obtained, e.g., by expression of polynucleotides encoding randomized hypervariable regions of an antibody or antigen-binding fragment thereof in a eukaryotic or prokaryotic cell. This can be achieved, e.g., using gene expression techniques described herein or known in the art. Heterogeneous mixtures of antibodies can be purified, e.g., by Protein A or Protein G selection, sizing column chromatography), centrifugation, differential solubility, and/or by any other standard technique for the purification of proteins. Libraries of combinatorial libraries thus obtained can be screened, e.g., by incubating a heterogeneous mixture of these antibodies with a peptide derived from a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) that has been immobilized to a surface (e.g., a peptide having the amino acid sequence of any one of SEQ ID NOs: 25-66, or a variant thereof with up to 80% or greater sequence identity thereto, immobilized to the surface of a solid-phase resin or a well of a microtiter plate) for a period of time sufficient to allow antibody-antigen binding. Non-binding antibodies or fragments thereof can be removed by washing the surface with an appropriate buffer (e.g., a solution buffered at physiological pH (approximately 7.4) and containing physiological salt concentrations and ionic strength, and optionally containing a detergent, such as TWEEN-20). Antibodies that remain bound can subsequently be detected, e.g., using an ELISA-based detection protocol (see, e.g., U.S. Pat. No. 4,661,445; incorporated herein by reference).

Additional techniques for screening combinatorial libraries of polypeptides for those that specifically bind TNFRSF member-derived peptides (e.g., a peptide containing the amino acid sequence of any one of SEQ ID NOs: 25-66, or a variant thereof with up to 80% or greater sequence identity thereto) include the screening of one-bead-one-compound libraries of single-chain polypeptides or antibody fragments. Single-chain polypeptides and antibody fragments can be chemically synthesized on a solid bead (e.g., using established split-and-pool solid-phase peptide synthesis protocols) composed of a hydrophilic, water-swellable material such that each bead displays a single antibody fragment. Heterogeneous bead mixtures can then be incubated with a TNFRSF member-derived peptide that is optionally labeled with a detectable moiety (e.g., a fluorescent dye) or that is conjugated to an epitope tag (e.g., biotin, avidin, FLAG tag, HA tag) that can later be detected by treatment with a complementary tag (e.g., avidin, biotin, anti-FLAG antibody, anti-HA antibody, respectively). Beads containing antibody fragments that specifically bind a TNFRSF member-derived peptide (e.g., a peptide containing the amino acid sequence of any one of SEQ ID NOs: 25-66, or a variant thereof with up to 80% or greater sequence identity thereto) can be identified by analyzing the fluorescent properties of the beads following incubation with a fluorescently-labeled antigen or complementary tag (e.g., by confocal fluorescent microscopy or by fluorescence-activated bead sorting; see, e.g., Muller et al. (J. Biol. Chem., 16500-16505, 1996); incorporated herein by reference). Beads containing antibody fragments that specifically bind TNFRSF member-derived peptides can thus be separated from those that do not contain high-affinity antibody fragments. The sequence of an antibody fragment that specifically binds a TNFRSF member-derived peptide can be determined by techniques known in the art, including, e.g., Edman degradation, tandem mass spectrometry, matrix-assisted laser-desorption time-of-flight mass spectrometry (MALDI-TOF MS), nuclear magnetic resonance (NMR), and 2D gel electrophoresis, among others (see, e.g., WO 2004/062553; incorporated herein by reference).

### Immunization of a Non-Human Mammal

Another strategy that can be used to produce antagonistic TNFRSF member antibodies or antibody fragments of the disclosure (e.g., single-chain polypeptide, antibody, or antigen-binding fragment thereof, such as anti-CD40 polypeptides that bind CD40) includes immunizing a non-human mammal. Examples of non-human mammals that can be immunized in order to produce antagonistic TNFRSF member antibodies and fragments thereof of the disclosure include rabbits, mice, rats, goats, guinea pigs, hamsters, horses, and sheep, as well as non-human primates. For instance, established procedures for immunizing primates are known in the art (see, e.g., WO 1986/6004782; incorporated herein by reference). Immunization represents a robust method of producing monoclonal antibodies by exploiting the antigen specificity of B lymphocytes. For example, monoclonal antibodies can be prepared by the Kohler-Millstein procedure (described, e.g., in EP 0110716; incorporated herein by reference), wherein spleen cells from a non-human animal (e.g., a primate) immunized with a peptide that presents a TNFRSF member-derived antigen that promotes receptor antagonism (e.g., a peptide containing the amino acid sequence of any one or more of SEQ ID NOs: 25-66 or 67-154 or a variant thereof with up to 80% or greater sequence identity thereto). A clonally-expanded B lymphocyte produced by immunization can be isolated from the serum of the animal and subsequently fused with a myeloma cell in order to form a hybridoma. Hybridomas are particularly useful agents for antibody production, as these immortalized cells can provide a lasting supply of an antigen-specific antibody. Antibodies from such hybridomas can subsequently be isolated using techniques known in the art, e.g., by purifying the antibodies from the cell culture medium by affinity chromatography, using reagents such as Protein A or Protein G.

Furthermore, anti-CD40 polypeptides (e.g., single-chain polypeptides, antibodies, and antigen-binding fragments) of the disclosure may be produced by immunizing a non-human mammal with a peptide including at least five continuous or discontinuous amino acid residues of amino acids 104-172 of SEQ ID NO: 4 (e.g., at least five continuous or discontinuous amino acid residues of amino acids 114-142 of SEQ ID NO: 4 and/or at least five continuous or discontinuous amino acid residues of amino acids 123-172 of SEQ ID NO: 4, or at least five continuous or discontinuous amino acid residues of amino acids 124-132 of SEQ ID NO: 4 and/or at least five continuous or discontinuous amino acid residues of amino acids 133-162 of SEQ ID NO: 4). In some embodiments, the antibody or antigen-binding fragment thereof is produced by immunizing a non-human mammal with a peptide comprising five or more of amino acids 124-132 of SEQ ID NO: 4. In some embodiments, the antibody or antigen-binding fragment thereof is produced by immunizing a non-human mammal with a peptide comprising five or more of amino acids 143-152 of SEQ ID NO: 4.

Furthermore, anti-4-1 BB polypeptides (e.g., single-chain polypeptides, antibodies, and antigen-binding fragments) of the disclosure may be produced by immunizing a non-human mammal with a peptide including at least five continuous or discontinuous amino acid residues of amino acids 81-149 of SEQ ID NO: 1 (e.g., at least five continuous or discontinuous amino acid residues of amino acids 91-139 of SEQ ID NO: 1, or at least five continuous or discontinuous amino acid residues of amino acids 101-129 of SEQ ID NO: 1). In some embodiments, the antibody or antigen-binding fragment thereof is produced by immunizing a non-human mammal with a peptide comprising five or more of amino acids 101-129 of SEQ ID NO: 1.

Furthermore, anti-CD27 polypeptides (e.g., single-chain polypeptides, antibodies, and antigen-binding fragments) of the disclosure may be produced by immunizing a non-human mammal with a peptide including at least five continuous or discontinuous amino acid residues of amino acids 21-92 of SEQ ID NO: 2 (e.g., at least five continuous or discontinuous amino acid residues of amino acids 31-57 and/or positions 51-82 of SEQ ID NO: 2, or at least five continuous or discontinuous amino acid residues of amino acids 41-47 and/or positions 61-72 of SEQ ID NO: 2). In some embodiments, the antibody or antigen-binding fragment thereof is produced by immunizing a non-human mammal with a peptide comprising five or more of amino acids 41-47 of SEQ ID NO: 2. In some embodiments, the antibody or antigen-binding fragment thereof is produced by immunizing a non-human mammal with a peptide comprising five or more of amino acids 61-72 of SEQ ID NO: 2.

Furthermore, anti-CD30 polypeptides (e.g., single-chain polypeptides, antibodies, and antigen-binding fragments) of the disclosure may be produced by immunizing a non-human mammal with a peptide including at least five continuous or discontinuous amino acid residues of amino acids 109-177 of SEQ ID NO: 3 (e.g., at least five continuous or discontinuous amino acid residues of amino acids 119-150 and/or positions 139-167 of SEQ ID NO: 3, or at least five continuous or discontinuous amino acid residues of amino acids 129-140 and/or positions 149-157 of SEQ ID NO: 3). In some embodiments, the antibody or antigen-binding fragment thereof is produced by immunizing a non-human mammal with a peptide comprising five or more of amino acids 129-140 of SEQ ID NO: 3. In some embodiments, the antibody or antigen-binding fragment thereof is produced by immunizing a non-human mammal with a peptide comprising five or more of amino acids 149-157 of SEQ ID NO: 3.

Furthermore, anti-DR6 polypeptides (e.g., single-chain polypeptides, antibodies, and antigen-binding fragments) of the disclosure may be produced by immunizing a non-human mammal with a peptide including at least five continuous or discontinuous amino acid residues of amino acids 121-197 of SEQ ID NO: 5 (e.g., at least five continuous or discontinuous amino acid residues of amino acids 131-166 and/or positions 159-187 of SEQ ID NO: 5, or at least five continuous or discontinuous amino acid residues of amino acids 141-156 and/or positions 169-177 of SEQ ID NO: 5). In some embodiments, the antibody or antigen-binding fragment thereof is produced by immunizing a non-human mammal with a peptide comprising five or more of amino acids 141-156 of SEQ ID NO: 5. In some embodiments, the antibody or antigen-binding fragment thereof is produced by immunizing a non-human mammal with a peptide comprising five or more of amino acids 169-177 of SEQ ID NO: 5.

Furthermore, anti-EDAR polypeptides (e.g., single-chain polypeptides, antibodies, and antigen-binding fragments) of the disclosure may be produced by immunizing a non-human mammal with a peptide including at least five continuous or discontinuous amino acid residues of amino acids 91-142 of SEQ ID NO: 6 (e.g., at least five continuous or discontinuous amino acid residues of amino acids 81-112 and/or positions 101-132 of SEQ ID NO: 6, or at least five continuous or discontinuous amino acid residues of amino acids 91-102 and/or positions 111-122 of SEQ ID NO: 6). In some embodiments, the antibody or antigen-binding fragment thereof is produced by immunizing a non-human mammal with a peptide comprising five or more of amino acids 91-102 of SEQ ID NO: 6. In some embodiments, the antibody or antigen-binding fragment thereof is produced by immunizing a non-human mammal with a peptide comprising five or more of amino acids 111-122 of SEQ ID NO: 6.

Furthermore, anti-Fas polypeptides (e.g., single-chain polypeptides, antibodies, and antigen-binding fragments) of the disclosure may be produced by immunizing a non-human mammal with a peptide including at least five continuous or discontinuous amino acid residues of amino acids 128-193 of SEQ ID NO: 7 (e.g., at least five continuous or discontinuous amino acid residues of amino acids 138-166 and/or positions 155-183 of SEQ ID NO: 7, or at least five continuous or discontinuous amino acid residues of amino acids 148-156 and/or positions 165-173 of SEQ ID NO: 7). In some embodiments, the antibody or antigen-binding fragment thereof is produced by immunizing a non-human mammal with a peptide comprising five or more of amino acids 148-156 of SEQ ID NO: 7. In some embodiments, the antibody or antigen-binding fragment thereof is produced by immunizing a non-human mammal with a peptide comprising five or more of amino acids 165-173 of SEQ ID NO: 7.

Furthermore, anti-GITR polypeptides (e.g., single-chain polypeptides, antibodies, and antigen-binding fragments) of the disclosure may be produced by immunizing a non-human mammal with a peptide including at least five continuous or discontinuous amino acid residues of amino acids 72-141 of SEQ ID NO: 8 (e.g., at least five continuous or discontinuous amino acid residues of amino acids 82-111 and/or positions 102-131 of SEQ ID NO: 8, or at least five continuous or discontinuous amino acid residues of amino acids 92-101 and/or positions 112-121 of SEQ ID NO: 8). In some embodiments, the antibody or antigen-binding fragment thereof is produced by immunizing a non-human mammal with a peptide comprising five or more of amino acids 92-101 of SEQ ID NO: 8. In some embodiments, the antibody or antigen-binding fragment thereof is produced by immunizing a non-human mammal with a peptide comprising five or more of amino acids 112-121 of SEQ ID NO: 8.

Furthermore, anti-HVEM polypeptides (e.g., single-chain polypeptides, antibodies, and antigen-binding fragments) of the disclosure may be produced by immunizing a non-human mammal with a peptide including at least five continuous or discontinuous amino acid residues of amino acids 122-190 of SEQ ID NO: 9 (e.g., at least five continuous or discontinuous amino acid residues of amino acids 132-162 and/or positions 152-180 of SEQ ID NO: 9, or at least five continuous or discontinuous amino acid residues of amino acids 142-152 and/or positions 162-170 of SEQ ID NO: 9). In some embodiments, the antibody or antigen-binding fragment thereof is produced by immunizing a non-human mammal with a peptide comprising five or more of amino acids 142-152 of SEQ ID NO: 9. In some embodiments, the antibody or antigen-binding fragment thereof is produced by immunizing a non-human mammal with a peptide comprising five or more of amino acids 162-170 of SEQ ID NO: 9.

Furthermore, anti-LT beta receptor polypeptides (e.g., single-chain polypeptides, antibodies, and antigen-binding fragments) of the disclosure may be produced by immunizing a non-human mammal with a peptide including at least five continuous or discontinuous amino acid residues of amino acids 127-196 of SEQ ID NO: 10 (e.g., at least five continuous or discontinuous amino acid residues of amino acids 137-165 and/or positions 157-186 of SEQ ID NO: 10, or at least five continuous or discontinuous amino acid residues of amino acids 147-155 and/or positions 167-176 of SEQ ID NO: 10). In some embodiments, the antibody or antigen-binding fragment thereof is produced by immunizing a non-human mammal with a peptide comprising five or more of amino acids 147-155 of SEQ ID NO: 10. In some embodiments, the antibody or antigen-binding fragment thereof is produced by immunizing a non-human mammal with a peptide comprising five or more of amino acids 167-176 of SEQ ID NO: 10.

Furthermore, anti-NGFR polypeptides (e.g., single-chain polypeptides, antibodies, and antigen-binding fragments) of the disclosure may be produced by immunizing a non-human mammal with a peptide including at least five continuous or discontinuous amino acid residues of amino acids 108-176 of SEQ ID NO: 11 (e.g., at least five continuous or discontinuous amino acid residues of amino acids 118-145 and/or positions 136-166 of SEQ ID NO: 11, or at least five continuous or discontinuous amino acid residues of amino acids 128-135 and/or positions 146-156 of SEQ ID NO: 11). In some embodiments, the antibody or antigen-binding fragment thereof is produced by immunizing a non-human mammal with a peptide comprising five or more of amino acids 128-135 of SEQ ID NO: 11. In some embodiments, the antibody or antigen-binding fragment thereof is produced by immunizing a non-human mammal with a peptide comprising five or more of amino acids 146-156 of SEQ ID NO: 11.

Furthermore, anti-OPG polypeptides (e.g., single-chain polypeptides, antibodies, and antigen-binding fragments) of the disclosure may be produced by immunizing a non-human mammal with a peptide including at least five continuous or discontinuous amino acid residues of amino acids 103--171 of SEQ ID NO: 12 (e.g., at least five continuous or discontinuous amino acid residues of amino acids 113-141 and/or positions 132-161 of SEQ ID NO: 12, or at least five continuous or discontinuous amino acid residues of amino acids 123-131 and/or positions 142-151 of SEQ ID NO: 12). In some embodiments, the antibody or antigen-binding fragment thereof is produced by immunizing a non-human mammal with a peptide comprising five or more of amino acids 123-131 of SEQ ID NO: 12. In some embodiments, the antibody or antigen-binding fragment thereof is produced by immunizing a non-human mammal with a peptide comprising five or more of amino acids 142-151 of SEQ ID NO: 12.

Furthermore, anti-OX40 polypeptides (e.g., single-chain polypeptides, antibodies, and antigen-binding fragments) of the disclosure may be produced by immunizing a non-human mammal with a peptide including at least five continuous or discontinuous amino acid residues of amino acids 98-154 of SEQ ID NO: 13 (e.g., at least five continuous or discontinuous amino acid residues of amino acids 108-144 of SEQ ID NO: 13, or at least five continuous or discontinuous amino acid residues of amino acids 118-134 of SEQ ID NO: 13). In some embodiments, the antibody or antigen-binding fragment thereof is produced by immunizing a non-human mammal with a peptide comprising five or more of amino acids 118-134 of SEQ ID NO: 13.

Furthermore, anti-RANK polypeptides (e.g., single-chain polypeptides, antibodies, and antigen-binding fragments of the disclosure may be produced by immunizing a non-human mammal with a peptide including at least five continuous or discontinuous amino acid residues of amino acids 112-180 of SEQ ID NO: 14 (e.g., at least five continuous or discontinuous amino acid residues of amino acids 122-149 and/or positions 141-170 of SEQ ID NO: 14, or at least five continuous or discontinuous amino acid residues of amino acids 132-139 and/or positions 151-160 of SEQ ID NO: 14). In some embodiments, the antibody or antigen-binding fragment thereof is produced by immunizing a non-human mammal with a peptide comprising five or more of amino acids 132-139 of SEQ ID NO: 14. In some embodiments, the antibody or antigen-binding fragment thereof is produced by immunizing a non-human mammal with a peptide comprising five or more of amino acids 151-160 of SEQ ID NO: 14.

Furthermore, anti-RELT (19L) polypeptides (e.g., single-chain polypeptides, antibodies, and antigen-binding fragments) of the disclosure may be produced by immunizing a non-human mammal with a peptide including at least five continuous or discontinuous amino acid residues of amino acids 50-118 of SEQ ID NO: 15 (e.g., at least five continuous or discontinuous amino acid residues of amino acids 60-87 and/or positions 80-108 of SEQ ID NO: 15, or at least five continuous or discontinuous amino acid residues of amino acids 70-77 and/or positions 90-98 of SEQ ID NO: 15). In some embodiments, the antibody or antigen-binding fragment thereof is produced by immunizing a non-human mammal with a peptide comprising five or more of amino acids 70-77 of SEQ ID NO 15. In some embodiments, the antibody or antigen-binding fragment thereof is produced by immunizing a non-human mammal with a peptide comprising five or more of amino acids 90-98 of SEQ ID NO: 15.

Furthermore, anti-TNFR1 polypeptides (e.g., single-chain polypeptides, antibodies, and antigen-binding fragments) of the disclosure may be produced by immunizing a non-human mammal with a peptide including at least five continuous or discontinuous amino acid residues of amino acids 84-153 of SEQ ID NO: 16 (e.g., at least five continuous or discontinuous amino acid residues of amino acids 94-122 and/or positions 114-143 of SEQ ID NO: 16, or at least five continuous or discontinuous amino acid residues of amino acids 104-112 and/or positions 124-133 of SEQ ID NO: 16). In some embodiments, the antibody or antigen-binding fragment thereof is produced by immunizing a non-human mammal with a peptide comprising five or more of amino acids 104-112 of SEQ ID NO: 16. In some embodiments, the antibody or antigen-binding fragment thereof is produced by immunizing a non-human mammal with a peptide comprising five or more of amino acids 124-133 of SEQ ID NO: 16.

Furthermore, anti-TRAIL-R2 (TNFRSF10B) polypeptides (e.g., single-chain polypeptides, antibodies, and antigen-binding fragments) of the disclosure may be produced by immunizing a non-human mammal with a peptide including at least five continuous or discontinuous amino acid residues of amino acids 135-206 of SEQ ID NO: 17 (e.g., at least five continuous or discontinuous amino acid residues of amino acids 145-175 and/or positions 168-196 of SEQ ID NO: 17, or at least five continuous or discontinuous amino acid residues of amino acids 155-165 and/or positions 178-186 of SEQ ID NO: 17). In some embodiments, the antibody or antigen-binding fragment thereof is produced by immunizing a non-human mammal with a peptide comprising five or more of amino acids 155-165 of SEQ ID NO: 17. In some embodiments, the antibody or antigen-binding fragment thereof is produced by immunizing a non-human mammal with a peptide comprising five or more of amino acids 178-186 of SEQ ID NO: 17.

Furthermore, anti-TRAIL-R1 (TNFRSF10A) polypeptides (e.g., single-chain polypeptides, antibodies, and antigen-binding fragments) of the disclosure may be produced by immunizing a non-human mammal with a peptide including at least five continuous or discontinuous amino acid residues of amino acids 149-216 of SEQ ID NO: 18 (e.g., at least five continuous or discontinuous amino acid residues of amino acids 159-187 and/or positions 178-206 of SEQ ID NO: 18, or at least five continuous or discontinuous amino acid residues of amino acids 169-177 and/or positions 188-196 of SEQ ID NO: 18). In some embodiments, the antibody or antigen-binding fragment thereof is produced by immunizing a non-human mammal with a peptide comprising five or more of amino acids 169-177 of SEQ ID NO: 18. In some embodiments, the antibody or antigen-binding fragment thereof is produced by immunizing a non-human mammal with a peptide comprising five or more of amino acids 188-196 of SEQ ID NO: 18.

Furthermore, anti-TRAIL-R4 polypeptides (e.g., single-chain polypeptides, antibodies, and antigen-binding fragments) of the disclosure may be produced by immunizing a non-human mammal with a peptide including at least five continuous or discontinuous amino acid residues of amino acids 141-210 of SEQ ID NO: 19 (e.g., at least five continuous or discontinuous amino acid residues of amino acids 151-179 and/or positions 171-200 of SEQ ID NO: 19, or at least five continuous or discontinuous amino acid residues of amino acids 161-169 and/or positions 181-190 of SEQ ID NO: 19). In some embodiments, the antibody or antigen-binding fragment thereof is produced by immunizing a non-human mammal with a peptide comprising five or more of amino acids 161-169 of SEQ ID NO: 19. In some embodiments, the antibody or antigen-binding fragment thereof is produced by immunizing a non-human mammal with a peptide comprising five or more of amino acids 181-190 of SEQ ID NO: 19.

Furthermore, anti-TRAMP polypeptides (e.g., single-chain polypeptides, antibodies, and antigen-binding fragments) of the disclosure may be produced by immunizing a non-human mammal with a peptide including at least five continuous or discontinuous amino acid residues of amino acids 122-191 of SEQ ID NO: 20 (e.g., at least five continuous or discontinuous amino acid residues of amino acids 132-160 and/or positions 152-181 of SEQ ID NO: 20, or at least five continuous or discontinuous amino acid residues of amino acids 142-150 and/or positions 162-171 of SEQ ID NO: 20). In some embodiments, the antibody or antigen-binding fragment thereof is produced by immunizing a non-human mammal with a peptide comprising five or more of amino acids 142-150 of SEQ ID NO: 20. In some embodiments, the antibody or antigen-binding fragment thereof is produced by immunizing a non-human mammal with a peptide comprising five or more of amino acids 162-171 of SEQ ID NO: 20.

Furthermore, anti-TROY polypeptides (e.g., single-chain polypeptides, antibodies, and antigen-binding fragments) of the disclosure may be produced by immunizing a non-human mammal with a peptide including at least five continuous or discontinuous amino acid residues of amino acids 31-102 of SEQ ID NO: 21 (e.g., at least five continuous or discontinuous amino acid residues of amino acids 41-69 and/or positions 62-92 of SEQ ID NO: 21, or at least five continuous or discontinuous amino acid residues of amino acids 51-59 and/or positions 72-82 of SEQ ID NO: 21). In some embodiments, the antibody or antigen-binding fragment thereof is produced by immunizing a non-human mammal with a peptide comprising five or more of amino acids 51-59 of SEQ ID NO: 21. In some embodiments, the antibody or antigen-binding fragment thereof is produced by immunizing a non-human mammal with a peptide comprising five or more of amino acids 72-82 of SEQ ID NO: 21.

Furthermore, anti-XEDAR polypeptides (e.g., single-chain polypeptides, antibodies, and antigen-binding fragments) of the disclosure may be produced by immunizing a non-human mammal with a peptide including at least five continuous or discontinuous amino acid residues of amino acids 1-69 of SEQ ID NO: 22 (e.g., at least five continuous or discontinuous amino acid residues of amino acids 10-37 and/or positions 32-59 of SEQ ID NO: 22, or at least five continuous or discontinuous amino acid residues of amino acids 20-27 and/or positions 42-49 of SEQ ID NO: 22). In some embodiments, the antibody or antigen-binding fragment thereof is produced by immunizing a non-human mammal with a peptide comprising five or more of amino acids 20-27 of SEQ ID NO: 22 In some embodiments, the antibody or antigen-binding fragment thereof is produced by immunizing a non-human mammal with a peptide comprising five or more of amino acids 42-49 of SEQ ID NO: 22.

### Exemplary TNFRSF Member Antagonist Antibodies or Antigen-Binding Fragments

The disclosure features the production of antagonistic antibodies or antigen-binding fragments thereof of the disclosure, for example, by expressing a polynucleotide engineered to encode the antagonistic antibodies or antigen-binding fragments thereof. For instance, nucleic acids encoding such antagonistic CD40 antibodies or antigen-binding fragments thereof of the disclosure may bind an epitope.

For instance, a nucleic acid molecule encoding an antagonistic CD40 antibody or antigen-binding fragments thereof that binds an epitope within amino acids 104-172 of SEQ ID NO: 4 can be delivered to and expressed in a cell to produce the antibody or antigen-binding fragment thereof. For example, the antagonistic CD40 antibodies or antigen-binding fragments thereof encoded by the nucleic acid molecule may bind an epitope of, within, or including one or more of amino acids 104-152 (e.g., amino acids 124-132, SCSPGFGVK, SEQ ID NO: 25) of the CD40 amino acid sequence (SEQ ID NO:4) and/or amino acids 123-172 (e.g., amino acids 143-152, CEPCPVGFFS, SEQ ID NO: 26) of the CD40 amino acid sequence (SEQ ID NO: 4). In an embodiment, the epitope bound by the antibody or antigen-binding fragment thereof is or includes one or more of amino acids 124-132 of SEQ ID NO: 4. In an embodiment, the epitope bound by the antibody or antigen-binding fragment thereof is or includes one or more of amino acids 143-152 of SEQ ID NO: 4. The antagonistic CD40 antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-CD40 polypeptides (e.g., single-chain polypeptides, antibodies, and antigen-binding fragments) encoded by the nucleic acid molecule may also specifically bind an epitope within human CD40 that includes at least five continuous or discontinuous amino acid residues of amino acids 104-172 of SEQ ID NO: 4 (e.g., at least five continuous or discontinuous amino acid residues of amino acids 114-142 of SEQ ID NO: 4 and/or at least five continuous or discontinuous amino acid residues of amino acids 133-162 of SEQ ID NO: 4, or at least five continuous or discontinuous amino acid residues of amino acids 124-132 of SEQ ID NO: 4 and/or at least five continuous or discontinuous amino acid residues of amino acids 143-152 of SEQ ID NO: 4). Furthermore, anti-CD40 polypeptides (e.g., single-chain polypeptides, antibodies, and antigen-binding fragments) encoded by the nucleic acid molecule may also specifically bind an epitope within human CD40 that includes at least five continuous or discontinuous amino acid residues of amino acids 104-172 of SEQ ID NO: 4 (e.g., at least five continuous or discontinuous amino acid residues of amino acids 114-142 of SEQ ID NO: 4 and/or at least five continuous or discontinuous amino acid residues of amino acids 133-162 of SEQ ID NO: 4, or at least five continuous or discontinuous amino acid residues of amino acids 124-132 of SEQ ID NO: 4 and/or at least five continuous or discontinuous amino acid residues of amino acids 143-152 of SEQ ID NO: 4), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the CD40 antibodies or antigen-binding fragments thereof can be engineered to target cells that express CD40, such as, e.g., T cells, B cells, platelets, macrophages, dendritic cells, epithelial cells, endothelial cells, and mesenchymal cells.

A nucleic acid molecule encoding an antagonistic 4-1 BB antibody or antigen-binding fragments thereof that binds an epitope within amino acids 81-149 of SEQ ID NO: 1 can be delivered to and expressed in a cell to produce the antibody or antigen-binding fragment thereof. For example, the antagonistic 4-1 BB antibodies or antigen-binding fragments thereof encoded by the nucleic acid molecule may bind an epitope of, within, or including one or more of amino acids 81-149 (e.g., amino acids 101-129, MCEQDCKQGQELTKKGCKDCCFGTFNDQK, SEQ ID NO: 27) of the 4-1 BB amino acid sequence (SEQ ID NO: 1). In an embodiment, the epitope bound by the antibody or antigen-binding fragment thereof is or includes one or more of amino acids 101-129 of SEQ ID NO: 1. The antagonistic 4-1 BB antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to this sequence and an epitope(s) that contains conservative amino acid substitutions relative to this sequence. The anti-4-1 BB polypeptides (e.g., single-chain polypeptides, antibodies, and antigen-binding fragments) encoded by the nucleic acid molecule may also specifically bind an epitope within human 4-1 BB that includes at least five continuous or discontinuous amino acid residues of amino acids 81-149 of SEQ ID NO: 1 (e.g., at least five continuous or discontinuous amino acid residues of amino acids 91-139 of SEQ ID NO: 1, or at least five continuous or discontinuous amino acid residues of amino acids 101-129 of SEQ ID NO: 1). Furthermore, anti-4-1BB polypeptides (e.g., single-chain polypeptides, antibodies, and antigen-binding fragments) encoded by the nucleic acid molecule may also specifically bind an epitope within human 4-1 BB that includes at least five continuous or discontinuous amino acid residues of amino acids 81-149 of SEQ ID NO: 1 (e.g., at least five continuous or discontinuous amino acid residues of amino acids 91-139 of SEQ ID NO: 1, or at least five continuous or discontinuous amino acid residues of amino acids 101-129 of SEQ ID NO: 1), as well as an epitope(s) that exhibits at least 80% sequence identity (e.g., 80%, 85%, 90%, 95%, 97%, 99%, or 100% sequence identity) to this sequence and an epitope(s) that contains conservative amino acid substitutions relative to this sequence. A nucleic acid molecule encoding the 4-1 BB antibodies or antigen-binding fragments thereof can be engineered to target cells that express 4-1 BB, such as, e.g., CD4+ and CD8+ cytotoxic T cells, and NK cells.

A nucleic acid molecule encoding an antagonistic CD27 antibody or antigen-binding fragments thereof that binds an epitope within amino acids 21-92 of SEQ ID NO: 2 can be delivered to and expressed in a cell to produce the antibody or antigen-binding fragment thereof. For example, the antagonistic CD27 antibodies or antigen-binding fragments thereof encoded by the nucleic acid molecule may bind an epitope of, within, or including one or more of amino acids 21-67 (e.g., amino acids 41-47, QMCEPGT, SEQ ID NO: 28) of the CD27 amino acid sequence (SEQ ID NO: 2) and/or amino acids 41-92 (e.g., amino acids 61-72, QCDPCIPGVSFS, SEQ ID NO: 29) of the CD27 amino acid sequence (SEQ ID NO: 2). In an embodiment, the epitope bound by the antibody or antigen-binding fragment thereof is or includes one or more of amino acids 41-47 of SEQ ID NO: 2. In an embodiment, the epitope bound by the antibody or antigen-binding fragment thereof is or includes one or more of amino acids 61-72 of SEQ ID NO: 2. The antagonistic CD27 antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-CD27 polypeptides (e.g., single-chain polypeptides, antibodies, and antigen-binding fragments) encoded by the nucleic acid molecule may also specifically bind an epitope within human CD27 that includes at least five continuous or discontinuous amino acid residues of amino acids 21-92 of SEQ ID NO: 2 (e.g., at least five continuous or discontinuous amino acid residues of amino acids 31-57 and/or positions 51-82 of SEQ ID NO: 2, or at least five continuous or discontinuous amino acid residues of amino acids 41-47 and/or positions 61-72 of SEQ ID NO: 2. Furthermore, anti-CD27 polypeptides (e.g., single-chain polypeptides, antibodies, and antigen-binding fragments) encoded by the nucleic acid molecule may also specifically bind an epitope within human CD27 that includes at least five continuous or discontinuous amino acid residues of amino acids 21-92 of SEQ ID NO: 2 (e.g., at least five continuous or discontinuous amino acid residues of amino acids 31-57 and/or positions 51-82 of SEQ ID NO: 2, or at least five continuous or discontinuous amino acid residues of amino acids 41-47 and/or positions 61-72 of SEQ ID NO: 2), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the CD27 antibodies or antigen-binding fragments thereof can be engineered to target cells that express CD27, such as, e.g., CD4+ and CD8+ cytotoxic T cells.

A nucleic acid molecule encoding an antagonistic CD30 antibody or antigen-binding fragments thereof that binds an epitope within amino acids 109-177 of SEQ ID NO: 3 can be delivered to and expressed in a cell to produce the antibody or antigen-binding fragment thereof. For example, the antagonistic CD30 antibodies or antigen-binding fragments thereof encoded by the nucleic acid molecule may bind an epitope of, within, or including one or more of amino acids 109-160 (e.g., amino acids 129-140, SVCPAGMIVKFP, SEQ ID NO: 30) of the CD30 amino acid sequence (SEQ ID NO:3) and/or amino acids 129-177 (e.g., amino acids 149-157, CEPASPGVS, SEQ ID NO: 31) of the CD30 amino acid sequence (SEQ ID NO: 3). In an embodiment, the epitope bound by the antibody or antigen-binding fragment thereof is or includes one or more of amino acids 129-140 of SEQ ID NO: 3. In an embodiment, the epitope bound by the antibody or antigen-binding fragment thereof is or includes one or more of amino acids 149-157 of SEQ ID NO: 3. The antagonistic CD30 antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-CD30 polypeptides (e.g., single-chain polypeptides, antibodies, and antigen-binding fragments) encoded by the nucleic acid molecule may also specifically bind an epitope within human CD30 that includes at least five continuous or discontinuous amino acid residues of amino acids 109-177 of SEQ ID NO: 3 (e.g., at least five continuous or discontinuous amino acid residues of amino acids 119-150 and/or positions 139-167 of SEQ ID NO: 3, or at least five continuous or discontinuous amino acid residues of amino acids 129-140 and/or positions 149-157 of SEQ ID NO: 3). Furthermore, anti-CD30 polypeptides (e.g., single-chain polypeptides, antibodies, and antigen-binding fragments) encoded by the nucleic acid molecule may also specifically bind an epitope within human CD30 that includes at least five continuous or discontinuous amino acid residues of amino acids 109-177 of SEQ ID NO: 3 (e.g., at least five continuous or discontinuous amino acid residues of amino acids 119-150 and/or positions 139-167 of SEQ ID NO: 3, or at least five continuous or discontinuous amino acid residues of amino acids 129-140 and/or positions 149-157 of SEQ ID NO: 3), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the CD30 antibodies or antigen-binding fragments thereof can be engineered to target cells that express CD30, such as, e.g., T and B lymphocytes, T cell lymphomas, and B cell lymphomas.

A nucleic acid molecule encoding an antagonistic DR6 antibody or antigen-binding fragments thereof that binds an epitope within amino acids 121-197 of SEQ ID NO: 5 can be delivered to and expressed in a cell to produce the antibody or antigen-binding fragment thereof. For example, produced antagonistic DR6 antibodies or antigen-binding fragments thereof encoded by the nucleic acid molecule may bind an epitope of, within, or including one or more of amino acids 121-176 (e.g., amino acids 141-156, NGTCAPHTVCPVGWGV, SEQ ID NO: 32) of the DR6 amino acid sequence (SEQ ID NO: 5) and/or amino acids 149-197 (e.g., amino acids 169-177, KQCARGTFS, SEQ ID NO: 33) of the DR6 amino acid sequence (SEQ ID NO: 5). In an embodiment, the epitope bound by the antibody or antigen-binding fragment thereof is or includes one or more of amino acids 141-156 of SEQ ID NO: 5. In an embodiment, the epitope bound by the antibody or antigen-binding fragment thereof is or includes one or more of amino acids 169-177 of SEQ ID NO: 5. The antagonistic DR6 antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-DR6 polypeptides (e.g., single-chain polypeptides, antibodies, and antigen-binding fragments) encoded by the nucleic acid molecule may also specifically bind an epitope within human DR6 that includes at least five continuous or discontinuous amino acid residues of amino acids 121-197 of SEQ ID NO: 5 (e.g., at least five continuous or discontinuous amino acid residues of amino acids 131-166 and/or positions 159-187 of SEQ ID NO: 5, or at least five continuous or discontinuous amino acid residues of amino acids 141-156 and/or positions 169-177 of SEQ ID NO: 5). Furthermore, anti-DR6 polypeptides (e.g., single-chain polypeptides, antibodies, and antigen-binding fragments) encoded by the nucleic acid molecule may also specifically bind an epitope within human DR6 that includes at least five continuous or discontinuous amino acid residues of amino acids 121-197 of SEQ ID NO: 5 (e.g., at least five continuous or discontinuous amino acid residues of amino acids 131-166 and/or positions 159-187 of SEQ ID NO: 5, or at least five continuous or discontinuous amino acid residues of amino acids 141-156 and/or positions 169-177 of SEQ ID NO: 5), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the DR6 antibodies or antigen-binding fragments thereof can be engineered to target cells that express DR6, such as, e.g., thymus cells, spleen cells, T cells, nerve cells, and gliomas.

A nucleic acid molecule encoding an antagonistic EDAR antibody or antigen-binding fragments thereof that binds an epitope within amino acids 71-142 of SEQ ID NO: 6 can be delivered to and expressed in a cell to produce the antibody or antigen-binding fragment thereof. For example, the antagonistic EDAR antibodies or antigen-binding fragments thereof encoded by the nucleic acid molecule may bind an epitope of, within, or including one or more of amino acids 71-122 (e.g., amino acids 91-102, KDCEGFFRATVL, SEQ ID NO: 34) of the EDAR amino acid sequence (SEQ ID NO: 6) and/or amino acids 91-142 (e.g., amino acids 111-122, AECGPCLPGYYM, SEQ ID NO: 35) of the EDAR amino acid sequence (SEQ ID NO: 6). In an embodiment, the epitope bound by the antibody or antigen-binding fragment thereof is or includes one or more of amino acids 91-102 of SEQ ID NO: 6. In an embodiment, the epitope bound by the antibody or antigen-binding fragment thereof is or includes one or more of amino acids 111-122 of SEQ ID NO: 6. The antagonistic EDAR antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-EDAR polypeptides (e.g., single-chain polypeptides, antibodies, and antigen-binding fragments) encoded by the nucleic acid molecule may also specifically bind an epitope within human EDAR that includes at least five continuous or discontinuous amino acid residues of amino acids 71-142 of SEQ ID NO: 6 (e.g., at least five continuous or discontinuous amino acid residues of amino acids 81-112 and/or positions 101-132 of SEQ ID NO: 6, or at least five continuous or discontinuous amino acid residues of amino acids 91-102 and/or positions 111-122 of SEQ ID NO: 6). Furthermore, anti-EDAR polypeptides (e.g., single-chain polypeptides, antibodies, and antigen-binding fragments) encoded by the nucleic acid molecule may also specifically bind an epitope within human EDAR that includes at least five continuous or discontinuous amino acid residues of amino acids 91-142 of SEQ ID NO: 6 (e.g., at least five continuous or discontinuous amino acid residues of amino acids 81-112 and/or positions 101-132 of SEQ ID NO: 6, or at least five continuous or discontinuous amino acid residues of amino acids 91-102 and/or positions 111-122 of SEQ ID NO: 6), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the EDAR antibodies or antigen-binding fragments thereof can be engineered to target cells that express EDAR, such as, e.g., embryonic cells.

A nucleic acid molecule encoding an antagonistic Fas antibody or antigen-binding fragments thereof that binds an epitope within amino acids 128-193 of SEQ ID NO: 7 can be delivered to and expressed in a cell to produce the antibody or antigen-binding fragment thereof. For example, the antagonistic Fas antibodies or antigen-binding fragments thereof encoded by the nucleic acid molecule may bind an epitope of, within, or including one or more of amino acids 128-176 (e.g., amino acids 148-156, KCEHGIIKE, SEQ ID NO: 36) of the Fas amino acid sequence (SEQ ID NO: 7) and/or amino acids 145-193 (e.g., amino acids 165-173, CKEEGSRSN, SEQ ID NO: 37) of the Fas amino acid sequence (SEQ ID NO: 7). In an embodiment, the epitope bound by the antibody or antigen-binding fragment thereof is or includes one or more of amino acids 148-156 of SEQ ID NO: 7. In an embodiment, the epitope bound by the antibody or antigen-binding fragment thereof is or includes one or more of amino acids 165-173 of SEQ ID NO: 7. The antagonistic Fas antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-Fas polypeptides (e.g., single-chain polypeptides, antibodies, and antigen-binding fragments) encoded by the nucleic acid molecule may also specifically bind an epitope within human Fas that includes at least five continuous or discontinuous amino acid residues of amino acids 128-193 of SEQ ID NO: 7 (e.g., at least five continuous or discontinuous amino acid residues of amino acids 138-166 and/or positions 155-183 of SEQ ID NO: 7, or at least five continuous or discontinuous amino acid residues of amino acids 148-156 and/or positions 165-173 of SEQ ID NO: 7). Furthermore, anti-Fas polypeptides (e.g., single-chain polypeptides, antibodies, and antigen-binding fragments) encoded by the nucleic acid molecule may also specifically bind an epitope within human Fas that includes at least five continuous or discontinuous amino acid residues of amino acids 128-193 of SEQ ID NO: 7 (e.g., at least five continuous or discontinuous amino acid residues of amino acids 138-166 and/or positions 155-183 of SEQ ID NO: 7, or at least five continuous or discontinuous amino acid residues of amino acids 148-156 and/or positions 165-173 of SEQ ID NO: 7), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the Fas antibodies or antigen-binding fragments thereof can be engineered to target cells that express Fas, such as, e.g., thymus cells, liver cells, kidney cells, and B cells.

A nucleic acid molecule encoding an antagonistic GITR antibody or antigen-binding fragments thereof that binds an epitope within amino acids 72-141 of SEQ ID NO: 8 can be delivered to and expressed in a cell to produce the antibody or antigen-binding fragment thereof. For example, produced antagonistic GITR antibodies or antigen-binding fragments thereof encoded by the nucleic acid molecule may bind an epitope of, within, or including one or more of amino acids 72-121 (e.g., amino acids 92-101, HPCPPGQGVQ, SEQ ID NO: 38) of the GITR amino acid sequence (SEQ ID NO: 8) and/or amino acids 92-141 (e.g., amino acids 112-121, CIDCASGTFS, SEQ ID NO: 39) of the GITR amino acid sequence (SEQ ID NO: 8). In an embodiment, the epitope bound by the antibody or antigen-binding fragment thereof is or includes one or more of amino acids 92-101 of SEQ ID NO: 8. In an embodiment, the epitope bound by the antibody or antigen-binding fragment thereof is or includes one or more of amino acids 112-121 of SEQ ID NO: 8. The antagonistic GITR antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-GITR polypeptides (e.g., single-chain polypeptides, antibodies, and antigen-binding fragments) encoded by the nucleic acid molecule may also specifically bind an epitope within human GITR that includes at least five continuous or discontinuous amino acid residues of amino acids 72-141 of SEQ ID NO: 8 (e.g., at least five continuous or discontinuous amino acid residues of amino acids 82-111 and/or positions 102-131 of SEQ ID NO: 8, or at least five continuous or discontinuous amino acid residues of amino acids 92-101 and/or positions 112-121 of SEQ ID NO: 8). Furthermore, anti-GITR polypeptides (e.g., single-chain polypeptides, antibodies, and antigen-binding fragments) encoded by the nucleic acid molecule may also specifically bind an epitope within human GITR that includes at least five continuous or discontinuous amino acid residues of amino acids 72-141 of SEQ ID NO: 8 (e.g., at least five continuous or discontinuous amino acid residues of amino acids 82-111 and/or positions 102-131 of SEQ ID NO: 8, or at least five continuous or discontinuous amino acid residues of amino acids 92-101 and/or positions 112-121 of SEQ ID NO: 8), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the GITR antibodies or antigen-binding fragments thereof can be engineered to target cells that express GITR, such as, e.g., T cells, and NK cells.

A nucleic acid molecule encoding an antagonistic HVEM antibody or antigen-binding fragments thereof that binds an epitope within amino acids 122-190 of SEQ ID NO: 9 can be delivered to and expressed in a cell to produce the antibody or antigen-binding fragment thereof. For example, the antagonistic HVEM antibodies or antigen-binding fragments thereof encoded by the nucleic acid molecule may bind an epitope of, within, or including one or more of amino acids 122-172 (e.g., amino acids 142-152, TTCPPGQRVEK, SEQ ID NO: 40) of the HVEM amino acid sequence (SEQ ID NO: 9) and/or amino acids 142-190 (e.g., amino acids 162-170, CADCLTGTF, SEQ ID NO: 41) of the HVEM amino acid sequence (SEQ ID NO: 9). In an embodiment, the epitope bound by the antibody or antigen-binding fragment thereof is or includes one or more of amino acids 142-152 of SEQ ID NO: 9. In an embodiment, the epitope bound by the antibody or antigen-binding fragment thereof is or includes one or more of amino acids 162-170 of SEQ ID NO: 9. The antagonistic HVEM antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-HVEM polypeptides (e.g., single-chain polypeptides, antibodies, and antigen-binding fragments) encoded by the nucleic acid molecule may also specifically bind an epitope within human HVEM that includes at least five continuous or discontinuous amino acid residues of amino acids 122-190 of SEQ ID NO: 9 (e.g., at least five continuous or discontinuous amino acid residues of amino acids 132-162 and/or positions 152-180 of SEQ ID NO: 9, or at least five continuous or discontinuous amino acid residues of amino acids 142-152 and/or positions 162-170 of SEQ ID NO: 9). Furthermore, anti-HVEM polypeptides (e.g., single-chain polypeptides, antibodies, and antigen-binding fragments) encoded by the nucleic acid molecule may also specifically bind an epitope within human HVEM that includes at least five continuous or discontinuous amino acid residues of amino acids 122-190 of SEQ ID NO: 9 (e.g., at least five continuous or discontinuous amino acid residues of amino acids 132-162 and/or positions 152-180 of SEQ ID NO: 9, or at least five continuous or discontinuous amino acid residues of amino acids 142-152 and/or positions 162-170 of SEQ ID NO: 9), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the HVEM antibodies or antigen-binding fragments thereof can be engineered to target cells that express HVEM, such as, e.g., colorectal cancers, esophageal carcinomas, gastric cancers, hepatocarcinomas, breast cancers, lymphomas, spleen cells, thymus cells, bone marrow cells, T-reg cells, T cells, B cells, lung cells, and intestinal cells.

A nucleic acid molecule encoding an antagonistic LT beta receptor antibody or antigen-binding fragments thereof that binds an epitope within amino acids 127-196 of SEQ ID NO: 10 can be delivered to and expressed in a cell to produce the antibody or antigen-binding fragment thereof. For example, the antagonistic LT beta receptor antibodies or antigen-binding fragments thereof encoded by the nucleic acid molecule may bind an epitope of, within, or including one or more of amino acids 127-175 (e.g., amino acids 147-155, DCPPGTEAE, SEQ ID NO: 42) of the LT beta receptor amino acid sequence (SEQ ID NO: 10) and/or amino acids 147-196 (e.g., amino acids 167-176, CVPCKAGHFQ, SEQ ID NO: 43) of the LT beta receptor amino acid sequence (SEQ ID NO: 10). In an embodiment, the epitope bound by the antibody or antigen-binding fragment thereof is or includes one or more of amino acids 147-155 of SEQ ID NO: 10. In an embodiment, the epitope bound by the antibody or antigen-binding fragment thereof is or includes one or more of amino acids 167-176 of SEQ ID NO: 10. The antagonistic LT beta receptor antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-LT beta receptor polypeptides (e.g., single-chain polypeptides, antibodies, and antigen-binding fragments) encoded by the nucleic acid molecule may also specifically bind an epitope within human LT beta receptor that includes at least five continuous or discontinuous amino acid residues of amino acids 147-196 of SEQ ID NO: 10 (e.g., at least five continuous or discontinuous amino acid residues of amino acids 137-165 and/or positions 157-186 of SEQ ID NO: 10, or at least five continuous or discontinuous amino acid residues of amino acids 147-155 and/or positions 167-176 of SEQ ID NO: 10). Furthermore, anti-LT beta receptor polypeptides (e.g., single-chain polypeptides, antibodies, and antigen-binding fragments) encoded by the nucleic acid molecule may also specifically bind an epitope within human LT beta receptor that includes at least five continuous or discontinuous amino acid residues of amino acids 127-196 of SEQ ID NO: 10 (e.g., at least five continuous or discontinuous amino acid residues of amino acids 137-165 and/or positions 157-186 of SEQ ID NO: 10, or at least five continuous or discontinuous amino acid residues of amino acids 147-155 and/or positions 167-176 of SEQ ID NO: 10), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the LT beta receptor antibodies or antigen-binding fragments thereof can be engineered to target cells that express LT beta receptor, such as, e.g., cancer cells, stromal cells in lymphoid tissue, myeloid lineage cells, monocytes, alveolar macrophages, mast cells, and dendritic cells.

A nucleic acid molecule encoding an antagonistic NG FR antibody or antigen-binding fragments thereof that binds an epitope within amino acids 108-176 of SEQ ID NO: 11 can be delivered to and expressed in a cell to produce the antibody or antigen-binding fragment thereof. For example, the antagonistic NGFR antibodies or antigen-binding fragments thereof encoded by the nucleic acid molecule may bind an epitope of, within, or including one or more of amino acids 108-155 (e.g., amino acids 128-135, CEAGSGLV, SEQ ID NO: 44) of the NGFR amino acid sequence (SEQ ID NO: 11) and/or amino acids 126-176 (e.g., amino acids 146-156, CEECPDGTYSD, SEQ ID NO: 45) of the NGFR amino acid sequence (SEQ ID NO: 11). In an embodiment, the epitope bound by the antibody or antigen-binding fragment thereof is or includes one or more of amino acids 128-135 of SEQ ID NO: 11. In an embodiment, the epitope bound by the antibody or antigen-binding fragment thereof is or includes one or more of amino acids 146-156 of SEQ ID NO: 11. The antagonistic NGFR antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-NGFR polypeptides (e.g., single-chain polypeptides, antibodies, and antigen-binding fragments) encoded by the nucleic acid molecule may also specifically bind an epitope within human NGFR that includes at least five continuous or discontinuous amino acid residues of amino acids 108-176 of SEQ ID NO: 11 (e.g., at least five continuous or discontinuous amino acid residues of amino acids 118-145 and/or positions 136-166 of SEQ ID NO: 11, or at least five continuous or discontinuous amino acid residues of amino acids 128-135 and/or positions 146-156 of SEQ ID NO: 11). Furthermore, anti-NGFR polypeptides (e.g., single-chain polypeptides, antibodies, and antigen-binding fragments) encoded by the nucleic acid molecule may also specifically bind an epitope within human NGFR that includes at least five continuous or discontinuous amino acid residues of amino acids 108-176 of SEQ ID NO: 11 (e.g., at least five continuous or discontinuous amino acid residues of amino acids 118-145 and/or positions 136-166 of SEQ ID NO: 11, or at least five continuous or discontinuous amino acid residues of amino acids 128-135 and/or positions 146-156 of SEQ ID NO: 11), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the NGFR antibodies or antigen-binding fragments thereof can be engineered to target cells that express NGFR, such as, e.g., neural cells and tumor cells.

A nucleic acid molecule encoding an antagonistic OPG antibody or antigen-binding fragments thereof that binds an epitope within amino acids 103-171 of SEQ ID NO: 12 can be delivered to and expressed in a cell to produce the antibody or antigen-binding fragment thereof. For example, the antagonistic OPG antibodies or antigen-binding fragments thereof encoded by the nucleic acid molecule may bind an epitope of, within, or including one or more of amino acids 103-151 (e.g., amino acids 123-131, SCPPGFGVV, SEQ ID NO: 46) of the OPG amino acid sequence (SEQ ID NO: 12) and/or amino acids 122-171 (e.g., amino acids 142-151, CKRCPDGFFS, SEQ ID NO: 47) of the OPG amino acid sequence (SEQ ID NO: 12). In an embodiment, the epitope bound by the antibody or antigen-binding fragment thereof is or includes one or more of amino acids 123-131 of SEQ ID NO: 12. In an embodiment, the epitope bound by the antibody or antigen-binding fragment thereof is or includes one or more of amino acids 142-151 of SEQ ID NO: 12. The antagonistic OPG antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-OPG polypeptides (e.g., single-chain polypeptides, antibodies, and antigen-binding fragments) encoded by the nucleic acid molecule may also specifically bind an epitope within human OPG that includes at least five continuous or discontinuous amino acid residues of amino acids 103-171 of SEQ ID NO: 12 (e.g., at least five continuous or discontinuous amino acid residues of amino acids 113-141 and/or positions 132-161 of SEQ ID NO: 12, or at least five continuous or discontinuous amino acid residues of amino acids 123-131 and/or positions 142-151 of SEQ ID NO: 12). Furthermore, anti-OPG polypeptides (e.g., single-chain polypeptides, antibodies, and antigen-binding fragments) encoded by the nucleic acid molecule may also specifically bind an epitope within human OPG that includes at least five continuous or discontinuous amino acid residues of amino acids 103-171 of SEQ ID NO: 12 (e.g., at least five continuous or discontinuous amino acid residues of amino acids 113-141 and/or positions 132-161 of SEQ ID NO: 12, or at least five continuous or discontinuous amino acid residues of amino acids 123-131 and/or positions 142-151 of SEQ ID NO: 12), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the OPG antibodies or antigen-binding fragments thereof can be engineered to target cells that express OPG, such as, e.g., osteoblast lineage cells, epithelial cells (e.g., of the gastrointestinal tract, lung, breast, and skin), vascular endothelial cells, B cells, and dendritic cells.

A nucleic acid molecule encoding an antagonistic OX40 antibody or antigen-binding fragments thereof that binds an epitope within amino acids 98-154 of SEQ ID NO: 13 can be delivered to and expressed in a cell to produce the antibody or antigen-binding fragment thereof. For example, the antagonistic OX40 antibodies or antigen-binding fragments thereof encoded by the nucleic acid molecule may bind an epitope of, within, or including one or more of amino acids 98-154 (e.g., amino acids 118-134, SYKPGVDCAPCPPGHFS, SEQ ID NO: 48) of the OX40 amino acid sequence (SEQ ID NO: 13). In an embodiment, the epitope bound by the antibody or antigen-binding fragment thereof is or includes one or more of amino acids 118-134 of SEQ ID NO: 13. The antagonistic OX40 antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to this sequence and an epitope(s) that contains conservative amino acid substitutions relative to this sequence. The anti-OX40 polypeptides (e.g., single-chain polypeptides, antibodies, and antigen-binding fragments) encoded by the nucleic acid molecule may also specifically bind an epitope within human OX40 that includes at least five continuous or discontinuous amino acid residues of amino acids 98-154 of SEQ ID NO: 13 (e.g., at least five continuous or discontinuous amino acid residues of amino acids 108-144 of SEQ ID NO: 13, or at least five continuous or discontinuous amino acid residues of amino acids 118-134 of SEQ ID NO: 13). Furthermore, anti-OX40 polypeptides (e.g., single-chain polypeptides, antibodies, and antigen-binding fragments) encoded by the nucleic acid molecule may also specifically bind an epitope within human OX40 that includes at least five continuous or discontinuous amino acid residues of amino acids 98-154 of SEQ ID NO: 13 (e.g., at least five continuous or discontinuous amino acid residues of amino acids 108-144 of SEQ ID NO: 13, or at least five continuous or discontinuous amino acid residues of amino acids 118-134 of SEQ ID NO: 13), as well as an epitope(s) that exhibits at least 80% sequence identity (e.g., 80%, 85%, 90%, 95%, 97%, 99%, or 100% sequence identity) to this sequence and an epitope(s) that contains conservative amino acid substitutions relative to this sequence. A nucleic acid molecule encoding the OX40 antibodies or antigen-binding fragments thereof can be engineered to target cells that express OX40, such as activated CD4+ and CD8+ cytotoxic T cells and a number of lymphoid and non-lymphoid cells.

A nucleic acid molecule encoding an antagonistic RANK antibody or antigen-binding fragments thereof that binds an epitope within amino acids 112-180 of SEQ ID NO: 14 can be delivered to and expressed in a cell to produce the antibody or antigen-binding fragment thereof. For example, the antagonistic RANK antibodies or antigen-binding fragments thereof encoded by the nucleic acid molecule may bind an epitope of, within, or including one or more of amino acids 112-159 (e.g., amino acids 132-139, ECAPGLGA, SEQ ID NO: 49) of the RANK amino acid sequence (SEQ ID NO: 14) and/or amino acids 131-180 (e.g., amino acids 151-160, CKPCLAGYFS, SEQ ID NO: 50) of the RANK amino acid sequence (SEQ ID NO: 14). In an embodiment, the epitope bound by the antibody or antigen-binding fragment thereof is or includes one or more of amino acids 132-139 of SEQ ID NO: 14. In an embodiment, the epitope bound by the antibody or antigen-binding fragment thereof is or includes one or more of amino acids 151-160 of SEQ ID NO: 14. The antagonistic RANK antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-RANK polypeptides (e.g., single-chain polypeptides, antibodies, and antigen-binding fragments) encoded by the nucleic acid molecule may also specifically bind an epitope within human RANK that includes at least five continuous or discontinuous amino acid residues of amino acids 112-180 of SEQ ID NO: 14 (e.g., at least five continuous or discontinuous amino acid residues of amino acids 122-149 and/or positions 141-170 of SEQ ID NO: 14, or at least five continuous or discontinuous amino acid residues of amino acids 132-139 and/or positions 151-160 of SEQ ID NO: 14). Furthermore, anti-RANK polypeptides (e.g., single-chain polypeptides, antibodies, and antigen-binding fragments) encoded by the nucleic acid molecule may also specifically bind an epitope within human RANK that includes at least five continuous or discontinuous amino acid residues of amino acids 112-180 of SEQ ID NO: 14 (e.g., at least five continuous or discontinuous amino acid residues of amino acids 122-149 and/or positions 141-170 of SEQ ID NO: 14, or at least five continuous or discontinuous amino acid residues of amino acids 132-139 and/or positions 151-160 of SEQ ID NO: 14), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the RANK antibodies or antigen-binding fragments thereof can be engineered to target cells that express RANK, such as, e.g., skeletal muscle, thymus, liver, colon, small intestine, adrenal gland, osteoclast, mammary gland epithelial, prostate, vascular, and pancreatic cells.

A nucleic acid molecule encoding an antagonistic RELT (19L) antibody or antigen-binding fragments thereof that binds an epitope within amino acids 50-118 of SEQ ID NO: 15 can be delivered to and expressed in a cell to produce the antibody or antigen-binding fragment thereof. For example, the antagonistic RELT (19L) antibodies or antigen-binding fragments thereof encoded by the nucleic acid molecule may bind an epitope of, within, or including one or more of amino acids 50-97 (e.g., amino acids 70-77, RCSLWRRL, SEQ ID NO: 51) of the RELT (19L) amino acid sequence (SEQ ID NO: 15) and/or amino acids 70-118 (e.g., amino acids 90-98, CGDCWPGWF, SEQ ID NO: 52) of the RELT (19L) amino acid sequence (SEQ ID NO: 15). In an embodiment, the epitope bound by the antibody or antigen-binding fragment thereof is or includes one or more of amino acids 70-77 of SEQ ID NO 15. In an embodiment, the epitope bound by the antibody or antigen-binding fragment thereof is or includes one or more of amino acids 90-98 of SEQ ID NO: 15. The antagonistic RELT (19L) antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-RELT (19L) polypeptides (e.g., single-chain polypeptides, antibodies, and antigen-binding fragments) encoded by the nucleic acid molecule may also specifically bind an epitope within human RELT (19L) that includes at least five continuous or discontinuous amino acid residues of amino acids 50-118 of SEQ ID NO: 15 (e.g., at least five continuous or discontinuous amino acid residues of amino acids 60-87 and/or positions 80-108 of SEQ ID NO: 15, or at least five continuous or discontinuous amino acid residues of amino acids 70-77 and/or positions 90-98 of SEQ ID NO: 15). Furthermore, anti-RELT (19L) polypeptides (e.g., single-chain polypeptides, antibodies, and antigen-binding fragments) encoded by the nucleic acid molecule may also specifically bind an epitope within human RELT (19L) that includes at least five continuous or discontinuous amino acid residues of amino acids 50-118 of SEQ ID NO: 15 (e.g., at least five continuous or discontinuous amino acid residues of amino acids 60-87 and/or positions 80-108 of SEQ ID NO: 15, or at least five continuous or discontinuous amino acid residues of amino acids 70-77 and/or positions 90-98 of SEQ ID NO: 15), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the RELT (19L) antibodies or antigen-binding fragments thereof can be engineered to target cells that express RELT (19L), such as, e.g., hematologic tissue cells (e.g., tissues of the blood leukocytes, lymph, spleen, and bone marrow), T cells, B cells, and myeloid cells.

A nucleic acid molecule encoding an antagonistic TNFR1 antibody or antigen-binding fragments thereof that binds an epitope within amino acids 84-153 of SEQ ID NO: 16 can be delivered to and expressed in a cell to produce the antibody or antigen-binding fragment thereof. For example, the antagonistic TNFR1 antibodies or antigen-binding fragments thereof encoded by the nucleic acid molecule may bind an epitope of, within, or including one or more of amino acids 84-132 (e.g., amino acids 104-112, KCRKEMGQV, SEQ ID NO: 53) of the TNFR1 amino acid sequence (SEQ ID NO: 16) and/or amino acids 104-153 (e.g., amino acids 124-133, VCGCRKNQYR, SEQ ID NO: 54) of the TNFR1 amino acid sequence (SEQ ID NO: 16). In an embodiment, the epitope bound by the antibody or antigen-binding fragment thereof is or includes one or more of amino acids 104-112 of SEQ ID NO: 16. In an embodiment, the epitope bound by the antibody or antigen-binding fragment thereof is or includes one or more of amino acids 124-133 of SEQ ID NO: 16. The antagonistic TNFR1 antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-TNFR1 polypeptides (e.g., single-chain polypeptides, antibodies, and antigen-binding fragments) encoded by the nucleic acid molecule may also specifically bind an epitope within human TNFR1 that includes at least five continuous or discontinuous amino acid residues of amino acids 84-153 of SEQ ID NO: 16 (e.g., at least five continuous or discontinuous amino acid residues of amino acids 94-122 and/or positions 114-143 of SEQ ID NO: 16, or at least five continuous or discontinuous amino acid residues of amino acids 104-112 and/or positions 124-133 of SEQ ID NO: 16). Furthermore, anti-TNFR1 polypeptides (e.g., single-chain polypeptides, antibodies, and antigen-binding fragments) encoded by the nucleic acid molecule may also specifically bind an epitope within human TNFR1 that includes at least five continuous or discontinuous amino acid residues of amino acids 84-153 of SEQ ID NO: 16 (e.g., at least five continuous or discontinuous amino acid residues of amino acids 94-122 and/or positions 114-143 of SEQ ID NO: 16, or at least five continuous or discontinuous amino acid residues of amino acids 104-112 and/or positions 124-133 of SEQ ID NO: 16), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the TNFR1 antibodies or antigen-binding fragments thereof can be engineered to target cells that express TNFR1, i.e., nearly all cells since TNFR1 is ubiquitously expressed on the surface of most cells.

A nucleic acid molecule encoding an antagonistic TRAIL-R2 (TNFRSF10B) antibody or antigen-binding fragments thereof that binds an epitope within amino acids 135-206 of SEQ ID NO: 17 can be delivered to and expressed in a cell to produce the antibody or antigen-binding fragment thereof. For example, the antagonistic TRAIL-R2 (TNFRSF10B) antibodies or antigen-binding fragments thereof encoded by the nucleic acid molecule may bind an epitope of, within, or including one or more of amino acids 135-185 (e.g., amino acids 155-165, KCRTGCPRGMV, SEQ ID NO: 55) of the TRAIL-R2 (TNFRSF10B) amino acid sequence (SEQ ID NO: 17) and/or amino acids 158-206 (e.g., amino acids 178-186, CVHKESGTK, SEQ ID NO: 56) of the TRAIL-R2 (TNFRSF10B) amino acid sequence (SEQ ID NO: 17). In an embodiment, the epitope bound by the antibody or antigen-binding fragment thereof is or includes one or more of amino acids 155-165 of SEQ ID NO: 17. In an embodiment, the epitope bound by the antibody or antigen-binding fragment thereof is or includes one or more of amino acids 178-186 of SEQ ID NO: 17. The antagonistic TRAIL-R2 (TNFRSF10B) antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-TRAIL-R2 (TNFRSF10B) polypeptides (e.g., single-chain polypeptides, antibodies, and antigen-binding fragments) encoded by the nucleic acid molecule may also specifically bind an epitope within human TRAIL-R2 (TNFRSF10B) that includes at least five continuous or discontinuous amino acid residues of amino acids 135-206 of SEQ ID NO: 17 (e.g., at least five continuous or discontinuous amino acid residues of amino acids 145-175 and/or positions 168-196 of SEQ ID NO: 17, or at least five continuous or discontinuous amino acid residues of amino acids 155-165 and/or positions 178-186 of SEQ ID NO: 17). Furthermore, anti-TRAIL-R2 (TNFRSF10B) polypeptides (e.g., single-chain polypeptides, antibodies, and antigen-binding fragments) encoded by the nucleic acid molecule may also specifically bind an epitope within human TRAIL-R2 (TNFRSF10B) that includes at least five continuous or discontinuous amino acid residues of amino acids 135-206 of SEQ ID NO: 17 (e.g., at least five continuous or discontinuous amino acid residues of amino acids 145-175 and/or positions 168-196 of SEQ ID NO: 17, or at least five continuous or discontinuous amino acid residues of amino acids 155-165 and/or positions 178-186 of SEQ ID NO: 17), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the TRAIL-R2 (TNFRSF 10B) antibodies or antigen-binding fragments thereof can be engineered to target cells that express TRAIL-R2 (TNFRSF 10B), such as, e.g., hepatocytes, brain cells, kidney cells, heart myocytes, colon cells, germ cells, Leydig cells, alveolar septum cells, bronchial epithelial cells, and brain vascular epithelial cells.

A nucleic acid molecule encoding an antagonistic TRAIL-R1 (TNFRSF10A) antibody or antigen-binding fragments thereof that binds an epitope within amino acids 149-216 of SEQ ID NO: 18 can be delivered to and expressed in a cell to produce the antibody or antigen-binding fragment thereof. For example, the antagonistic TRAIL-R1 (TNFRSF10A) antibodies or antigen-binding fragments thereof encoded by the nucleic acid molecule may bind an epitope of, within, or including one or more of amino acids 149-197 (e.g., amino acids 169-177, ACKSDEEER, SEQ ID NO: 57) of the TRAIL-R1 (TNFRSF10A) amino acid sequence (SEQ ID NO: 18) and/or amino acids 168-216 (e.g., amino acids 188-196, CQCKPGTFR, SEQ ID NO: 58) of the TRAIL-R1 (TNFRSF10A) amino acid sequence (SEQ ID NO: 18). In an embodiment, the epitope bound by the antibody or antigen-binding fragment thereof is or includes one or more of amino acids 169-177 of SEQ ID NO: 18. In an embodiment, the epitope bound by the antibody or antigen-binding fragment thereof is or includes one or more of amino acids 188-196 of SEQ ID NO: 18. The antagonistic TRAIL-R1 (TNFRSF10A) antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-TRAIL-R1 (TNFRSF10A) polypeptides (e.g., single-chain polypeptides, antibodies, and antigen-binding fragments) encoded by the nucleic acid molecule may also specifically bind an epitope within human TRAIL-R1 (TNFRSF10A) that includes at least five continuous or discontinuous amino acid residues of amino acids 149-216 of SEQ ID NO: 18 (e.g., at least five continuous or discontinuous amino acid residues of amino acids 159-187 and/or positions 178-206 of SEQ ID NO: 18, or at least five continuous or discontinuous amino acid residues of amino acids 169-177 and/or positions 188-196 of SEQ ID NO: 18). Furthermore, anti-TRAIL-R1 (TNFRSF10A) polypeptides (e.g., single-chain polypeptides, antibodies, and antigen-binding fragments) encoded by the nucleic acid molecule may also specifically bind an epitope within human TRAIL-R1 (TNFRSF10A) that includes at least five continuous or discontinuous amino acid residues of amino acids 149-216 of SEQ ID NO: 18 (e.g., at least five continuous or discontinuous amino acid residues of amino acids 159-187 and/or positions 178-206 of SEQ ID NO: 18, or at least five continuous or discontinuous amino acid residues of amino acids 169-177 and/or positions 188-196 of SEQ ID NO: 18), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the TRAIL-R1 (TNFRSF10A) antibodies or antigen-binding fragments thereof can be engineered to target cells that express TRAIL-R1 (TNFRSF10A), such as, e.g., hepatocytes, bile duct epithelial cells, brain cells, kidney cells, heart myocytes, colon cells, germ cells, and Leydig cells.

A nucleic acid molecule encoding an antagonistic TRAIL-R4 antibody or antigen-binding fragments thereof that binds an epitope within amino acids 141-210 of SEQ ID NO: 19 can be delivered to and expressed in a cell to produce the antibody or antigen-binding fragment thereof. For example, the antagonistic TRAIL-R4 antibodies or antigen-binding fragments thereof encoded by the nucleic acid molecule may bind an epitope of, within, or including one or more of amino acids 141-189 (e.g., amino acids 161-169, GCPRGMVKV, SEQ ID NO: 59) of the TRAIL-R4 amino acid sequence (SEQ ID NO:19) and/or amino acids 161-210 (e.g., amino acids 181-190, KNESAASSTG, SEQ ID NO: 60) of the TRAIL-R4 amino acid sequence (SEQ ID NO:19). In an embodiment, the epitope bound by the antibody or antigen-binding fragment thereof is or includes one or more of amino acids 161-169 of SEQ ID NO: 19. In an embodiment, the epitope bound by the antibody or antigen-binding fragment thereof is or includes one or more of amino acids 181-190 of SEQ ID NO: 19. The antagonistic TRAIL-R4 antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-TRAIL-R4 polypeptides (e.g., single-chain polypeptides, antibodies, and antigen-binding fragments) encoded by the nucleic acid molecule may also specifically bind an epitope within human TRAIL-R4 that includes at least five continuous or discontinuous amino acid residues of amino acids 141-210 of SEQ ID NO: 19 (e.g., at least five continuous or discontinuous amino acid residues of amino acids 151-179 and/or positions 171-200 of SEQ ID NO: 19, or at least five continuous or discontinuous amino acid residues of amino acids 161-169 and/or positions 181-190 of SEQ ID NO: 19). Furthermore, anti-TRAIL-R4 polypeptides (e.g., single-chain polypeptides, antibodies, and antigen-binding fragments) encoded by the nucleic acid molecule may also specifically bind an epitope within human TRAIL-R4 that includes at least five continuous or discontinuous amino acid residues of amino acids 141-210 of SEQ ID NO: 19 (e.g., at least five continuous or discontinuous amino acid residues of amino acids 151-179 and/or positions 171-200 of SEQ ID NO: 19, or at least five continuous or discontinuous amino acid residues of amino acids 161-169 and/or positions 181-190 of SEQ ID NO: 19), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the TRAIL-R4 antibodies or antigen-binding fragments thereof can be engineered to target cells that express TRAIL-R4, such as, e.g., natural killer cells, CD8+ cytotoxic T cells, and tumor cells.

A nucleic acid molecule encoding an antagonistic TRAMP antibody or antigen-binding fragments thereof that binds an epitope within amino acids 122-191 of SEQ ID NO: 20. For example, the antagonistic TRAMP antibodies or antigen-binding fragments thereof encoded by the nucleic acid molecule may bind an epitope of, within, or including one or more of amino acids 122-170 (e.g., amino acids 142-150, LDCGALHRH, SEQ ID NO: 61) of the TRAMP amino acid sequence (SEQ ID NO: 20) and/or amino acids 142-191 (e.g., amino acids 162-171, CGTCLPGFYE, SEQ ID NO: 62) of the TRAMP amino acid sequence (SEQ ID NO: 20). In an embodiment, the epitope bound by the antibody or antigen-binding fragment thereof is or includes one or more of amino acids 142-150 of SEQ ID NO: 20. In an embodiment, the epitope bound by the antibody or antigen-binding fragment thereof is or includes one or more of amino acids 162-171 of SEQ ID NO: 20. The antagonistic TRAMP antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-TRAMP polypeptides (e.g., single-chain polypeptides, antibodies, and antigen-binding fragments) encoded by the nucleic acid molecule may also specifically bind an epitope within human TRAMP that includes at least five continuous or discontinuous amino acid residues of amino acids 122-191 of SEQ ID NO: 20 (e.g., at least five continuous or discontinuous amino acid residues of amino acids 132-160 and/or positions 152-181 of SEQ ID NO: 20, or at least five continuous or discontinuous amino acid residues of amino acids 142-150 and/or positions 162-171 of SEQ ID NO: 20). Furthermore, anti-TRAMP polypeptides (e.g., single-chain polypeptides, antibodies, and antigen-binding fragments) encoded by the nucleic acid molecule may also specifically bind an epitope within human TRAMP that includes at least five continuous or discontinuous amino acid residues of amino acids 122-191 of SEQ ID NO: 20 (e.g., at least five continuous or discontinuous amino acid residues of amino acids 132-160 and/or positions 152-181 of SEQ ID NO: 20, or at least five continuous or discontinuous amino acid residues of amino acids 142-150 and/or positions 162-171 of SEQ ID NO: 20), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the TRAMP antibodies or antigen-binding fragments thereof can be engineered to target cells that express TRAMP, such as, e.g., activated T cells and FoxP3+ T-reg cells.

A nucleic acid molecule encoding an antagonistic TROY antibody or antigen-binding fragments thereof that binds an epitope within amino acids 31-102 of SEQ ID NO: 21 can be delivered to and expressed in a cell to produce the antibody or antigen-binding fragment thereof. For example, the antagonistic TROY antibodies or antigen-binding fragments thereof encoded by the nucleic acid molecule may bind an epitope of, within, or including one or more of amino acids 31-79 (e.g., amino acids 51-59, QCGPGMELS, SEQ ID NO: 63) of the TROY amino acid sequence (SEQ ID NO: 21) and/or amino acids 52-102 (e.g., amino acids 72-82, CVTCRLHRFKE, SEQ ID NO: 64) of the TROY amino acid sequence (SEQ ID NO: 21). In an embodiment, the epitope bound by the antibody or antigen-binding fragment thereof is or includes one or more of amino acids 51-59 of SEQ ID NO: 21. In an embodiment, the epitope bound by the antibody or antigen-binding fragment thereof is or includes one or more of amino acids 72-82 of SEQ ID NO: 21. The antagonistic TROY antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-TROY polypeptides (e.g., single-chain polypeptides, antibodies, and antigen-binding fragments) encoded by the nucleic acid molecule may also specifically bind an epitope within human TROY that includes at least five continuous or discontinuous amino acid residues of amino acids 31-102 of SEQ ID NO: 21 (e.g., at least five continuous or discontinuous amino acid residues of amino acids 41-69 and/or positions 62-92 of SEQ ID NO: 21, or at least five continuous or discontinuous amino acid residues of amino acids 51-59 and/or positions 72-82 of SEQ ID NO: 21). Furthermore, anti-TROY polypeptides (e.g., single-chain polypeptides, antibodies, and antigen-binding fragments) encoded by the nucleic acid molecule may also specifically bind an epitope within human TROY that includes at least five continuous or discontinuous amino acid residues of amino acids 31-102 of SEQ ID NO: 21 (e.g., at least five continuous or discontinuous amino acid residues of amino acids 41-69 and/or positions 62-92 of SEQ ID NO: 21, or at least five continuous or discontinuous amino acid residues of amino acids 51-59 and/or positions 72-82 of SEQ ID NO: 21), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the TROY antibodies or antigen-binding fragments thereof can be engineered to target cells that express TROY, such as, e.g., embryonic cells, colon cells, hair follicle cells, and brain cells.

A nucleic acid molecule encoding an antagonistic XEDAR antibody or antigen-binding fragments thereof that binds an epitope within amino acids 1-69 of SEQ ID NO: 22 can be delivered to and expressed in a cell to produce the antibody or antigen-binding fragment thereof. For example, the antagonistic XEDAR antibodies or antigen-binding fragments thereof encoded by the nucleic acid molecule may bind an epitope of, within, or including one or more of amino acids 1-47 (e.g., amino acids 20-27, RCGPGQEL, SEQ ID NO: 65) of the XEDAR amino acid sequence (SEQ ID NO:22) and/or amino acids 22-69 (e.g., amino acids 42-29, TACPPRRY, SEQ ID NO: 66) of the XEDAR amino acid sequence (SEQ ID NO:22). In an embodiment, the epitope bound by the antibody or antigen-binding fragment thereof is or includes one or more of amino acids 20-27 of SEQ ID NO: 22. In an embodiment, the epitope bound by the antibody or antigen-binding fragment thereof is or includes one or more of amino acids 42-49 of SEQ ID NO: 22. The antagonistic XEDAR antibodies or antigen-binding fragments thereof encoded by the nucleic acid molecule may also bind an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. The anti-XEDAR polypeptides (e.g., single-chain polypeptides, antibodies, and antigen-binding fragments) encoded by the nucleic acid molecule may also specifically bind an epitope within human XEDAR that includes at least five continuous or discontinuous amino acid residues of amino acids 1-69 of SEQ ID NO: 22 (e.g., at least five continuous or discontinuous amino acid residues of amino acids 10-37 and/or positions 32-59 of SEQ ID NO: 22, or at least five continuous or discontinuous amino acid residues of amino acids 20-27 and/or positions 42-49 of SEQ ID NO: 22). Furthermore, anti-XEDAR polypeptides (e.g., single-chain polypeptides, antibodies, and antigen-binding fragments) encoded by the nucleic acid molecule may also specifically bind an epitope within human XEDAR that includes at least five continuous or discontinuous amino acid residues of amino acids 1-69 of SEQ ID NO: 22 (e.g., at least five continuous or discontinuous amino acid residues of amino acids 10-37 and/or positions 32-59 of SEQ ID NO: 22, or at least five continuous or discontinuous amino acid residues of amino acids 20-27 and/or positions 42-49 of SEQ ID NO: 22), as well as an epitope(s) that exhibits at least 80% sequence identity (e.g., 80%, 85%, 90%, 95%, 97%, 99%, or 100% sequence identity) to one or both of these sequences and an epitope(s) that contains conservative amino acid substitutions relative to one or both of these sequences. A nucleic acid molecule encoding the XEDAR antibodies or antigen-binding fragments thereof can be engineered to target cells that express XEDAR, such as, e.g., epidermal cells.

### Antagonistic TNFRSF Member Polypeptide Conjugates

Prior to administration of antagonistic TNFRSF member polypeptides of the disclosure to a mammalian subject (e.g., a human), it may be desirable to conjugate the polypeptide (e.g., single-chain polypeptide, antibody, or antigen-binding fragment thereof, such as anti-CD40 polypeptides that bind CD40) to a second molecule, e g., to modulate the activity of the polypeptide in vivo. Antagonistic TNFRSF member single-chain polypeptides, antibodies, and fragments thereof can be conjugated to other molecules at either the N-terminus or C-terminus of a light or heavy chain of the polypeptide using any one of a variety of established conjugation strategies that are well-known in the art. Examples of pairs of reactive functional groups that can be used to covalently tether an antagonistic TNFRSF member single-chain polypeptide, antibody, or fragment thereof to another molecule include, without limitation, thiol pairs, carboxylic acids and amino groups, ketones and amino groups, aldehydes and amino groups, thiols and alpha,beta-unsaturated moieties (such as maleimides or dehydroalanine), thiols and alpha-halo amides, carboxylic acids and hydrazides, aldehydes and hydrazides, and ketones and hydrazides.

Antagonistic TNFRSF member single-chain polypeptides, antibodies, and fragments thereof can be covalently appended directly to another molecule by chemical conjugation as described. Alternatively, fusion proteins containing antagonistic TNFRSF member single-chain polypeptides, antibodies, and fragments thereof can be expressed recombinantly from a cell (e.g., a eukaryotic cell or prokaryotic cell). This can be accomplished, for example, by incorporating a polynucleotide encoding the fusion protein into the nuclear genome of a cell (e.g., using techniques described herein or known in the art). Optionally, single-chain polypeptides, antibodies, and fragments thereof of the disclosure can be joined to a second molecule by forming a covalent bond between the antibody and a linker. This linker can then be subsequently conjugated to another molecule, or the linker can be conjugated to another molecule prior to ligation to the anti-TNFRSF member single-chain polypeptide, antibody, or fragment thereof. Examples of linkers that can be used for the formation of a conjugate include polypeptide linkers, such as those that contain naturally occurring or non-naturally occurring amino acids. In some cases, it may be desirable to include D-amino acids in the linker, as these residues are not present in naturally-occurring proteins and are thus more resistant to degradation by endogenous proteases. Fusion proteins containing polypeptide linkers can be made using chemical synthesis techniques, such as those described herein, or through recombinant expression of a polynucleotide encoding the fusion protein in a cell (e.g., a prokaryotic or eukaryotic cell). Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

### Drug-Polypeptide Conjugates

An antagonistic TNFRSF member polypeptide (e.g., single-chain polypeptide, antibody, or antigen-binding fragment thereof) of the disclosure can additionally be conjugated to, admixed with, or administered separately from a therapeutic agent, such as a cytotoxic molecule. Conjugates of the disclosure may be applicable to the treatment or prevention of a disease associated with aberrant cell proliferation, such as a cancer described herein. Exemplary cytotoxic agents that can be conjugated to, admixed with, or administered separately from an antagonistic TNFRSF member polypeptide include, without limitation, antineoplastic agents such as: acivicin; aclarubicin; acodazole hydrochloride; acronine; adozelesin; adriamycin; aldesleukin; altretamine; ambomycin; a. metantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; camptothecin; caracemide; carbetimer; carboplatin; carmustine; carubicin hydrochloride; carzelesin; cedefingol; chlorambucil; cirolemycin; cisplatin; cladribine; combretestatin a-4; crisnatol mesylate; cyclophosphamide; cytarabine; dacarbazine; daca (n-[2-(dimethyl-amino) ethyl]acridine-4-carboxamide); dactinomycin; daunorubicin hydrochloride; daunomycin; decitabine; dexormaplatin; dezaguanine; dezaguanine mesylate; diaziquone; docetaxel; dolasatins; doxorubicin; doxorubicin hydrochloride; droloxifene; droloxifene citrate; dromostanolone propionate; duazomycin; edatrexate; eflornithine hydrochloride; ellipticine; elsamitrucin; enloplatin; enpromate; epipropidine; epirubicin hydrochloride; erbulozole; esorubicin hydrochloride; estramustine; estramustine phosphate sodium; etanidazole; ethiodized oil i 131; etoposide; etoposide phosphate; etoprine; fadrozole hydrochloride; fazarabine; fenretinide; floxuridine; fludarabine phosphate; fluorouracil; 5-fdump; flurocitabine; fosquidone; fostriecin sodium; gemcitabine; gemcitabine hydrochloride; gold au 198; homocamptothecin; hydroxyurea; idarubicin hydrochloride; ifosfamide; ilmofosine; interferon alfa-2a; interferon alfa-2b; interferon alfa-nl; interferon alfa-n3; interferon beta-i a; interferon gamma-i b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peploycinsulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; rhizoxin; rhizoxin d; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; strontium chloride sr 89; sulofenur; talisomycin; taxane; taxoid; tecogalan sodium; tegafur; teloxantrone hydrochloride; temoporfin; teniposide; teroxirone; testolactone; thiamiprine; thioguanine; thiotepa; thymitaq; tiazofurin; tirapazamine; tomudex; top53; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine; vinblastine sulfate; vincristine; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 2-chlorodeoxyadenosine; 2′ deoxyformycin; 9-aminocamptothecin; raltitrexed; N-propargyl-5,8-dideazafolic acid; 2chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlor ethamine); cyclophosphamide; melphalan; chlorambucil; ifosfamide; busulfan; N-methyl-N-nitrosourea (MNU); N, N′-Bis (2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl) ethylphosphonate-N-nitrosourea (fotemustine); streptozotocin; diacarbazine (DTIC); mitozolomide; temozolomide; thiotepa; mitomycin C; AZQ; adozelesin; cisplatin; carboplatin; ormaplatin; oxaliplatin; C1-973; DWA 2114R; JM216; JM335; Bis (platinum); tomudex; azacitidine; cytarabine; gemcitabine; 6-mercaptopurine; 6-thioguanine; hypoxanthine; teniposide 9-amino camptothecin; topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-hydroxyphenyl) retinamide; 13-cis retinoic acid; 3-methyl TTNEB; 9-cis retinoic acid; fludarabine (2-F-ara-AMP); or 2-chlorodeoxyadenosine (2-Cda).

Other therapeutic compounds that can be conjugated to, admixed with, or administered separately from an antagonistic TNFRSF member single-chain polypeptide, antibody, or antigen-binding fragment thereof of the disclosure in order to treat, prevent, or study the progression of a disease associated with aberrant cell proliferation include, but are not limited to, cytotoxic agents such as 20-pi-1,25 dihydroxyvitamin D3; 5-ethynyluracil; abiraterone; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones (A, R=H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; rnerbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single-chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

### Labeled Anti-TNFRSF Member Polypeptides

Antagonistic TNFRSF member single-chain polypeptides, antibodies, or antigen-binding fragments thereof may be conjugated to another molecule (e.g., an epitope tag) for the purpose of purification or detection. Examples of such molecules that are useful in protein purification include those that present structural epitopes capable of being recognized by a second molecule. This is a common strategy that is employed in protein purification by affinity chromatography, in which a molecule is immobilized on a solid support and exposed to a heterogeneous mixture containing a target protein conjugated to a molecule capable of binding the immobilized compound. Examples of epitope tag molecules that can be conjugated to antagonistic TNFRSF member single-chain polypeptides, antibodies, or fragments thereof for the purposes of molecular recognition include, without limitation, maltose-binding protein, glutathione-S-transferase, a poly-histidine tag, a FLAG-tag, a myc-tag, human influenza hemagglutinin (HA) tag, biotin, streptavidin. Conjugates containing the epitopes presented by these molecules are capable of being recognized by such complementary molecules as maltose, glutathione, a nickel-containing complex, an anti-FLAG antibody, an anti-myc antibody, an anti-HA antibody, streptavidin, or biotin, respectively. For example, one can purify an antagonistic TNFRSF member single-chain polypeptide, antibody, or fragment thereof of the disclosure that has been conjugated to an epitope tag from a complex mixture of other proteins and biomolecules (e.g., DNA, RNA, carbohydrates, phospholipids, etc.) by treating the mixture with a solid-phase resin containing an complementary molecule that can selectively recognize and bind the epitope tag of the antagonistic anti-TNFRSF member antibody or fragment thereof. Examples of solid-phase resins include agarose beads, which are compatible with purifications in aqueous solution.

An antagonistic TNFRSF member polypeptide of the disclosure can also be covalently appended to a fluorescent molecule, e.g., to detect the antibody or antigen-binding fragment thereof by fluorimetry and/or by direct visualization using fluorescence microscopy. Exemplary fluorescent molecules that can be conjugated to polypeptides of the disclosure include green fluorescent protein, cyan fluorescent protein, yellow fluorescent protein, red fluorescent protein, phycoerythrin, allophycocyanin, hoescht, 4′,6-diamidino-2-phenylindole (DAPI), propidium iodide, fluorescein, coumarin, rhodamine, tetramethylrhodamine, and cyanine. Additional examples of fluorescent molecules suitable for conjugation to polypeptides of the disclosure are well-known in the art and have been described in detail in, e.g., U.S. Pat. Nos. 7,417,131 and 7,413,874, each of which is incorporated by reference herein.

Antagonistic TNFRSF member polypeptides containing a fluorescent molecule are particularly useful for monitoring the cell-surface localization properties of polypeptides, such as single-chain polypeptides, antibodies, and fragments thereof of the disclosure. For instance, one can expose cultured mammalian cells (e.g., T-reg cells, T cells, B cells, monocytes, neutrophils, platelets, granulocytes, bone marrow-derived lymphoid cells, and parenchymal cells) to antagonistic TNFRSF member single-chain polypeptides, antibodies, or fragments thereof of the disclosure that have been covalently conjugated to a fluorescent molecule and subsequently analyze these cells using conventional fluorescent microscopy techniques known in the art. Confocal fluorescent microscopy is a particularly powerful method for determining cell-surface localization of antagonistic anti-TNFRSF member single-chain polypeptides, antibodies, or fragments thereof, as individual planes of a cell can be analyzed in order to distinguish antibodies or fragments thereof that have been internalized into a cell's interior, e.g., by receptor-mediated endocytosis, from those that are bound to the external face of the cell membrane. Additionally, cells can be treated with antagonistic TNFRSF member antibodies conjugated to a fluorescent molecule that emits visible light of a particular wavelength (e.g., fluorescein, which fluoresces at about 535 nm) and an additional fluorescent molecule that is known to localize to a particular site on the T-reg cell surface and that fluoresces at a different wavelength (e.g., a molecule that localizes to CD25 and that fluoresces at about 599 nm). The resulting emission patterns can be visualized by confocal fluorescence microscopy and the images from these two wavelengths can be merged in order to reveal information regarding the location of the antagonistic TNFRSF member single-chain polypeptide, antibody, or antigen-binding fragment thereof on the T-reg cell surface with respect to other receptors.

Bioluminescent proteins can also be incorporated into a fusion protein for the purposes of detection and visualization of an antagonistic anti-TNFRSF member polypeptide. Bioluminescent proteins, such as Luciferase and aequorin, emit light as part of a chemical reaction with a substrate (e.g., luciferin and coelenterazine). Exemplary bioluminescent proteins suitable for use as a diagnostic sequence and methods for their use are described in, e.g., U.S. Pat. Nos. 5,292,658, 5,670,356, 6,171,809, and 7,183,092, each of which is herein incorporated by reference. Antagonistic TNFRSF member single-chain polypeptides, antibodies, or fragments thereof labeled with bioluminescent proteins are a useful tool for the detection of antibodies of the disclosure following an in vitro assay. For instance, the presence of an antagonistic TNFRSF member antibody that has been conjugated to a bioluminescent protein can be detected among a complex mixture of additional proteins by separating the components of the mixture using gel electrophoresis methods known in the art (e.g., native gel analysis) and subsequently transferring the separated proteins to a membrane in order to perform a Western blot. Detection of the antagonistic TNFRSF member antibody among the mixture of other proteins can be achieved by treating the membrane with an appropriate Luciferase substrate and subsequently visualizing the mixture of proteins on film using established protocols.

The polypeptides (e.g., single-chain polypeptides, antibodies, and fragments thereof, such as anti-CD40 polypeptides that bind CD40) of the disclosure can also be conjugated to a molecule comprising a radioactive nucleus, such that an antibody or fragment thereof of the disclosure can be detected by analyzing the radioactive emission pattern of the nucleus. Alternatively, an antagonistic TNFRSF member antibody or fragment thereof can be modified directly by incorporating a radioactive nucleus within the antibody during the preparation of the protein. Radioactive isotopes of methionine (³⁵S), nitrogen (¹⁵N), or carbon (¹³C) can be incorporated into antibodies or fragments thereof of the disclosure by, e.g., culturing bacteria in media that has been supplemented with nutrients containing these isotopes. Optionally, tyrosine derivatives containing a radioactive halogen can be incorporated into an antagonistic TNFRSF member antibody or fragment thereof by, e.g., culturing bacterial cells in media supplemented with radiolabeled tyrosine. It has been shown that tyrosine functionalized with a radioactive halogen at the C2 position of the phenol system are rapidly incorporated into elongating polypeptide chains using the endogenous translation enzymes in vivo (U.S. Pat. No. 4,925,651; incorporated herein by reference). The halogens include fluorine, chlorine, bromine, iodine, and astatine. Additionally, antagonistic TNFRSF member antibodies or fragments thereof can be modified following isolation and purification from cell culture by functionalizing antibodies or fragments thereof of the disclosure with a radioactive isotope. The halogens represent a class of isotopes that can be readily incorporated into a purified protein by aromatic substitution at tyrosine or tryptophan, e.g., via reaction of one or more of these residues with an electrophilic halogen species. Examples of radioactive halogen isotopes include ¹⁸F, ⁷⁸Br, ⁷⁷Br, ¹²²I, ¹²³I, ¹²⁴I, ¹²⁵I, ¹²⁹I, ¹³¹I or ²¹¹At.

Another alternative strategy for the incorporation of a radioactive isotope is the covalent attachment of a chelating group to the antagonistic anti-TNFRSF member polypeptide (e.g., single-chain polypeptide, antibody, or fragment thereof). Chelating groups can be covalently appended to an antagonistic TNFRSF member polypeptide by attachment to a reactive functional group, such as a thiol, amino group, alcohol, or carboxylic acid. The chelating groups can then be modified to contain any of a variety of metallic radioisotopes, including, without limitation, such radioactive nuclides as ¹²⁵I, ⁶⁷Ga, ¹¹¹In, ⁹⁹TC, ¹⁶⁹Yb, ¹⁸⁶Re, ¹²³I, ¹²⁴I, ¹²⁵I, ¹³¹I, ^{99m}Tc, ¹¹¹In, ⁶⁴Cu, ⁶⁷Cu, ¹⁸⁶Re, ¹⁸⁸Re, ¹⁷⁷Lu, ⁹⁰Y, ⁷⁷As, ⁷²AS, ⁸⁶Y, ⁸⁹Zr, ²¹¹At, ²¹²Bi, ²¹³Bi, or ²²⁵AC.

In some examples, it may be desirable to covalently conjugate the polypeptides (e.g., single-chain polypeptides, antibodies, or fragments thereof, such as anti-CD40 polypeptides that bind CD40) of the disclosure with a chelating group capable of binding a metal ion from heavy elements or rare earth ions, such as Gd³⁺, Fe³⁺, Mn³⁺, or Cr²⁺. Conjugates containing chelating groups that are coordinated to such paramagnetic metals are useful as in MRI imaging applications. Paramagnetic metals include, but are not limited to, chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III), and ytterbium (III). In this way, antagonistic TNFRSF member antibodies can be detected by MRI spectroscopy. For instance, one can administer antagonistic TNFRSF member antibodies or fragments thereof conjugated to chelating groups bound to paramagnetic ions to a mammalian subject (e.g., a human subject) in order to monitor the distribution of the antibody following administration. This can be achieved by administration of the antibody to a subject by any of the administration routes described herein, such as intravenously, and subsequently analyzing the location of the administered antibody by recording an MRI of the subject according to established protocols.

Antagonistic TNFRSF member polypeptides (e.g., single-chain polypeptides, antibodies, or fragments thereof, such as anti-CD40 polypeptides that bind CD40) can additionally be conjugated to other molecules for the purpose of improving the solubility and stability of the protein in aqueous solution. Examples of such molecules include PEG, PSA, bovine serum albumin (BSA), and human serum albumin (HSA), among others. For instance, one can conjugate an antagonistic TNFRSF member antibody or fragment thereof to carbohydrate moieties in order to evade detection of the antibody or fragment thereof by the immune system of the subject receiving treatment. This process of hyperglycosylation reduces the immunogenicity of therapeutic proteins by sterically inhibiting the interaction of the protein with B-cell receptors in circulation. Alternatively, antagonistic TNFRSF member antibodies or fragments thereof can be conjugated to molecules that prevent clearance from human serum and improve the pharmacokinetic profile of antibodies of the disclosure. Exemplary molecules that can be conjugated to or inserted within anti-TNFRSF member antibodies or fragments thereof of the disclosure so as to attenuate clearance and improve the pharmacokinetic profile of these antibodies and fragments include salvage receptor binding epitopes. These epitopes are found within the Fc region of an IgG immunoglobulin and have been shown to bind Fc receptors and prolong antibody half-life in human serum. The insertion of salvage receptor binding epitopes into anti-TNFRSF member antibodies or fragments thereof can be achieved, e.g., as described in U.S. Pat. No. 5,739,277; incorporated herein by reference.

### Modified Antagonistic TNFRSF Member Polypeptides

In addition to conjugation to other therapeutic agents and labels for identification or visualization, anti-TNFRSF member polypeptides (e.g., single-chain polypeptides, antibodies, or fragments thereof, such as anti-CD40 polypeptides that bind CD40) of the disclosure can also be modified so as to improve their pharmacokinetic profile, biophysical stability, or inhibitory capacity. For instance, any cysteine residue not involved in maintaining the proper conformation of the anti-TNFRSF member antibody or fragment thereof may be substituted with an isosteric amino acid (e.g., serine) in order to improve the oxidative stability of the molecule and prevent aberrant crosslinking. Conversely, cystine bond(s) may be added to the antibody or fragment thereof to improve its stability (particularly where the antibody is an antibody fragment, such as an Fv fragment). This can be accomplished, e.g., by altering a polynucleotide encoding the antibody heavy and light chains or a polynucleotide encoding an antibody fragment so as to encode one or more additional pairs of cysteine residues that can form disulfide bonds under oxidative conditions in order to reinforce antibody tertiary structure (see, e.g., U.S. Pat. No. 7,422,899; incorporated herein by reference).

Another useful modification that may be made to anti-TNFRSF member polypeptides (e.g., single-chain polypeptides, antibodies, or fragments thereof, such as anti-CD40 polypeptides that bind CD40) of the disclosure includes altering the glycosylation profile of these antibodies and fragments thereof. This can be achieved, e.g., by substituting, inserting, or deleting amino acids in an antagonistic TNFRSF member antibody so as to insert or remove a glycosylation site. Glycosylation of antibodies typically occurs in N-linked or O-linked fashion. N-linked glycosylation is a process whereby the attachment of a carbohydrate moiety to an antibody occurs at the side-chain of an asparagine residue. Consensus amino acid sequences for N-linked glycosylation include the tripeptide sequences asparagine-X-serine (NXS) and asparagine-X-threonine (NXT), where X is any amino acid except proline. The insertion of either of these tripeptide sequences in a polypeptide (e.g., an anti-TNFRSF member antibody) creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine are also competent substrates for glycoside formation. Addition of glycosylation sites to an anti-TNFRSF member antibody can thus be accomplished by altering the amino acid sequence of the antibody (e.g., using recombinant expression techniques as described herein) such that it contains one or more of the above-described tripeptide sequences to promote N-linked glycosylation, or one or more serine or threonine residues to the sequence of the original antibody engender O-linked glycosylation (see, e.g., U.S. Pat. No. 7,422,899; incorporated herein by reference).

In alternative cases, it may be desirable to modify the antibody or fragment thereof of the disclosure with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. For instance, cysteine residues may be introduced in the Fc region of an anti-TNFRSF member antibody or fragment thereof (e.g., by recombinant expression techniques as described herein), so as to facilitate additional inter-chain disulfide bond formation in this region. The homodimeric antibody thus generated may have increased conformational constraint, which may foster improved internalization capability and/or increased complement-mediated cell killing and antibody-dependent cellular cytotoxicity (ADCC). Homodimeric antibodies with enhanced anti-tumor activity may also be prepared using heterobifunctional cross-linkers as described, for example, in Wolff et al. (Canc. Res., 53:2560-2565, 1993); incorporated herein by reference. Alternatively, an antibody can be engineered which has dual Fc regions and may thereby have enhanced complement lysis and ADCC capabilities (see Stevenson et al. (Anti-Canc. Drug Des., 3:219-230, 1989); incorporated herein by reference).

The serum half-life of anti-TNFRSF member polypeptides (e.g., single-chain polypeptides, antibodies, or fragments thereof, such as anti-CD40 polypeptides that bind CD40) of the disclosure can be improved in some examples by incorporating one more amino acid modifications, such as by altering the CH1 or C_{L} region of the Fab domain to introduce a salvage receptor motif, e.g., that found in the two loops of a CH2 domain of an Fc region of an IgG. Such alterations are described, for instance, in U.S. Pat. Nos. 5,869,046 and 6,121,022; incorporated herein by reference. Additional framework modifications can also be made to reduce immunogenicity of the antibody or fragment thereof or to reduce or remove T cell epitopes that reside therein, as described for instance in US2003/0153043; incorporated herein by reference.

### Methods of Treatment

### Methods of Treating Cell Proliferation Disorders

Antagonistic TNFRSF member polypeptides (e.g., single-chain polypeptides, antibodies, or fragments thereof) of the disclosure (e.g., a TNFRSF member listed in Tables 2 or 3, in which antagonism thereof may be efficacious for treating diseases such as, e.g., cancer and infectious diseases) are useful therapeutics for the treatment of a wide array of cancers and cell proliferation disorders. Certain polypeptides of the disclosure (e.g., polypeptides which inhibit TNFRSF members in Tables 2 and 3, on which antagonism thereof may be efficacious for treating cancer) can be administered to a mammalian subject, such as a human, suffering from a cell proliferation disorder, such as cancer, e.g., to enhance the effectiveness of the adaptive immune response against the target cancer cells.

In particular, antagonistic TRAF-binding TNFRSF member polypeptides (e.g., single-chain polypeptides, antibodies, or fragments thereof) of the disclosure can be administered to a mammalian subject, such as a human, to reduce or inhibit T-reg cell growth and activation, which allows tumor-infiltrating T-lymphocytes to localize to cells presenting tumor-associated antigens and to promote cytotoxicity. In addition, polypeptides of the disclosure may synergize with existing adoptive T-cell therapy platforms, as one of the limitations on the effectiveness of this strategy has been the difficulty of prolonging cytotoxicity of tumor-reactive T-cells following infusion into a mammalian subject (e.g., a human).

Antagonist TRAF-binding TNFRSF member antibodies and antigen-binding fragments thereof of the disclosure can mitigate the T-reg-mediated depletion of tumor-reactive T-cells by suppressing the growth and proliferation of T-reg cells that typically accompanies T-cell infusion. For instance, polypeptides of the disclosure (e.g., polypeptides which antagonize TRAF-binding TNFRSF member proteins) may be capable of reducing the growth of a population of T-reg cells by about 50% to about 200% relative to untreated cells (e.g., 50%, 75%, 100%, 125%, 150%, 175%, or 200%). The reduction in cellular growth occurs even in the presence of a cognate or natural ligand of the receptor. In some examples, polypeptides of the disclosure may be capable of restricting the growth of a population of T-reg cells in the presence of the natural ligand of the TNFRSF member to between 90% and 150% relative to untreated cells (e.g., 90%, 100%, 110%, 120%, 130%, 140%, or 150%). Antagonistic TNFRSF member polypeptides of the disclosure are also capable of restricting the proliferation of a population of T-reg cells to less than 70% (e.g., 60%, 50%, 40%, 30%, 20%, 10%, 5%, or 1%) of that of an untreated population of T-reg cells. Antagonistic TRAF-binding TNFRSF member polypeptides of the disclosure are also capable of decreasing the survival of a population of T-reg cells by about 10% (e.g., by about 20%, 30%, 40%, or 50%, or more) relative to an untreated population of T-reg cells.

Antagonist TRAF-binding TNFRSF member antibodies and antigen-binding fragments thereof of the disclosure can mitigate the MDSC-mediated depletion of tumor-reactive T-cells by suppressing the growth and proliferation of MDSCs that may accompany T-cell infusion. For instance, polypeptides of the disclosure (e.g., polypeptides which antagonize DD-containing TNFRSF member proteins) may be capable of reducing the growth of a population of MDSCs by about 50% to about 200% relative to untreated cells (e.g., 50%, 75%, 100%, 125%, 150%, 175%, or 200%). The reduction in cellular growth occurs even in the presence of a cognate or natural ligand of the receptor. In some examples, polypeptides of the disclosure may be capable of restricting the growth of a population of MDSCs in the presence of the natural ligand of the TNFRSF member to between 90% and 150% relative to untreated cells (e.g., 90%, 100%, 110%, 120%, 130%, 140%, or 150%). Antagonistic TNFRSF member polypeptides of the disclosure are also capable of restricting the proliferation of a population of MDSCs to less than 70% (e.g., 60%, 50%, 40%, 30%, 20%, 10%, 5%, or 1%) of that of an untreated population of MDSCs. Antagonistic TRAF-binding TNFRSF member polypeptides of the disclosure are also capable of decreasing the survival of a population of MDSCs by about 10% (e.g., by about 20%, 30%, 40%, or 50%, or more) relative to an untreated population of MDSCs.

Antagonist TRAF-binding TNFRSF member antibodies and antigen-binding fragments thereof of the disclosure can directly kill cells expressing the TNFRSF member, such as B cells, parenchymal cells, dendritic cells, platelets, and granulocytes. For instance, polypeptides of the disclosure (e.g., polypeptides which antagonize TRAF-binding TNFRSF member proteins) may be capable of reducing the growth of a population of TNFRSF member expressing cells by about 50% to about 200% relative to untreated cells (e.g., 50%, 75%, 100%, 125%, 150%, 175%, or 200%). The reduction in cellular growth may occur even in the presence of a cognate or natural ligand of the receptor. In some examples, polypeptides of the disclosure may be capable of restricting the growth of a population of TNFRSF member expressing cells in the presence of the natural ligand of the TNFRSF member to between 90% and 150% relative to untreated cells (e.g., 90%, 100%, 110%, 120%, 130%, 140%, or 150%). Antagonistic TNFRSF member polypeptides of the disclosure are also capable of restricting the proliferation of a population of TNFRSF member expressing cells to less than 70% (e.g., 60%, 50%, 40%, 30%, 20%, 10%, 5%, or 1%) of that of an untreated population of TNFRSF member expressing cells. Antagonistic TRAF-binding TNFRSF member polypeptides of the disclosure are also capable of decreasing the survival of a population of TNFRSF member expressing cells by about 10% (e.g., by about 20%, 30%, 40%, or 50%, or more) relative to an untreated population of TNFRSF member expressing cells.

Antagonist DD-containing TNFRSF member polypeptides of the disclosure (e.g., a TNFRSF member listed in Table 2, in which antagonism thereof can be efficacious for treating cancer) may also promote the activity of allogeneic T-lymphocytes. The T-lymphocytes may express foreign MHC proteins and may be increasingly susceptible to inactivation by the host immune system.

Antagonistic TNFRSF member polypeptides of the disclosure (e.g., single-chain polypeptides, antibodies, or fragments thereof) can be administered to a mammalian subject (e.g., a human) suffering from cancer in order to improve the condition of the subject by promoting the immune response against cancer cells and tumorigenic material. Antibodies of the disclosure can be administered to a subject, e.g., via any of the routes of administration described herein. Polypeptides of the disclosure (e.g., single-chain polypeptides, antibodies, or fragments thereof) can also be formulated with excipients, biologically acceptable carriers, and may be optionally conjugated to, admixed with, or co-administered separately (e.g., sequentially) with additional therapeutic agents, such as anti-cancer agents. Cancers that can be treated by administration of polypeptides of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-LT beta receptor polypeptides that bind anti-LT beta receptor; anti HVEM polypeptides that bind HVEM; anti-CD30 polypeptides that bind CD30; anti-BCMA polypeptides that bind BCMA; anti-TACI polypeptides that bind TACI; anti-BAFF-R polypeptides that bind BAFF-R; anti-FN14 polypeptides that bind FN14; anti-RELT polypeptides that bind RELT; anti-DR6 polypeptides that bind DR6; anti-RANK polypeptides that bind RANK; or anti-TROY polypeptides that bind TROY) include such cancers as leukemia, lymphoma, liver cancer, bone cancer, lung cancer, brain cancer (e.g., anti-TROY, anti-RELT (19L), and anti-FN14 polypeptides of the disclosure can be administered for treatment), bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer (e.g., anti-TROY polypeptides of the disclosure can be administered for treatment), testicular cancer, and throat cancer. Particular cancers that can be treated by administration of antibodies or antigen-binding fragments thereof of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-LT beta receptor polypeptides that bind LT beta receptor; anti HVEM polypeptides that bind HVEM; anti-CD30 polypeptides that bind CD30; anti-BCMA polypeptides that bind BCMA; anti-TACI polypeptides that bind TACI; anti-BAFF-R polypeptides that bind BAFF-R; anti-FN14 polypeptides that bind FN14; anti-RELT (19L) polypeptides that bind RELT (19L); anti-DR6 polypeptides that bind DR6; anti-RANK polypeptides that bind RANK; or anti-TROY polypeptides that bind TROY) include, without limitation, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer (e.g., anti-TROY polypeptides of the disclosure can be administered for treatment), appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer (e.g., anti-TROY polypeptides of the disclosure can be administered for treatment), extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma (e.g., anti-TROY polypeptides of the disclosure can be administered for treatment), childhood brain stem glioma (e.g., anti-TROY polypeptides of the disclosure can be administered for treatment), hairy cell leukemia, hepatocellular cancer, langerhans cell histiocytosis, Hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm (e.g., anti-BCMA, anti-TACI, and anti-BAFF-R polypeptides of the disclosure can be administered for treatment (e.g., anti-BCMA polypeptides of the disclosure can inhibit the proliferation of rapidly dividing cells in B cell tumors such as, e.g., multiple myeloma or refractory multiple myeloma)), myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer (e.g., anti-TROY polypeptides of the disclosure can be administered for treatment), retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia. Additionally, anti-RELT (19L) polypeptides of the disclosure can be administered to a subject to treat nerve/central nervous system tumors.

An anti-CD30 polypeptide of the disclosure can be administered for treatment (e.g., as a monotherapy or in combination with a checkpoint inhibitor (e.g., an anti-PD-1, PD-L1, or CTLA-4 agent)) for Hodgkin lymphoma or anaplastic large cell lymphoma (ALCL) with less side effects than current CD30 therapies. Current anti-CD30 therapies are validated for Hodgkin lymphoma and ALCL, but have associated adverse side effects such as, e.g., respiratory tract infection, nausea, anemia, fever, vomiting, low white blood cell counts, joint pain, hair loss, among others. Furthermore, side effects from current anti-CD30 therapies can continue to occur months after therapy ends and may further induce weakness, confusion, skin reactions, among others. The most serious adverse events that current CD30 related therapies may cause include permanent neuropathy, pulmonary embolism pneumothorax, pyelonephritis, septic shock, arrhythmia, Stevens Johnson syndrome, and tumor lysis syndrome.

An anti-TNFRSF member polypeptide of the disclosure (e.g., a single-chain polypeptide, an antibody, or an antigen-binding fragment thereof) can also be co-administered with a therapeutic antibody that exhibits reactivity towards a cancer cell. In this way, antagonistic TNFRSF member polypeptides of the disclosure may synergize not only with the adaptive immune response, e.g., by prolonging T-lymphocyte tumor reactivity, but also with other inhibitors of tumor cell growth. Examples of additional therapeutic antibodies that can be used to treat cancer and other cell proliferation disorders include those that exhibit reactivity with a tumor antigen or a cell-surface protein that is overexpressed on the surface of a cancer cell. Exemplary antibodies that can be admixed, co-administered, or sequentially administered with antagonistic TNFRSF member polypeptides of the disclosure include, without limitation, Trastuzumab (HERCEPTIN®), Bevacizumab (AVASTIN®), Cetuximab (ERBITUX®), Panitumumab (VECTIBIX®), Ipilimumab (YERVOY®), Rituximab (RITUXAN® and MABTHERA®), Alemtuzumab (CAMPATH®), Ofatumumab (ARZERRA®), Gemtuzumab ozogamicin (MYLOTARG®), Brentuximab vedotin (ADCETRIS®), 90 Y-Ibritumomab Tiuxetan (ZEVALIN®), and 131 I-Tositumomab (BEXXAR®), which are described in detail in Scott et al. (Cancer Immun., 12:14-21, 2012); incorporated herein by reference.

A physician having ordinary skill in the art can readily determine an effective amount of an antagonistic TNFRSF member polypeptide (e.g., a single-chain polypeptide, an antibody, or an antigen-binding fragment thereof) for administration to a mammalian subject (e.g., a human) in need thereof. For example, a physician could start prescribing doses of a polypeptide of the disclosure at levels lower than that required in order to achieve the desired therapeutic effect and gradually increase the dosage until the desired effect is achieved. Alternatively, a physician may begin a treatment regimen by administering an antagonistic TNFRSF member antibody or antibody fragment at a high dose and subsequently administer progressively lower doses until a therapeutic effect is achieved (e.g., a reduction in the volume of one or more tumors, a decrease in the population of T-reg cells and/or MDSCs, or remission of a cell proliferation disorder). In general, a suitable daily dose of an antibody or antigen-binding fragment thereof of the disclosure will be an amount of the antibody which is the lowest dose effective to produce a therapeutic effect. A single-chain polypeptide, antibody, or antigen-binding fragment thereof of the disclosure may be administered by injection, e.g., by intravenous, intramuscular, intraperitoneal, or subcutaneous injection, optionally proximal to the site of the target tissue (e.g., a tumor). A daily dose of a therapeutic composition of an antibody or antigen-binding fragment thereof of the disclosure may be administered as a single dose or as two, three, four, five, six or more doses administered separately at appropriate intervals throughout the day, week, month, or year, optionally, in unit dosage forms. While it is possible for an antibody or fragment thereof of the disclosure to be administered alone, it may also be administered as a pharmaceutical formulation in combination with excipients, carriers, and optionally, additional therapeutic agents.

Antagonistic polypeptides of the disclosure (e.g., single-chain polypeptides, antibodies, or fragments thereof) can be monitored for their ability to attenuate the progression of a cell proliferation disease, such as cancer, by any of a variety of methods known in the art. For instance, a physician may monitor the response of a mammalian subject (e.g., a human) to treatment with an antibody, antibody fragment, or single-chain polypeptide of the disclosure by analyzing the volume of one or more tumors in the subject. For example, polypeptides of the disclosure may be capable of reducing tumor volume by between 1% and 100% (e.g., 1%, 5%, 10%, 20%, 30%, 40%, 50%, 60%, 70%, 80%, 90%, 95%, 99%, or 100%). Alternatively, a physician may monitor the responsiveness of a subject (e.g., a human) to treatment with antagonistic TNFRSF member single-chain polypeptides, antibodies, or antigen-binding fragments thereof of the disclosure by analyzing the T-reg cell population in the lymph of a particular subject. For instance, a physician may withdraw a sample of blood from a mammalian subject (e.g., a human who was administered the antagonist) and determine the quantity or density of a population of T-reg cells (e.g., CD4+ CD25+ FOXP3+ T-reg cells or CD17+ T-reg cells) using established procedures, such as fluorescence activated cell sorting.

### Methods of Treating Infectious Diseases

Antagonistic TNFRSF member polypeptides of the disclosure (e.g., single-chain polypeptides, antibodies, or fragments thereof) can also be used for treating infectious diseases, such as those caused by any one or more of a virus, a bacterium, a fungus, or a parasite. The polypeptides of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) bind TNFRSF members selected from the group consisting of TRAMP, TRAIL-R3, TRAIL-R4, HVEM, DR6, or RELT (19L). For instance, antagonistic TNFRSF member polypeptides (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) can be administered to a mammalian subject (e.g., a human) suffering from an infectious disease in order to treat the disease, as well as to alleviate one or more symptoms of the disease.

For example, antagonistic TNFRSF member polypeptides of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) can be used for treating, or alleviating one or more symptoms of, viral infections in a mammalian subject, such as a human, that are caused by, e.g., a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus; a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, California encephalitis virus, and Crimean-Congo hemorrhagic fever (CCHF) virus; a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively); Friend Leukemia Virus; and transmissible spongiform encephalopathy, such as chronic wasting disease. Particularly, methods of the disclosure include administering an antagonistic TNFRSF member antibody to a human in order to treat an HIV infection (such as a human suffering from AIDS). Antagonistic TNFRSF member polypeptides of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) can also be used for treating, or alleviating one or more symptoms of, bacterial infections in a mammalian subject (e.g., a human). Examples of bacterial infections that may be treated by administration of an antagonistic TNFRSF member antibody or antibody fragment of the disclosure include, without limitation, those caused by bacteria within the genera Streptococcus, Bacillus, Listeria, Corynebacterium, Nocardia, Neisseria, Actinobacter, Moraxella, Enterobacteriacece (e.g., E. coli, such as O157:H7), Pseudomonas (such as Pseudomonas aeruginosa), Escherichia, Klebsiella, Serratia, Enterobacter, Proteus, Salmonella, Shigella, Yersinia, Haemophilus, Bordetella (such as Bordetella pertussis), Legionella, Pasteurella, Francisella, Brucella, Bartonella, Clostridium, Vibrio, Campylobacter, Staphylococcus, Mycobacterium (such as Mycobacterium tuberculosis and Mycobacterium avium paratuberculosis, and Helicobacter (such as Helicobacter pylori and Helicobacter hepaticus). Particularly, methods of the disclosure include administering an antagonistic TNFRSF member antibody (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) to a human or a non-human mammal in order to treat a Mycobacterium tuberculosis infection. Particular methods of the disclosure include administering an antagonistic TNFRSF member antibody (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) to bovine mammals or bison in order to treat a Mycobacterium tuberculosis infection. Additionally, methods of the disclosure include administering an antagonistic TNFRSF member antibody (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) to a human or a non-human mammal in order to treat a Mycobacterium avium paratuberculosis infection. Particular methods of the disclosure include administering an antagonistic TNFRSF member antibody (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) to bovine mammals or bison in order to treat a Mycobacterium avium paratuberculosis infection.

Antagonistic TNFRSF member polypeptides of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) can also be administered to a mammalian subject (e.g., a human) for treating, or alleviating one or more symptoms of, parasitic infections caused by a protozoan parasite (e.g., an intestinal protozoa, a tissue protozoa, or a blood protozoa) or a helminthic parasite (e.g., a nematode, a helminth, an adenophorea, a secementea, a trematode, a fluke (blood flukes, liver flukes, intestinal flukes, and lung flukes), or a cestode). Exemplary protozoan parasites that can be treated according to the methods of the disclosure include, without limitation, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Leishmania major, Toxoplasma gondii, Plasmodium vivax, Plasmodium ovale, Plasmodium malariae, Plasmodium falciparum, Plasmodium yoelli, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus. Additional parasitic infections that can be treated according to the methods of the disclosure include Onchocercas volvulus.

Antagonistic TNFRSF member polypeptides (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT (19L) polypeptides that bind RELT (19L)) can also be administered to a mammalian subject (e.g., a human) in order to treat, or to alleviate one or more symptoms of, fungal infections. Examples of fungal infections that may be treated according to the methods of the disclosure include, without limitation, those caused by, e.g., Aspergillus, Candida, Malassezia, Trichosporon, Fusarium, Acremonium, Rhizopus, Mucor, Pneumocystis, and Absidia. Exemplary fungal infections that can be treated according to the methods of the disclosure also include Pneumocystis carinii, Paracoccidioides brasiliensis and Histoplasma capsulatum.

### Methods of Treating Autoimmune Diseases

Antagonistic TNFRSF member polypeptides (e.g., single-chain polypeptides, antibodies, or fragments thereof) may be administered to a subject (e.g., a human) to treat an autoimmune disease. Antagonistic DD-containing TNFRSF member polypeptides of the disclosure can be used, for example, to treat a subject in need of organ repair or regeneration, e.g., by inducing the proliferation of cells within a damaged tissue or organ. Antagonistic DD-containing TNFRSF member polypeptides described herein can be administered to a mammalian subject, such as a human, to stimulate the proliferation of T-reg cells (e.g., CD4+, CD25+, FOXP3+ T-reg cells) and/or MDSCs. This response can have the effect of reducing populations of cytotoxic T-lymphocytes (e.g., CD8+ T-cells), B cells, monocytes, neutrophils, dendritic cells, platelets, macrophages, granulocytes, and mesenchymal cells that are often associated with mounting an inappropriate immune response that can cause an immunological disorder.

Antagonistic TRAF-binding TNFRSF member polypeptides (e.g., single-chain polypeptides, antibodies, or fragments thereof, such as anti-CD40 polypeptides that bind CD40) may, additionally or alternatively, directly kill T effector cells, such as CD8+ T effector cells, and may promote the proliferation, regeneration, healing, and/or protection of TNFRSF member-expressing parenchymal cells, as described herein.

Antagonistic TNFRSF member polypeptides (e.g., single-chain polypeptides, antibodies, or fragments thereof, such as anti-CD40 polypeptides that bind CD40; anti-TNFR1 polypeptide that binds TNFR1; anti-TRAIL-R1 (TNFRSF10A) polypeptides that bind TRAIL-R1 (TNFRSF10A); anti-TRAIL-R2 (TNFRSF10B) polypeptides that bind TRAIL-R2 (TNFRSF10B); anti-CD27 polypeptides that bind CD27; anti-4-1 BB polypeptides that bind 4-1 BB; anti-OX40 polypeptides that bind OX40; anti-GITR polypeptides that bind GITR; anti-RANK polypeptides that bind RANK; anti-Fas polypeptides that bind Fas; or anti-XEDAR polypeptides that bind XEDAR) described herein can be administered to a subject, e.g., a mammalian subject, such as a human, in order to treat such conditions as autoimmune diseases, neurological diseases, metabolic diseases (e.g., diabetes), macular diseases (e.g., macular degeneration), muscular atrophy, diseases related to miscarriage, vascular diseases (e.g., atherosclerosis), diseases related to bone loss (e.g., bone loss as a result of menopause or osteoporosis), allergies, asthma, a blood disorder (e.g., hemophilia), a musculoskeletal disorder, a disease related to growth receptor expression or activity, obesity, graft-versus-host disease (GVHD), or an allograft rejection, among other indications.

For example, anti-TNFR1 polypeptides, anti-4-1 BB polypeptides, anti-OX40 polypeptides, anti-GITR polypeptides, or anti-XEDAR polypeptides of the disclosure can be administered to a subject to treat GVHD. Anti-TNFR1 polypeptides, anti-CD40 polypeptides, anti-CD27 polypeptides, anti-4-1 BB polypeptides, or anti-OX40 polypeptides of the disclosure can be administered to a subject to treat asthma.

Antagonistic TNFRSF member polypeptides described herein (e.g., single-chain polypeptides, antibodies, or fragments thereof, such as anti-CD40 polypeptides that bind CD40; anti-TNFR1 polypeptide that binds TNFR1; anti-TRAIL-R1 (TNFRSF10A) polypeptides that bind TRAIL-R1 (TNFRSF10A); anti-TRAIL-R2 (TNFRSF10B) polypeptides that bind TRAIL-R2 (TNFRSF10B); anti-CD27 polypeptides that bind CD27; anti-4-1 BB polypeptides that bind 4-1 BB; anti-OX40 polypeptides that bind OX40; anti-GITR polypeptides that bind GITR; anti-RANK polypeptides that bind RANK; anti-Fas polypeptides that bind Fas; or anti-XEDAR polypeptides that bind XEDAR) can be administered to a subject, e.g., a mammalian subject, such as a human, suffering from a graft rejection. Examples of graft rejections that can be treated by administration of the polypeptides described herein include, without limitation, skin graft rejection, bone graft rejection, vascular tissue graft rejection, ligament graft rejection (e.g., cricothyroid ligament graft rejection, periodontal ligament graft rejection, suspensory ligament of the lens graft rejection, palmar radiocarpal ligament graft rejection, dorsal radiocarpal ligament graft rejection, ulnar collateral ligament graft rejection, radial collateral ligament graft rejection, suspensory ligament of the breast graft rejection, anterior sacroiliac ligament graft rejection, posterior sacroiliac ligament graft rejection, sacrotuberous ligament graft rejection, sacrospinous ligament graft rejection, inferior pubic ligament graft rejection, superior pubic ligament graft rejection, anterior cruciate ligament graft rejection, lateral collateral ligament graft rejection, posterior cruciate ligament graft rejection, medial collateral ligament graft rejection, cranial cruciate ligament graft rejection, caudal cruciate ligament graft rejection, patellar ligament graft rejection) and organ graft rejection (e.g., heart, lung, kidney, liver, pancreas, intestine, and thymus graft rejection, among others).

Antagonistic TNFRSF member polypeptides described herein (e.g., single-chain polypeptides, antibodies, or fragments thereof, such as anti-CD40 polypeptides that bind CD40; anti-TNFR1 polypeptide that binds TNFR1; anti-TRAIL-R1 (TNFRSF10A) polypeptides that bind TRAIL-R1 (TNFRSF10A); anti-TRAIL-R2 (TNFRSF10B) polypeptides that bind TRAIL-R2 (TNFRSF10B); anti-CD27 polypeptides that bind CD27; anti-4-1 BB polypeptides that bind 4-1 BB; anti-OX40 polypeptides that bind OX40; anti-GITR polypeptides that bind GITR; anti-RANK polypeptides that bind RANK; anti-Fas polypeptides that bind Fas; or anti-XEDAR polypeptides that bind XEDAR) may, additionally or alternatively, be administered to a subject, e.g., a mammalian subject, such as a human) suffering from a graft-versus-host disease (GVHD). Exemplary graft-versus-host diseases that can be treated using the compositions and methods of the disclosure include those that arises from a bone marrow transplant, as well as from the transplantation of blood cells, such as hematopoietic stem cells, common myeloid progenitor cells, common lymphoid progenitor cells, megakaryocytes, monocytes, basophils, eosinophils, neutrophils, macrophages, T-cells, B-cells, natural killer cells, and/or dendritic cells.

Antagonistic TNFRSF member polypeptides described herein (e.g., single-chain polypeptides, antibodies, or fragments thereof, such as anti-CD40 polypeptides that bind CD40; anti-TNFR1 polypeptide that binds TNFR1; anti-TRAIL-R1 (TNFRSF10A) polypeptides that bind TRAIL-R1 (TNFRSF10A); anti-TRAIL-R2 (TNFRSF10B) polypeptides that bind TRAIL-R2 (TNFRSF10B); anti-CD27 polypeptides that bind CD27; anti-4-1 BB polypeptides that bind 4-1 BB; anti-OX40 polypeptides that bind OX40; anti-GITR polypeptides that bind GITR; anti-RANK polypeptides that bind RANK; anti-Fas polypeptides that bind Fas; or anti-XEDAR polypeptides that bind XEDAR) can also be administered to a subject, e.g., a mammalian subject, such as a human, suffering from an immunological disease. Antibodies of the disclosure can be administered to a subject, e.g., via any of the routes of administration described herein.

Immunological diseases that can be treated by administration of polypeptides described herein include allergies, such as food allergy, seasonal allergy, pet allergy, hives, hay fever, allergic conjunctivitis, poison ivy allergy oak allergy, mold allergy, drug allergy, dust allergy, cosmetic allergy, and chemical allergy. For example, the above allergies can be treated by administering an anti-CD40 polypeptide, an anti-CD27 polypeptide, or an anti-XEDAR polypeptide of the disclosure.

Diseases that can be treated by administration of an antagonistic TNFRSF member polypeptide described herein (e.g., a single-chain polypeptide, an antibody, or an antigen-binding fragment thereof, such as an anti-CD40 polypeptide that binds CD40; an anti-TNFR1 polypeptide that binds TNFR1; an anti-TRAIL-R1 (TNFRSF10A) polypeptide that binds TRAIL-R1 (TNFRSF10A); an anti-TRAIL-R2 (TNFRSF10B) polypeptide that binds TRAIL-R2 (TNFRSF10B); an anti-CD27 polypeptide that binds CD27; an anti-4-1 BB polypeptide that binds 4-1 BB; an anti-OX40 polypeptide that binds OX40; an anti-GITR polypeptide that binds GITR; an anti-RANK polypeptide that binds RANK; an anti-Fas polypeptide that binds Fas; or an anti-XEDAR polypeptide that binds XEDAR) include autoimmune diseases, such as type I diabetes, alopecia areata, ankylosing spondylitis, antiphospholipid syndrome, autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, limited scleroderma (CREST Syndrome), cold agglutinin disease, Crohn's Disease, discoid lupus, essential mixed cryoglobulinemia, fibromyalgia-fibromyositis, Graves' Disease, Guillain-Barré Syndrome, Hashimoto's Thyroiditis, hypothyroidism, Inflammatory Bowel Disease, autoimmune lymphoproliferative syndrome (ALPS), idiopathic pulmonary fibrosis, idiopathic thrombocytopenia purpura (ITP), IgA nephropathy, juvenile arthritis, lichen planus, lupus, Meniere's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, Stiff-Man syndrome, Takayasu Arteritis, temporal arteritis/giant cell arteritis, ulcerative colitis, uveitis, vasculitis, vitiligo, and Wegener's Granulomatosis.

Antagonistic TNFRSF member polypeptides described herein (e.g., single-chain polypeptides, antibodies, or fragments thereof, such as anti-CD40 polypeptides that bind CD40; anti-TNFR1 polypeptide that binds TNFR1; anti-TRAIL-R1 (TNFRSF10A) polypeptides that bind TRAIL-R1 (TNFRSF10A); anti-TRAIL-R2 (TNFRSF10B) polypeptides that bind TRAIL-R2 (TNFRSF10B); anti-CD27 polypeptides that bind CD27; anti-4-1 BB polypeptides that bind 4-1 BB; anti-OX40 polypeptides that bind OX40; anti-GITR polypeptides that bind GITR; anti-RANK polypeptides that bind RANK; anti-Fas polypeptides that bind Fas; or anti-XEDAR polypeptides that bind XEDAR) can additionally be used to treat subjects in need of organ repair or regeneration. For instance, antagonistic TNFRSF member antibodies or antigen-binding fragments thereof of the disclosure (e.g., single-chain polypeptides, antibodies, or fragments thereof, such as anti-CD40 polypeptides that bind CD40; anti-TNFR1 polypeptide that binds TNFR1; anti-TRAIL-R1 (TNFRSF10A) polypeptides that bind TRAIL-R1 (TNFRSF10A); anti-TRAIL-R2 (TNFRSF10B) polypeptides that bind TRAIL-R2 (TNFRSF10B); anti-CD27 polypeptides that bind CD27; anti-4-1 BB polypeptides that bind 4-1 BB; anti-OX40 polypeptides that bind OX40; anti-GITR polypeptides that bind GITR; anti-RANK polypeptides that bind RANK; anti-Fas polypeptides that bind Fas; or anti-XEDAR polypeptides that bind XEDAR) may be used to stimulate organ repair or regeneration. Examples of tissues and organs that may be induced to regenerate by administration of an antagonistic TNFRSF member antibody or antigen-binding fragment thereof of the disclosure to a subject (e.g., a mammalian subject, such as a human) include the pancreas, salivary gland, pituitary gland, kidney, heart, lung, hematopoietic system, cranial nerves, heart, blood vessels including the aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

Antagonistic TNFRSF member polypeptides described herein (e.g., single-chain polypeptides, antibodies, or fragments thereof, such as anti-CD40 polypeptides that bind CD40; anti-TNFR1 polypeptide that binds TNFR1; anti-TRAIL-R1 (TNFRSF10A) polypeptides that bind TRAIL-R1 (TNFRSF10A); anti-TRAIL-R2 (TNFRSF10B) polypeptides that bind TRAIL-R2 (TNFRSF10B); anti-CD27 polypeptides that bind CD27; anti-4-1 BB polypeptides that bind 4-1 BB; anti-OX40 polypeptides that bind OX40; anti-GITR polypeptides that bind GITR; anti-RANK polypeptides that bind RANK; anti-Fas polypeptides that bind Fas; anti-DR6 polypeptides that bind DR6; or anti-XEDAR polypeptides that bind XEDAR) can also be administered to a subject (e.g., a mammalian subject, such as a human) in order to treat a neurological disease or condition, such as a spinal cord injury, schizophrenia, epilepsy, Amyotrophic lateral sclerosis (ALS), Parkinson's disease, Alzheimer's disease, Huntington's disease, or stroke. For example, anti-DR6 polypeptides that bind DR6 can be administered to a subject to treat Alzheimer's disease.

A physician of ordinary skill in the art can readily determine an effective amount of an antagonistic TNFRSF member polypeptide described herein (e.g., single-chain polypeptides, antibodies, or fragments thereof) for administration to a mammalian subject (e.g., a human) in need thereof. For example, a physician could start prescribing doses of an antagonistic TNFRSF member polypeptide at levels lower than that required to achieve the desired therapeutic effect and gradually increase the dosage until the desired effect is achieved. Alternatively, a physician may begin a treatment regimen by administering an antagonistic TNFRSF member polypeptide at a high dose and subsequently administer progressively lower doses until a therapeutic effect is achieved (e.g., a reduction in the proliferation of a population of CD8+ T cells, CD4+ T cells, and/or B cells) or a decrease in the peripheral secretion of IFNγ). In general, a suitable daily dose of an antibody or antigen-binding fragment thereof of the disclosure will be an amount of the antibody which is the lowest dose effective to produce a therapeutic effect. An antibody or antigen-binding fragment thereof of the disclosure may be administered by injection, e.g., by intravenous, intramuscular, intraperitoneal, or subcutaneous injection, optionally proximal to the site of a target tissue. A daily dose of a therapeutic composition of an antibody or antigen-binding fragment thereof of the disclosure may be administered as a single dose or as two, three, four, five, six or more doses administered separately at appropriate intervals throughout the day, week, month, or year, optionally, in unit dosage forms. While it is possible for an antibody or fragment thereof of the disclosure to be administered alone, it may also be administered as a pharmaceutical formulation in combination with excipients, carriers, and optionally, additional therapeutic agents.

Antagonistic polypeptides of the disclosure (e.g., single-chain polypeptides, antibodies, or fragments thereof) can be monitored for their ability to attenuate the progression of an immunological disease, such as an autoimmune disease, by any of a variety of methods known in the art. For instance, a physician may monitor the responsiveness of a subject (e.g., a human who was administered the antagonist) to treatment with antagonistic DD-containing TNFRSF member antibodies or antigen-binding fragments thereof of the disclosure by analyzing the T-reg cell population in the lymph of a particular subject. For instance, a physician may withdraw a sample of blood from a mammalian subject (e.g., a human who was administered the antagonist) and determine the quantity or density of a population of T-reg cells (e.g., CD4+ CD25+ FOXP3+ T-reg cells or CD17+ T-reg cells) using established procedures, such as fluorescence activated cell sorting. In these cases, high counts of T-reg cells is indicative of efficacious therapy, while lower T-reg cell counts may indicate that the subject is to be prescribed or administered higher dosages of the TNFRSF member antibody of the disclosure (e.g., single-chain polypeptides, antibodies, or fragments thereof, such as anti-TNFR1 polypeptide that binds TNFR1; anti-TRAIL-R1 (TNFRSF10A) polypeptides that bind TRAIL-R1 (TNFRSF10A); or anti-TRAIL-R2 (TNFRSF10B) polypeptides that bind TRAIL-R2 (TNFRSF10B)) until, e.g., an ideal T-reg cell count is achieved. In addition, a physician of skill in the art may monitor the effect of treatment by administration of antagonistic TNFRSF member antibodies or antigen-binding fragments thereof to a subject suffering from an immunological disorder, such as an autoimmune disease described herein, by analyzing the quantity of autoreactive CD8+ T-cells within a lymph sample isolated from the subject.

Antagonistic TRAF-binding TNFRSF member antibodies and antigen-binding fragments thereof of the disclosure may attenuate the proliferation of autoreactive T-cells. Treatment with antagonistic TNFRSF member antibodies or antigen-binding fragments thereof can lead to reduced quantities of autoreactive T-cells within the lymph isolated from a subject receiving treatment, and a rapid decline in the population of autoreactive T-cells in a lymph sample isolated from such a subject indicates effective treatment. In cases where a lymph sample isolated from a subject exhibits an autoreactive T-cell count that has not declined in response to antagonistic TRAF-binding TNFRSF member antibody therapy, a physician may prescribe the subject higher doses of the antibody or an antigen-binding fragment thereof or may administer the antagonistic TNFRSF member antibody or antigen-binding fragment thereof with higher frequency, e.g., multiple times per day, week, or month.

### Methods of Treating Obesity, Hyperlipidemia, and Type 2 Diabetes

Antagonistic Fas polypeptides (e.g., single-chain polypeptides, antibodies, or fragments thereof) may be administered to a subject (e.g., a human) to treat obesity, hyperlipidemia, and/or type 2 diabetes. Antagonistic Fas polypeptides of the disclosure can be used, e.g., to reduce inflammation in adipose tissue and liver, and improve insulin sensitivity (see Blûther et al., J. Clin. Endocrinol. Metab. 99:E36-44, 2014, which is incorporated herein by reference). A physician of skill in the art may monitor the effect of treatment by administration of antagonistic Fas antibodies or antigen-binding fragments thereof to a subject suffering from obesity, hyperlipidemia, and/or type 2 diabetes, by analyzing the level of blood sugar, triglycerides, lipids, and/or soluble Fas within a blood sample isolated from the subject.

### Methods of Treating Neurological Disorders

Antagonistic DR6 polypeptides (e.g., single-chain polypeptides, antibodies, or fragments thereof) may be administered to a subject (e.g., a human) to treat a neurological disorder such as a brain tumor, a brain metastasis, a spinal cord injury, schizophrenia, epilepsy, Parkinson's disease, autism, Huntington's disease, stroke, Alzheimer's disease, and amyotrophic lateral sclerosis (ALS). Antagonistic DR6 polypeptides of the disclosure can be used as a neuroprotective agent, e.g., by preventing a cleaved amino-terminal fragment of amyloid precursor protein (N-APP) from binding DR6, which has been demonstrated by Nikolaev et al. to cause axon degeneration and neuron death (see Nature 457:981-989, 2009, which is incorporated herein by reference). A physician of skill in the art may monitor the effect of treatment by administration of antagonistic DR6 antibodies or antigen-binding fragments thereof to a subject suffering from a neurodegenerative disorder, by analyzing the quantity of soluble DR6 within a blood sample isolated from the subject (or analyzing the quantity of phosphorylated neurofilament heavy subunit (pNfH) within a blood sample from a subject suffering from ALS (see Ganesalingam et al., Amyotroph. Lateral Soler. Frontotemporal Degener. 14:146-149, 2013, which is incorporated herein by reference)).

### Methods of Treating Osteoporosis

Antagonistic RANK polypeptides (e.g., single-chain polypeptides, antibodies, or fragments thereof) may be administered to a subject (e.g., a human) to treat osteoporosis or decreased bone loss in cancer. Antagonistic RANK polypeptides of the disclosure can be used, e.g., to protect bone from excessive resorption, similar to how osteoprotegerin binds to RANKL (see Boyce et al., Arch. Biochem. Biophys. 473:139-146, 2008, which is incorporated herein by reference). A physician of skill in the art may monitor the effect of treatment by administration of antagonistic RANK antibodies or antigen-binding fragments thereof to a subject suffering from osteoporosis (or decreased bone loss in cancer), by analyzing the level of soluble RANKL, OPG, and/or RANK within a blood sample isolated from the subject; and/or by measuring bone density within the subject (e.g., before and/or after treatment).

### Measuring Levels of Soluble TNFRSF Member Protein as a Biomarker

Levels of secreted soluble TNFRSF member protein (e.g., CD40, TRAIL-R1 (TNFRSF10A), TRAIL-R2 (TNFRSF10B), TRAMP, DR6, NGFR, TNFR1, FAS, EDAR, TRAIL-R3, TRAIL-R4, RANK, FN14, LT beta receptor, HVEM, CD30, CD27, 4-1 BB, OX40, GITR, BCMA, TACT, BAFF-R, XEDAR, TROY, or RELT (19L)) can be measured in a subject to determine disease progression and/or efficacy of treatment. The level of secreted soluble TNFRSF member protein can be measured again after administration of the antibody of the disclosure to a subject to determine if administration has caused a decrease in the level of secreted soluble TNFRSF member protein. For example, the level of secreted soluble TNFRSF member protein in a subject can be measured one day, two days, three days, four days, five days, six days, one week, eight days, nine days, ten days, eleven days, twelve days, thirteen days, fourteen days, fifteen days, sixteen days, seventeen days, eighteen days, nineteen days, twenty days, three weeks, four weeks, five weeks, six weeks, seven weeks, eight weeks, or more after administration of the antibody of the disclosure. The subject can be administered another dose of the antibody or polypeptide of the disclosure if the level of secreted soluble TNFRSF member protein is about the same or higher than the normal level of TNFRSF member or a previously measured level of secreted soluble TNFRSF member protein.

### Pharmaceutical Compositions

Therapeutic compositions containing an antagonistic TNFRSF member polypeptide, such as a single-chain polypeptide, antibody, or antigen-binding fragment thereof of the disclosure can be prepared using methods known in the art. The polypeptides of the disclosure (e.g., single-chain polypeptides, antibodies, or fragments thereof that can be used to form therapeutic compositions are those that bind TNFRSF members selected from the group consisting of CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, and XEDAR. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions. The compositions can also be prepared so as to contain the active agent (e.g., an antagonistic anti-TNFRSF member antibody or fragment thereof) at a desired concentration. For example, a pharmaceutical composition of the disclosure may contain at least 10% (e.g., 10%, 20%, 30%, 40%, 50%, 60%, 70%, 80%, 90%, 95%, 97%, 98%, 99%, 99.5%, 99.9%, or 100%) active agent by weight (w/w).

Additionally, an active agent (e.g., an antagonistic TNFRSF member antibody or fragment thereof of the disclosure) that can be incorporated into a pharmaceutical formulation can itself have a desired level of purity. For example, an antibody or antigen-binding fragment thereof of the disclosure may be characterized by a certain degree of purity after isolating the antibody from cell culture media or after chemical synthesis, e.g., of a single-chain antibody fragment (e.g., scFv) by established solid-phase peptide synthesis methods or native chemical ligation as described herein. An antagonistic TNFRSF member antibody of the disclosure may be at least 10% pure prior to incorporating the antibody into a pharmaceutical composition (e.g., 10°/0, 20%, 30%, 40%, 50%, 60%, 70%, 80%, 90%, 95%, 97%, 98%, 99%, 99.5%, 99.9%, 99.99%, or 100% pure).

Pharmaceutical compositions of anti-TNFRSF member polypeptides (e.g., single-chain polypeptides, antibodies, or fragments thereof, such as anti-CD40 polypeptides that bind CD40) of the disclosure can be prepared for storage as lyophilized formulations or aqueous solutions by mixing the antibody having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences, 16th edition (Osol, ed. 1980; incorporated herein by reference). Such additives must be nontoxic to the recipients at the dosages and concentrations employed.

### Buffering Agents

Buffering agents help to maintain the pH in the range which approximates physiological conditions. They can be present at concentration ranging from about 2 mM to about 50 mM. Suitable buffering agents for use with antagonistic TNFRSF member polypeptides (e.g., single-chain polypeptides, antibodies, or fragments thereof, such as anti-CD40 polypeptides that bind CD40) of the disclosure include both organic and inorganic acids and salts thereof such as citrate buffers {e.g., monosodium citrate-disodium citrate mixture, citric acid-trisodium citrate mixture, citric acid-monosodium citrate mixture, etc.), succinate buffers {e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers {e.g., fumaric acid-monosodium fumarate mixture, fumaric acid-disodium fumarate mixture, monosodium fumarate-disodium fumarate mixture, etc.), gluconate buffers {e.g., gluconic acid-sodium glyconate mixture, gluconic acid-sodium hydroxide mixture, gluconic acid-potassium gluconate mixture, etc.), oxalate buffer {e.g., oxalic acid-sodium oxalate mixture, oxalic acid-sodium hydroxide mixture, oxalic acid-potassium oxalate mixture, etc.), lactate buffers {e.g., lactic acid-sodium lactate mixture, lactic acid-sodium hydroxide mixture, lactic acid-potassium lactate mixture, etc.) and acetate buffers {e.g., acetic acid-sodium acetate mixture, acetic acid-sodium hydroxide mixture, etc.). Additionally, phosphate buffers, histidine buffers and trimethylamine salts such as Tris can be used.

### Preservatives

Preservatives can be added to a composition of the disclosure to retard microbial growth, and can be added in amounts ranging from 0.2%-l % (w/v). Suitable preservatives for use with antagonistic TNFRSF member polypeptides (e.g., single-chain polypeptides, antibodies, or fragments thereof, such as anti-CD40 polypeptides that bind CD40) of the disclosure include phenol, benzyl alcohol, meta-cresol, methyl paraben, propyl paraben, octadecyldimethylbenzyl ammonium chloride, benzalkonium halides {e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the disclosure and include polyhydric sugar alcohols, for example trihydric or higher sugar alcohols, such as glycerin, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (e.g., peptides of 10 residues or fewer); proteins such as human serum albumin, bovine serum albumin, gelatin or immunoglobulins; hydrophilic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in the range from 0.1 to 10,000 weights per part of weight active protein.

### Detergents

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants can be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### Other Pharmaceutical Carriers

Alternative pharmaceutically acceptable carriers that can be incorporated into a composition of the disclosure may include dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. A composition containing an antagonistic TNFRSF member polypeptide of the disclosure may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

### Compositions for Combination Therapy

Pharmaceutical compositions of the disclosure may optionally include more than one active agent. For instance, compositions of the disclosure may contain an antagonistic TNFRSF member polypeptide, such as a single-chain polypeptide, antibody, or fragment thereof conjugated to, admixed with, or administered separately from another pharmaceutically active molecule, e.g., a cytotoxic agent, an antibiotic, or a T-lymphocyte (e.g., a gene-edited T-lymphocyte for use in CAR-T therapy). For instance, an antagonistic TNFRSF member single-chain polypeptide, antibody, antibody fragment, or therapeutic conjugate thereof (e.g., a drug-antibody conjugate described herein), may be admixed with one or more additional active agents that can be used to treat cancer or another cell proliferation disorder (e.g., neoplasm). Alternatively, pharmaceutical compositions of the disclosure may be formulated for co-administration or sequential administration with one or more additional active agents that can be used to treat cancer or other cell proliferation disorders. Examples of additional active agents that can be used to treat cancer and other cell proliferation disorders and that can be conjugated to, admixed with, or administered separately from an antagonistic TNFRSF member single-chain polypeptide, antibody, or antibody fragment of the disclosure include cytotoxic agents (e.g., those described herein), as well as antibodies that exhibit reactivity with a tumor antigen or a cell-surface protein that is overexpressed on the surface of a cancer cell. Exemplary antibodies that can be conjugated to, admixed with, or administered separately from antagonistic TNFRSF member antibodies of the disclosure include, without limitation, Trastuzamb (HERCEPTIN®), Bevacizumab (AVASTIN®), Cetuximab (ERBITUX®), Panitumumab (VECTIBIX®), Ipilimumab (YERVOY®), Rituximab (RITUXAN® and MABTHERA®), Alemtuzumab (CAMPATH®), Ofatumumab (ARZERRA®), Gemtuzumab ozogamicin (MYLOTARG®), Brentuximab vedotin (ADCETRIS®), 90 Y-Ibritumomab Tiuxetan (ZEVALIN®), and ¹³¹I-Tositumomab (BEXXAR®), which are described in detail in Scott et al. (Cancer Immun., 12:14-21, 2012); incorporated herein by reference.

Additional agents that can be conjugated to, admixed with, or administered separately from antagonistic TNFRSF member polypeptides (e.g., single-chain polypeptides, antibodies, or fragments thereof, such as anti-CD40 polypeptides that bind CD40) of the disclosure include T-lymphocytes that exhibit reactivity with a specific antigen associated with a particular pathology. For instance, antagonistic TNFRSF member polypeptides (e.g., single-chain polypeptides, antibodies, or fragments thereof, such as anti-CD40 polypeptides that bind CD40) of the disclosure can be formulated for administration with a T-cell that expresses a chimeric antigen receptor (CAR-T) in order to treat a cell proliferation disorder, such as a cancer described herein. Antagonistic TNFRSF member polypeptides (e.g., single-chain polypeptides, antibodies, or fragments thereof, such as anti-CD40 polypeptides that bind CD40) can synergize with CAR-T therapy by preventing T-reg cells from deactivating T-lymphocytes that have been genetically modified so as to express tumor-reactive antigen receptors. In this way, CAR-T cells can be administered to a subject prior to, concurrently with, or after administration of an antagonistic TNFRSF member single-chain polypeptide, antibody, or antigen-binding fragment thereof in order to treat a mammalian subject (e.g., a human) suffering from a cell proliferation disorder, such as cancer.

CAR-T therapy is a particularly robust platform for targeting cancer cells in view of the ability to genetically engineer T-lymphocytes to express an antigen receptor specific to a tumor-associated antigen. For instance, identification of antigens overexpressed on the surfaces of tumors and other cancer cells can inform the design and discovery of chimeric T-cell receptors, which are often composed of cytoplasmic and transmembrane domains derived from a naturally-occurring T-cell receptor operatively linked to an extracellular scFv fragment that specifically binds to a particular antigenic peptide. T-cells can be genetically modified in order to express an antigen receptor that specifically binds to a particular tumor antigen by any of a variety of genome editing techniques described herein or known in the art. Exemplary techniques for modifying a T-cell genome so as to incorporate a gene encoding a chimeric antigen receptor include the CRISPER/Cas, zinc finger nuclease, TALEN, ARCUS™ platforms described herein. Methods for the genetic engineering of CAR-T lymphocytes have been described, e.g., in WO 2014/127261, WO 2014/039523, WO 2014/099671, and WO 20120790000; the disclosures of each of which are incorporated by reference herein.

CAR-T cells useful in the compositions and methods of the disclosure include those that have been genetically modified such that the cell does not express the endogenous T-cell receptor. For instance, a CAR-T cell may be modified by genome-editing techniques, such as those described herein, so as to suppress expression of the endogenous T-cell receptor in order to prevent graft-versus-host reactions in a subject receiving a CAR-T infusion. Additionally or alternatively, CAR-T cells can be genetically modified so as to reduce the expression of one or more endogenous MHC proteins. This is a particularly useful technique for the infusion of allogeneic T-lymphocytes, as recognition of foreign MHC proteins represents one mechanism that promotes allograft rejection. One of skill in the art can also modify a T-lymphocyte so as to suppress the expression of immune suppressor proteins, such as programmed cell death protein 1 (PD-1) and cytotoxic T-lymphocyte-associated protein 4 (CTLA-4). These proteins are cell surface receptors that, when activated, attenuate T-cell activation. Infusion of CAR-T cells that have been genetically modified so as to diminish the expression of one or more immunosuppressor proteins represents one strategy that can be used to prolong the T-lymphocyte-mediated cytotoxicity in vivo.

In addition to deleting specific genes, one can also modify CAR-T cells in order to express a T-cell receptor with a desired antigen specificity. For instance, one can genetically modify a T-lymphocyte in order to express a T-cell receptor that specifically binds to a tumor-associated antigen in order to target infused T-cells to cancerous cells. An exemplary T-cell receptor that may be expressed by a CAR-T cell is one that binds PD-L1, a cell surface protein that is often overexpressed on various tumor cells. As PD-L1 activates PD-1 on the surface of T-lymphocytes, targeting this tumor antigen with CAR-T therapy can synergize with antagonistic TNFRSF member antibodies or antibody fragments of the disclosure in order to increase the duration of an immune response mediated by a T-lymphocyte in vivo. CAR-T cells can also be modified so as to express a T-cell receptor that specifically binds an antigen associated with one or more infectious disease, such as an antigen derived from a viral protein, a bacterial cell, a fungus, or other parasitic organism.

Other pharmaceutical compositions of the disclosure include those that contain an antagonistic TNFRSF member antibody or antibody fragment, interferon alpha, and/or one or more antibiotics that can be administered to a subject (e.g., a human subject) suffering from an infectious disease. For instance, an antagonistic TNFRSF member antibody or antibody fragment can be conjugated to, admixed with, or administered separately from an antibiotic useful for treating one or more infectious diseases, such as amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, meropenem, cefadroxil, cefazolin, cefazlexin, cefaclor, cefoxitin, cefprozil, cefuroxime, cefdinir, cefditoren, cefoperazone, clindamycin, lincomycin, daptomycin, erythromycin, linezolid, torezolid, amoxicillin, ampicillin, bacitracin, ciprofloxacin, doxycycline, and tetracycline, among others.

### Immunotherapy Agents

An antagonistic TNFRSF member polypeptide (e.g., a single-chain polypeptide, antibody, antigen-binding fragment thereof, or construct thereof) described herein may be admixed with, conjugated to, administered with, or administered separately from, an immunotherapy agent, for instance, for the treatment of a cancer or infectious disease, such as a cancer or infectious disease described herein. For instance, an anti-CD40 polypeptide of the disclosure may be admixed with, conjugated to, administered with or separately from, an immunotherapy agent. For instance, an anti-4-1 BB polypeptide of the disclosure may be admixed with, conjugated to, administered with or separately from, an immunotherapy agent. For instance, an anti-CD27 polypeptide of the disclosure may be admixed with, conjugated to, administered with or separately from, an immunotherapy agent. For instance, an anti-CD30 polypeptide of the disclosure may be admixed with, conjugated to, administered with or separately from, an immunotherapy agent. For instance, an anti-DR6 polypeptide of the disclosure may be admixed with, conjugated to, administered with or separately from, an immunotherapy agent. For instance, an anti-EDAR polypeptide of the disclosure may be admixed with, conjugated to, administered with or separately from, an immunotherapy agent. For instance, an anti-Fas polypeptide of the disclosure may be admixed with, conjugated to, administered with or separately from, an immunotherapy agent. For instance, an anti-GITR polypeptide of the disclosure may be admixed with, conjugated to, administered with or separately from, an immunotherapy agent. For instance, an anti-HVEM polypeptide of the disclosure may be admixed with, conjugated to, administered with or separately from, an immunotherapy agent. For instance, an anti-LT beta receptor polypeptide of the disclosure may be admixed with, conjugated to, administered with or separately from, an immunotherapy agent. For instance, an anti-NGFR polypeptide of the disclosure may be admixed with, conjugated to, administered with or separately from, an immunotherapy agent. For instance, an anti-OPG polypeptide of the disclosure may be admixed with, conjugated to, administered with or separately from, an immunotherapy agent. For instance, an anti-OX40 polypeptide of the disclosure may be admixed with, conjugated to, administered with or separately from, an immunotherapy agent. For instance, an anti-RANK polypeptide of the disclosure may be admixed with, conjugated to, administered with or separately from, an immunotherapy agent. For instance, an anti-RELT (19L) polypeptide of the disclosure may be admixed with, conjugated to, administered with or separately from, an immunotherapy agent. For instance, an anti-TNFR1 polypeptide of the disclosure may be admixed with, conjugated to, administered with or separately from, an immunotherapy agent. For instance, an anti-TRAIL-R2 (TNFRSF10B) polypeptide of the disclosure may be admixed with, conjugated to, administered with or separately from, an immunotherapy agent. For instance, an anti-TRAIL-R1 (TNFRSF10A) polypeptide of the disclosure may be admixed with, conjugated to, administered with or separately from, an immunotherapy agent. For instance, an anti-TRAIL-R4 polypeptide of the disclosure may be admixed with, conjugated to, administered with or separately from, an immunotherapy agent. For instance, an anti-TRAMP polypeptide of the disclosure may be admixed with, conjugated to, administered with or separately from, an immunotherapy agent. For instance, an anti-TROY polypeptide of the disclosure may be admixed with, conjugated to, administered with or separately from, an immunotherapy agent. For instance, an anti-XEDAR polypeptide of the disclosure may be admixed with, conjugated to, administered with or separately from, an immunotherapy agent. Exemplary immunotherapy agents useful in conjunction with the compositions and methods described herein include, without limitation, an anti-CTLA-4 agent, an anti-PD-1 agent, an anti-PD-L1 agent, an anti-PD-L2 agent, an anti-TNF-α cross-linking agent, an anti-TRAIL cross-linking agent, and an anti-TWEAKR agent, as well as, for example, agents directed toward the immunological targets described in Table 1 of Mahoney et al., Cancer Immunotherapy, 14:561-584 (2015), the disclosure of which is incorporated herein by reference in its entirety. For example, the immunotherapy agent may be an anti-CTLA-4 antibody or antigen-binding fragment thereof, such as ipilimumab and tremelimumab. The immunotherapy agent may be an anti-PD-1 antibody or antigen-binding fragment thereof, such as nivolumab, pembrolizumab, avelumab, durvalumab, and atezolizumab. The immunotherapy agent may be an anti-PD-L1 antibody or antigen-binding fragment thereof, such as atezolizumab or avelumab. As other examples, immunological target TL1A may be targeted with an anti-TL1A antibody; immunological target LIGHT may be targeted with an anti-LIGHT antibody; immunological target BTLA may be targeted with an anti-BTLA antibody; immunological target LAG3 may be targeted with an anti-LAG3 antibody; immunological target TIM3 may be targeted with an anti-TIM3 antibody; immunological target Singlecs may be targeted with an anti-Singlecs antibody; immunological target ICOS ligand may be targeted with an anti-ICOS ligand antibody; immunological target B7-H3 may be targeted with an anti-B7-H3 antibody; immunological target B7-H4 may be targeted with an anti-B7-H4 antibody; immunological target VISTA may be targeted with an anti-VISTA antibody; immunological target TMIGD2 may be targeted with an anti-TMIGD2 antibody; immunological target BTNL2 may be targeted with an anti-BTNL2 antibody; immunological target CD48 may be targeted with an anti-CD48 antibody; immunological target KIR may be targeted with an anti-KIR antibody; immunological target LIR may be targeted with an anti-LIR antibody; immunological target ILT may be targeted with an anti-ILT antibody; immunological target NKG2D may be targeted with an anti-NKG2D antibody; immunological target NKG2A may be targeted with an anti-NKG2A antibody; immunological target MICA may be targeted with an anti-MICA antibody; immunological target MICB may be targeted with an anti-MICB antibody; immunological target CD244 may be targeted with an anti-CD244 antibody; immunological target CSF1R may be targeted with an anti-CSF1R antibody; immunological target IDO may be targeted with an anti-IDO antibody; immunological target TGFβ may be targeted with an anti-TGFβ antibody; immunological target CD39 may be targeted with an anti-CD39 antibody; immunological target CD73 may be targeted with an anti-CD73 antibody; immunological target CXCR4 may be targeted with an anti-CXCR4 antibody; immunological target CXCL12 may be targeted with an anti-CXCL12 antibody; immunological target SIRPA may be targeted with an anti-SIRPA antibody; immunological target CD47 may be targeted with an anti-CD47 antibody; immunological target VEGF may be targeted with an anti-VEGF antibody; and immunological target neuropilin may be targeted with an anti-neuropilin antibody (see, e.g., Table 1 of Mahoney et al.).

Immunotherapy agents that may be used in conjunction with the compositions and methods described herein include, for instance, an anti-TWEAK agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-LIGHT agent, an anti-DR4 agent, an anti-CR5 agent, an anti-CD95 agent, an anti-TRAIL agent, an anti-RANKL agent, an anti-BCMA agent, an anti-TACI agent, and an anti-BAFFR agent. For instance, the immunotherapy agent may be an anti-TWEAK antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-LIGHT antibody or antigen-binding fragment thereof, an anti-DR4 antibody or antigen-binding fragment thereof, an anti-CR5 antibody or antigen-binding fragment thereof, an anti-CD95 antibody or antigen-binding fragment thereof, an anti-TRAIL antibody or antigen-binding fragment thereof, an anti-RANKL antibody or antigen-binding fragment thereof, an anti-BCMA antibody or antigen-binding fragment thereof, an anti-TACI antibody or antigen-binding fragment thereof, or an anti-BAFFR antibody or antigen-binding fragment thereof.

In some examples, the immunotherapy agent is an anti-cell surface lymphocyte protein antibody or antigen-binding fragment thereof, such as an antibody or antigen-binding fragment thereof that binds one or more of CD1, CD2, CD3, CD4, CD5, CD6, CD7, CD8, CD9, CD10, CD11, CD12, CD13, CD14, CD15, CD16, CD17, CD18, CD19, CD20, CD21, CD22, CD23, CD24, CD25, CD26, CD28, CD29, CD31, CD32, CD33, CD34, CD35, CD36, CD37, CD38, CD39, CD41, CD42, CD43, CD44, CD45, CD46, CD47, CD48, CD49, CD50, CD51, CD52, CD53, CD54, CD55, CD56, CD57, CD58, CD59, CD60, CD61, CD62, CD63, CD64, CD65, CD66, CD67, CD68, CD69, CD70, CD71, CD72, CD73, CD74, CD75, CD76, CD77, CD78, CD79, CD80, CD81, CD82, CD83, CD84, CD85, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD108, CD109, CD110, CD111, CD112, CD113, CD114, CD115, CD116, CD117, CD118, CD119, CD120, CD121, CD122, CD123, CD124, CD125, CD126, CD127, CD128, CD129, CD130, CD131, CD132, CD133, CD134, CD135, CD136, CD138, CD139, CD140, CD141, CD142, CD143, CD144, CD145, CD146, CD147, CD148, CD149, CD150, CD151, CD152, CD153, CD154, CD155, CD156, CD157, CD158, CD159, CD160, CD161, CD162, CD163, CD164, CD165, CD166, CD167, CD168, CD169, CD170, CD171, CD172, CD173, CD174, CD175, CD176, CD177, CD178, CD179, CD180, CD181, CD182, CD183, CD184, CD185, CD186, CD187, CD188, CD189, CD190, CD191, CD192, CD193, CD194, CD195, CD196, CD197, CD198, CD199, CD200, CD201, CD202, CD203, CD204, CD205, CD206, CD207, CD208, CD209, CD210, CD211, CD212, CD213, CD214, CD215, CD216, CD217, CD218, CD219, CD220, CD221, CD222, CD223, CD224, CD225, CD226, CD227, CD228, CD229, CD230, CD231, CD232, CD233, CD234, CD235, CD236, CD237, CD238, CD239, CD240, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CD293, CD294, CD295, CD296, CD297, CD298, CD299, CD300, CD301, CD302, CD303, CD304, CD305, CD306, CD307, CD308, CD309, CD310, CD311, CD312, CD313, CD314, CD315, CD316, CD317, CD318, CD319, and/or CD320.

In some examples, the immunotherapy agent is an agent (e.g., a polypeptide, antibody, antigen-binding fragment thereof, a single-chain polypeptide, or construct thereof) that binds a chemokine or lymphokine, such as a chemokine or lymphokine involved in tumor growth. For instance, exemplary immunotherapy agents that may be used in conjunction with the compositions and methods described herein include agents (e.g., polypeptides, antibodies, antigen-binding fragments thereof, single-chain polypeptides, and constructs thereof) that bind and inhibit the activity of one or more, or all, of CXCL1, CXCL2, CXCL3, CXCL8, CCL2 and CCLS. Exemplary chemokines involved in tumor growth and that may be targeted using an immunotherapy agent as described herein include those described, for instance, in Chow et al., Cancer Immunol. Res., 2:1125-1131, 2014, the disclosure of which is incorporated herein by reference. Exemplary immunotherapy agents that may be used in conjunction with the compositions and methods described herein additionally include agents (e.g., polypeptides, antibodies, antigen-binding fragments thereof, single-chain polypeptides, and constructs thereof) that bind and inhibit the activity of one or more, or all, of CCL3, CCL4, CCL8, and CCL22, which are described, for instance, in Balkwill, Nat. Rev. Cancer, 4:540-550, 2004, the disclosure of which is incorporated herein by reference.

Additional examples of immunotherapy agents that can be used in conjunction with the compositions and methods described herein include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies such as Vorinostat.

Using the methods described herein, an antagonistic TNFRSF member polypeptide (e.g., a single-chain polypeptide, antibody, antigen-binding fragment thereof, or construct thereof) described herein may be co-administered with (e.g., admixed with) or administered separately from an immunotherapy agent. For example, an antagonistic TNFRSF member polypeptide described herein (such as a single-chain polypeptide, antibody, antigen-binding fragment thereof, or construct described herein) may be administered to a subject, such as a human subject suffering from a cancer or infectious disease, simultaneously or at different times. In some examples, the antagonistic TNFRSF member polypeptide (e.g., a single-chain polypeptide, antibody, antigen-binding fragment thereof, or construct described herein) is administered to the subject prior to administration of an immunotherapy agent to the subject. Alternatively, the antagonistic TNFRSF member polypeptide (e.g., a single-chain polypeptide, antibody, antigen-binding fragment thereof, or construct described herein) may be administered to the subject after an immunotherapy agent. For example, the antagonistic TNFRSF member polypeptide (e.g., a single-chain polypeptide, antibody, antigen-binding fragment thereof, or construct described herein) may be administered to the subject after a failed immunotherapy treatment. A physician of skill in the art can monitor the efficacy of immunotherapy treatment to determine whether the therapy has successfully ameliorated the pathology being treated (such as a cancer or infectious disease, e.g., a cancer or infectious disease described herein) using methods described herein and known in the art.

For instance, a physician of skill in the art may monitor the quantity of cancer cells in a sample isolated from a subject (e.g., a blood sample or biopsy sample), such as a human subject, for instance, using flow cytometry or FACS analysis. Additionally, or alternatively, a physician of skill in the art can monitor the progression of a cancerous disease in a subject, for instance, by monitoring the size of one or more tumors in the subject, for example, by CT scan, MRI, or X-ray analysis. A physician of skill in the art may monitor the progression of a cancer, such as a cancer described herein, by evaluating the quantity and/or concentration of tumor biomarkers in the subject, such as the quantity and/or concentration of cell surface-bound tumor associated antigens or secreted tumor antigens present in the blood of the subject as an indicator of tumor presence. A finding that the quantity of cancer cells, the size of a tumor, and/or the quantity or concentration of one or more tumor antigens present in the subject or in a sample isolated from the subject has not decreased, for instance, by a statistically significant amount following administration of the immunotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the immunotherapy treatment has failed to ameliorate the cancer. Based on this indication, a physician of skill in the art may administer an antagonistic TNFRSF member polypeptide described herein, such as a single-chain polypeptide, antibody, antigen-binding fragment thereof, or construct described herein. Similarly, a physician a physician of skill in the art may monitor the quantity of bacterial, fungal, or parasitic cells, or the quantity of viral particles in a sample isolated from a subject suffering from an infectious disease, such as an infectious disease described herein. Additionally, or alternatively, a physician of skill in the art may monitor the progression of an infectious disease by evaluating the symptoms of a subject suffering from such a pathology. For instance, a physician may monitor the subject by determining whether the frequency and/or severity of one or more symptoms of the infectious disease have stabilized (e.g., remained the same) or decreased following treatment with an immunotherapy agent. A finding that the quantity of bacterial, fungal, or parasitic cells or viral particles in a sample isolated from the subject and/or a finding that the frequency or severity of one or more symptoms of the infectious disease have not decreased, for instance, by a statistically significant amount following administration of the immunotherapy agent within a specified time period (e.g., from 1 day to 6 months, such as 1 day, 2 days, 3 days, 4 days, 5 days, 6 days, 7 days, 2 weeks, 3 weeks, 4 weeks, 2 months, 3 months, 4 months, 5 months, or 6 months) can indicate that the immunotherapy treatment has failed to ameliorate the infectious disease. Based on this indication, a physician of skill in the art may administer an antagonistic TNFRSF member polypeptide described herein, such as a single-chain polypeptide, antibody, antigen-binding fragment thereof, or construct described herein.

### Blood-Brain Barrier Penetration

In certain examples, antagonistic TNFRSF member polypeptides (e.g., single-chain polypeptides, antibodies, or fragments thereof, such as anti-CD40 polypeptides that bind CD40) of the disclosure can be formulated to ensure proper distribution in vivo. For example, the blood-brain barrier (BBB) excludes many highly hydrophilic compounds. To ensure that the therapeutic compositions of the disclosure cross the BBB (if desired), they can be formulated, for example, in liposomes. Methods of manufacturing liposomes have been described, e.g., U.S. Pat. Nos. 4,522,811; 5,374,548; and 5,399,331. The liposomes may comprise one or more moieties that are selectively transported into specific cells or organs, thereby enhancing targeted drug delivery (see, e.g., V. V. Ranade (J. Clin. Pharmacol. 29:685, 1989)). Exemplary targeting moieties include, e.g., folate or biotin (see, e.g., U.S. Pat. No. 5,416,016); mannosides (Umezawa et al., Biochem. Biophys. Res. Commun. 153:1038, 1988); antibodies (P. G. Bloeman et al., FEBS Lett. 357:140, 1995); M. Owais et al., Antimicrob. Agents Chemother. 39:180, 1995); surfactant protein A receptor (Briscoe et al., Am. J. Physiol. 1233:134, 1995); the disclosures of each of which are incorporated herein by reference.

### Routes of Administration and Dosing

Antagonistic TNFRSF member polypeptides (e.g., single-chain polypeptides, antibodies, or fragments thereof, such as anti-CD40 polypeptides that bind CD40) of the disclosure can be administered to a mammalian subject (e.g., a human) by a variety of routes such as orally, transdermally, subcutaneously, intranasally, intravenously, intramuscularly, intraocularly, intratumorally, parenterally, topically, intrathecally and intracerebroventricularly. The most suitable route for administration in any given case will depend on the particular antibody or antigen-binding fragment administered, the subject, pharmaceutical formulation methods, administration methods (e.g., administration time and administration route), the subject's age, body weight, sex, severity of the diseases being treated, the subject's diet, and the subject's excretion rate.

The effective dose of an anti-TNFRSF member single-chain polypeptide, antibody, or antigen-binding fragment thereof of the disclosure can range from about 0.0001 to about 100 mg/kg of body weight per single (e.g., bolus) administration, multiple administrations or continuous administration, or to achieve a serum concentration of 0.0001-5000 μg/mL serum concentration per single (e.g., bolus) administration, multiple administrations or continuous administration, or any effective range or value therein depending on the condition being treated, the route of administration and the age, weight, and condition of the subject. In certain examples, e.g., for the treatment of cancer, each dose can range from about 0.0001 mg to about 500 mg/kg of body weight. For instance, a pharmaceutical composition of the disclosure may be administered in a daily dose in the range of 0.001-100 mg/kg (body weight). The dose may be administered one or more times (e.g., 2-10 times) per day, week, month, or year to a mammalian subject (e.g., a human) in need thereof.

Therapeutic compositions can be administered with medical devices known in the art. For example, in an embodiment, a therapeutic composition of the disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the disclosure include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

### Kits Containing Antagonistic Anti-TNFRSF Member Polypeptides

This disclosure also includes kits that contain antagonistic anti-TNFRSF member polypeptides (e.g., single-chain polypeptides, antibodies, or fragments thereof, such as anti-CD40 polypeptides that bind CD40). The kits provided herein may contain any of the antagonistic TNFRSF member polypeptides described above, as well as any of the polynucleotides encoding these polypeptides, vectors containing these polypeptides, or cells engineered to express and secrete polypeptides of the disclosure (e.g., prokaryotic or eukaryotic cells). A kit of this disclosure may include reagents that can be used to produce the compositions of the disclosure (e.g., antagonistic anti-TNFRSF member polypeptides, such as single-chain polypeptides, antibodies, or fragments thereof, conjugates containing antagonistic anti-TNFRSF member polypeptides, polynucleotides encoding antagonistic anti-TNFRSF member polypeptides, vectors containing these polypeptides). Optionally, kits of the disclosure may include reagents that can induce the expression of antagonistic TNFRSF member antibodies within cells (e.g., mammalian cells), such as doxycycline or tetracycline. In other cases, a kit of the disclosure may contain a compound capable of binding and detecting a fusion protein that contains an antagonistic TNFRSF member polypeptide and an epitope tag. For instance, in such cases a kit of the disclosure may contain maltose, glutathione, a nickel-containing complex, an anti-FLAG antibody, an anti-myc antibody, an anti-HA antibody, biotin, or streptavidin.

Kits of the disclosure may also include reagents that are capable of detecting an antagonistic TNFRSF member single-chain polypeptide, antibody, or fragment thereof directly. Examples of such reagents include secondary antibodies that selectively recognize and bind particular structural features within the Fc region of an anti-TNFRSF member antibody of the disclosure. Kits of the disclosure may contain secondary antibodies that recognize the Fc region of an antagonistic TNFRSF member antibody and that are conjugated to a fluorescent molecule. These antibody-fluorophore conjugates provide a tool for analyzing the localization of antagonistic anti-TNFRSF member antibodies, e.g., in a particular tissue or cultured mammalian cell using established immunofluorescence techniques. In some examples, kits of the disclosure may include additional fluorescent compounds that exhibit known sub-cellular localization patterns. These reagents can be used in combination with another antibody-fluorophore conjugate, e.g., one that specifically recognizes a different receptor on the cell surface in order to analyze the localization of an anti-TNFRSF member antibody relative to other cell-surface proteins.

Kits of the disclosure may also contain a reagent that can be used for the analysis of a subject's response to treatment by administration of antagonistic TNFRSF member polypeptides (e.g., single-chain polypeptides, antibodies, or fragments thereof, such as anti-CD40 polypeptides that bind CD40) of the disclosure. For instance, kits of the disclosure may include an antagonistic TNFRSF member polypeptide, such as a single-chain polypeptide, antibody, or antibody fragment, and one or more reagents that can be used to determine the quantity of T-reg cells in a blood sample withdrawn from a subject (e.g., a human who was administered the antagonist) that is undergoing treatment with an antibody of the disclosure. Such a kit may contain, e.g., antibodies that selectively bind cell-surface antigens presented by T-reg cells, such as CD4 and CD25. Optionally, these antibodies may be labeled with a fluorescent dye, such as fluorescein or tetramethylrhodamine, in order to facilitate analysis of a population of T-reg cells by fluorescence-activated cell sorting (FACS) methods known in the art. Kits of the disclosure may optionally contain one or more reagents that can be used to quantify a population of tumor-reactive T-lymphocytes in order to determine the effectiveness of an antagonistic TNFRSF member polypeptide of the disclosure in restoring tumor-infiltrating lymphocyte proliferation. For instance, kits of the disclosure may contain an antibody that selectively binds cell-surface markers on the surface of a cytotoxic T-cell, such as CD8 or CD3. Optionally, these antibodies may be labeled with fluorescent molecules so as to enable quantitation by FACS analysis.

A kit of the disclosure may also contain one or more reagents useful for determining the affinity and selectivity of an antagonistic TNFRSF member single-chain polypeptide, antibody, or antigen-binding fragment thereof of the disclosure for one or more peptides derived from a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40), e.g., a peptide containing the sequence of any one of SEQ ID NOs: 25-66, or a variant thereof with up to 80% or greater sequence identity thereto. For instance, a kit may contain an antagonistic TNFRSF member antibody and one or more reagents that can be used in an ELISA assay to determine the K_{d} of an antibody of the disclosure for one or more peptides that present a TNFRSF member epitope in a conformation similar to that of the epitope in the native protein. A kit may contain, e.g., a microtiter plate containing wells that have been previously conjugated to avidin, and may contain a library of TNFRSF member member-derived peptides, each of which conjugated to a biotin moiety. Such a kit may optionally contain a secondary antibody that specifically binds to the Fc region of an antagonistic TNFRSF member antibody of the disclosure, and the secondary antibody may be conjugated to an enzyme (e.g., horseradish peroxidase) that catalyzes a chemical reaction that results in the emission of luminescent light.

Kits of the disclosure may also contain antagonistic TNFRSF member polypeptides (e.g., single-chain polypeptides, antibodies, or fragments thereof, such as anti-CD40 polypeptides that bind CD40) of the disclosure and reagents that can be conjugated to such a polypeptide, including those previously described (e.g., a cytotoxic agent, a fluorescent molecule, a bioluminescent molecule, a molecule containing a radioactive isotope, a molecule containing a chelating group bound to a paramagnetic ion, etc.). These kits may additionally contain instructions for how the conjugation of an antagonistic TNFRSF member polypeptide of the disclosure to a second molecule, such as those described above, can be achieved.

A kit of the disclosure may also contain a vector containing a polynucleotide that encodes an antagonistic anti-TNFRSF member single-chain polypeptide, antibody, or fragment thereof, such as any of the vectors described herein. Alternatively, a kit may include mammalian cells (e.g., CHO cells) that have been genetically altered to express and secrete antagonistic TNFRSF member polypeptides (e.g., single-chain polypeptides, antibodies, or fragments thereof, such as anti-CD40 polypeptides that bind CD40) from the nuclear genome of the cell. Such a kit may also contain instructions describing how expression of the antagonistic TNFRSF member single-chain polypeptide, antibody, or fragment thereof from a polynucleotide can be induced, and may additionally include reagents (such as, e.g., doxycycline or tetracycline) that can be used to promote the transcription of these polynucleotides. Such kits may be useful for the manufacture of antagonistic TNFRSF member antibodies or antigen-binding fragments thereof of the disclosure.

Other kits of the disclosure may include tools for engineering a prokaryotic or eukaryotic cell (e.g., a CHO cell or a BL21(DE3) E. coli cell) so as to express and secrete an antagonistic TNFRSF member single-chain polypeptide, antibody, or fragment thereof of the disclosure from the nuclear genome of the cell. For example, a kit may contain CHO cells stored in an appropriate media and optionally frozen according to methods known in the art. The kit may also provide a vector containing a polynucleotide that encodes a nuclease (e.g., such as the CRISPER/Cas, zinc finger nuclease, TALEN, ARCUS™ nucleases described herein) as well as reagents for expressing the nuclease in the cell. The kit can additionally provide tools for modifying the polynucleotide that encodes the nuclease so as to enable one to alter the DNA sequence of the nuclease in order to direct the cleavage of a specific target DNA sequence of interest. Examples of such tools include primers for the amplification and site-directed mutagenesis of the polynucleotide encoding the nuclease of interest. The kit may also include restriction enzymes that can be used to selectively excise the nuclease-encoding polynucleotide from the vector and subsequently re-introduce the modified polynucleotide back into the vector once the user has modified the gene. Such a kit may also include a DNA ligase that can be used to catalyze the formation of covalent phosphodiester linkages between the modified nuclease-encoding polynucleotide and the target vector. A kit of the disclosure may also provide a polynucleotide encoding an antagonistic anti-TNFRSF member single-chain polypeptide, antibody, or fragment thereof, as well as a package insert describing the methods one can use to selectively cleave a particular DNA sequence in the genome of the cell in order to incorporate the polynucleotide encoding an antagonistic TNFRSF member polypeptide into the genome at this site. Optionally, the kit may provide a polynucleotide encoding a fusion protein that contains an antagonistic TNFRSF member single-chain polypeptide, antibody, or fragment thereof and an additional polypeptide, such as, e.g., those described herein.

A kit of the disclosure can be used to measure the soluble secreted TNFRSF member. Such a kit may contain an antibody of the disclosure that selectively binds cell-surface a specific TNFRSF member protein (e.g., CD40, TRAIL-R1 (TNFRSF10A), TRAIL-R2 (TNFRSF10B), TRAMP, DR6, NGFR, TNFR1, FAS, EDAR, TRAIL-R3, TRAIL-R4, RANK, FN14, LT beta receptor, HVEM, CD30, CD27, 4-1 BB, OX40, GITR, BCMA, TACT, BAFF-R, XEDAR, TROY, or RELT (19L)) and reagents that can be conjugated to such a polypeptide. Optionally, these antibodies may include those previously described (e.g., a fluorescent molecule, a bioluminescent molecule, a molecule containing a radioactive isotope, a molecule containing a chelating group bound to a paramagnetic ion, etc.) to quantify the level of TNFRSF member protein in a subject. The level of secreted soluble TNFRSF member protein can be measured again after administration of the antibody of the disclosure to a subject to determine if administration has caused a decrease in the level of secreted soluble TNFRSF member protein. For example, the level of secreted soluble TNFRSF member protein in a subject can be measured one day, two days, three days, four days, five days, six days, one week, eight days, nine days, ten days, eleven days, twelve days, thirteen days, fourteen days, fifteen days, sixteen days, seventeen days, eighteen days, nineteen days, twenty days, three weeks, four weeks, five weeks, six weeks, seven weeks, eight weeks, or more after administration of the antibody of the disclosure. The subject can be administered another dose of the antibody or polypeptide of the disclosure if the level of secreted soluble TNFRSF member protein is about the same or higher than the normal level of TNFRSF member or a previously measured level of secreted soluble TNFRSF member protein.

### Examples

The following examples are put forth so as to provide those of ordinary skill in the art with a description of how the compositions and methods claimed herein are performed, made, and evaluated, and are intended to be purely exemplary of the disclosure and are not intended to limit the scope of what the inventor regards as her invention.

### Example 1. Mapping the Discrete Epitopes within TNFRSF Members

Libraries of linear, cyclic, and bicyclic peptides derived from human TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) were screened for distinct sequences within the protein that exhibit affinity (e.g., K_{d}<1 μM) for TNFRSF member antibodies. In order to screen conformational epitopes within TNFRSF members, peptides from distinct regions of the primary protein sequence were conjugated to one another to form chimeric peptides. These peptides contained cysteine residues at strategic positions within their primary sequences. This facilitated an intramolecular cross-linking strategy that was used to constrain individual peptides to a one of a wide array of three dimensional conformations. Unprotected thiols of cysteine residues were cross-linked via nucleophilic substitution reactions with divalent and trivalent electrophiles, such as 2,6-bis(bromomethyl)pyridine and 1,3,5-tris(bromomethyl)benzene, so as to form conformationally restricted cyclic and bicyclic peptides, respectively. In this way, peptides containing unique combinations of amino acids from disparate regions of the TNFRSF member primary sequence were constrained so as to structurally pre-organize epitopes that may resemble those presented in the native TNFRSF member tertiary structure. Libraries containing these peptides were screened by immobilizing peptides to distinct regions of a solid surface and treating the surface in turn with a TNFRSF member antibody, secondary antibody conjugated to horseradish peroxidase (HRP), and HRP substrate (2,2′-azino-di-3-ethylbenzthiazoline sulfonate) in the presence of hydrogen peroxide. The solid surface was washed in between treatment with successive reagents so as to remove excess or non-specifically bound materials. The luminescence of each region of each surface was subsequently analyzed using a charge coupled device (CCD)—camera and an image processing system.

The “Constrained Libraries of Peptides on Surfaces” (CLIPS) platform starts with the conversion of the target protein, e.g., a TNFRSF member, into a library of up to 10,000 overlapping peptide constructs, using a combinatorial matrix design (Timmerman et al., J. Mol. Recognit. 20: 283-29, 2007). On a solid carrier, a matrix of linear peptides is synthesized, which are subsequently shaped into spatially defined CLIPS constructs. Constructs representing multiple parts of the discontinuous epitope in the correct conformation bind the antibody with high affinity, which is detected and quantified. Constructs presenting the incomplete epitope bind the antibody with lower affinity, whereas constructs not containing the epitope do not bind at all. Affinity information is used in iterative screens to define the sequence and conformation of epitopes in detail. These results informed the analysis of epitopes present on the surface of TNFRSF members that bind antagonistic TNFRSF member antibodies as shown in FIGS. 1A-1V.

### Peptide Synthesis

To reconstruct epitopes of the target molecule a library of peptides was synthesized. An amino functionalized polypropylene support was obtained by grafting a proprietary hydrophilic polymer formulation via reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS® liquid handling stations (Perkin Elmer). CLIPS technology allows one to structure peptides into single loops, double-loops, triple loops, sheet-like folds, helix-like folds and combinations thereof. CLIPS templates are coupled to cysteine residues. The side-chains of multiple cysteines in the peptides are coupled to one or two CLIPS templates. For example, a 0.5 mM solution of the CLIPS template (2,6-bis(bromomethyl)pyridine) is dissolved in ammonium bicarbonate (20 mM, pH 7.8)/acetonitrile (1:3(v/v)). This solution is added to a surface-bound peptide array. The CLIPS template will react with side-chains of two cysteines as present in the solid-phase bound peptides of the peptide-arrays (455 wells plate with 3 μl wells). The peptide arrays are gently shaken in the solution for 30 to 60 minutes while completely covered in solution. Finally, the peptide arrays are washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes.

### Analysis of Binding Affinities of Antagonistic TNFRSF Member Antibodies by Surface Plasmon Resonance

The affinities of antagonistic TNFRSF member antibodies for recombinant human TNFRSF members can be measured using BIACORE™ Analysis Services (Precision Antibody). Briefly, the antibody is biotinylated at a 5:1 stoichiometric ratio using biotinyl-LC-LC-NOSE (Thermo-Fisher) in PBS. Excess biotinylation reagent can be removed by centrifugation chromatography and the biotinylated antibody is captured on 3000 RU of streptavidin surface to a level of 100 RU. Theoretical maximum of signal with a TNFRSF member (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40) with that level of antibody capture is 20-30 RU and that signal can be reached with a preliminary experiment using 500 nM TNFRSF member in the running buffer. Analysis of the kinetics of antigen binding can be performed at a flow of 60 μL/min with 2 min injections. Antibodies can be injected at a concentration of 1 mg/ml to the final capture of 100 RU. BIACORE™ 3000 is a typical instrument used for these measurements and can be used with the BioCap chip (GE Healthcare). Double reference method can be used for analysis. Reference channel should contain the identical level of streptavidin.

### ELISA Screening

The binding of antibody to each of the synthesized peptides can be tested in an ELISA format. Surface-immobilized peptide arrays are incubated with primary antibody solution (overnight at 4° C.). After washing, the peptide arrays are incubated with a 1/1000 dilution of an appropriate antibody peroxidase conjugate (SBA) for one hour at 25° C. After washing, the peroxidase substrate 2,2′-azino-di-3-ethylbenzthiazoline sulfonate (ABTS) and 2 μl/m1 of 3 percent H₂O₂ are added. After one hour, the color development can be measured. The color development can be quantified with a charge coupled device (CCD)—camera and an image processing system. The values to be obtained from the CCD camera can range from 0 to 3000 mAU, similar to a standard 96-well plate ELISA-reader. Occasionally a well may contain an air-bubble resulting in a false-positive value. In this case, the cards can be manually inspected and any values caused by an air-bubble are scored as 0.

To verify the quality of the synthesized peptides, a separate set of positive and negative control peptides can be synthesized in parallel. These can be screened with a negative control, antibody 57.9, an antibody that does not specifically bind a TNFRSF member (Posthumus et al., J. Virology. 64:3304-3309, 1990).

### Epitope Mapping

ELISA can be used to determine linear epitopes present on the extracellular surface of TNFRSF members (e.g., CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, or XEDAR, such as CD40). Linear peptides corresponding to various regions within the TNFRSF member primary sequence can be designed and obtained from a suitable vendor (e.g., GenScript (Piscataway, NJ)), diluted in coating buffer, and placed on Immulon 4HBX Flat Bottom Microtiter Plates (Thermo Scientific) at a concentration of 1 μg/well. Primary TNFRSF member antagonistic antibodies (0.1 μg/well) can be incubated with substrates. Secondary antibodies against the IgG antibodies can be used to detect the primary antibodies. Absorbance can be measured using the SPECTRAMAX® 190 Absorbance Plate Reader and analyzed with SoftMax Pro 6.3 (Molecular Devices).

### Example 2. Generating Antagonistic TNFRSF Member Antibodies by Phage Display

An exemplary method for in vitro protein evolution of antagonistic TNFRSF member antibodies of the disclosure is phage display, a technique which is well known in the art. Phage display libraries can be created by making a designed series of mutations or variations within a coding sequence for the CDRs of an antibody or the analogous regions of an antibody-like scaffold (e.g., the BC, CD, and DE loops of ¹⁰Fn3 domains). The template antibody-encoding sequence into which these mutations are introduced may be, e.g., a naive human germline sequence as described herein. These mutations can be performed using standard mutagenesis techniques described herein or known in the art. Each mutant sequence thus encodes an antibody corresponding in overall structure to the template except having one or more amino acid variations in the sequence of the template. Retroviral and phage display vectors can be engineered using standard vector construction techniques as described herein or known in the art. P3 phage display vectors along with compatible protein expression vectors, as is well known in the art, can be used to generate phage display vectors for antibody diversification as described herein.

The mutated DNA provides sequence diversity, and each transformant phage displays one variant of the initial template amino acid sequence encoded by the DNA, leading to a phage population (library) displaying a vast number of different but structurally related amino acid sequences. Due to the well-defined structure of antibody hypervariable regions, the amino acid variations introduced in a phage display screen are expected to alter the binding properties of the binding peptide or domain without significantly altering its structure.

In a typical screen, a phage library is contacted with and allowed to bind a TNFRSF member-derived peptide (e.g., a peptide having the sequence of any one of SEQ ID NOs: 25-66, or a variant thereof with up to 80% or greater sequence identity thereto), or a particular subcomponent thereof. To facilitate separation of binders and non-binders, it is convenient to immobilize the target on a solid support. Phage bearing a TNFRSF member-binding moiety can form a complex with the target on the solid support whereas non-binding phage remain in solution and can be washed away with excess buffer. Bound phage can then be liberated from the target by changing the buffer to an extreme pH (pH 2 or pH 10), changing the ionic strength of the buffer, adding denaturants, or other known means. To isolate the binding phage exhibiting the polypeptides of the present disclosure, a protein elution is performed.

The recovered phage can then be amplified through infection of bacterial cells and the screening process can be repeated with the new pool that is now depleted in non-binding antibodies and enriched for antibodies that bind the target peptide. The recovery of even a few binding phage is sufficient to amplify the phage for a subsequent iteration of screening. After a few rounds of selection, the gene sequences encoding the antibodies or antigen-binding fragments thereof derived from selected phage clones in the binding pool are determined by conventional methods, thus revealing the peptide sequence that imparts binding affinity of the phage to the target. During the panning process, the sequence diversity of the population diminishes with each round of selection until desirable peptide-binding antibodies remain. The sequences may converge on a small number of related antibodies or antigen-binding fragments thereof, typically 10-50 out of about 10⁹ to 10¹⁰ original candidates from each library. An increase in the number of phage recovered at each round of selection is a good indication that convergence of the library has occurred in a screen. After a set of binding polypeptides is identified, the sequence information can be used to design other secondary phage libraries, biased for members having additional desired properties (See WO 2014/152660; incorporated herein by reference).

### Example 3. Treatment of Cancer in a Human Subject by Administration of Antagonistic Anti-TNFRSF Member Antibodies

The antagonistic TNFRSF member antibodies of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-LT beta receptor polypeptides that bind LT beta receptor; anti HVEM polypeptides that bind HVEM; anti-CD30 polypeptides that bind CD30; anti-BCMA polypeptides that bind BCMA; anti-TACI polypeptides that bind TACI; anti-BAFF-R polypeptides that bind BAFF-R; anti-FN14 polypeptides that bind FN14; anti-RELT polypeptides that bind RELT; anti-DR6 polypeptides that bind DR6; anti-RANK polypeptides that bind RANK; or anti-TROY polypeptides that bind TROY) can be administered to a human subject in order to treat a cell proliferation disorder, such as cancer. Administration of these antibodies suppresses the growth and proliferation of a population of T-reg cells. Antibodies of the disclosure can also be administered to a subject in order to suppress a T-reg-mediated immune response. For instance, a human subject suffering from cancer, e.g., a cancer described herein, can be treated by administering an antagonistic TNFRSF member antibody of the disclosure by an appropriate route (e.g., intravenously) at a particular dosage (e.g., between 0.001 and 100 mg/kg/day) over a course of days, weeks, months, or years. If desired, the anti-TNFRSF member antibody can be modified, e.g., by hyperglycosylation or by conjugation with PEG, so as to evade immune recognition and/or to improve the pharmacokinetic profile of the antibody.

The progression of the cancer that is treated with an antagonistic TNFRSF member antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-TNFRSF member antibody of the disclosure. Optionally, cells can be extracted from the subject and a quantitative biochemical analysis can be conducted in order to determine the relative cell-surface concentrations of various growth factor receptors, such as the epidermal growth factor receptor. Based on the results of these analyses, a physician may prescribe higher/lower dosages or more/less frequent dosing of the antagonistic TNFRSF member antibody in subsequent rounds of treatment.

### Example 4. Producing a scFv TNFRSF Member Antagonist

Antibody fragments of the disclosure include scFv fragments, which consist of the antibody variable regions of the light and heavy chains combined in a single peptide chain. A TNFRSF member antibody can be used as a framework for the development of a scFv antibody fragment by recombinantly expressing a polynucleotide encoding the variable region of a light chain of the TNFRSF member antibody operatively linked to the variable region of a heavy chain of that antibody. This can be accomplished using established mutagenesis protocols as described herein or known in the art. This polynucleotide can then be expressed in a cell (e.g., a CHO cell) and the scFv fragment can subsequently be isolated from the cell culture media.

Alternatively, scFv fragments derived from a TNFRSF member antagonist can be produced by chemical synthetic methods (e.g., by Fmoc-based solid-phase peptide synthesis, as described herein). One of skill in the art can chemically synthesize a peptide chain consisting of the variable region of a light chain of the TNFRSF member antibody operatively linked to the variable region of a heavy chain of that antibody. Native chemical ligation can be used as a strategy for the synthesis of long peptides (e.g., greater than 50 amino acids). Native chemical ligation protocols are known in the art and have been described, e.g., by Dawson et al. (Science, 266:776-779, 1994); incorporated herein by reference.

### Example 5. Producing a Humanized TNFRSF Member Antibody

One method for producing humanized TNFRSF member antibodies of the disclosure is to import the CDRs of a TNFRSF member antibody into a human antibody consensus sequence. Consensus human antibody heavy chain and light chain sequences are known in the art (see e.g., the “VBASE” human germline sequence database; Kabat et al. (Sequences of Proteins of Immunological Interest, Fifth Edition, U.S. Department of Health and Human Services, NIH Publication No. 91-3242, 1991); Tomlinson et al. (J. Mol. Biol. 227:776-798, 1992); and Cox et al. (Eur. J. Immunol. 24:827-836, 1994); incorporated herein by reference). Using established procedures, one can identify the variable domain framework residues and CDRs of a consensus antibody sequence (e.g., by sequence alignment (see Kabat, supra)). One can substitute one or more CDRs of the heavy chain and/or light chain variable domains of consensus human antibody with one or more corresponding CDRs of a TNFRSF member antagonist antibody described herein, in order to produce a humanized anti-TNFRSF member antibody variant using gene editing techniques described herein or known in the art. One example of a variable domain of a consensus human antibody includes the heavy chain variable domain EVQLVESGGGLVQPGGSLRLSCAASGFTFSDYAMSWVRQAPGKGLEWVAVISENGSDTYYADSVKGR FTISRDDSKNTLYLQMNSLRAEDTAVYYCARDRGGAVSYFDVWGQGTLVTVSS and the light chain variable domain DIQMTQSPSSLSASVGDRVTITCRASQDVSSYLAWYQQKPGKAPKLLIYAASSLESGVPSRFSGSGSGT DFTLTISSLQPEDFATYYCQQYNSLPYTFGQGTKVEIKRT, identified in U.S. Pat. No. 6,054,297; incorporated herein by reference (CDRs are shown in bold).

In order to produce humanized TNFRSF member antibodies, one can recombinantly express a polynucleotide encoding the above consensus sequence in which one or more variable region CDRs have been replaced with one or more variable region CDR sequences of a TNFRSF member-specific antibody.

A polynucleotide encoding the above heavy chain and light chain variable domains operatively linked to one another can be incorporated into an expression vector (e.g., an expression vector optimized for protein expression in prokaryotic or eukaryotic cells as described herein or known in the art). The single-chain antibody fragment (scFv) can thus be expressed in a host cell and subsequently purified from the host cell medium using established techniques, such as size-exclusion chromatography and/or affinity chromatography as described herein.

### Example 6. Treatment of Cancer in a Human Subject by Administration of Antagonistic Anti-TRAIL-R3 and Anti-TRAIL-R4 Antibodies

The antagonistic anti-TRAIL-R3 and anti-TRAIL-R4 antibodies of the disclosure (e.g., single-chain polypeptides, antibodies, or fragments thereof) can be administered to a human subject in order to treat a cell proliferation disorder, such as cancer (e.g., breast cancer, pancreatic cancer, or adenocarcinoma). Administration of these antibodies suppresses the growth and proliferation of a population of T-reg cells. Antibodies of the disclosure can also be administered to a subject in order to suppress a T-reg-mediated immune response. For instance, a human subject suffering from cancer, e.g., a cancer described herein, can be treated by administering an antagonistic anti-TRAIL-R3 or anti-TRAIL-R4 antibody of the disclosure by an appropriate route (e.g., intravenously) at a particular dosage (e.g., between 0.001 and 100 mg/kg/day) over a course of days, weeks, months, or years. If desired, the anti-TRAIL-R3 or anti-TRAIL-R4 antibody can be modified, e.g., by hyperglycosylation or by conjugation with PEG, so as to evade immune recognition and/or to improve the pharmacokinetic profile of the antibody.

The progression of the cancer that is treated with an antagonistic anti-TRAIL-R3 or anti-TRAIL-R4 antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-TRAIL-R3 or anti-TRAIL-R4 antibody of the disclosure. Optionally, cells can be extracted from the subject and a quantitative biochemical analysis can be conducted in order to determine the relative cell-surface concentrations of various growth factor receptors, such as the epidermal growth factor receptor. Based on the results of these analyses, a physician may prescribe higher/lower dosages or more/less frequent dosing of the antagonistic anti-TRAIL-R3 or anti-TRAIL-R4 antibody in subsequent rounds of treatment.

### Example 7. Treatment of a B Cell Lymphoma, a Melanoma, or a Fibrosarcoma in a Human Subject by Administration of Antagonistic Anti-LT Beta Receptor Antibodies

The antagonistic anti-LT beta receptor antibodies of the disclosure (e.g., single-chain polypeptides, antibodies, or fragments thereof) can be administered to a human subject in order to treat a cell proliferation disorder, such as a B cell lymphoma, a melanoma, or a fibrosarcoma. Administration of these antibodies suppresses the growth and proliferation of a population of T-reg cells. Antibodies of the disclosure can also be administered to a subject in order to suppress a T-reg-mediated immune response. For instance, a human subject suffering from a B cell lymphoma, a melanoma, or a fibrosarcoma can be treated by administering an antagonistic anti-LT beta receptor antibody of the disclosure by an appropriate route (e.g., intravenously) at a particular dosage (e.g., between 0.001 and 100 mg/kg/day) over a course of days, weeks, months, or years. If desired, the anti-LT beta receptor antibody can be modified, e.g., by hyperglycosylation or by conjugation with PEG, so as to evade immune recognition and/or to improve the pharmacokinetic profile of the antibody.

The progression of the B cell lymphoma, the melanoma, or the fibrosarcoma that is treated with an antagonistic anti-LT beta receptor antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-LT beta receptor antibody of the disclosure. Optionally, cells can be extracted from the subject and a quantitative biochemical analysis can be conducted in order to determine the relative cell-surface concentrations of various growth factor receptors, such as epidermal growth factor. Based on the results of these analyses, a physician may prescribe higher/lower dosages or more/less frequent dosing of the antagonistic anti-LT beta receptor antibody in subsequent rounds of treatment.

### Example 8. Treatment of a Cancer in a Human Subject by Administration of Antagonistic Anti-HVEM Antibodies

The antagonistic anti-HVEM antibodies of the disclosure (e.g., single-chain polypeptides, antibodies, or fragments thereof) can be administered to a human subject in order to treat a cell proliferation disorder, such as a cancer (such as, e.g., colorectal cancer, esophageal cancer, gastric cancer, hepatocarcinoma, breast cancer, lymphoma, or a cancer with a low chance of survival). Administration of these antibodies suppresses the growth and proliferation of a population of T-reg cells. Antibodies of the disclosure can also be administered to a subject in order to suppress a T-reg-mediated immune response. For instance, a human subject suffering from mesothelioma, pancreatic cancer, or brain cancer can be treated by administering an antagonistic anti-HVEM antibody of the disclosure by an appropriate route (e.g., intravenously) at a particular dosage (e.g., between 0.001 and 100 mg/kg/day) over a course of days, weeks, months, or years. If desired, the anti-HVEM antibody can be modified, e.g., by hyperglycosylation or by conjugation with PEG, so as to evade immune recognition and/or to improve the pharmacokinetic profile of the antibody.

The progression of mesothelioma, pancreatic cancer, or brain cancer that is treated with an antagonistic anti-HVEM antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-HVEM antibody of the disclosure. Optionally, cells can be extracted from the subject and a quantitative biochemical analysis can be conducted in order to determine the relative cell-surface concentrations of various growth factor receptors, such as epidermal growth factor. Optionally, the extracted cells from the subject may be further analyzed for an increase of CD4+ and CD8+ tumor-infiltrating lymphocytes relative to a prior measurement. Based on the results of these analyses, a physician may prescribe higher/lower dosages or more/less frequent dosing of the antagonistic anti-HVEM antibody in subsequent rounds of treatment.

### Example 9. Treatment of Multiple Myeloma in a Human Subject by Administration of Antagonistic Anti-BCMA Antibodies

The antagonistic anti-BCMA antibodies of the disclosure (e.g., single-chain polypeptides, antibodies, or fragments thereof) can be administered to a human subject in order to treat a cell proliferation disorder, such as multiple myeloma (e.g., a human subject diagnosed with early, late, or refractory multiple myeloma; e.g., the human subject may have experienced a failure of treatment with autologous or allogenic stem cell transplantation, treatment with an alkaloid, and/or chemotherapy). Administration of these antibodies suppresses the growth and proliferation of B cell tumors. For instance, a human subject suffering from multiple myeloma can be treated by administering an antagonistic anti-BCMA antibody of the disclosure by an appropriate route (e.g., intravenously) at a particular dosage (e.g., between 0.001 and 100 mg/kg/day) over a course of days, weeks, months, or years. If desired, the anti-BCMA antibody can be modified, e.g., by hyperglycosylation or by conjugation with PEG, so as to evade immune recognition and/or to improve the pharmacokinetic profile of the antibody.

The progression of multiple myeloma that is treated with an antagonistic anti-BCMA antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-BCMA antibody of the disclosure. Optionally, cells can be extracted from the subject and a quantitative biochemical analysis can be conducted in order to determine the relative cell-surface concentrations of various growth factor receptors, such as epidermal growth factor. Based on the results of these analyses, a physician may prescribe higher/lower dosages or more/less frequent dosing of the antagonistic anti-BCMA antibody in subsequent rounds of treatment.

### Example 10. Treatment of Refractory Multiple Myeloma and Breast Cancer in a Human Subject by Administration of Antagonistic Anti-TACI Antibodies

The antagonistic anti-TACI antibodies of the disclosure (e.g., single-chain polypeptides, antibodies, or fragments thereof) can be administered to a human subject in order to treat a cell proliferation disorder, such as refractory multiple myeloma and breast cancer. Administration of these antibodies suppresses the growth and proliferation of a population of T-reg cells. Antibodies of the disclosure can also be administered to a subject in order to suppress a T-reg-mediated immune response. For instance, a human subject suffering from refractory multiple myeloma or breast cancer can be treated by administering an antagonistic anti-TACI antibody of the disclosure by an appropriate route (e.g., intravenously) at a particular dosage (e.g., between 0.001 and 100 mg/kg/day) over a course of days, weeks, months, or years. If desired, the anti-TACI antibody can be modified, e.g., by hyperglycosylation or by conjugation with PEG, so as to evade immune recognition and/or to improve the pharmacokinetic profile of the antibody.

The progression of refractory multiple myeloma or breast cancer that is treated with an antagonistic anti-TACI antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-TACI antibody of the disclosure. Optionally, cells can be extracted from the subject and a quantitative biochemical analysis can be conducted in order to determine the relative cell-surface concentrations of various growth factor receptors, such as epidermal growth factor. Based on the results of these analyses, a physician may prescribe higher/lower dosages or more/less frequent dosing of the antagonistic anti-TACI antibody in subsequent rounds of treatment.

### Example 11. Treatment of B Cell Acute Lymphoblastic Leukemia in a Human Subject by Administration of Antagonistic Anti-BAFF-R Antibodies

The antagonistic anti-BAFF-R antibodies of the disclosure (e.g., single-chain polypeptides, antibodies, or fragments thereof) can be administered to a human subject in order to treat a cell proliferation disorder, such as B cell acute lymphoblastic leukemia (B-ALL). Administration of these antibodies suppresses the growth and proliferation of a population of T-reg cells. Antibodies of the disclosure can also be administered to a subject in order to suppress a T-reg-mediated immune response. For instance, a human subject suffering from B-ALL can be treated by administering an antagonistic anti-BAFF-R antibody of the disclosure by an appropriate route (e.g., intravenously) at a particular dosage (e.g., between 0.001 and 100 mg/kg/day) over a course of days, weeks, months, or years. If desired, the anti-BAFF-R antibody can be modified, e.g., by hyperglycosylation or by conjugation with PEG, so as to evade immune recognition and/or to improve the pharmacokinetic profile of the antibody.

The progression of B-ALL that is treated with an antagonistic anti-BAFF-R antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-BAFF-R antibody of the disclosure. Optionally, cells can be extracted from the subject and a quantitative biochemical analysis can be conducted in order to determine the relative cell-surface concentrations of various growth factor receptors, such as epidermal growth factor. Based on the results of these analyses, a physician may prescribe higher/lower dosages or more/less frequent dosing of the antagonistic anti-BAFF-R antibody in subsequent rounds of treatment.

### Example 12. Treatment of Metastatic Disease in a Human Subject by Administration of Antagonistic Anti-RANK Antibodies

The antagonistic anti-RANK antibodies of the disclosure (e.g., single-chain polypeptides, antibodies, or fragments thereof) can be administered to a human subject in order to treat a cell proliferation disorder, such as metastatic disease. Administration of these antibodies suppresses the growth and proliferation of a population of T-reg cells. Antibodies of the disclosure can also be administered to a subject in order to suppress a T-reg-mediated immune response. For instance, a human subject suffering from metastatic disease can be treated by administering an antagonistic anti-RANK antibody of the disclosure by an appropriate route (e.g., intravenously) at a particular dosage (e.g., between 0.001 and 100 mg/kg/day) over a course of days, weeks, months, or years. If desired, the anti-RANK antibody can be modified, e.g., by hyperglycosylation or by conjugation with PEG, so as to evade immune recognition and/or to improve the pharmacokinetic profile of the antibody.

The progression of metastatic disease that is treated with an antagonistic anti-RANK antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has further metastasized or if the size of a tumor has changed, e.g., decreased in response to treatment with an anti-RANK antibody of the disclosure. Optionally, cells can be extracted from the subject and a quantitative biochemical analysis can be conducted in order to determine the relative cell-surface concentrations of various growth factor receptors, such as epidermal growth factor. Based on the results of these analyses, a physician may prescribe higher/lower dosages or more/less frequent dosing of the antagonistic anti-RANK antibody in subsequent rounds of treatment.

### Example 13. Treatment of Hodgkin Lymphoma in a Human Subject by Administration of Antagonistic Anti-CD30 Antibodies

An antagonistic anti-CD30 polypeptide of the disclosure (e.g., single-chain polypeptides, antibodies, or fragments thereof) can be administered (e.g., as a monotherapy or as an adjunctive therapy in combination with another therapy, e.g., a checkpoint inhibitor (e.g., an anti-PD-1 agent, anti-PD-L1 agent, or an anti-CTLA-4 agent)) to a human subject in order to treat a cell proliferation disorder, such as Hodgkin lymphoma (or refractory Hodgkin lymphoma). Administration of the antagonistic anti-CD30 polypeptide(s) may suppress the growth and proliferation of cancer cells (e.g., tumor cells referred to as Reed Steinberg cells (typically of B cell lineage)). Additionally or alternatively, administration of the antagonistic anti-CD30 polypeptide(s) may attenuate the activity of T-reg cells, thereby enhancing the ability of the subject to mount a CD8+ effector T cell response against the cancerous cells. This phenotype is particularly beneficial in a subject (or a sample from the subject) with an elevated ratio of T-reg cells to CD8+ T effector cells relative to a ratio of T-reg cells to CD8+ T effector cells in a human (or a sample from the human) that does not have a proliferation disorder such as Hodgkin lymphoma (e.g., the ratio in the subject (or sample) with Hodgkin lymphoma (or a cell proliferation disorder) is greater than the ratio in the human (or sample) without Hodgkin lymphoma (or a cell proliferation disorder) by 5%, 10%, 15%, 20%, 25%, 30%, 35%, 40%, 45%, 50%, 55%, 60%, 65%, 70%, 75%, 80%, 85%, 90%, 95%, 100%, 200%, or more). For instance, a human subject suffering from Hodgkin lymphoma (e.g., a human subject having an elevated ratio (e.g., greater than the ratio relative to a healthy subject by 5% to 200%, or more) of Treg cells to CD8+ T effector cells relative to a healthy subject not suffering from Hodgkin lymphoma) can be treated by administering an antagonistic anti-CD30 antibody of the disclosure by an appropriate route (e.g., intravenously) at a particular dosage (e.g., between 0.001 and 100 mg/kg/day) over a course of days, weeks, months, or years. If desired, the anti-CD30 antibody can be modified, e.g., by hyperglycosylation or by conjugation with PEG, so as to evade immune recognition and/or to improve the pharmacokinetic profile of the antibody.

The progression of Hodgkin lymphoma that is treated with an antagonistic anti-CD30 antibody of the disclosure can be monitored by any one or more of several established methods. A physician can monitor the subject by direct observation in order to evaluate how the symptoms exhibited by the subject have changed in response to treatment. A subject may also be examined by MRI, CT scan, or PET analysis in order to determine if a tumor has metastasized or if the size of a tumor has changed, e.g., decreased in response to treatment with an anti-CD30 antibody of the disclosure. Optionally, cells can be extracted from the subject and a quantitative biochemical analysis can be conducted in order to determine the relative cell-surface concentrations of various growth factor receptors, such as epidermal growth factor. Based on the results of these analyses, a physician may prescribe higher/lower dosages or more/less frequent dosing of the antagonistic anti-CD30 antibody in subsequent rounds of treatment.

### Example 14. Treatment of HIV in a Human Subject by Administration of Antagonistic Anti-TNFRSF Member Antibodies

The antagonistic TNFRSF member antibodies of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT polypeptides that bind RELT) can be administered to a human subject in order to treat a viral infection, such as HIV. Administration of these antibodies suppresses the growth and proliferation of a population of T-reg cells, which enhances the immune response of a subject by allowing the growth and proliferation of cytotoxic T-lymphocytes capable of mounting an attack on infected cells. For instance, a human subject suffering from HIV can be treated by administering an antagonistic TNFRSF member antibody of the disclosure by an appropriate route (e.g., intravenously) at a particular dosage (e.g., between 0.001 and 100 mg/kg/day) over a course of days, weeks, months, or years. If desired, the anti-TNFRSF member antibody can be modified, e.g., by hyperglycosylation or by conjugation with PEG, so as to evade immune recognition and/or to improve the pharmacokinetic profile of the antibody.

The progression of HIV that is treated with an antagonistic TNFRSF member antibody of the disclosure can be monitored by any one or more of several established methods. A physician can monitor the subject by direct observation in order to evaluate how the symptoms exhibited by the subject have changed in response to treatment. A blood sample can also be withdrawn from the subject in order to analyze the cell count of one or more white blood cells in order to determine if the quantity of infected cells has changed (e.g., decreased) in response to treatment with an anti-TNFRSF member antibody of the disclosure. Based on the results of these analyses, a physician may prescribe higher/lower dosages or more/less frequent dosing of the antagonistic TNFRSF member antibody in subsequent rounds of treatment.

### Example 15. Treatment of Mycobacterium tuberculosis in a Non-Human Mammal by Administration of Antagonistic Anti-TNFRSF Member Antibodies

The antagonistic TNFRSF member antibodies of the disclosure (e.g., single-chain polypeptides, antibodies, or fragments thereof, such as anti-TRAMP polypeptides that bind TRAMP; anti-TRAIL-R3 polypeptides that bind TRAIL-R3; anti-TRAIL-R4 polypeptides that bind TRAIL-R4; anti-HVEM polypeptides that bind HVEM; anti-DR6 polypeptides that bind DR6; or anti-RELT polypeptides that bind RELT) can be administered to a non-human mammal (e.g., a bovine mammal, pig, bison, horse, sheep, goat, cow, cat, dog, rabbit, hamster, guinea pig, or other non-human mammal) in order to treat a bacterial infection, such as Mycobacterium tuberculosis. Administration of these antibodies suppresses the growth and proliferation of a population of T-reg cells, which enhances the immune response of a subject by allowing the growth and proliferation of cytotoxic T-lymphocytes capable of mounting an attack on the pathogenic organism. For instance, a non-human mammal suffering from Mycobacterium tuberculosis can be treated by administering an antagonistic TNFRSF member antibody of the disclosure by an appropriate route (e.g., intravenously) at a particular dosage (e.g., between 0.001 and 100 mg/kg/day) over a course of days, weeks, months, or years. If desired, the anti-TNFRSF member antibody can be modified, e.g., by hyperglycosylation or by conjugation with PEG, so as to evade immune recognition and/or to improve the pharmacokinetic profile of the antibody.

The progression of the Mycobacterium tuberculosis infection that is treated with an antagonistic TNFRSF member antibody of the disclosure can be monitored by any one or more of several established methods. A physician can monitor the subject by direct observation in order to evaluate how the symptoms exhibited by the subject have changed in response to treatment. A blood sample can also be withdrawn from the subject in order to analyze the cell count of one or more white blood cells in order to determine if the immune response has changed (e.g., increased) in response to treatment with an anti-TNFRSF member antibody of the disclosure. Based on the results of these analyses, a physician may prescribe higher/lower dosages or more/less frequent dosing of the antagonistic TNFRSF member antibody in subsequent rounds of treatment.

### Example 16. Treatment of an Allograft Rejection in a Human Subject by Administration of Antagonistic TNFRSF Member Polypeptides

The antagonistic TNFRSF member polypeptides of the disclosure (e.g., single-chain polypeptides, antibodies, or fragments thereof such as anti-CD40 polypeptides that bind CD40; anti-TRAIL-R1 polypeptides that bind TRAIL-R1; anti-TRAIL-R2 polypeptides that bind TRAIL-R2; anti-4-1 BB polypeptides that bind 4-1 BB; anti-CD27 polypeptides that bind CD27; and anti-OX40 polypeptides that bind OX40) can be administered to a human subject in order to treat allograft rejection. Administration of these antibodies suppresses the proliferation of T-reg cells, which attenuates immune responses mounted by self-reactive cytotoxic T-cells that are associated with the rejection of a tissue graft following transplantation. For instance, a human subject presenting with allograft rejection can be treated by administering an antagonistic TNFRSF member antibody of the disclosure by an appropriate route (e.g., intravenously) at a particular dosage (e.g., between 0.001 and 100 mg/kg/day) over a course of days, weeks, months, or years. If desired, the antagonistic TNFRSF member antibody can be modified, e.g., by hyperglycosylation or by conjugation with PEG, so as to evade immune recognition and/or to improve the pharmacokinetic profile of the antibody.

The progression of the allograft rejection that is treated with an antagonistic TNFRSF member antibody of the disclosure can be monitored by any one or more of several established methods. A physician can monitor the subject by direct observation in order to evaluate how the symptoms exhibited by the subject have changed in response to treatment. A blood sample can also be withdrawn from the subject in order to analyze the cell count of one or more CD8+ T-cells in order to determine if the quantity of cells has changed (e.g., decreased) in response to treatment with an antagonistic TNFRSF member antibody of the disclosure. A physician may also monitor the fluctuation in the volume of the allograft within the subject during the course of TNFRSF member antibody therapy. Based on the results of these analyses, a physician may prescribe higher/lower dosages or more/less frequent dosing of the antagonistic TNFRSF member antibody in subsequent rounds of treatment in order to preserve the allograft.

### Example 17. Treatment of Alzheimer's Disease in a Human Subject by Administration of an Antagonistic DR6 Polypeptide

An antagonistic DR6 polypeptide of the disclosure (e.g., single-chain polypeptides, antibodies, or fragments thereof) can be administered to a human subject in order to treat a neurological disorder, such as Alzheimer's disease. Administration of these polypeptides (e.g., an antibody or antigen-binding fragment thereof) could reduce or prevent N-APP binding to DR6, which would suppress axon degeneration and neuron death in the subject. For instance, a human subject presenting with Alzheimer's disease could be treated by administering an antagonistic DR6 antibody of the disclosure to the subject by an appropriate route (e.g., intravenously) at a particular dosage (e.g., between 0.001 and 100 mg/kg/day) over a course of days, weeks, months, or years. If desired, the antagonistic DR6 antibody can be modified, e.g., by hyperglycosylation or by conjugation with PEG, so as to improve the pharmacokinetics (e.g., half-life) of the antibody (e.g., by reducing immune recognition) and/or to improve the in vivo distribution of the antibody.

The progression of Alzheimer's disease in the treated subject could be monitored by any one or more of several established methods. For example, a physician could monitor the subject by direct observation in order to evaluate any changes in the symptoms exhibited by the subject following treatment. A blood sample could also be withdrawn from the subject in order to analyze the level of soluble DR6 and/or N-APP in order to determine if the quantity of protein has changed (e.g., decreased) in response to treatment with an antagonistic DR6 antibody of the disclosure. Based on the results of these analyses, a physician may prescribe higher/lower dosages or more/less frequent dosing of the antagonistic DR6 antibody in subsequent rounds of treatment.

### Example 18. Treatment of Type 2 Diabetes in a Human Subject by Administration of an Antagonistic Fas Polypeptide

An antagonistic Fas polypeptide of the disclosure (e.g., single-chain polypeptides, antibodies, or fragments thereof) can be administered to a human subject in order to treat metabolic disorders, such as type 2 diabetes. Administration of these polypeptides (e.g., an antibody or antigen-binding fragment thereof) could suppress inflammation in the liver, which would improve insulin sensitivity in the subject. For instance, a human subject presenting with type 2 diabetes could be treated by administering an antagonistic Fas antibody of the disclosure to the subject by an appropriate route (e.g., intravenously) at a particular dosage (e.g., between 0.001 and 100 mg/kg/day) over a course of days, weeks, months, or years. If desired, the antagonistic Fas antibody can be modified, e.g., by hyperglycosylation or by conjugation with PEG, so as to improve the pharmacokinetics (e.g., half-life) of the antibody (e.g., by reducing immune recognition) and/or to improve the in vivo distribution of the antibody.

The progression of type 2 diabetes in the treated subject could be monitored by any one or more of several established methods. For example, a physician could monitor the subject by direct observation in order to evaluate any changes in the symptoms exhibited by the subject following treatment. A blood sample could also be withdrawn from the subject in order to analyze the level of blood sugar and/or soluble Fas in order to determine if the quantity of sugar or protein has changed (e.g., decreased) in response to treatment with an antagonistic Fas antibody of the disclosure. Based on the results of these analyses, a physician may prescribe higher/lower dosages or more/less frequent dosing of the antagonistic Fas antibody in subsequent rounds of treatment.

### Example 19. Treatment of Osteoporosis in a Human Subject by Administration of an Antagonistic RANK Polypeptide

An antagonistic RANK polypeptide of the disclosure (e.g., single-chain polypeptides, antibodies, or fragments thereof) can be administered to a human subject in order to treat osteoporosis. Administration of these polypeptides (e.g., an antibody or antigen-binding fragment thereof) could suppress the proliferation of a population of osteoclasts, which would reduce bone resorption in the subject. For instance, a human subject presenting with osteoporosis (or decreased bone density) could be treated by administering an antagonistic RANK antibody of the disclosure to the subject by an appropriate route (e.g., intravenously) at a particular dosage (e.g., between 0.001 and 100 mg/kg/day) over a course of days, weeks, months, or years. If desired, the antagonistic RANK antibody can be modified, e.g., by hyperglycosylation or by conjugation with PEG, so as to improve the pharmacokinetics (e.g., half-life) of the antibody (e.g., by reducing immune recognition) and/or to improve the in vivo distribution of the antibody.

The progression of osteoporosis in the treated subject could be monitored by any one or more of several established methods. For example, a physician could monitor the subject by direct observation in order to evaluate any changes in the symptoms exhibited by the subject following treatment. A blood sample could also be withdrawn from the subject in order to analyze the level of soluble RANKL, OPG, and/or RANK in order to determine if the quantity of protein has changed (e.g., decreased) in response to treatment with an antagonistic RANK antibody of the disclosure. A physician could also monitor the change in bone density within the subject during the course of RANK antibody therapy. Based on the results of these analyses, a physician may prescribe higher/lower dosages or more/less frequent dosing of the antagonistic RANK antibody in subsequent rounds of treatment.

### Example 20. Producing a Humanized CD40 Antibody

One method for producing humanized CD40 antibodies of the disclosure is to import the CDRs of a CD40 antibody into a human antibody consensus sequence. Consensus human antibody heavy chain and light chain sequences are known in the art (see e.g., the “VBASE” human germline sequence database; Kabat et al. (Sequences of Proteins of Immunological Interest, Fifth Edition, U.S. Department of Health and Human Services, NIH Publication No. 91-3242, 1991); Tomlinson et al. (J. Mol. Biol. 227:776-798, 1992); and Cox et al. (Eur. J. Immunol. 24:827-836, 1994); incorporated herein by reference). Using established procedures, one can identify the variable domain framework residues and CDRs of a consensus antibody sequence (e.g., by sequence alignment (see Kabat, supra)). One can substitute one or more CDRs of the heavy chain and/or light chain variable domains of consensus human antibody with one or more corresponding CDRs of a CD40 antagonist antibody described herein, in order to produce a humanized anti-CD40 antibody variant using gene editing techniques described herein or known in the art.

In order to produce humanized CD40 antibodies, one can recombinantly express a polynucleotide encoding the above consensus sequence in which one or more variable region CDRs have been replaced with one or more variable region CDR sequences of a CD40-specific antibody. A polynucleotide encoding the above heavy chain and light chain variable domains operatively linked to one another can be incorporated into an expression vector (e.g., an expression vector optimized for protein expression in prokaryotic or eukaryotic cells as described herein or known in the art). The single-chain antibody fragment (scFv) can thus be expressed in a host cell and subsequently purified from the host cell medium using established techniques, such as size-exclusion chromatography and/or affinity chromatography as described herein.

### Example 21. Treatment of Allograft Rejection in a Human Subject by Administration of an Antagonistic CD40 Polypeptide

An antagonistic CD40 polypeptide of the disclosure (e.g., single-chain polypeptides, antibodies, or fragments thereof) can be administered to a human subject in order to treat allograft rejection. Administration of these polypeptides (e.g., an antibody or antigen-binding fragment thereof) could suppress the proliferation of T effectors, which would attenuate immune responses mounted by self-reactive cytotoxic T-cells that are associated with the rejection of a tissue graft following transplantation. For instance, a human subject presenting with allograft rejection could be treated by administering an antagonistic CD40 antibody of the disclosure to the subject by an appropriate route (e.g., intravenously) at a particular dosage (e.g., between 0.001 and 100 mg/kg/day) over a course of days, weeks, months, or years. If desired, the antagonistic CD40 antibody can be modified, e.g., by hyperglycosylation or by conjugation with PEG, so as to improve the pharmacokinetics (e.g., half-life) of the antibody (e.g. by reducing immune recognition) and/or to improve the in vivo distribution of the antibody.

The progression of the allograft rejection in the treated subject could be monitored by any one or more of several established methods. For example, a physician could monitor the subject by direct observation in order to evaluate any changes in the symptoms exhibited by the subject following treatment. A blood sample could also be withdrawn from the subject in order to analyze the cell count of one or more CD8+ T-cells in order to determine if the quantity of cells has changed (e.g., decreased) in response to treatment with an antagonistic CD40 antibody of the disclosure. A physician could also monitor the fluctuation in the volume of the allograft within the subject during the course of CD40 antibody therapy. Based on the results of these analyses, a physician may prescribe higher/lower dosages or more/less frequent dosing of the antagonistic CD40 antibody in subsequent rounds of treatment in order to preserve the allograft.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. An antibody or antigen-binding fragment thereof that specifically binds a human tumor necrosis factor receptor superfamily (TNFRSF) member protein selected from CD40, 4-1 BB, CD27, CD30, DR6, EDAR, Fas, GITR, HVEM, LT beta receptor, NGFR, OPG, OX40, RANK, RELT (19L), TNFR1, TRAIL-R2 (TNFRSF10B), TRAIL-R1 (TNFRSF10A), TRAIL-R4, TRAMP, TROY, and XEDAR, wherein the antibody or antigen-binding fragment thereof specifically binds:
(a) CD40, wherein the antibody or antigen-binding fragment thereof specifically binds an epitope comprising five or more of amino acids 124-132 of SEQ ID NO: 4 and/or five or more of amino acids 143-152 of SEQ ID NO: 4, or an epitope with at least 80% or greater sequence identity thereto; or
(b) 4-1 BB, wherein the antibody or antigen-binding fragment thereof specifically binds an epitope comprising five or more of amino acids 101-129 of SEQ ID NO: 1, or an epitope with at least 80% or greater sequence identity thereto; or
(c) CD27, wherein the antibody or antigen-binding fragment thereof specifically binds an epitope comprising five or more of amino acids 41-47 of SEQ ID NO: 2 and/or five or more of amino acids 61-72 of SEQ ID NO: 2, or an epitope with at least 80% or greater sequence identity thereto; or
(d) CD30, wherein the antibody or antigen-binding fragment thereof specifically binds an epitope comprising five or more of amino acids 129-140 of SEQ ID NO: 3 and/or five or more of amino acids 149-157 of SEQ ID NO: 3, or an epitope with at least 80% or greater sequence identity thereto; or
(e) DR6, wherein the antibody or antigen-binding fragment thereof specifically binds an epitope comprising five or more of amino acids 141-156 of SEQ ID NO: 5 and/or five or more of amino acids 169-177 of SEQ ID NO: 5, or an epitope with at least 80% or greater sequence identity thereto; or
(f) EDAR, wherein the antibody or antigen-binding fragment thereof specifically binds an epitope comprising five or more of amino acids 91-102 of SEQ ID NO: 6 and/or five or more of amino acids 111-122 of SEQ ID NO: 6, or an epitope with at least 80% or greater sequence identity thereto; or
(g) Fas, wherein the antibody or antigen-binding fragment thereof specifically binds an epitope comprising five or more of amino acids 148-156 of SEQ ID NO: 7 and/or five or more of amino acids 165-173 of SEQ ID NO: 7, or an epitope with at least 80% or greater sequence identity thereto; or
(h) GITR, wherein the antibody or antigen-binding fragment thereof specifically binds an epitope comprising five or more of amino acids 92-101 of SEQ ID NO: 8 and/or five or more of amino acids 112-121 of SEQ ID NO: 8, or an epitope with at least 80% or greater sequence identity thereto; or
(i) HVEM, wherein the antibody or antigen-binding fragment thereof specifically binds an epitope comprising five or more of amino acids 142-152 of SEQ ID NO: 9 and/or five or more of amino acids 162-170 of SEQ ID NO: 9, or an epitope with at least 80% or greater sequence identity thereto; or
(j) LT beta receptor, wherein the antibody or antigen-binding fragment thereof specifically binds an epitope comprising five or more of amino acids 147-155 of SEQ ID NO: 10 and/or five or more of amino acids 167-176 of SEQ ID NO: 10, or an epitope with at least 80% or greater sequence identity thereto; or
(k) NGFR, wherein the antibody or antigen-binding fragment thereof specifically binds an epitope comprising five or more of amino acids 128-135 of SEQ ID NO: 11 and/or five or more of amino acids 146-156 of SEQ ID NO: 11, or an epitope with at least 80% or greater sequence identity thereto; or
(l) OPG, wherein the antibody or antigen-binding fragment thereof specifically binds an epitope comprising five or more of amino acids 123-131 of SEQ ID NO: 12 and/or five or more of amino acids 142-151 of SEQ ID NO: 12, or an epitope with at least 80% or greater sequence identity thereto; or
(m) OX40, wherein the antibody or antigen-binding fragment thereof specifically binds an epitope comprising five or more of amino acids 118-134 of SEQ ID NO: 13, or an epitope with at least 80% or greater sequence identity thereto; or
(n) RANK, wherein the antibody or antigen-binding fragment thereof specifically binds an epitope comprising five or more of amino acids 132-139 of SEQ ID NO: 14 and/or five or more of amino acids 151-160 of SEQ ID NO: 14, or an epitope with at least 80% or greater sequence identity thereto; or
(o) RELT (19L), wherein the antibody or antigen-binding fragment thereof specifically binds an epitope comprising five or more of amino acids 70-77 of SEQ ID NO: 15 and/or five or more of amino acids 90-98 of SEQ ID NO: 15, or an epitope with at least 80% or greater sequence identity thereto; or
(p) TNFR1, wherein the antibody or antigen-binding fragment thereof specifically binds an epitope comprising five or more of amino acids 104-112 of SEQ ID NO: 16 and/or five or more of amino acids 124-133 of SEQ ID NO: 16, or an epitope with at least 80% or greater sequence identity thereto; or
(q) TRAIL-R2 (TNFRSF10B), wherein the antibody or antigen-binding fragment thereof specifically binds an epitope comprising five or more of amino acids 155-165 of SEQ ID NO: 17 and/or five or more of amino acids 178-186 of SEQ ID NO: 17, or an epitope with at least 80% or greater sequence identity thereto; or
(r) TRAIL-R1 (TNFRSF10A), wherein the antibody or antigen-binding fragment thereof specifically binds an epitope comprising five or more of amino acids 169-177 of SEQ ID NO: 18 and/or five or more of amino acids 188-196 of SEQ ID NO: 18, or an epitope with at least 80% or greater sequence identity thereto; or
(s) TRAIL-R4, wherein the antibody or antigen-binding fragment thereof specifically binds an epitope comprising five or more of amino acids 161-169 of SEQ ID NO: 19 and/or five or more of amino acids 181-190 of SEQ ID NO: 19, or an epitope with at least 80% or greater sequence identity thereto; or
(t) TRAMP, wherein the antibody or antigen-binding fragment thereof specifically binds an epitope comprising five or more of amino acids 142-150 of SEQ ID NO: 20 and/or five or more of amino acids 162-171 of SEQ ID NO: 20, or an epitope with at least 80% or greater sequence identity thereto; or
(u) TROY, wherein the antibody or antigen-binding fragment thereof specifically binds an epitope comprising five or more of amino acids 51-59 of SEQ ID NO: 21 and/or five or more of amino acids 72-82 of SEQ ID NO: 21, or an epitope with at least 80% or greater sequence identity thereto; or
(v) XEDAR, wherein the antibody or antigen-binding fragment thereof specifically binds an epitope comprising five or more of amino acids 20-27 of SEQ ID NO: 22 and/or five or more of amino acids 42-49 of SEQ ID NO: 22, or an epitope with at least 80% or greater sequence identity thereto.

2. The antibody or antigen-binding fragment thereof of claim 1, wherein the antibody or antigen-binding fragment thereof comprises a non-native constant region.

3. The antibody or antigen-binding fragment thereof of claim 1 or 2, wherein the antibody or antigen-binding fragment thereof inhibits signaling associated with the TNFRSF member protein.

4. The antibody or antigen-binding fragment thereof of any one of claims 1-3, wherein the antibody or antigen-binding fragment thereof binds the TNFRSF member protein with a K_{d} of no greater than about 10 nM.

5. The antibody or antigen-binding fragment thereof of claim 4, wherein the antibody or antigen-binding fragment thereof binds the TNFRSF member protein with a K_{d} of no greater than about 1 nM.

6. The antibody or antigen-binding fragment thereof of any one of claims 1-5, wherein the antibody or antigen-binding fragment thereof binds the TNFRSF member protein to form an antibody-antigen complex with a kₒₙ of at least about 10⁴ M⁻¹s⁻¹.

7. The antibody or antigen-binding fragment thereof of any one of claims 1-6, wherein the antibody or antigen-binding fragment thereof binds the TNFRSF member protein to form an antibody-antigen complex, and wherein the complex dissociates with a k_{off} of no greater than about 10⁻³ s⁻¹.

8. The antibody or antigen-binding fragment thereof of any one of claims 1-7, wherein the antibody or antigen-binding fragment thereof has an isotype selected from the group consisting of IgG, IgA, IgM, IgD, and IgE.

9. The antibody or antigen-binding fragment thereof of claim 8, wherein the antibody or antigen-binding fragment thereof is an IgG isotype.

10. The antibody or antigen-binding fragment thereof of claim 9, wherein the antibody or antigen-binding fragment thereof is an IgG2 isotype.

11. The antibody or antigen-binding fragment thereof of any one of claims 1-10, wherein the antibody or antigen-binding fragment thereof comprises a human IgG2 hinge region that lacks a cysteine residue at positions 232 and/or 233 of the amino acid sequence of the IgG2 hinge region.

12. The antibody or antigen-binding fragment thereof of claim 11, wherein the antibody or antigen-binding fragment thereof comprises a human IgG2 hinge region that lacks a cysteine residue at position 232 of the amino acid sequence of the IgG2 hinge region.

13. The antibody or antigen-binding fragment thereof of claim 11 or 12, wherein the antibody or antigen-binding fragment thereof comprises a human IgG2 hinge region that lacks a cysteine residue at position 233 of the amino acid sequence of the IgG2 hinge region.

14. The antibody or antigen-binding fragment thereof of any one of claims 11-13, wherein the antibody or antigen-binding fragment thereof comprises a human IgG2 hinge region having an amino acid other than cysteine at positions 232 and/or 233 of the amino acid sequence of the IgG2 hinge region.

15. The antibody or antigen-binding fragment thereof of claim 14, wherein the antibody or antigen-binding fragment thereof comprises a human IgG2 hinge region having an amino acid other than cysteine at position 232 of the amino acid sequence of the IgG2 hinge region.

16. The antibody or antigen-binding fragment thereof of claim 14 or 15, wherein the antibody or antigen-binding fragment thereof comprises a human IgG2 hinge region having an amino acid other than cysteine at position 233 of the amino acid sequence of the IgG2 hinge region.

17. The antibody or antigen-binding fragment thereof of any one of claims 11-16, wherein the antibody or antigen-binding fragment thereof comprises a human IgG2 hinge region having a serine residue at positions 232 and/or 233 of the amino acid sequence of the IgG2 hinge region.

18. The antibody or antigen-binding fragment thereof of claim 17, wherein the antibody or antigen-binding fragment thereof comprises a human IgG2 hinge region having a serine residue at position 232 of the amino acid sequence of the IgG2 hinge region.

19. The antibody or antigen-binding fragment thereof of claim 17 or 18, wherein the antibody or antigen-binding fragment thereof comprises a human IgG2 hinge region having a serine residue at position 233 of the amino acid sequence of the IgG2 hinge region.

20. The antibody or antigen-binding fragment thereof of any one of claims 11-19, wherein the antibody or antigen-binding fragment thereof comprises a human IgG2 hinge region comprising an amino acid substitution or deletion at one or both of cysteine residues 232 and 233.

21. The antibody or antigen-binding fragment thereof of claim 20, wherein the IgG2 hinge region comprises an amino acid substitution at one or both of cysteine residues 232 and 233.

22. The antibody or antigen-binding fragment thereof of claim 21, wherein the IgG2 hinge region comprises an amino acid substitution at cysteine residue 232.

23. The antibody or antigen-binding fragment thereof of claim 21 or 22, wherein the IgG2 hinge region comprises an amino acid substitution at cysteine residue 233.

24. The antibody or antigen-binding fragment thereof of any one of claims 20-23, wherein the amino acid substitution is a conservative amino acid substitution.

25. The antibody or antigen-binding fragment thereof of claim 24, wherein the IgG2 hinge region comprises a C232S substitution.

26. The antibody or antigen-binding fragment thereof of claim 24 or 25, wherein the IgG2 hinge region comprises a C233S substitution.

27. The antibody or antigen-binding fragment thereof of any one of claims 1-26, wherein the antibody or antigen-binding fragment thereof comprises antigen-binding sites separated from one another by a distance of at least about 133 Å.

28. The antibody or antigen-binding fragment thereof of claim 27, wherein the antigen-binding sites are separated from one another by a distance of at least about 134 Å.

29. The antibody or antigen-binding fragment thereof of claim 28, wherein the antigen-binding sites are separated from one another by a distance of at least about 139 Å.

30. The antibody or antigen-binding fragment thereof of claim 29, wherein the antigen-binding sites are separated from one another by a distance of at least about 150 Å.

31. The antibody or antigen-binding fragment thereof of any one of claims 1-26, wherein the antigen-binding sites are separated from one another by a distance of from about 133 Å to about 150 Å.

32. The antibody or antigen-binding fragment thereof of claim 31, wherein the antigen-binding sites are separated from one another by a distance of from about 133 Å to about 145 Å.

33. The antibody or antigen-binding fragment thereof of claim 31, wherein the antigen-binding sites are separated from one another by a distance of from about 133 Å to about 139 Å.

34. The antibody or antigen-binding fragment thereof of claim 31, wherein the antigen-binding sites are separated from one another by a distance of from about 134 Å to about 139 Å.

35. A method of producing the antibody of any one of claims 1-34, said method comprising immunizing a non-human mammal with a peptide comprising the amino acid sequence of any one of SEQ ID NOs: 25-153, collecting serum TNFR2 antibodies, and retaining TNFR2 antibodies or antigen-binding fragments thereof that bind TNFR2 with a K_{D} of less than about 40 pM.

36. The method of claim 35, wherein said non-human mammal is selected from the group consisting of a rabbit, mouse, rat, goat, guinea pig, hamster, horse, and sheep.

37. The method of claim 35 or 36, wherein said peptide comprises the amino acid sequence KCRPG (SEQ ID NO: 68).

38. The method of any one of claims 35-37, wherein said peptide comprises the amino acid sequence CAPLRKCR (SEQ ID NO: 67).

39. The method of any one of claims 35-38, wherein said peptide comprises the amino acid sequence KCRPGFGV (SEQ ID NO: 69).

40. An antibody or antigen-binding fragment thereof that is produced by the method of any one of claims 35-39.

41. The antibody or antigen-binding fragment thereof of any one of claims 1-34, wherein the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv).

42. The antibody or antigen-binding fragment thereof of claim 41, wherein the antibody or antigen-binding fragment thereof is a human, humanized, or chimeric antibody or antigen-binding fragment thereof.

43. The antibody or antigen-binding fragment thereof of any one of claims 1-34 and 40-42, wherein the antibody is conjugated to a therapeutic agent.

44. The antibody or antigen-binding fragment thereof of claim 43, wherein the therapeutic agent is a cytotoxic agent.

45. The antibody or antigen-binding fragment thereof of any one of claims 1-34 and 40-44, wherein the antibody or antigen-binding fragment thereof comprises a framework region from a human antibody or chimeric antibody.

46. The antibody or antigen-binding fragment thereof of any one of claims 1-34 and 40-45, wherein the antibody or antigen binding fragment thereof stabilizes an anti-parallel dimer conformation of the TNFRSF member.

47. The antibody or antigen-binding fragment thereof of any one of claims 1-34 and 40-46, wherein the antibody or antigen-binding fragment thereof destabilizes a trimeric conformation of the TNFRSF member.

48. The antibody or antigen-binding fragment thereof of any one of claims 1-34 and 40-47, wherein the antibody or antigen-binding fragment thereof reduces secretion of a soluble version of the TNFRSF member protein.

49. The antibody or antigen-binding fragment thereof of any one of claims 1-34 and 40-48, wherein the antibody or antigen-binding fragment thereof inhibits expression of one of more genes selected from the group consisting of CHUK, NFκBIE, NFκBIA, MAP3K11, TRAF2, TRAF3, relB, and cIAP2/BIRC3.

50. The antibody or antigen-binding fragment thereof of any one of claims 1-34 and 40-49, wherein the antibody or antigen-binding fragment thereof inhibits NFκB activation.

51. The antibody or antigen-binding fragment thereof of any one of claims 1-34 and 40-50, wherein the antibody or antigen-binding fragment thereof is capable of reducing or inhibiting proliferation of a population of T-reg cells and/or is capable of inducing proliferation of a population of CD8+ effector T cells.

52. The antibody or antigen-binding fragment thereof of any one of claims 1-34 and 40-51, wherein the antibody or antigen-binding fragment thereof is capable of reducing or inhibiting proliferation of a population of cancer cells, optionally wherein the cancer cells express the TNFRSF member protein.

53. The antibody or antigen-binding fragment thereof of claim 52, wherein the cancer cells are selected from the group consisting of Hodgkin lymphoma cells, cutaneous non-Hodgkin lymphoma cells, T cell lymphoma cells, ovarian cancer cells, colon cancer cells, multiple myeloma cells, and renal cell carcinoma cells.

54. The antibody or antigen-binding fragment thereof of any one of claims 1-34 and 40-53, wherein the antibody or antigen-binding fragment thereof inhibits TNFRSF signaling in proliferating cells.

55. The antibody or antigen-binding fragment thereof of any one of claims 1-34 and 40-54, wherein the antibody or antigen-binding fragment thereof does not inhibit TNFRSF signaling in resting cells.

56. The antibody or antigen-binding fragment thereof of any one of claims 1-34 and 40-55, wherein the antibody or antigen-binding fragment thereof is capable of reducing or inhibiting proliferation of a population of myeloid-derived suppressor cells.

57. The antibody or antigen-binding fragment thereof of any one of claims 1-34 and 40-56, wherein the antibody or antigen-binding fragment thereof is capable of selectively reducing or inhibiting the proliferation of a population of T-reg cells expressing CD25h.

58. The antibody or antigen-binding fragment thereof of any one of claims 1-34 and 40-57, wherein the antibody or antigen-binding fragment thereof is capable of reducing or inhibiting proliferation of a population of T-reg cells in the presence of a ligand for the TNFRSF member protein.

59. The antibody or antigen-binding fragment thereof of any one of claims 1-34 and 40-49, wherein the antibody or antigen-binding fragment thereof promotes proliferation of T-reg cells.

60. The antibody or antigen-binding fragment thereof of claim 59, wherein the antibody or antigen-binding fragment thereof directly kills, or promotes the death of, CD8+ cytotoxic T cells.

61. The antibody or antigen-binding fragment thereof of claim 59 or 60, wherein the antibody or antigen-binding fragment thereof is capable of reducing or inhibiting proliferation of a population of CD8+ cytotoxic T cells in the presence of a ligand for the TNFRSF member protein.

62. A method of producing the antibody or antigen-binding fragment thereof of any one of claims 1-34 and 40-61, said method comprising expressing a polynucleotide encoding said antibody or antigen-binding fragment thereof in a host cell and recovering the antibody or antigen-binding fragment thereof from host cell medium.

63. A construct comprising a first polypeptide domain and a second polypeptide domain, wherein the first polypeptide domain and the second polypeptide domain are each, independently, an antigen-binding fragment of any one of claims 1-34 and 40-61.

64. The construct of claim 63, wherein the first polypeptide domain and the second polypeptide domain are bound by a covalent linker.

65. The construct of claim 64, wherein the covalent linker comprises an amide bond.

66. The construct of claim 64, wherein the covalent linker comprises a disulfide bond.

67. A polynucleotide encoding the antibody or antigen-binding fragment thereof of any one of claims 1-34 and 40-66.

68. A polynucleotide encoding the construct of any one of claims 63-66.

69. A vector comprising the polynucleotide of claim 67 or 68.

70. The vector of claim 69, wherein the vector is an expression vector.

71. The vector of claim 70, wherein the expression vector is a eukaryotic expression vector.

72. The vector of claim 69, wherein the vector is a viral vector.

73. The vector of claim 72, wherein the viral vector is selected from the group consisting of adenovirus (Ad), retrovirus, poxvirus, adeno-associated virus, baculovirus, herpes simplex virus, and a vaccinia virus.

74. The vector of claim 73, wherein the adenovirus is a serotype 1-60 adenovirus.

75. The vector of claim 74, wherein the adenovirus is a serotype 5, 26, 35, or 48 adenovirus.

76. The vector of claim 73, wherein the retrovirus is a γ-retrovirus or a lentivirus.

77. The vector of claim 73, wherein the vaccinia virus is a modified vaccinia Ankara (MVA).

78. An isolated host cell comprising the vector of any one of claims 69-77.

79. The host cell of claim 78, wherein the host cell is a prokaryotic cell.

80. The host cell of claim 78, wherein the host cell is a eukaryotic cell.

81. The host cell of claim 80, wherein the eukaryotic cell is a mammalian cell.

82. The host cell of claim 81, wherein the mammalian cell is a CHO cell.

83. A pharmaceutical composition comprising the antibody or antigen-binding fragment thereof of any one of claims 1-34 and 40-61, the construct of any one of claims 63-66, the polynucleotide of claim 67 or 68, the vector of any one of claims 69-77, or the host cell of any one of claims 78 and 80-82, and a pharmaceutically acceptable carrier or excipient.

84. The pharmaceutical composition of claim 83, wherein the pharmaceutical composition comprises the antibody or antigen biding fragment thereof of any one of claims 1-34 and 40-61.

85. The pharmaceutical composition of claim 84, wherein at least 50% of the antibody or antigen-binding fragment thereof in the pharmaceutical composition is present in a single disulfide-bonded isoform.

86. The pharmaceutical composition of claim 85, wherein at least 75% of the antibody or antigen-binding fragment thereof in the pharmaceutical composition is present in a single disulfide-bonded isoform.

87. The pharmaceutical composition of claim 86, wherein at least 80% of the antibody or antigen-binding fragment thereof in the pharmaceutical composition is present in a single disulfide-bonded isoform.

88. The pharmaceutical composition of claim 87, wherein at least 85% of the antibody or antigen-binding fragment thereof in the pharmaceutical composition is present in a single disulfide-bonded isoform.

89. The pharmaceutical composition of claim 88, wherein at least 90% of the antibody or antigen-binding fragment thereof in the pharmaceutical composition is present in a single disulfide-bonded isoform.

90. The pharmaceutical composition of claim 89, wherein at least 95% of the antibody or antigen-binding fragment thereof in the pharmaceutical composition is present in a single disulfide-bonded isoform.

91. The pharmaceutical composition of claim 86, wherein from about 75% to about 99.9% of the antibody or antigen-binding fragment thereof in the pharmaceutical composition is present in a single disulfide-bonded isoform.

92. The pharmaceutical composition of claim 91, wherein from about 80% to about 99.9% of the antibody or antigen-binding fragment thereof in the pharmaceutical composition is present in a single disulfide-bonded isoform.

93. The pharmaceutical composition of claim 92, wherein from about 85% to about 99.9% of the antibody or antigen-binding fragment thereof in the pharmaceutical composition is present in a single disulfide-bonded isoform.

94. The pharmaceutical composition of claim 93, wherein from about 90% to about 99.9% of the antibody or antigen-binding fragment thereof in the pharmaceutical composition is present in a single disulfide-bonded isoform.

95. The pharmaceutical composition of claim 94, wherein from about 95% to about 99.9% of the antibody or antigen-binding fragment thereof in the pharmaceutical composition is present in a single disulfide-bonded isoform.

96. The pharmaceutical composition of any one of claims 83-95, wherein the antibody or antigen-binding fragment thereof yields a single detectable band upon gel electrophoresis analysis performed under non-reducing conditions.

97. The pharmaceutical composition of any one of claims 85-96, wherein the single disulfide-bonded isoform is IgG2-A.

98. The pharmaceutical composition of any one of claims 83-97, wherein the antibody or antigen-binding fragment thereof is present in the pharmaceutical composition in an amount of from about 0.001 mg/ml to about 100 mg/ml.

99. The pharmaceutical composition of any one of claims 83-98, wherein the pharmaceutical composition further comprises an additional therapeutic agent.

100. The pharmaceutical composition of claim 99, wherein the additional therapeutic agent is an immunotherapy agent.

101. The pharmaceutical composition of claim 100, wherein the immunotherapy agent is selected from the group consisting of an anti-CTLA-4 agent, an anti-PD-1 agent, an anti-PD-L1 agent, an anti-PD-L2 agent, a TNF-α cross-linking agent, a TRAIL cross-linking agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-LIGHT agent, an anti-DR4 agent, an anti-CR5 agent an anti-CD95 agent, an anti-TRAIL agent, an anti-BCMA agent, an anti-TACI agent, an anti-RANKL agent, and an anti-BAFFR agent, optionally wherein the immunotherapy agent is an anti-PD-1 antibody or an anti-PD-L1 antibody.

102. The pharmaceutical composition of claim 101, wherein the immunotherapy agent is selected from the group consisting of an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-TWEAK antibody or antigen-binding fragment thereof, an anti-TWEAKR antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-LIGHT antibody or antigen-binding fragment thereof, an anti-DR4 antibody or antigen-binding fragment thereof, an anti-CR5 antibody or antigen-binding fragment thereof, an anti-CD95 antibody or antigen-binding fragment thereof, an anti-TRAIL antibody or antigen-binding fragment thereof, an anti-BCMA antibody or antigen-binding fragment thereof, an anti-TACI antibody or antigen-binding fragment thereof, an anti-RANKL antibody or antigen-binding fragment thereof, and an anti-BAFFR antibody or antigen-binding fragment thereof.

103. The pharmaceutical composition of claim 102, wherein the immunotherapy agent is an anti-CTLA-4 agent or an anti-PD-1 agent.

104. The pharmaceutical composition of claim 103, wherein the immunotherapy agent is an anti-CTLA-4 antibody or antigen-binding fragment thereof or an anti-PD-1 antibody or antigen-binding fragment thereof.

105. The pharmaceutical composition of claim 103 or 104, wherein the anti-CTLA-4 antibody is ipilimumab or tremelimumab.

106. The pharmaceutical composition of claim 103 or 104, wherein the anti-PD-1 antibody is nivolumab, pembrolizumab, avelumab, durvalumab, or atezolizumab.

107. The pharmaceutical composition of claim 99, wherein the additional therapeutic agent is a chimeric antigen receptor (CAR-T) agent, a chemotherapeutic agent, a small molecule anti-cancer agent, or a cancer vaccine.

108. A method of reducing or inhibiting an immune response mediated by a T-reg cell in a human, the method comprising administering to the human the antibody or antigen-binding fragment thereof of any one of claims 1-34 and 40-58, the construct of any one of claims 63-66, the polynucleotide of claim 67 or 68, the vector of any one of claims 69-77, the host cell of any one of claims 78 and 80-82, or the pharmaceutical composition of any one of claims 83-107, wherein the antibody or antigen-binding fragment thereof is an antagonist of TRAMP, DR6, TRAIL-R3, TRAIL-R4, RANK, LT Beta, HVEM, CD30, TROY, or RELT (19L).

109. A method of treating a cell proliferation disorder in a human, the method comprising administering to the human the antibody or antigen-binding fragment thereof of any one of claims 1-34 and 40-58, the construct of any one of claims 63-66, the polynucleotide of claim 67 or 68, the vector of any one of claims 69-77, the host cell of any one of claims 78 and 80-82, or the pharmaceutical composition of any one of claims 83-107, wherein the antibody or antigen-binding fragment thereof is an antagonist of TRAMP, DR6, TRAIL-R3, TRAIL-R4, RANK, LT Beta, HVEM, CD30, TROY, or RELT (19L).

110. The method of claim 109, wherein the cell proliferation disorder is a cancer selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

111. The method of claim 109, wherein the cell proliferation disorder is a cancer selected from the group consisting of Hodgkin lymphoma, cutaneous non-Hodgkin lymphoma, T cell lymphoma, ovarian cancer, colon cancer, multiple myeloma, renal cell carcinoma, skin cancer, lung cancer, liver cancer, endometrial cancer, a cancer of the hematopoietic or lymphatic system, a cancer of the central nervous system, breast cancer, pancreatic cancer, stomach cancer, esophageal cancer, and a cancer of the upper gastrointestinal tract.

112. The method of claim 111, wherein the cell proliferation disorder is a cancer selected from the group consisting of T cell lymphoma, ovarian cancer, and colon cancer.

113. The method of claim 109, wherein the cell proliferation disorder is a cancer selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, Hodgkin lymphoma, anaplastic large cell lymphoma, hypopharyngeal cancer, islet cell tumors, pancreatic neuroendocrine tumors, wilms tumor and other childhood kidney tumors, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, renal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia.

114. The method of claim 109, wherein a sample obtained from the human has a ratio of T-reg cells to CD8+ T effector cells that is greater than a ratio of T-reg cells to CD8+ T effector cells in a sample obtained from a human that does not have the cell proliferation disorder, optionally wherein the ratio in the sample from the human having the cell proliferation disorder is greater than the ratio in the sample from the human that does not have the cell proliferation disorder by 5%, 10%, 15%, 20%, 25%, 30%, 35%, 40%, 45%, 50%, 55%, 60%, 65%, 70%, 75%, 80%, 85%, 90%, 95%, 100%, 200%, or more.

115. The method of claim 114, wherein the sample is a blood sample.

116. The method of claim 114 or 115, wherein the sample is obtained from a tumor microenvironment.

117. The method of claim 114, wherein the sample is a tumor biopsy.

118. A method of treating an infectious disease in a human, the method comprising administering to the human the antibody or antigen-binding fragment thereof of any one of claims 1-34 and 40-58, the construct of any one of claims 63-66, the polynucleotide of claim 67 or 68, the vector of any one of claims 69-77, the host cell of any one of claims 78 and 80-82, or the pharmaceutical composition of any one of claims 83-107, wherein the antibody or antigen-binding fragment thereof is an antagonist of TRAMP, DR6, TRAIL-R3, TRAIL-R4, HVEM, or RELT (19L).

119. The method of claim 118, wherein the infectious disease is caused by one or more agents selected from the group consisting of a virus, a bacterium, a fungus, or a parasite.

120. The method of claim 119, wherein the infectious disease is caused by a virus selected from the group consisting of hepatitis C virus, Yellow fever virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus, Negishi virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, cell fusing agent virus, Ippy virus, Lassa virus, lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabiá virus, Tacaribe virus, Tamiami virus, Whitewater Arroyo virus, Chapare virus, Lujo virus, Hantaan virus, Sin Nombre virus, Dugbe virus, Bunyamwera virus, Rift Valley fever virus, La Crosse virus, California encephalitis virus, Crimean-Congo hemorrhagic fever (CCHF) virus, Ebola virus, Marburg virus, Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus, smallpox virus, monkeypox virus, vaccinia virus, herpes simplex virus, human herpes virus, cytomegalovirus (CMV), Epstein-Barr virus (EBV), Varicella-Zoster virus, Kaposi's sarcoma associated-herpesvirus (KSHV), influenza virus, severe acute respiratory syndrome (SARS) virus, rabies virus, vesicular stomatitis virus (VSV), human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, mumps virus, poliovirus, human enterovirus (A, B, C, and D), hepatitis A virus, coxsackievirus, hepatitis B virus, human papilloma virus, adeno-associated virus, astrovirus, JC virus, BK virus, SV40 virus, Norwalk virus, rotavirus, human immunodeficiency virus (HIV), human T-lymphotropic virus Types I and II.

121. The method of claim 119, wherein the infectious disease is caused by a bacterium belonging to a genus selected from the group consisting of Salmonella, Streptococcus, Bacillus, Listeria, Corynebacterium, Nocardia, Neisseria, Actinobacter, Moraxella, Enterobacteriacece, Pseudomonas, Escherichia, Klebsiella, Serratia, Enterobacter, Proteus, Salmonella, Shigella, Yersinia, Haemophilus, Bordatella, Legionella, Pasteurella, Francisella, Brucella, Bartonella, Clostridium, Vibrio, Campylobacter, and Staphylococcus.

122. The method of claim 119, wherein the infectious disease is caused by a fungus selected from the group consisting of Aspergillus, Candida, Malassezia, Trichosporon, Fusarium, Acremonium, Rhizopus, Mucor, Pneumocystis, and Absidia.

123. The method of claim 119, wherein the infectious disease is caused by a parasite selected from the group consisting of Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. Exemplary helminthic parasites include richuris trichiura, Ascaris lumbricoides, Enterobius vermicularis, Ancylostoma duodenale, Necator americanus, Strongyloides stercoralis, Wuchereria bancrofti, and Dracunculus medinensis, Schistosoma mansoni, Schistosoma haematobium, Schistosoma japonicum, Fasciola hepatica, Fasciola gigantica, Heterophyes, Paragonimus westermani, Taenia solium, Taenia saginata, Hymenolepis nana, and Echinococcus granulosus.

124. The method of any one of claims 108-123, wherein the human is further administered an additional therapeutic agent.

125. The method of claim 124, wherein the additional therapeutic agent is an immunotherapy agent.

126. The method of claim 125, wherein the immunotherapy agent is selected from the group consisting of an anti-CTLA-4 agent, an anti-PD-1 agent, an anti-PD-L1 agent, an anti-PD-L2 agent, a TNF-α cross-linking agent, a TRAIL cross-linking agent, an anti-TWEAK agent, an anti-TWEAKR agent, an anti-cell surface lymphocyte protein agent, an anti-BRAF agent, an anti-MEK agent, an anti-CD33 agent, an anti-CD20 agent, an anti-HLA-DR agent, an anti-HLA class I agent, an anti-CD52 agent, an anti-A33 agent, an anti-GD3 agent, an anti-PSMA agent, an anti-Ceacan 1 agent, an anti-Galedin 9 agent, an anti-VISTA agent, an anti-B7 H4 agent, an anti-HHLA2 agent, an anti-CD155 agent, an anti-CD80 agent, an anti-BTLA agent, an anti-CD160 agent, an anti-CD28 agent, an anti-CD226 agent, an anti-CEACAM1 agent, an anti-TIM3 agent, an anti-TIGIT agent, an anti-CD96 agent, an anti-CD70 agent, an anti-LIGHT agent, an anti-DR4 agent, an anti-CR5 agent an anti-CD95 agent, an anti-TRAIL agent, an anti-BCMA agent, an anti-TACI agent, an anti-RANKL agent, and an anti-BAFFR agent, optionally wherein the immunotherapy agent is an anti-PD-1 antibody or an anti-PD-L1 antibody.

127. The method of claim 126, wherein the immunotherapy agent is selected from the group consisting of an anti-CTLA-4 antibody or antigen-binding fragment thereof, an anti-PD-1 antibody or antigen-binding fragment thereof, an anti-PD-L1 antibody or antigen-binding fragment thereof, an anti-PD-L2 antibody or antigen-binding fragment thereof, a TNF-α cross-linking antibody or antigen-binding fragment thereof, a TRAIL cross-linking antibody or antigen-binding fragment thereof, an anti-TWEAK antibody or antigen-binding fragment thereof, an anti-TWEAKR antibody or antigen-binding fragment thereof, an anti-cell surface lymphocyte protein antibody or antigen-binding fragment thereof, an anti-BRAF antibody or antigen-binding fragment thereof, an anti-MEK antibody or antigen-binding fragment thereof, an anti-CD33 antibody or antigen-binding fragment thereof, an anti-CD20 antibody or antigen-binding fragment thereof, an anti-HLA-DR antibody or antigen-binding fragment thereof, an anti-HLA class I antibody or antigen-binding fragment thereof, an anti-CD52 antibody or antigen-binding fragment thereof, an anti-A33 antibody or antigen-binding fragment thereof, an anti-GD3 antibody or antigen-binding fragment thereof, an anti-PSMA antibody or antigen-binding fragment thereof, an anti-Ceacan 1 antibody or antigen-binding fragment thereof, an anti-Galedin 9 antibody or antigen-binding fragment thereof, an anti-VISTA antibody or antigen-binding fragment thereof, an anti-B7 H4 antibody or antigen-binding fragment thereof, an anti-HHLA2 antibody or antigen-binding fragment thereof, an anti-CD155 antibody or antigen-binding fragment thereof, an anti-CD80 antibody or antigen-binding fragment thereof, an anti-BTLA antibody or antigen-binding fragment thereof, an anti-CD160 antibody or antigen-binding fragment thereof, an anti-CD28 antibody or antigen-binding fragment thereof, an anti-CD226 antibody or antigen-binding fragment thereof, an anti-CEACAM1 antibody or antigen-binding fragment thereof, an anti-TIM3 antibody or antigen-binding fragment thereof, an anti-TIGIT antibody or antigen-binding fragment thereof, an anti-CD96 antibody or antigen-binding fragment thereof, an anti-CD70 antibody or antigen-binding fragment thereof, an anti-LIGHT antibody or antigen-binding fragment thereof, an anti-DR4 antibody or antigen-binding fragment thereof, an anti-CR5 antibody or antigen-binding fragment thereof, an anti-CD95 antibody or antigen-binding fragment thereof, an anti-TRAIL antibody or antigen-binding fragment thereof, an anti-BCMA antibody or antigen-binding fragment thereof, an anti-TACI antibody or antigen-binding fragment thereof, an anti-RANKL antibody or antigen-binding fragment thereof, and an anti-BAFFR antibody or antigen-binding fragment thereof.

128. The method of claim 126, wherein the immunotherapy agent is an anti-CTLA-4 agent or an anti-PD-1 agent.

129. The method of claim 127, wherein the immunotherapy agent is an anti-CTLA-4 antibody or antigen-binding fragment thereof or an anti-PD-1 antibody or antigen-binding fragment thereof.

130. The method of claim 128 or 129, wherein the anti-CTLA-4 agent or antibody is ipilimumab or tremelimumab.

131. The method of claim 128 or 129, wherein the anti-PD-1 agent or antibody is nivolumab, pembrolizumab, avelumab, durvalumab, or atezolizumab.

132. The method of claim 124, wherein the additional therapeutic agent is a chimeric antigen receptor (CAR-T) agent, a chemotherapeutic agent, a small molecule anti-cancer agent, or a cancer vaccine.

133. A method of inhibiting an immune response mediated by a B cell or CD8+ T cell in a human subject comprising administering to the subject the antibody or antigen-binding fragment thereof of any one of claims 1-34, 40-49, and 59-61, the construct of any one of claims 63-66, the polynucleotide of claim 67 or 68, the vector of any one of claims 69-77, the host cell of any one of claims 78 and 80-82, or the pharmaceutical composition of any one of claims 83-99, wherein the antibody or antigen-binding fragment thereof is an antagonist of CD40, TRAIL-R1 (TNFRSF10A), TRAIL-R2 (TNFRSF10B), DR6, NGFR, TNFR1, Fas, EDAR, RANK, CD27, 4-1 BB, OX40, GITR, or XEDAR.

134. A method of treating an immunological disease in a human subject comprising administering to the subject the antibody or antigen-binding fragment thereof of any one of claims 1-34, 40-49, and 59-61, the construct of any one of claims 63-66, the polynucleotide of claim 67 or 68, the vector of any one of claims 69-77, the host cell of any one of claims 78 and 80-82, or the pharmaceutical composition of any one of claims 83-99, wherein the antibody or antigen-binding fragment thereof is an antagonist of CD40, TRAIL-R1 (TNFRSF10A), TRAIL-R2 (TNFRSF10B), DR6, NGFR, TNFR1, Fas, EDAR, RANK, CD27, 4-1 BB, OX40, GITR, or XEDAR.

135. The method of claim 134, wherein the subject is in need of a tissue or organ regeneration.

136. The method of claim 135, wherein the tissue or organ is selected from the group consisting of a pancreas, salivary gland, pituitary gland, kidney, heart, lung, hematopoietic system, cranial nerves, heart, aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

137. The method of any one of claims 134-136, wherein the immunological disease is selected from the group consisting of an autoimmune disease, a neurological condition, an allergy, asthma, macular degeneration, muscular atrophy, a disease related to miscarriage, atherosclerosis, bone loss, a musculoskeletal disease, obesity, a graft-versus-host disease, and an allograft rejection.

138. A method of treating obesity, hyperlipidemia, and/or type 2 diabetes in a human subject, the method comprising administering to the subject the antibody or antigen-binding fragment thereof of any one of claims 1-34 and 40-58, the construct of any one of claims 63-66, the polynucleotide of claim 67 or 68, the vector of any one of claims 69-77, the host cell of any one of claims 78 and 80-82, or the pharmaceutical composition of any one of claims 83-99, wherein the antibody or antigen-binding fragment thereof is an antagonist of Fas.

139. A method of treating a neurological disorder in a human subject, the method comprising administering to the subject the antibody or antigen-binding fragment thereof of any one of claims 1-34 and 40-58, the construct of any one of claims 63-66, the polynucleotide of claim 67 or 68, the vector of any one of claims 69-77, the host cell of any one of claims 78 and 80-82, or the pharmaceutical composition of any one of claims 83-99, wherein the antibody or antigen-binding fragment thereof is an antagonist of DR6.

140. The method of claim 139, wherein the neurological disorder is selected from the group consisting of a brain tumor, a brain metastasis, a spinal cord injury, schizophrenia, epilepsy, Parkinson's disease, autism, Huntington's disease, stroke, Alzheimer's disease, and amyotrophic lateral sclerosis (ALS).

141. A method of treating osteoporosis or decreased bone loss in a human subject, the method comprising administering to the subject the antibody or antigen-binding fragment thereof of any one of claims 1-34 and 40-58, the construct of any one of claims 63-64, the polynucleotide of claim 67 or 68, the vector of any one of claims 69-77, the host cell of any one of claims 78 and 80-82, or the pharmaceutical composition of any one of claims 83-99, wherein the antibody or antigen-binding fragment thereof is an antagonist of RANK.

142. The method of any one of claims 108-141, wherein the method further comprises measuring a level of a secreted soluble TNFRSF member protein in a subject.

143. The method of claim 142, wherein the method further comprises measuring a level of a secreted version of the TNFRSF member protein in a subject after the subject is administered the antibody or antigen-binding fragment thereof, optionally wherein the level of the secreted TNFRSF member protein is measured one day, two days, three days, four days, five days, six days, one week, or more after the antibody or antigen-binding fragment thereof is administered.

144. The method of claim 143, wherein the method further comprises administering another dose of the antibody or antigen-binding fragment thereof to the subject if the level of the secreted version of the TNFRSF member protein is the same as, or greater than, a level of the secreted version of the TNFRSF member protein measured in the subject prior to administration of the antibody or antigen-binding fragment thereof.

145. A kit comprising an agent selected from the group consisting of the antibody or antigen-binding fragment thereof of any one of claims 1-34 and 40-61, the construct of any one of claims 63-66, the polynucleotide of claim 67 or 68, the vector of any one of claims 69-77, the host cell of any one of claims 78 and 80-82, or the pharmaceutical composition of any one of claims 83-107.

146. The kit of claim 145, wherein the kit comprises the antibody or antigen-binding fragment thereof of any one of claims 1-34 and 40-61.

147. The kit of claim 146, wherein the kit comprises the construct of any of claims 63-66.

148. The kit of claim 145, wherein the kit comprises the polynucleotide of claim 67 or 68.

149. The kit of claim 145, wherein the kit comprises the vector of any one of claims 69-77.

150. The kit of claim 149, wherein the kit further comprises instructions for transfecting the vector into a host cell.

151. The kit of claim 150, wherein the kit further comprises instructions for expressing the antibody, antigen-binding fragment thereof, or construct in the host cell.

152. The kit of claim 145, wherein the kit comprises the host cell of any one of claims 78 and 80-82.

153. The kit of claim 152, wherein the kit further comprises a reagent that can be used to express the antibody, antigen-binding fragment thereof, or construct in the host cell.

154. The kit of claim 145, wherein the kit comprises the pharmaceutical composition of any one of claims 83-107.

155. The kit of claim 145, further comprising instructions for administering the agent to a human subject.

156. The kit of claim 145, further comprising instructions for making or using the agent.

157. The kit of any one of claims 145-156, further comprising instructions for measuring a level of secreted soluble TNFRSF member protein in a subject.

158. An antibody or antigen-binding fragment thereof that specifically binds CD40, wherein the antibody or antigen-binding fragment thereof specifically binds an epitope comprising five or more of amino acids 124-132 of SEQ ID NO: 4 and/or five or more of amino acids 143-152 of SEQ ID NO: 4.

159. The antibody or antigen-binding fragment thereof of claim 158, wherein the antibody or antigen-binding fragment thereof comprises a non-native constant region.

160. The antibody or antigen-binding fragment thereof of claim 158 or 159, wherein the antibody or antigen-binding fragment thereof inhibits signaling associated with CD40.

161. The antibody or antigen-binding fragment thereof of any one of claims 158-160, wherein the antibody or antigen-binding fragment thereof binds CD40 with a K_{d} of no greater than about 10 nM.

162. The antibody or antigen-binding fragment thereof of claim 161, wherein the antibody or antigen-binding fragment thereof binds CD40 with a K_{d} of no greater than about 1 nM.

163. The antibody or antigen-binding fragment thereof of any one of claims 158-162, wherein the antibody or antigen-binding fragment thereof binds CD40 to form an antibody-antigen complex with a kₒₙ of at least about 10⁴ M⁻¹s⁻¹.

164. The antibody or antigen-binding fragment thereof of any one of claims 158-163, wherein the antibody or antigen-binding fragment thereof binds CD40 to form an antibody-antigen complex, and wherein the complex dissociates with a kₒₙ of no greater than about 10⁻³ s⁻¹.

165. The antibody or antigen-binding fragment thereof of any one of claims 158-164, wherein the antibody or antigen-binding fragment thereof has an isotype selected from the group consisting of IgG, IgA, IgM, IgD, and IgE.

166. The antibody or antigen-binding fragment thereof of claim 165, wherein the antibody or antigen-binding fragment thereof is an IgG isotype.

167. The antibody or antigen-binding fragment thereof of claim 166, wherein the antibody or antigen-binding fragment thereof is an IgG2 isotype.

168. The antibody or antigen-binding fragment thereof of any one of claims 158-167, wherein the antibody or antigen-binding fragment thereof comprises a human IgG2 hinge region that lacks a cysteine residue at positions 232 and/or 233 of the amino acid sequence of the IgG2 hinge region.

169. The antibody or antigen-binding fragment thereof of claim 168, wherein the antibody or antigen-binding fragment thereof comprises a human IgG2 hinge region that lacks a cysteine residue at position 232 of the amino acid sequence of the IgG2 hinge region.

170. The antibody or antigen-binding fragment thereof of claim 168 or 169, wherein the antibody or antigen-binding fragment thereof comprises a human IgG2 hinge region that lacks a cysteine residue at position 233 of the amino acid sequence of the IgG2 hinge region.

171. The antibody or antigen-binding fragment thereof of any one of claims 168-170, wherein the antibody or antigen-binding fragment thereof comprises a human IgG2 hinge region having an amino acid other than cysteine at positions 232 and/or 233 of the amino acid sequence of the IgG2 hinge region.

172. The antibody or antigen-binding fragment thereof of claim 171, wherein the antibody or antigen-binding fragment thereof comprises a human IgG2 hinge region having an amino acid other than cysteine at position 232 of the amino acid sequence of the IgG2 hinge region.

173. The antibody or antigen-binding fragment thereof of claim 171 or 172, wherein the antibody or antigen-binding fragment thereof comprises a human IgG2 hinge region having an amino acid other than cysteine at position 233 of the amino acid sequence of the IgG2 hinge region.

174. The antibody or antigen-binding fragment thereof of any one of claims 168-173, wherein the antibody or antigen-binding fragment thereof comprises a human IgG2 hinge region having a serine residue at positions 232 and/or 233 of the amino acid sequence of the IgG2 hinge region.

175. The antibody or antigen-binding fragment thereof of claim 174, wherein the antibody or antigen-binding fragment thereof comprises a human IgG2 hinge region having a serine residue at position 232 of the amino acid sequence of the IgG2 hinge region.

176. The antibody or antigen-binding fragment thereof of claim 174 or 175, wherein the antibody or antigen-binding fragment thereof comprises a human IgG2 hinge region having a serine residue at position 233 of the amino acid sequence of the IgG2 hinge region.

177. The antibody or antigen-binding fragment thereof of any one of claims 168-176, wherein the antibody or antigen-binding fragment thereof comprises a human IgG2 hinge region comprising an amino acid substitution or deletion at one or both of cysteine residues 232 and 233.

178. The antibody or antigen-binding fragment thereof of claim 177, wherein the IgG2 hinge region comprises an amino acid substitution at one or both of cysteine residues 232 and 233.

179. The antibody or antigen-binding fragment thereof of claim 178, wherein the IgG2 hinge region comprises an amino acid substitution at cysteine residue 232.

180. The antibody or antigen-binding fragment thereof of claim 178 or 179, wherein the IgG2 hinge region comprises an amino acid substitution at cysteine residue 233.

181. The antibody or antigen-binding fragment thereof of any one of claims 177-180, wherein the amino acid substitution is a conservative amino acid substitution.

182. The antibody or antigen-binding fragment thereof of claim 181, wherein the IgG2 hinge region comprises a C232S substitution.

183. The antibody or antigen-binding fragment thereof of claim 181 or 182, wherein the IgG2 hinge region comprises a C233S substitution.

184. The antibody or antigen-binding fragment thereof of any one of claims 158-183, wherein the antibody or antigen-binding fragment thereof comprises antigen-binding sites separated from one another by a distance of at least about 133 Å.

185. The antibody or antigen-binding fragment thereof of claim 184, wherein the antigen-binding sites are separated from one another by a distance of at least about 134 Å.

186. The antibody or antigen-binding fragment thereof of claim 185, wherein the antigen-binding sites are separated from one another by a distance of at least about 139 Å.

187. The antibody or antigen-binding fragment thereof of claim 186, wherein the antigen-binding sites are separated from one another by a distance of at least about 150 Å.

188. The antibody or antigen-binding fragment thereof of any one of claims 158-183, wherein the antigen-binding sites are separated from one another by a distance of from about 133 Å to about 150 Å.

189. The antibody or antigen-binding fragment thereof of claim 188, wherein the antigen-binding sites are separated from one another by a distance of from about 133 Å to about 145 Å.

190. The antibody or antigen-binding fragment thereof of claim 188, wherein the antigen-binding sites are separated from one another by a distance of from about 133 Å to about 139 Å.

191. The antibody or antigen-binding fragment thereof of claim 188, wherein the antigen-binding sites are separated from one another by a distance of from about 134 Å to about 139 Å.

192. The antibody or antigen-binding fragment thereof of any one of claims 158-191, wherein the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv).

193. The antibody or antigen-binding fragment thereof of claim 192, wherein the antibody or antigen-binding fragment thereof is a human, humanized, or chimeric antibody or antigen-binding fragment thereof.

194. The antibody or antigen-binding fragment thereof of any one of claims 158-193, wherein the antibody is conjugated to a therapeutic agent.

195. The antibody or antigen-binding fragment thereof of claim 194, wherein the therapeutic agent is a cytotoxic agent.

196. The antibody or antigen-binding fragment thereof of any one of claims 158-195, wherein the antibody or antigen-binding fragment thereof comprises a framework region from a human antibody or chimeric antibody.

197. The antibody or antigen-binding fragment thereof of any one of claims 158-196, wherein the antibody or antigen binding fragment thereof stabilizes an anti-parallel dimer conformation of CD40.

198. The antibody or antigen-binding fragment thereof of any one of claims 158-197, wherein the antibody or antigen-binding fragment thereof destabilizes a trimeric conformation of CD40.

199. The antibody or antigen-binding fragment thereof of any one of claims 158-198, wherein the antibody or antigen-binding fragment thereof reduces secretion of a soluble version of CD40.

200. The antibody or antigen-binding fragment thereof of any one of claims 158-199, wherein the antibody or antigen-binding fragment thereof inhibits expression of one of more genes selected from the group consisting of CHUK, NFκBIE, NFκBIA, MAP3K11, TRAF2, TRAF3, relB, and cIAP2/BIRC3.

201. The antibody or antigen-binding fragment thereof of any one of claims 158-200, wherein the antibody or antigen-binding fragment thereof promotes proliferation of T-reg cells.

202. The antibody or antigen-binding fragment thereof of any one of claims 158-201, wherein the antibody or antigen-binding fragment thereof directly kills, or promotes the death of, CD8+ cytotoxic T cells.

203. The antibody or antigen-binding fragment thereof of any one of claims 158-202, wherein the antibody or antigen-binding fragment thereof is capable of reducing or inhibiting proliferation of a population of CD8+ cytotoxic T cells in the presence of CD40L.

204. A construct comprising a first polypeptide domain and a second polypeptide domain, wherein the first polypeptide domain and the second polypeptide domain are each, independently, an antigen-binding fragment of any one of claims 158-203.

205. The construct of claim 204, wherein the first polypeptide domain and the second polypeptide domain are bound by a covalent linker.

206. The construct of claim 205, wherein the covalent linker comprises an amide bond.

207. The construct of claim 205, wherein the covalent linker comprises a disulfide bond.

208. A polynucleotide encoding the antibody or antigen-binding fragment thereof of any one of claims 158-203.

209. A polynucleotide encoding the construct of any one of claims 204-207.

210. A vector comprising the polynucleotide of claim 208 or 209.

211. The vector of claim 210, wherein the vector is an expression vector.

212. The vector of claim 211, wherein the expression vector is a eukaryotic expression vector.

213. The vector of claim 210, wherein the vector is a viral vector.

214. The vector of claim 213, wherein the viral vector is selected from the group consisting of adenovirus (Ad), retrovirus, poxvirus, adeno-associated virus, baculovirus, herpes simplex virus, and a vaccinia virus.

215. The vector of claim 214, wherein the adenovirus is a serotype 1-60 adenovirus.

216. The vector of claim 215, wherein the adenovirus is a serotype 5, 26, 35, or 48 adenovirus.

217. The vector of claim 214, wherein the retrovirus is a γ-retrovirus or a lentivirus.

218. The vector of claim 214, wherein the vaccinia virus is a modified vaccinia Ankara (MVA).

219. An isolated host cell comprising the vector of any one of claims 206-214.

220. The host cell of claim 219, wherein the host cell is a prokaryotic cell.

221. The host cell of claim 219, wherein the host cell is a eukaryotic cell.

222. The host cell of claim 221, wherein the eukaryotic cell is a mammalian cell.

223. The host cell of claim 222, wherein the mammalian cell is a CHO cell.

224. A pharmaceutical composition comprising the antibody or antigen-binding fragment thereof of any one of claims 158-203, the construct of any one of claims 204-207, the polynucleotide of claim 208 or 209, the vector of any one of claims 210-218, or the host cell of any one of claims 219 and 221-223, and a pharmaceutically acceptable carrier or excipient.

225. The pharmaceutical composition of claim 224, wherein the pharmaceutical composition comprises the antibody or antigen biding fragment thereof of any one of claims 158-203.

226. The pharmaceutical composition of claim 225, wherein at least 50% of the antibody or antigen-binding fragment thereof in the pharmaceutical composition is present in a single disulfide-bonded isoform.

227. The pharmaceutical composition of claim 226, wherein at least 75% of the antibody or antigen-binding fragment thereof in the pharmaceutical composition is present in a single disulfide-bonded isoform.

228. The pharmaceutical composition of claim 227, wherein at least 80% of the antibody or antigen-binding fragment thereof in the pharmaceutical composition is present in a single disulfide-bonded isoform.

229. The pharmaceutical composition of claim 228, wherein at least 85% of the antibody or antigen-binding fragment thereof in the pharmaceutical composition is present in a single disulfide-bonded isoform.

230. The pharmaceutical composition of claim 229, wherein at least 90% of the antibody or antigen-binding fragment thereof in the pharmaceutical composition is present in a single disulfide-bonded isoform.

231. The pharmaceutical composition of claim 230, wherein at least 95% of the antibody or antigen-binding fragment thereof in the pharmaceutical composition is present in a single disulfide-bonded isoform.

232. The pharmaceutical composition of claim 227, wherein from about 75% to about 99.9% of the antibody or antigen-binding fragment thereof in the pharmaceutical composition is present in a single disulfide-bonded isoform.

233. The pharmaceutical composition of claim 232, wherein from about 80% to about 99.9% of the antibody or antigen-binding fragment thereof in the pharmaceutical composition is present in a single disulfide-bonded isoform.

234. The pharmaceutical composition of claim 233, wherein from about 85% to about 99.9% of the antibody or antigen-binding fragment thereof in the pharmaceutical composition is present in a single disulfide-bonded isoform.

235. The pharmaceutical composition of claim 234, wherein from about 90% to about 99.9% of the antibody or antigen-binding fragment thereof in the pharmaceutical composition is present in a single disulfide-bonded isoform.

236. The pharmaceutical composition of claim 235, wherein from about 95% to about 99.9% of the antibody or antigen-binding fragment thereof in the pharmaceutical composition is present in a single disulfide-bonded isoform.

237. The pharmaceutical composition of any one of claims 224-236, wherein the antibody or antigen-binding fragment thereof yields a single detectable band upon gel electrophoresis analysis performed under non-reducing conditions.

238. The pharmaceutical composition of any one of claims 226-237, wherein the single disulfide-bonded isoform is IgG2-A.

239. The pharmaceutical composition of any one of claims 224-238, wherein the antibody or antigen-binding fragment thereof is present in the pharmaceutical composition in an amount of from about 0.001 mg/ml to about 100 mg/ml.

240. The pharmaceutical composition of any one of claims 224-239, wherein the pharmaceutical composition further comprises an additional therapeutic agent.

241. A method of inhibiting an immune response mediated by a B cell or CD8+ T cell in a human subject, the method comprising administering to the subject the antibody or antigen-binding fragment thereof of any one of claims 158-203, the construct of any one of claims 204-207, the polynucleotide of claim 208 or 209, the vector of any one of claims 210-218, the host cell of any one of claims 219 and 221-223, or the pharmaceutical composition of any one of claims 224-240.

242. A method of treating an immunological disease in a human subject, the method comprising administering to the subject the antibody or antigen-binding fragment thereof of any one of claims 158-203, the construct of any one of claims 204-207, the polynucleotide of claim 208 or 209, the vector of any one of claims 210-218, the host cell of any one of claims 219 and 221-223, or the pharmaceutical composition of any one of claims 224-240.

243. The method of claim 242, wherein the subject is in need of a tissue or organ regeneration.

244. The method of claim 243, wherein the tissue or organ is selected from the group consisting of a pancreas, salivary gland, pituitary gland, kidney, heart, lung, hematopoietic system, cranial nerves, heart, aorta, olfactory gland, ear, nerves, structures of the head, eye, thymus, tongue, bone, liver, small intestine, large intestine, gut, lung, brain, skin, peripheral nervous system, central nervous system, spinal cord, breast, embryonic structures, embryos, and testes.

245. The method of any one of claims 242-244, wherein the immunological disease is selected from the group consisting of an autoimmune disease, a neurological condition, an allergy, asthma, macular degeneration, muscular atrophy, a disease related to miscarriage, atherosclerosis, bone loss, a musculoskeletal disease, obesity, a graft-versus-host disease, and an allograft rejection.

246. The method of any one of claims 241-245, wherein the method further comprises measuring a level of secreted soluble CD40 in a subject.

247. The method of claim 246, wherein the method further comprises measuring a level of a secreted version of CD40 in a subject after the subject is administered the antibody or antigen-binding fragment thereof, optionally wherein the level of the secreted CD40 is measured one day, two days, three days, four days, five days, six days, one week, or more after the antibody or antigen-binding fragment thereof is administered.

248. The method of claim 247, wherein the method further comprises administering another dose of the antibody or antigen-binding fragment thereof to the subject if the level of the secreted version of CD40 is the same as, or greater than, a level of the secreted version of CD40 measured in the subject prior to administration of the antibody or antigen-binding fragment thereof.

249. A kit comprising an agent selected from the group consisting of the antibody or antigen-binding fragment thereof of any one of claims 158-203, the construct of any one of claims 204-207, the polynucleotide of claim 208 or 209, the vector of any one of claims 210-218, the host cell of any one of claims 219 and 221-223, or the pharmaceutical composition of any one of claims 224-240.

250. The kit of claim 249, wherein the kit comprises the antibody or antigen-binding fragment thereof of any one of claims 158-203.

251. The kit of claim 249, wherein the kit comprises the construct of any of claims 204-207.

252. The kit of claim 249, wherein the kit comprises the polynucleotide of claim 208 or 209.

253. The kit of claim 249, wherein the kit comprises the vector of any one of claims 210-218.

254. The kit of claim 253, wherein the kit further comprises instructions for transfecting the vector into a host cell.

255. The kit of claim 254, wherein the kit further comprises instructions for expressing the antibody, antigen-binding fragment thereof, or construct in the host cell.

256. The kit of claim 249, wherein the kit comprises the host cell of any one of claims 219 and 221-223.

257. The kit of claim 256, wherein the kit further comprises a reagent that can be used to express the antibody, antigen-binding fragment thereof, or construct in the host cell.

258. The kit of claim 249, wherein the kit comprises the pharmaceutical composition of any one of claims 224-240.

259. The kit of claim 249, further comprising instructions for administering the agent to a human subject.

260. The kit of claim 249, further comprising instructions for making or using the agent.

261. The kit of any one of claims 249-260, further comprising instructions for measuring a level of secreted soluble CD40 in a subject.

262. The antibody or antigen-binding fragment thereof of claim 1, wherein the antibody or antigen-binding fragment thereof has an isotype selected from the group consisting of IgG, IgA, IgM, IgD, and IgE.

263. The antibody or antigen-binding fragment thereof of claim 1, wherein the antibody or antigen-binding fragment thereof comprises a human IgG2 hinge region that lacks a cysteine residue at positions 232 and/or 233 of the amino acid sequence of the IgG2 hinge region.

264. The antibody or antigen-binding fragment thereof of claim 11, wherein the antibody or antigen-binding fragment thereof comprises a human IgG2 hinge region having a serine residue at positions 232 and/or 233 of the amino acid sequence of the IgG2 hinge region.

265. The antibody or antigen-binding fragment thereof of claim 1, wherein the antibody or antigen-binding fragment thereof comprises antigen-binding sites separated from one another by a distance of at least about 133 Å.

266. The antibody or antigen-binding fragment thereof of claim 1, wherein the antigen-binding sites are separated from one another by a distance of from about 133 Å to about 150 Å.

267. The antibody or antigen-binding fragment thereof of claim 1-34, wherein the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv).

268. The antibody or antigen-binding fragment thereof of claim 1, wherein the antibody or antigen binding fragment thereof stabilizes an anti-parallel dimer conformation of the TNFRSF member.

269. A method of reducing or inhibiting an immune response mediated by a T-reg cell in a human, the method comprising administering to the human the antibody or antigen-binding fragment thereof of claim 1, wherein the antibody or antigen-binding fragment thereof is an antagonist of TRAMP, DR6, TRAIL-R3, TRAIL-R4, RANK, LT Beta, HVEM, CD30, TROY, or RELT (19L).

270. A method of treating a cell proliferation disorder in a human, the method comprising administering to the human the antibody or antigen-binding fragment thereof of claim 1, wherein the antibody or antigen-binding fragment thereof is an antagonist of TRAMP, DR6, TRAIL-R3, TRAIL-R4, RANK, LT Beta, HVEM, CD30, TROY, or RELT (19L).

271. A method of treating an infectious disease in a human, the method comprising administering to the human the antibody or antigen-binding fragment thereof of claim 1, wherein the antibody or antigen-binding fragment thereof is an antagonist of TRAMP, DR6, TRAIL-R3, TRAIL-R4, HVEM, or RELT (19L).

272. A method of inhibiting an immune response mediated by a B cell or CD8+ T cell in a human subject comprising administering to the subject the antibody or antigen-binding fragment thereof of claim 1, wherein the antibody or antigen-binding fragment thereof is an antagonist of CD40, TRAIL-R1 (TNFRSF10A), TRAIL-R2 (TNFRSF10B), DR6, NGFR, TNFR1, Fas, EDAR, RANK, CD27, 4-1BB, OX40, GITR, or XEDAR.

273. A method of treating an immunological disease in a human subject comprising administering to the subject the antibody or antigen-binding fragment thereof of claim 1, wherein the antibody or antigen-binding fragment thereof is an antagonist of CD40, TRAIL-R1 (TNFRSF10A), TRAIL-R2 (TNFRSF10B), DR6, NGFR, TNFR1, Fas, EDAR, RANK, CD27, 4-1BB, OX40, GITR, or XEDAR.

274. A method of treating obesity, hyperlipidemia, and/or type 2 diabetes in a human subject, the method comprising administering to the subject the antibody or antigen-binding fragment thereof of claim 1, wherein the antibody or antigen-binding fragment thereof is an antagonist of Fas.

275. A method of treating a neurological disorder in a human subject, the method comprising administering to the subject the antibody or antigen-binding fragment thereof of claim 1, wherein the antibody or antigen-binding fragment thereof is an antagonist of DR6.

276. A method of treating osteoporosis or decreased bone loss in a human subject, the method comprising administering to the subject the antibody or antigen-binding fragment thereof of claim 1, wherein the antibody or antigen-binding fragment thereof is an antagonist of RANK.


SEQUENCE LISTING (154 sequences)

SEQ ID NO: 1 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 2 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 3 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 4 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 5 - protein, 655 aa, Homo sapiens
MGTRASSITALASCSRTAGQVGATMVAGSLLLLGFLSTITAQPEQKTLSLPGTYRHVDRTTGQVLTCDKCPAGTYVSEHCTNMSLRVCSSCPAGTFTRHENGIERCHDCSQPCPWPMIERLPCAALTDRECICPPGMYQSNGTCAPHTVCPVGWGVRKKGTENEDVRCKQCARGTFSDVPSSVMKCKAHTDCLGQNLEVVKPGTKETDNVCGMRLFFSSTNPPSSGTVTFSHPEHMESHDVPSSTYEPQGMNSTDSNSTASVRTKVPSGIEEGTVPDNTSSTSGKEGTNRTLPNPPQVTHQQAPHHRHILKLLPSSMEATGEKSSTAIKAPKRGHPRQNAHKHFDINEHLPWMIVLFLLLVLVLIVVCSIRKSSRTLKKGPRQDPSAIVEKAGLKKSLTPTQNREKWIYYRNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPMPSPNVKLENSTLLTVEPSPLDKNKCFFVDESEPLLRCDSTSSGSSALSRNGSFITKEKKDTVLRQVRLDPCDLQPIFDDMLHILNPEELRVIEEIPQAEDKLDRLFEIIGVKSQEASQTLLDSVYSHLPDLL

SEQ ID NO: 6 - protein, 448 aa, Homo sapiens
MAHVGDCTQTPWLPVLVVSLMCSARAEYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATALIIAMSTIFIMAIAIVLIIMFYILKTKPSAPACCTSHPGKSVEAQVSKDEEKKEAPDNVVMFSEKDEFEKLTATPAKPTKSENDASSENEQLLSRSVDSDEEPAPDKQGSPELCLLSLVHLAREKSATSNKSAGIQSRRKKILDVYANVCGVVEGLSPTELPFDCLEKTSRMLSSTYNSEKAVVKTWRHLAESFGLKRDEIGGMTDGMQLFDRISTAGYSIPELLTKLVQIERLDAVESLCADILEWAGVVPPASQPHAAS

SEQ ID NO: 7 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 8 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 9 - protein, 275 aa, Homo sapiens
MEPLPGWGSAPWSQAPTDNTFRLVPCVFLLNLLQRISAQPSCRQEEFLVGDECCPMCNPGYHVKQVCSEHTGTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYYVVSILLPLVIVGAGIAGFLICTRRHLHTSSVAKELEPFQEQQENTIRFPVTEVGFAETEEETASN

SEQ ID NO: 10 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 11 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 12 - protein, 400 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISC

SEQ ID NO: 13 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 14 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 15 - protein, 430 aa, Homo sapiens
MKPSLLCRPLSCFLMLLPWPLATLTSTTLWQCPPGEEPDLDPGQGTLCRPCPPGTFSAAWGSSPCQPHARCSLWRRLEAQVGMATRDTLCGDCWPGWFGPWGVPRVPCQPCSWAPLGTHGCDEWGRRARRGVEVAAGASSGGETRQPGNGTRAGGPEETAAQYAVIAIVPVFCLMGLLGILVCNLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 16 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 17 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 18 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 19 - protein, 386 aa, Homo sapiens
MGLWGQSVPTASSARAGRYPGARTASGTRPWLLDPKILKFVVFIVAVLLPVRVDSATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHYLIIIVVLVIILAVVVVGFSCRKKFISYLKGICSGGGGGPERVHRVLFRRRSCPSRVPGAEDNARNETLSNRYLQPTQVSEQEIQGQELAELTGVTVESPEEPQRLLEQAEAEGCQRRRLLVPVNDADSADISTLLDASATLEEGHAKETIQDQLVGSEKLFYEEDEAGSATSCL

SEQ ID NO: 20 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 21 - protein, 423 aa, Homo sapiens
MALKVLLEQEKTFFTLLVLLGYLSCKVTCESGDCRQQEFRDRSGNCVPCNQCGPGMELSKECGFGYGEDAQCVTCRLHRFKEDWGFQKCKPCLDCAVVNRFQKANCSATSDAICGDCLPGFYRKTKLVGFQDMECVPCGDPPPPYEPHCASKVNLVKIASTASSPRDTALAAVICSALATVLLALLILCVIYCKRQFMEKKPSWSLRSQDIQYNGSELSCFDRPQLHEYAHRACCQCRRDSVQTCGPVRLLPSMCCEEACSPNPATLGCGVHSAASLQARNAGPAGEMVPTFFGSLTQSICGEFSDAWPLMQNPMGGDNISFCDSYPELTGEDIHSLNPELESSTSLDSNSSQDLVGGAVPVQSHSENFTAATDLSRYNNTLVESASTQDALTMRSQLDQESGAVIHPATQTSLQVRQRLGSL

SEQ ID NO: 22 - protein, 287 aa, Homo sapiens
MDCQENEYWDQWGRCVTCQRCGPGQELSKDCGYGEGGDAYCTACPPRRYKSSWGHHRCQSCITCAVINRVQKVNCTATSNAVCGDCLPRFYRKTRIGGLQDQECIPCTKQTPTSEVQCAFQLSLVEADTPTVPPQEATLVALVSSLLVVFTLAFLGLFFLYCKQFFNRHCQRGGLLQFEADKTAKEESLFPVPPSKETSAESQVSENIFQTQPLNPILEDDCSSTSGFPTQESFTMASCTSESHSHWVHSPIECTELDLQAETLGGNTVESTGDRLELNVPFEVPSP

SEQ ID NO: 23 - protein, 12 aa, Artificial Sequence
  Synthetic Construct
ERKCCVESPPCP

SEQ ID NO: 24 - protein, 12 aa, Homo sapiens
ERKCCVECPPCP

SEQ ID NO: 25 - protein, 9 aa, Homo sapiens
SCSPGFGVK

SEQ ID NO: 26 - protein, 10 aa, Homo sapiens
CEPCPVGFFS

SEQ ID NO: 27 - protein, 29 aa, Homo sapiens
MCEQDCKQGQELTKKGCKDCCFGTFNDQK

SEQ ID NO: 28 - protein, 7 aa, Homo sapiens
QMCEPGT

SEQ ID NO: 29 - protein, 12 aa, Homo sapiens
QCDPCIPGVSFS

SEQ ID NO: 30 - protein, 12 aa, Homo sapiens
SVCPAGMIVKFP

SEQ ID NO: 31 - protein, 9 aa, Homo sapiens
CEPASPGVS

SEQ ID NO: 32 - protein, 16 aa, Homo sapiens
NGTCAPHTVCPVGWGV

SEQ ID NO: 33 - protein, 9 aa, Homo sapiens
KQCARGTFS

SEQ ID NO: 34 - protein, 12 aa, Homo sapiens
KDCEGFFRATVL

SEQ ID NO: 35 - protein, 12 aa, Homo sapiens
AECGPCLPGYYM

SEQ ID NO: 36 - protein, 9 aa, Homo sapiens
KCEHGIIKE

SEQ ID NO: 37 - protein, 9 aa, Homo sapiens
CKEEGSRSN

SEQ ID NO: 38 - protein, 10 aa, Homo sapiens
HPCPPGQGVQ

SEQ ID NO: 39 - protein, 10 aa, Homo sapiens
CIDCASGTFS

SEQ ID NO: 40 - protein, 11 aa, Homo sapiens
TTCPPGQRVEK

SEQ ID NO: 41 - protein, 9 aa, Homo sapiens
CADCLTGTF

SEQ ID NO: 42 - protein, 9 aa, Homo sapiens
DCPPGTEAE

SEQ ID NO: 43 - protein, 10 aa, Homo sapiens
CVPCKAGHFQ

SEQ ID NO: 44 - protein, 8 aa, Homo sapiens
CEAGSGLV

SEQ ID NO: 45 - protein, 11 aa, Homo sapiens
CEECPDGTYSD

SEQ ID NO: 46 - protein, 9 aa, Homo sapiens
SCPPGFGVV

SEQ ID NO: 47 - protein, 10 aa, Homo sapiens
CKRCPDGFFS

SEQ ID NO: 48 - protein, 17 aa, Homo sapiens
SYKPGVDCAPCPPGHFS

SEQ ID NO: 49 - protein, 8 aa, Homo sapiens
ECAPGLGA

SEQ ID NO: 50 - protein, 10 aa, Homo sapiens
CKPCLAGYFS

SEQ ID NO: 51 - protein, 8 aa, Homo sapiens
RCSLWRRL

SEQ ID NO: 52 - protein, 9 aa, Homo sapiens
CGDCWPGWF

SEQ ID NO: 53 - protein, 9 aa, Homo sapiens
KCRKEMGQV

SEQ ID NO: 54 - protein, 10 aa, Homo sapiens
VCGCRKNQYR

SEQ ID NO: 55 - protein, 11 aa, Homo sapiens
KCRTGCPRGMV

SEQ ID NO: 56 - protein, 9 aa, Homo sapiens
CVHKESGTK

SEQ ID NO: 57 - protein, 9 aa, Homo sapiens
ACKSDEEER

SEQ ID NO: 58 - protein, 9 aa, Homo sapiens
CQCKPGTFR

SEQ ID NO: 59 - protein, 9 aa, Homo sapiens
GCPRGMVKV

SEQ ID NO: 60 - protein, 10 aa, Homo sapiens
KNESAASSTG

SEQ ID NO: 61 - protein, 9 aa, Homo sapiens
LDCGALHRH

SEQ ID NO: 62 - protein, 10 aa, Homo sapiens
CGTCLPGFYE

SEQ ID NO: 63 - protein, 9 aa, Homo sapiens
QCGPGMELS

SEQ ID NO: 64 - protein, 11 aa, Homo sapiens
CVTCRLHRFKE

SEQ ID NO: 65 - protein, 8 aa, Homo sapiens
RCGPGQEL

SEQ ID NO: 66 - protein, 8 aa, Homo sapiens
TACPPRRY

SEQ ID NO: 67 - protein, 8 aa, Homo sapiens
CAPLRKCR

SEQ ID NO: 68 - protein, 5 aa, Homo sapiens
KCRPG

SEQ ID NO: 69 - protein, 8 aa, Homo sapiens
KCRPGFGV

SEQ ID NO: 70 - protein, 27 aa, Artificial Sequence
  Synethic Construct
  MOD_RES (3)..(3): Xaa is Cys residue protected with ACM protecting group
  MOD_RES (6)..(6): Xaa is Cys residue protected with ACM protecting group
  MOD_RES (22)..(22): Xaa is Cys residue protected with ACM protecting group
PEXLSXGSRCSGGSGGNRICTXRPGWY

SEQ ID NO: 71 - protein, 27 aa, Artificial Sequence
  Synthetic Construct
  MOD_RES (1)..(1): Xaa is Cys residue protected with ACM protecting group
  MOD_RES (4)..(4): Xaa is Cys residue protected with ACM protecting group
  MOD_RES (22)..(22): Xaa is Cys residue protected with ACM protecting group
XLSXGSRCSSDGGSGGNRICTXRPGWY

SEQ ID NO: 72 - protein, 27 aa, Artificial Sequence
  Synthetic Construct
  MOD_RES (4)..(4): Xaa is Cys residue protected with ACM protecting group
  MOD_RES (7)..(7): Xaa is Cys residue protected with ACM protecting group
  MOD_RES (22)..(22): Xaa is Cys residue protected with ACM protecting group
VPEXLSXGSRCGGSGGNRICTXRPGWY

SEQ ID NO: 73 - protein, 27 aa, Artificial Sequence
  Synthetic Construct
  MOD_RES (3)..(3): Xaa is Cys residue protected with ACM protecting group
  MOD_RES (22)..(22): Xaa is Cys residue protected with ACM protecting group
LSXGSRCSSDQGGSGGNRICTXRPGWY

SEQ ID NO: 74 - protein, 27 aa, Artificial Sequence
  Synthetic Construct
  MOD_RES (3)..(3): Xaa is Cys residue protected with ACM protecting group
  MOD_RES (6)..(6): Xaa is Cys residue protected with ACM protecting group
  MOD_RES (19)..(19): Xaa is Cys residue protected with ACM protecting group
  MOD_RES (25)..(25): Xaa is Cys residue protected with ACM protecting group
PEXLSXGSRCSGGSGGCTXRPGWYXAL

SEQ ID NO: 75 - protein, 27 aa, Artificial Sequence
  Synthetic Construct
  MOD_RES (2)..(2): Xaa is Cys residue protected with ACM protecting group
  MOD_RES (22)..(22): Xaa is Cys residue protected with ACM protecting group
SXGSRCSSDQVGGSGGNRICTXRPGWY

SEQ ID NO: 76 - protein, 27 aa, Artificial Sequence
  Synthetic Construct
  MOD_RES (1)..(1): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
XGSRCSSDQVEGGSGGRICTXRPGWYX

SEQ ID NO: 77 - protein, 27 aa, Artificial Sequence
  Synthetic Construct
  MOD_RES (2)..(2): Xaa is Cys residue protected with ACM protecting group
  MOD_RES (5)..(5): Xaa is Cys residue protected with ACM protecting group
  MOD_RES (19)..(19): Xaa is Cys residue protected with ACM protecting group
  MOD_RES (25)..(25): Xaa is Cys residue protected with ACM protecting group
EXLSXGSRCSSGGSGGCTXRPGWYXAL

SEQ ID NO: 78 - protein, 27 aa, Artificial Sequence
  Synthetic Construct
  MOD_RES (6)..(6): Xaa is Cys residue protected with ACM protecting group
  MOD_RES (17)..(17): Xaa is Cys residue protected with ACM protecting group
EGCRLXAPLRKGGSGGXAPLRKCRPGF

SEQ ID NO: 79 - protein, 27 aa, Artificial Sequence
  Synthetic Construct
  MOD_RES (1)..(1): Xaa is Cys residue protected with ACM protecting group
  MOD_RES (4)..(4): Xaa is Cys residue protected with ACM protecting group
  MOD_RES (19)..(19): Xaa is Cys residue protected with ACM protecting group
  MOD_RES (25)..(25): Xaa is Cys residue protected with ACM protecting group
XLSXGSRCSSDGGSGGCTXRPGWYXAL

SEQ ID NO: 80 - protein, 27 aa, Artificial Sequence
  Synthetic Construct
  MOD_RES (11)..(11): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
CSSDQVETQAXGGSGGRICTXRPGWYX

SEQ ID NO: 81 - protein, 27 aa, Artificial Sequence
  Synthetic Construct
  MOD_RES (21)..(21): Xaa is Cys residue protected with ACM protecting group
  MOD_RES (27)..(27): Xaa is Cys residue protected with ACM protecting group
GSRCSSDQVETGGSGGRICTXRPGWYX

SEQ ID NO: 82 - protein, 27 aa, Artificial Sequence
  Synthetic Construct
  MOD_RES (1)..(1): Xaa is Cys residue protected with ACM protecting group
  MOD_RES (22)..(22): Xaa is Cys residue protected with ACM protecting group
XGSRCSSDQVEGGSGGNRICTXRPGWY

SEQ ID NO: 83 - protein, 27 aa, Artificial Sequence
  Synthetic Construct
  MOD_RES (22)..(22): Xaa is Cys residue protected with ACM protecting group
GSRCSSDQVETGGSGGNRICTXRPGWY

SEQ ID NO: 84 - protein, 27 aa, Artificial Sequence
  Synthetic Construct
  MOD_RES (3)..(3): Xaa is Cys residue protected with ACM protecting group
  MOD_RES (6)..(6): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
PEXLSXGSRCSGGSGGRICTXRPGWYX

SEQ ID NO: 85 - protein, 27 aa, Artificial Sequence
  Synthetic Construct
  MOD_RES (4)..(4): Xaa is Cys residue protected with ACM protecting group
  MOD_RES (7)..(7): Xaa is Cys residue protected with ACM protecting group
  MOD_RES (23)..(23): Xaa is Cys residue protected with ACM protecting group
VPEXLSXGSRCGGSGGQNRICTXRPGW

SEQ ID NO: 86 - protein, 27 aa, Artificial Sequence
  Synthetic Construct
  MOD_RES (7)..(7): Xaa is Cys residue protected with ACM protecting group
  MOD_RES (17)..(17): Xaa is Cys residue protected with ACM protecting group
QEGCRLXAPLRGGSGGXAPLRKCRPGF

SEQ ID NO: 87 - protein, 27 aa, Artificial Sequence
  Synthetic Construct
  MOD_RES (21)..(21): Xaa is Cys residue protected with ACM protecting group
  MOD_RES (27)..(27): Xaa is Cys residue protected with ACM protecting group
SRCSSDQVETQGGSGGRICTXRPGWYX

SEQ ID NO: 88 - protein, 27 aa, Artificial Sequence
  Synthetic Construct
  MOD_RES (11)..(11): Xaa is Cys residue protected with ACM protecting group
  MOD_RES (17)..(17): Xaa is Cys residue protected with ACM protecting group
ALSKQEGCRLXGGSGGXAPLRKCRPGF

SEQ ID NO: 89 - protein, 27 aa, Artificial Sequence
  Synthetic Construct
  MOD_RES (7)..(7): Xaa is Cys residue protected with ACM protecting group
QEGCRLXAPLRGGSGGPLRKCRPGFGV

SEQ ID NO: 90 - protein, 27 aa, Artificial Sequence
  Synthetic Construct
  MOD_RES (2)..(2): Xaa is Cys residue protected with ACM protecting group
YXALSKQEGCRGGSGGAPLRKCRPGFG

SEQ ID NO: 91 - protein, 27 aa, Artificial Sequence
  Synthetic Construct
  MOD_RES (3)..(3): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
LSXGSRCSSDQGGSGGRICTXRPGWYX

SEQ ID NO: 92 - protein, 27 aa, Artificial Sequence
  Synthetic Construct
  MOD_RES (3)..(3): Xaa is Cys residue protected with ACM protecting group
  MOD_RES (6)..(6): Xaa is Cys residue protected with ACM protecting group
  MOD_RES (23)..(23): Xaa is Cys residue protected with ACM protecting group
PEXLSXGSRCSGGSGGQNRICTXRPGW

SEQ ID NO: 93 - protein, 27 aa, Artificial Sequence
  Synthetic Construct
  MOD_RES (21)..(21): Xaa is Cys residue protected with ACM protecting group
  MOD_RES (27)..(27): Xaa is Cys residue protected with ACM protecting group
RCSSDQVETQAGGSGGRICTXRPGWYX

SEQ ID NO: 94 - protein, 27 aa, Artificial Sequence
  Synthetic Construct
  MOD_RES (3)..(3): Xaa is Cys residue protected with ACM protecting group
  MOD_RES (6)..(6): Xaa is Cys residue protected with ACM protecting group
  MOD_RES (20)..(20): Xaa is Cys residue protected with ACM protecting group
  MOD_RES (26)..(26): Xaa is Cys residue protected with ACM protecting group
PEXLSXGSRCSGGSGGICTXRPGWYXA

SEQ ID NO: 95 - protein, 27 aa, Artificial Sequence
  Synthetic Construct
  MOD_RES (4)..(4): Xaa is Cys residue protected with ACM protecting group
  MOD_RES (7)..(7): Xaa is Cys residue protected with ACM protecting group
  MOD_RES (20)..(20): Xaa is Cys residue protected with ACM protecting group
  MOD_RES (26)..(26): Xaa is Cys residue protected with ACM protecting group
VPEXLSXGSRCGGSGGICTXRPGWYXA

SEQ ID NO: 96 - protein, 27 aa, Artificial Sequence
  Synthetic Construct
  MOD_RES (2)..(2): Xaa is Cys residue protected with ACM protecting group
  MOD_RES (19)..(19): Xaa is Cys residue protected with ACM protecting group
  MOD_RES (25)..(25): Xaa is Cys residue protected with ACM protecting group
SXGSRCSSDQVGGSGGCTXRPGWYXAL

SEQ ID NO: 97 - protein, 27 aa, Artificial Sequence
  Synthetic Construct
  MOD_RES (3)..(3): Xaa is Cys residue protected with ACM protecting group
WYXALSKQEGCGGSGGAPLRKCRPGFG

SEQ ID NO: 98 - protein, 27 aa, Artificial Sequence
  Synthetic Construct
  MOD_RES (10)..(10): Xaa is Cys residue protected with ACM protecting group
  MOD_RES (17)..(17): Xaa is Cys residue protected with ACM protecting group
LSKQEGCRLXAGGSGGXAPLRKCRPGF

SEQ ID NO: 99 - protein, 27 aa, Artificial Sequence
  Synthetic Construct
  MOD_RES (8)..(8): Xaa is Cys residue protected with ACM protecting group
  MOD_RES (17)..(17): Xaa is Cys residue protected with ACM protecting group
KQEGCRLXAPLGGSGGXAPLRKCRPGF

SEQ ID NO: 100 - protein, 27 aa, Artificial Sequence
  Synthetic Construct
  MOD_RES (3)..(3): Xaa is Cys residue protected with ACM protecting group
  MOD_RES (19)..(19): Xaa is Cys residue protected with ACM protecting group
  MOD_RES (25)..(25): Xaa is Cys residue protected with ACM protecting group
LSXGSRCSSDQGGSGGCTXRPGWYXAL

SEQ ID NO: 101 - protein, 27 aa, Artificial Sequence
  Synthetic Construct
  MOD_RES (23)..(23): Xaa is Cys residue protected with ACM protecting group
RCSSDQVETQAGGSGGQNRICTXRPGW

SEQ ID NO: 102 - protein, 27 aa, Artificial Sequence
  Synthetic Construct
  MOD_RES (9)..(9): Xaa is Cys residue protected with ACM protecting group
  MOD_RES (17)..(17): Xaa is Cys residue protected with ACM protecting group
SKQEGCRLXAPGGSGGXAPLRKCRPGF

SEQ ID NO: 103 - protein, 27 aa, Artificial Sequence
  Synthetic Construct
  MOD_RES (1)..(1): Xaa is Cys residue protected with ACM protecting group
  MOD_RES (23)..(23): Xaa is Cys residue protected with ACM protecting group
XGSRCSSDQVEGGSGGQNRICTXRPGW

SEQ ID NO: 104 - protein, 27 aa, Artificial Sequence
  Synthetic Construct
  MOD_RES (11)..(11): Xaa is Cys residue protected with ACM protecting group
  MOD_RES (23)..(23): Xaa is Cys residue protected with ACM protecting group
CSSDQVETQAXGGSGGQNRICTXRPGW

SEQ ID NO: 105 - protein, 27 aa, Artificial Sequence
  Synthetic Construct
  MOD_RES (11)..(11): Xaa is Cys residue protected with ACM protecting group
  MOD_RES (22)..(22): Xaa is Cys residue protected with ACM protecting group
CSSDQVETQAXGGSGGNRICTXRPGWY

SEQ ID NO: 106 - protein, 27 aa, Artificial Sequence
  Synthetic Construct
  MOD_RES (4)..(4): Xaa is Cys residue protected with ACM protecting group
  MOD_RES (7)..(7): Xaa is Cys residue protected with ACM protecting group
  MOD_RES (19)..(19): Xaa is Cys residue protected with ACM protecting group
  MOD_RES (25)..(25): Xaa is Cys residue protected with ACM protecting group
VPEXLSXGSRCGGSGGCTXRPGWYXAL

SEQ ID NO: 107 - protein, 27 aa, Artificial Sequence
  Synthetic Construct
  MOD_RES (2)..(2): Xaa is Cys residue protected with ACM protecting group
  MOD_RES (5)..(5): Xaa is Cys residue protected with ACM protecting group
  MOD_RES (23)..(23): Xaa is Cys residue protected with ACM protecting group
EXLSXGSRCSSGGSGGQNRICTXRPGW

SEQ ID NO: 108 - protein, 27 aa, Artificial Sequence
  Synthetic Construct
  MOD_RES (22)..(22): Xaa is Cys residue protected with ACM protecting group
SRCSSDQVETQGGSGGNRICTXRPGWY

SEQ ID NO: 109 - protein, 27 aa, Artificial Sequence
  Synthetic Construct
  MOD_RES (1)..(1): Xaa is Cys residue protected with ACM protecting group
  MOD_RES (19)..(19): Xaa is Cys residue protected with ACM protecting group
  MOD_RES (25)..(25): Xaa is Cys residue protected with ACM protecting group
XGSRCSSDQVEGGSGGCTXRPGWYXAL

SEQ ID NO: 110 - protein, 27 aa, Artificial Sequence
  Synthetic Construct
  MOD_RES (4)..(4): Xaa is Cys residue protected with ACM protecting group
  MOD_RES (7)..(7): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
VPEXLSXGSRCGGSGGRICTXRPGWYX

SEQ ID NO: 111 - protein, 27 aa, Artificial Sequence
  Synthetic Construct
  MOD_RES (2)..(2): Xaa is Cys residue protected with ACM protecting group
  MOD_RES (5)..(5): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
EXLSXGSRCSSGGSGGRICTXRPGWYX

SEQ ID NO: 112 - protein, 27 aa, Artificial Sequence
  Synthetic Construct
  MOD_RES (10)..(10): Xaa is Cys residue protected with ACM protecting group
LSKQEGCRLXAGGSGGRKCRPGFGVAR

SEQ ID NO: 113 - protein, 27 aa, Artificial Sequence
  Synthetic Construct
  MOD_RES (23)..(23): Xaa is Cys residue protected with ACM protecting group
GSRCSSDQVETGGSGGQNRICTXRPGW

SEQ ID NO: 114 - protein, 27 aa, Artificial Sequence
  Synthetic Construct
  MOD_RES (2)..(2): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
SXGSRCSSDQVGGSGGRICTXRPGWYX

SEQ ID NO: 115 - protein, 27 aa, Artificial Sequence
  Synthetic Construct
  MOD_RES (19)..(19): Xaa is Cys residue protected with ACM protecting group
  MOD_RES (25)..(25): Xaa is Cys residue protected with ACM protecting group
GSRCSSDQVETGGSGGCTXRPGWYXAL

SEQ ID NO: 116 - protein, 27 aa, Artificial Sequence
  Synthetic Construct
  MOD_RES (2)..(2): Xaa is Cys residue protected with ACM protecting group
YXALSKQEGCRGGSGGRKCRPGFGVAR

SEQ ID NO: 117 - protein, 27 aa, Artificial Sequence
  Synthetic Construct
  MOD_RES (8)..(8): Xaa is Cys residue protected with ACM protecting group
KQEGCRLXAPLGGSGGPLRKCRPGFGV

SEQ ID NO: 118 - protein, 27 aa, Artificial Sequence
  Synthetic Construct
  MOD_RES (11)..(11): Xaa is Cys residue protected with ACM protecting group
ALSKQEGCRLXGGSGGRKCRPGFGVAR

SEQ ID NO: 119 - protein, 27 aa, Artificial Sequence
  Synthetic Construct
  MOD_RES (2)..(2): Xaa is Cys residue protected with ACM protecting group
  MOD_RES (5)..(5): Xaa is Cys residue protected with ACM protecting group
  MOD_RES (20)..(20): Xaa is Cys residue protected with ACM protecting group
  MOD_RES (26)..(26): Xaa is Cys residue protected with ACM protecting group
EXLSXGSRCSSGGSGGICTXRPGWYXA

SEQ ID NO: 120 - protein, 27 aa, Artificial Sequence
  Synthetic Construct
  MOD_RES (11)..(11): Xaa is Cys residue protected with ACM protecting group
  MOD_RES (20)..(20): Xaa is Cys residue protected with ACM protecting group
  MOD_RES (26)..(26): Xaa is Cys residue protected with ACM protecting group
CSSDQVETQAXGGSGGICTXRPGWYXA

SEQ ID NO: 121 - protein, 27 aa, Artificial Sequence
  Synthetic Construct
  MOD_RES (6)..(6): Xaa is Cys residue protected with ACM protecting group
EGCRLXAPLRKGGSGGPLRKCRPGFGV

SEQ ID NO: 122 - protein, 27 aa, Artificial Sequence
  Synthetic Construct
  MOD_RES (11)..(11): Xaa is Cys residue protected with ACM protecting group
ALSKQEGCRLXGGSGGPLRKCRPGFGV

SEQ ID NO: 123 - protein, 27 aa, Artificial Sequence
  Synthetic Construct
  MOD_RES (1)..(1): Xaa is Cys residue protected with ACM protecting group
XALSKQEGCRLGGSGGRKCRPGFGVAR

SEQ ID NO: 124 - protein, 27 aa, Artificial Sequence
  Synthetic Construct
  MOD_RES (1)..(1): Xaa is Cys residue protected with ACM protecting group
  MOD_RES (17)..(17): Xaa is Cys residue protected with ACM protecting group
XALSKQEGCRLGGSGGXAPLRKCRPGF

SEQ ID NO: 125 - protein, 27 aa, Artificial Sequence
  Synthetic Construct
  MOD_RES (1)..(1): Xaa is Cys residue protected with ACM protecting group
  MOD_RES (4)..(4): Xaa is Cys residue protected with ACM protecting group
  MOD_RES (21)..(21): Xaa is Cys residue protected with ACM protecting group
  MOD_RES (27)..(27): Xaa is Cys residue protected with ACM protecting group
XLSXGSRCSSDGGSGGRICTXRPGWYX

SEQ ID NO: 126 - protein, 27 aa, Artificial Sequence
  Synthetic Construct
  MOD_RES (10)..(10): Xaa is Cys residue protected with ACM protecting group
LSKQEGCRLXAGGSGGPLRKCRPGFGV

SEQ ID NO: 127 - protein, 27 aa, Artificial Sequence
  Synthetic Construct
  MOD_RES (9)..(9): Xaa is Cys residue protected with ACM protecting group
SKQEGCRLXAPGGSGGRKCRPGFGVAR

SEQ ID NO: 128 - protein, 27 aa, Artificial Sequence
  Synthetic Construct
  MOD_RES (8)..(8): Xaa is Cys residue protected with ACM protecting group
KQEGCRLXAPLGGSGGRKCRPGFGVAR

SEQ ID NO: 129 - protein, 27 aa, Artificial Sequence
  Synthetic Construct
  MOD_RES (7)..(7): Xaa is Cys residue protected with ACM protecting group
QEGCRLXAPLRGGSGGRKCRPGFGVAR

SEQ ID NO: 130 - protein, 27 aa, Artificial Sequence
  Synthetic Construct
  MOD_RES (1)..(1): Xaa is Cys residue protected with ACM protecting group
XALSKQEGCRLGGSGGAPLRKCRPGFG

SEQ ID NO: 131 - protein, 27 aa, Artificial Sequence
  Synthetic Construct
  MOD_RES (1)..(1): Xaa is Cys residue protected with ACM protecting group
XALSKQEGCRLGGSGGPLRKCRPGFGV

SEQ ID NO: 132 - protein, 27 aa, Artificial Sequence
  Synthetic Construct
  MOD_RES (20)..(20): Xaa is Cys residue protected with ACM protecting group
  MOD_RES (26)..(26): Xaa is Cys residue protected with ACM protecting group
GSRCSSDQVETGGSGGICTXRPGWYXA

SEQ ID NO: 133 - protein, 27 aa, Artificial Sequence
  Synthetic Construct
  MOD_RES (23)..(23): Xaa is Cys residue protected with ACM protecting group
SRCSSDQVETQGGSGGQNRICTXRPGW

SEQ ID NO: 134 - protein, 27 aa, Artificial Sequence
  Synthetic Construct
  MOD_RES (1)..(1): Xaa is Cys residue protected with ACM protecting group
  MOD_RES (20)..(20): Xaa is Cys residue protected with ACM protecting group
  MOD_RES (26)..(26): Xaa is Cys residue protected with ACM protecting group
XGSRCSSDQVEGGSGGICTXRPGWYXA

SEQ ID NO: 135 - protein, 27 aa, Artificial Sequence
  Synthetic Construct
  MOD_RES (2)..(2): Xaa is Cys residue protected with ACM protecting group
  MOD_RES (5)..(5): Xaa is Cys residue protected with ACM protecting group
  MOD_RES (22)..(22): Xaa is Cys residue protected with ACM protecting group
EXLSXGSRCSSGGSGGNRICTXRPGWY

SEQ ID NO: 136 - protein, 27 aa, Artificial Sequence
  Synthetic Construct
  MOD_RES (20)..(20): Xaa is Cys residue protected with ACM protecting group
  MOD_RES (26)..(26): Xaa is Cys residue protected with ACM protecting group
SRCSSDQVETQGGSGGICTXRPGWYXA

SEQ ID NO: 137 - protein, 27 aa, Artificial Sequence
  Synthetic Construct
  MOD_RES (2)..(2): Xaa is Cys residue protected with ACM protecting group
YXALSKQEGCRGGSGGPLRKCRPGFGV

SEQ ID NO: 138 - protein, 27 aa, Artificial Sequence
  Synthetic Construct
  MOD_RES (20)..(20): Xaa is Cys residue protected with ACM protecting group
  MOD_RES (26)..(26): Xaa is Cys residue protected with ACM protecting group
RCSSDQVETQAGGSGGICTXRPGWYXA

SEQ ID NO: 139 - protein, 27 aa, Artificial Sequence
  Synthetic Construct
  MOD_RES (9)..(9): Xaa is Cys residue protected with ACM protecting group
SKQEGCRLXAPGGSGGPLRKCRPGFGV

SEQ ID NO: 140 - protein, 27 aa, Artificial Sequence
  Synthetic Construct
  MOD_RES (2)..(2): Xaa is Cys residue protected with ACM protecting group
  MOD_RES (23)..(23): Xaa is Cys residue protected with ACM protecting group
SXGSRCSSDQVGGSGGQNRICTXRPGW

SEQ ID NO: 141 - protein, 27 aa, Artificial Sequence
  Synthetic Construct
  MOD_RES (6)..(6): Xaa is Cys residue protected with ACM protecting group
EGCRLXAPLRKGGSGGRKCRPGFGVAR

SEQ ID NO: 142 - protein, 27 aa, Artificial Sequence
  Synthetic Construct
  MOD_RES (1)..(1): Xaa is Cys residue protected with ACM protecting group
  MOD_RES (4)..(4): Xaa is Cys residue protected with ACM protecting group
  MOD_RES (23)..(23): Xaa is Cys residue protected with ACM protecting group
XLSXGSRCSSDGGSGGQNRICTXRPGW

SEQ ID NO: 143 - protein, 27 aa, Artificial Sequence
  Synthetic Construct
  MOD_RES (3)..(3): Xaa is Cys residue protected with ACM protecting group
WYXALSKQEGCGGSGGPLRKCRPGFGV

SEQ ID NO: 144 - protein, 27 aa, Artificial Sequence
  Synthetic Construct
  MOD_RES (7)..(7): Xaa is Cys residue protected with ACM protecting group
QEGCRLXAPLRGGSGGAPLRKCRPGFG

SEQ ID NO: 145 - protein, 27 aa, Artificial Sequence
  Synthetic Construct
  MOD_RES (3)..(3): Xaa is Cys residue protected with ACM protecting group
  MOD_RES (17)..(17): Xaa is Cys residue protected with ACM protecting group
WYXALSKQEGCGGSGGXAPLRKCRPGF

SEQ ID NO: 146 - protein, 27 aa, Artificial Sequence
  Synthetic Construct
  MOD_RES (11)..(11): Xaa is Cys residue protected with ACM protecting group
ALSKQEGCRLXGGSGGAPLRKCRPGFG

SEQ ID NO: 147 - protein, 27 aa, Artificial Sequence
  Synthetic Construct
  MOD_RES (6)..(6): Xaa is Cys residue protected with ACM protecting group
EGCRLXAPLRKGGSGGAPLRKCRPGFG

SEQ ID NO: 148 - protein, 27 aa, Artificial Sequence
  Synthetic Construct
  MOD_RES (2)..(2): Xaa is Cys residue protected with ACM protecting group
  MOD_RES (17)..(17): Xaa is Cys residue protected with ACM protecting group
YXALSKQEGCRGGSGGXAPLRKCRPGF

SEQ ID NO: 149 - protein, 27 aa, Artificial Sequence
  Synthetic Construct
  MOD_RES (3)..(3): Xaa is Cys residue protected with ACM protecting group
  MOD_RES (23)..(23): Xaa is Cys residue protected with ACM protecting group
LSXGSRCSSDQGGSGGQNRICTXRPGW

SEQ ID NO: 150 - protein, 27 aa, Artificial Sequence
  Synthetic Construct
  MOD_RES (10)..(10): Xaa is Cys residue protected with ACM protecting group
LSKQEGCRLXAGGSGGAPLRKCRPGFG

SEQ ID NO: 151 - protein, 27 aa, Artificial Sequence
  Synthetic Construct
  MOD_RES (22)..(22): Xaa is Cys residue protected with ACM protecting group
RCSSDQVETQAGGSGGNRICTXRPGWY

SEQ ID NO: 152 - protein, 27 aa, Artificial Sequence
  Synthetic Construct
  MOD_RES (8)..(8): Xaa is Cys residue protected with ACM protecting group
KQEGCRLXAPLGGSGGAPLRKCRPGFG

SEQ ID NO: 153 - protein, 27 aa, Artificial Sequence
  Synthetic Construct
  MOD_RES (9)..(9): Xaa is Cys residue protected with ACM protecting group
SKQEGCRLXAPGGSGGAPLRKCRPGFG

SEQ ID NO: 154 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS